SEQUENCE LISTING (17 sequences)

SEQ ID NO: 1 - protein, 5 aa, synthetic construct
  REGION 1..5: note = Description of Artificial Sequence: Synthetic peptide
HYMMA

SEQ ID NO: 2 - protein, 17 aa, synthetic construct
  REGION 1..17: note = Description of Artificial Sequence: Synthetic peptide
RIGPSGGPTHYADSVKG

SEQ ID NO: 3 - protein, 19 aa, synthetic construct
  REGION 1..19: note = Description of Artificial Sequence: Synthetic peptide
YDSGYDYVAVAGPAEYFQH

SEQ ID NO: 4 - protein, 11 aa, synthetic construct
  REGION 1..11: note = Description of Artificial Sequence: Synthetic peptide
RASQSISTWLA

SEQ ID NO: 5 - protein, 7 aa, synthetic construct
  REGION 1..7: note = Description of Artificial Sequence: Synthetic peptide
KASNLHT

SEQ ID NO: 6 - protein, 9 aa, synthetic construct
  REGION 1..9: note = Description of Artificial Sequence: Synthetic peptide
QQYNSYSRT

SEQ ID NO: 7 - protein, 128 aa, synthetic construct
  REGION 1..128: note = Description of Artificial Sequence: Synthetic polypeptide
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSS

SEQ ID NO: 8 - protein, 107 aa, synthetic construct
  REGION 1..107: note = Description of Artificial Sequence: Synthetic polypeptide
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIK

SEQ ID NO: 9 - protein, 458 aa, synthetic construct
  REGION 1..458: note = Description of Artificial Sequence: Synthetic polypeptide
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 457 aa, synthetic construct
  REGION 1..457: note = Description of Artificial Sequence: Synthetic polypeptide
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, synthetic construct
  REGION 1..214: note = Description of Artificial Sequence: Synthetic polypeptide
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 458 aa, synthetic construct
  REGION 1..458: note = Description of Artificial Sequence: Synthetic polypeptide
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - protein, 457 aa, synthetic construct
  REGION 1..457: note = Description of Artificial Sequence: Synthetic polypeptide
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 14 - protein, 214 aa, synthetic construct
  REGION 1..214: note = Description of Artificial Sequence: Synthetic polypeptide
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 15 - protein, 458 aa, synthetic construct
  REGION 1..458: note = Description of Artificial Sequence: Synthetic polypeptide
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 16 - protein, 457 aa, synthetic construct
  REGION 1..457: note = Description of Artificial Sequence: Synthetic polypeptide
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 17 - protein, 214 aa, synthetic construct; identical to SEQ ID NO: 14
  REGION 1..214: note = Description of Artificial Sequence: Synthetic polypeptide

(19) United States
(12) Patent Application Publication
(10) Pub. No.: US 2024/0165251 A1
(43) Pub. Date: May 23, 2024

(54) IMMUNOSTIMULATORY COMPOUNDS AND CONJUGATES

(71) Applicants: Seagen Inc., Bothell, WA (US); Regents of the University of Minnesota, Minneapolis, MN (US)

(72) Inventors: Kung-Pern WANG, Woodinville, WA (US); Alyson SMITH, Snohomish, WA (US); Christopher Scott NEUMANN, Seattle, WA (US); Shyra J. GARDAI, Monroe, WA (US); David FERGUSON, Minneapolis, MN (US)

(21) Appl. No.: 18/228,550

(22) Filed: Jul. 31, 2023

Related U.S. Application Data
(63) Continuation of application No. PCT/US2022/015157, filed on Feb. 3, 2022.
(60) Provisional application No. 63/145,367, filed on Feb. 3, 2021.

(51) Int. Cl. A61K 47/68; A61P 35/00; C07D 471/04; C07H 15/203; C07K 16/28

(52) U.S. Cl. CPC A61K 47/6803; A61K 47/6849; A61P 35/00; C07D 471/04; C07H 15/203; C07K 16/2866

(57) ABSTRACT

The present disclosure provides, inter alia, antibody drug conjugates that are useful in treating various diseases such as cancer. The antibody drug conjugates can be configured to elicit tumor site-specific responses, including tumor microenvironment immunostimulation, while limiting off-target and systemic effects. In certain embodiments disclosed herein, the antibody drug conjugates are configured to release payloads following internalization by immune, cancer, or tumor associated cells.

### CROSS REFERENCE TO RELATED APPLICATION

This application claims priority from U.S. Provisional Application No. 63/145,367, filed Feb. 3, 2021, which is hereby incorporated by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Toll-like receptors (TLRs) play an important role in activating the immune system against invading pathogens and against damaged cells that may lead to cancer. See Kaczanowska, et al., J. Leukoc. Biol. 2013; 93(6): 847-863. TLRs are a family of single-pass membrane bound proteins that, when activated, recuit adaptor proteins to propagate the antigen-induced signal transduction pathway. As immunostimulatory compounds, TLR agonists have been developed as vaccine adjuvants, to boost production of immune cells that target the desired viral or bacterial antigen in the vaccine. TLRs also recognize endogenous markers of tumorigenesis such as cell death and chronic inflammation and activate an innate immune response to these cells. See, e.g., Ellerman, et al., Clin. Cancer Res. 2007; 13: 2836-2848; Hernandez, et al., Oncogene 2016; 35: 5931-5941; and Urban-Wojciuk, et al., Front. Immunol., Vol. 10, pp. 2388-2398 (2019). However, due to their immunostimulatory properties, systemic use of TLR agonists may be restricted by dose-limiting toxicities resulting from systemic cytokine induction. See Adams, S., Immunotherapy 2009; 1(6): 949-964. Thus, there remains a need for targeted immunostimulatory compounds to localize the immune response to the desired cells while minimizing off-target effects.

### SUMMARY

The present disclosure provides compounds and biomolecular complexes (e.g., antibody-drug conjugates) which elicit cell- and tissue-specific immune responses. As cancer immunosuppression is often localized within microenvironments proximal to the cancerous cells, such targeted molecules and complexes can activate immune responses at cancer sites while minimizing undesired responses in healthy tissues. To achieve this end, in certain embodiments, the present disclosure provides antibody-drug conjugates (ADCs) configured to selectively activate immune responses in the presence of cancer cells. In addition to localizing to targeted cancer sites, ADCs of the present disclosure can be configured to release their payloads (e.g., TLR agonists) only within the presence of or upon uptake by the cancerous (or cancer-associated) cells, thereby limiting immune activation to cancer sites, and preventing off-target (e.g., broad systemic) immune activation.

The ADCs described herein, as well as pharmaceutically acceptable salts thereof, can be configured for uptake by a target cell or tissue. In some embodiments, an ADC is configured to endocytose upon binding to a membrane bound and/or surface displayed antigen. In such cases, the ADC may target intracellular receptors such as TLR7 or TLR8, which are often primarily localized within endosomes. Endocytosis can be aided by lipophilic groups appended to the ADC, such as PEGylated or neutral and nonpolar peptidic linkers.

In addition to targeting, release of an ADC drug unit can be controlled such that the release occurs at a designated site (e.g, within a cell targeted by the antibody). As a linker (L) cleavable group can be configured for cleavage within particular physiological conditions or by specific enzymes, release of the Drug Unit can be limited primarily to the target site. Accordingly, the biological effects of the Drug Unit (such as immunostimulatory effects) can be localized to a target site. Alternatively, the Drug Unit can be configured to remain attached to the antibody, or a portion of the antibody and/or linker and induce its biological effect while coupled to the antibody.

In various embodiments, the present disclosure provides antibody drug conjugates (ADC) having the structure:

Ab-(L-D)ₚ
or a pharmaceutically acceptable salt thereof;
   wherein:
Ab is an antibody as defined herein;
each L is a linker as defined herein;
wherein each D is conjugated to a linker as described herein;
wherein each L is covalently attached to Ab via a sulfur atom of a cysteine residue or an ϵ-amino group of a lysine residue as described herein;
subscript p is as defined herein.

In some aspects, each D has the structure of Formula (A):

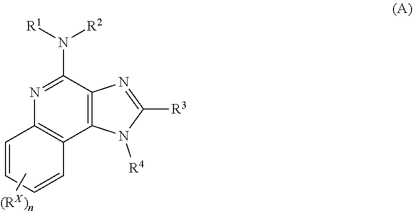

or a pharmaceutically acceptable salt thereof;
   wherein each of R¹, R², R³, R⁴, R^{x}, and n are as defined herein.

In other aspects, each D has the structure of Formula (I):

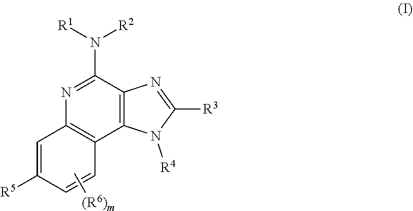

or a pharmaceutically acceptable salt thereof;
wherein R¹, R², R³, R⁴, R⁵, R⁶, and m are as defined here.

In further embodiments, each D has the structure of Formula (II):

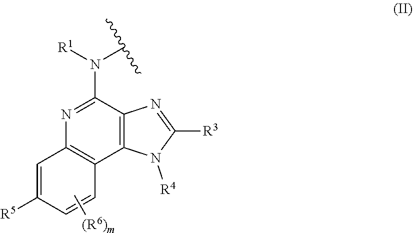

or a pharmaceutically acceptable salt thereof;
wherein R¹, R³, R⁴, R⁵, R⁶, and m are as defined herein.

In other embodiments, each D has the structure of Formula (III):

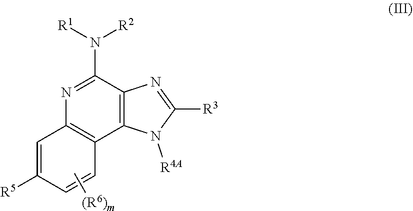

or a pharmaceutically acceptable salt thereof;
wherein R¹, R², R³, R^{4A}, R⁵, R⁶, and m are as defined herein.

In some embodiments, each D has the structure of Formula (IV):

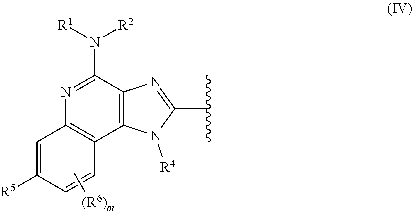

or a pharmaceutically acceptable salt thereof;
wherein R¹, R², R⁴, R⁵, R⁶, and m are as defined herein.

In further embodiments, each D has the structure of Formula (V):

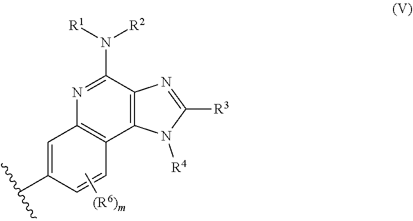

or a pharmaceutically acceptable salt thereof;
wherein R¹, R², R³, R⁴, R⁶, and m are as defined herein.

In some aspects, each D has the structure of Formula (VI):

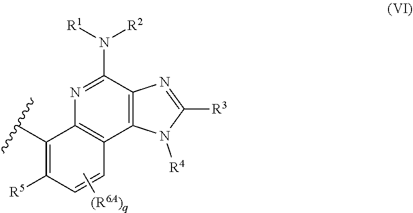

or a pharmaceutically acceptable salt thereof;
wherein R¹, R², R³, R⁴, R⁵, R^{6A}, and q are as defined herein.

In certain embodiments, each D has the structure of Formula (VII):

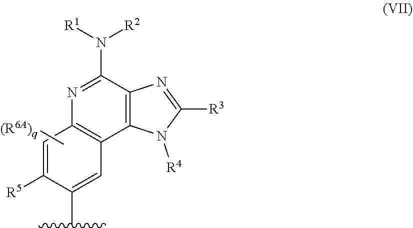

or a pharmaceutically acceptable salt thereof;
wherein R¹, R², R³, R⁴, R⁵, R^{6A}, and q are as defined herein.

In some aspects, each D has the structure of Formula (VIII):

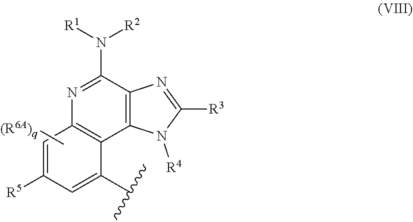

or a pharmaceutically acceptable salt thereof;
wherein R¹, R², R³, R⁴, R⁵, R^{6A}, and q are as defined herein.

In some embodiments, each D has the structure of Formula (A):

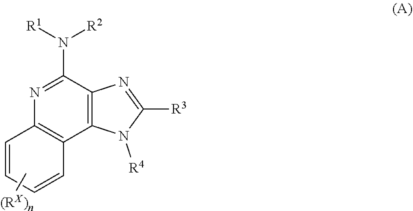

or a pharmaceutically acceptable salt thereof;
wherein R¹, R², R³, R⁴, R^{x}, and n are as defined herein.

In some aspects, each D has the structure of Formula (XI):

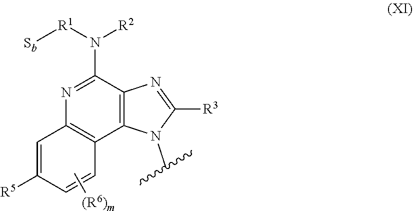

or a pharmaceutically acceptable salt thereof;
wherein Sb, R¹, R², R³, R⁵, R⁶, and m are as defined herein.

In further aspects, the present disclosure provides compounds of Formula (IX):

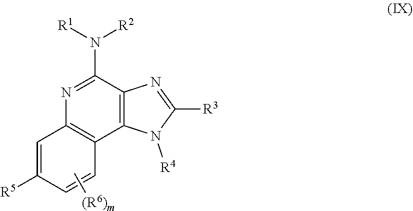

or a pharmaceutically acceptable salt thereof;
wherein R¹, R², R³, R⁴, R⁵, R⁶, and m are as defined herein.

In further aspects, the present disclosure provides compounds of Formula (IX-A):

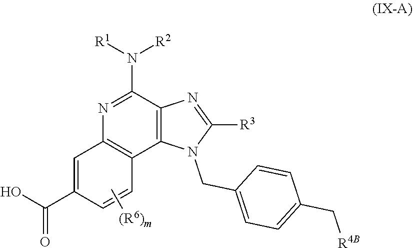

or a pharmaceutically acceptable salt thereof,
wherein R¹, R², R³, R^{4B}, R⁶, and m are as defined herein.

In other aspects, the present disclosure provides compounds having the structure of Formula (IX-B):

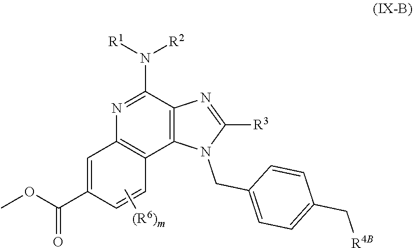

or a pharmaceutically acceptable salt thereof;
wherein R¹, R², R³, R^{4B}, R⁶, and m are as defined herein.

In other aspects, the present disclosure provides compounds having the formula L¹-D, or a pharmaceutically acceptable salt thereof, wherein:
L¹ is a linker intermediate as defined herein; and
D has the structure of Formula (X):

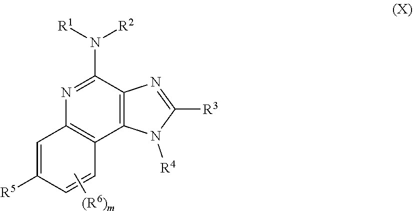

or a pharmaceutically acceptable salt thereof;
wherein R¹, R², R³, R⁴, R⁵, R⁶, and m are as defined herein.

In further aspects, the present disclosure provides compounds having the formula L^{I}-D, or a pharmaceutically acceptable salt thereof, wherein:
L¹ is a linker intermediate; and
D is a compound of Formula (X):

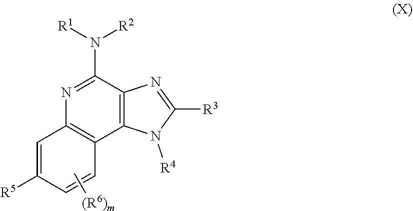

or a pharmaceutically acceptable salt thereof;
wherein R¹, R², R³, R⁴, R⁵, R⁶, and m are as defined herein.

Additional aspects of the present disclosure provide methods of making and using the compounds of the present disclosure.

### BRIEF DESCRIPTION OF THE FIGURES

FIG. 1 illustrates the responses of human PBMCs from a single donor to selected small molecule TLR7/8 agonists.

FIG. 2 illustrates the average response of human PBMCs isolated from 3 donors to selected small molecule TLR7/8 agonists.

FIG. 3 illustrates the responses of human PBMCs to selected TLR7/8 agonists having either a carboxylic acid (S5b, S8b, S11b) or methyl ester functional group (S5a, S8a, S11a).

FIG. 4 illustrates the responses of human immune cells to various small molecule TLR7/8 agonists.

FIG. 5 illustrates the responses of human immune cells to selected TLR7/8 agonists having either a carboxylic acid (S18b) or methyl ester functional group (S18a).

FIG. 6 illustrates the responses of human immune cells to various TLR7/8 agonists conjugated to a targeting antibody.

FIG. 7 illustrates the responses of human immune cells to selected TLR7/8 agonists having either a carboxylic acid (S18b) or methyl ester functional group (S18a) conjugated to a targeting antibody.

FIG. 8 illustrates the responses of human immune cells to selected TLR7/8 agonists having either a carboxylic acid (S14b, S18b, S76b) or methyl ester functional group (S14a, S18a, S76a) conjugated to a targeting antibody.

FIG. 9 illustrates the comparison of level of aggregation of various conjugated immunostimulatory ADCs.

FIG. 10 illustrates the anti-tumor responses of mice bearing CT26 tumors to treatment with S8a or S8b immune cell targeted antibody drug conjugates.

FIG. 11 illustrates the anti-tumor responses of mice bearing CT26 tumors to treatment with S14a or S14b immune cell targeted antibody drug conjugates.

FIG. 12 illustrates the anti-tumor responses of mice bearing MC38 tumors to treatment with S8a, S8b, S18a or S18b antibody drug conjugates.

FIG. 13 illustrates the anti-tumor responses of mice bearing CT26 tumors to treatment with S18b antibody drug conjugates with either a targeting or a non-targeting antibody.

FIG. 14 illustrates the anti-tumor responses of mice bearing Renca tumors to treatment with S18a free drug, and S18a and S18b antibody drug conjugates.

FIG. 15 illustrates cytokine responses of non-tumor bearing mice to treatment with S18b free drug and S18b antibody drug conjugates linked via N1 or C4 linkage.

FIG. 16 illustrates the cytokine response of non-tumor bearing mice to treatment with S18b free drug and S18b antibody drug conjugates linked via N1 or C4 linkage.

FIG. 17 illustrates the cytokine response of CT26-tumor bearing mice to treatment with S18b antibody drug conjugates linked via N1 or C4 linkage and with targeting or non-targeting antibody.

FIG. 18 illustrates the anti-tumor response of CT26-bearing mice to treatment with S18b antibody drug conjugates linked via N1 or C4 linkage and with targeting or non-targeting antibody.

FIG. 19 illustrates the cytokine response of Renca-tumor bearing mice to treatment with S18b drug conjugates linked via N1 or C4 linkage at 2 mg/kg dose.

FIG. 20 illustrates the anti-tumor response of Renca-bearing mice to treatment with S18b antibody drug conjugates linked via N1 or C4 linkage at 2 mg/kg dose.

FIG. 21 illustrates the activation of in vitro human immune cells in response to TLR7/8 antibody conjugates linked via N1 or C4 positions.

FIG. 22 illustrates the anti-tumor response of Renca-bearing mice to treatment with S18b antibody drug conjugates linked via N1 or C4 linkage at different dose levels.

FIG. 23 illustrates the cytokine response of Renca-tumor bearing mice to treatment with S18b antibody drug conjugates via N1 or C4 linkage at different dose levels.

FIG. 24 illustrates the in vitro assessment of TLR7 vs. TLR8 selectivity for various TLR7/8 small molecule agonists in HEK Blue hTLR7 and TLR8 cells.

FIG. 25 illustrate the in vivo assessment of TLR7 vs. TLR8 selectivity for various TLR7/8 small molecule agonists in C57BL/6 mice bearing subcutaneous MC38 tumors.

FIG. 26 illustrate the anti-tumor response of Renca-bearing mice to treatment with S18a antibody drug conjugates with different linkage sites and with or without a PEG group in the linker.

FIG. 27 illustrates the anti-tumor response of Renca-bearing mice to treatment with S18a antibody drug conjugates linked at the N1 or C4 positions, with different linker configurations, linkage sites and with or without a PEG group in the linker.

FIG. 28 provides tumor sizes in mice treated with targeted antibody-TLR7/8 agonist complexes.

FIG. 29 provides IL6 (top left), IL1b (top right), MIP1b (bottom left), and TNFα (bottom right) responses in human peripheral blood mononuclear cells (PBMCs) elicited with multiple imidazoquinoline complexes.

FIG. 30 summarizes cytokine responses in human immune cells following imidazoquinoline treatment. The top left panel summarizes IL6 levels following treatment, the top right panel summarizes TNFα levels following treatment, the bottom left panel summarizes MCP1 levels following treatment, and the bottom right panel summarizes IP10 levels following treatment.

FIG. 31 summarizes tumor size profiles over time post tumor implantation, in TLR7 positive and TLR7 knockout mice. The top left panel summarizes tumor growth in TLR7 positive mice, the bottom left panel summarizes tumor growth in TLR7 knockout mice, and the right panel summarizes tumor volumes on Day 36 post implantation for all mice.

FIG. 32 provides tumor sizes in mice bearing Renca tumors following treatment with TLR7/8 agonists.

FIG. 33 provides Renca tumor volumes in untreated mice, mice treated with an imidazoquinoline TLR agonist-coupled to a tumor targeted antibody or isotype control.

FIG. 34 summarizes Renca tumor volumes in untreated mice, mice treated with an imidazoquinoline TLR agonist C4-coupled to a tumor targeted antibody, or isotype control.

FIG. 35 summarizes CT26 tumor volumes in untreated mice, mice treated with an imidazoquinoline TLR agonist N1-coupled to a tumor targeted antibody, or isotype control.

FIG. 36 provides CT26 tumor volumes in untreated mice, mice treated with an imidazoquinoline TLR agonist C4-coupled to a tumor targeted antibody, or isotype control.

FIG. 37 summarizes 4T1 tumor volumes in untreated mice, mice treated with a bare EphA2-targeted antibody, and mice treated with a TLR7/8 agonist EphA2-targeted antibody conjugate.

FIG. 38 provides CT26 tumor volumes for untreated mice, mice treated with a tumor and immune targeting antibody alone, mice treated with a non-targeted isotype antibody conjugated to a TLR7/8 agonist, and mice treated with a TLR7/8 agonist conjugated to the tumor and immune targeting antibody.

### DETAILED DESCRIPTION

Many cancers are immunosuppresive, actively inhibiting immune cell proliferation, signalling, and activity to avoid detection and cytotoxic responses. While certain TLR agonists can reactivate cancer-suppressed immune cells, extensive use of TLR agonists is often restricted by dose-limiting toxicities resulting from systemic cytokine induction. Sustained TLR activation, even from mild agonist doses, can affect immune-related adverse events, including rheumatic and thyroid disorders, as well as nauseau, rashes, and general discomfort.

Provided herein are antibody immunostimulatory-drug conjugates (ADCs) that can elicit a localized immune response to target cells, and hence, reduced off-target toxicity, for example, as compared to the toxicity often observed with systemic administration of immunostimutory compounds, such as TLR agonists. The in vivo toxicity of such compounds is often linked to systemic cytokine activation, resulting in both on- and off-target immune responses. The ADCs described herein include TLR7/8 agonists that can provide selective induction of cytokines which may impart particular benefits for both monotherapy and combination therapies with ADCs. See, e.g., Schiaffo et al., J. Med. Chem. 2014; 57: 339-347; and Shi et al., Med. Chem. Lett. 2012; 3: 501-504. This approach can enable specific TLR activation, as well as localized immune cell recruitment, while reducing systemic cytokine release and its concomitant adverse effects and maintaining activity.

### Definitions

Unless otherwise defined, all technical and scientific terms used herein have the same meaning as commonly understood by one of ordinary skill in the art to which this disclosure belongs. Methods and materials are described herein for use in the present application; other, suitable methods and materials known in the art in some aspects of this disclosure are also used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control. When trade names are used herein, the trade name includes the product formulation, the generic drug, and the active pharmaceutical ingredient(s) of the trade name product, unless otherwise indicated by context.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a linker” includes reference to one or more such linkers, and reference to “the cell” includes reference to a plurality of such cells.

The term “about” when referring to a number or a numerical range means that the number or numerical range referred to is an approximation, for example, within experimental variability and/or statistical experimental error, and thus the number or numerical range may vary up to ±10% of the stated number or numerical range. In reference to an ADC composition comprising a distribution of ADCs as described herein, the average number of conjugated TLR agonist compounds to an antibody in the composition can be an integer or a non-integer, particularly when the antibody is to be partially loaded. Thus, the term “about” recited prior to an average drug loading value is intended to capture the expected variations in drug loading within an ADC composition.

The term “antibody” as used herein covers intact monoclonal antibodies, polyclonal antibodies, monospecific antibodies, multispecific antibodies (e.g., bispecific antibodies), including intact antibodies and antigen binding antibody fragments, and reduced forms thereof in which one or more of the interchain disulfide bonds are disrupted, that exhibit the desired biological activity and provided that the antigen binding antibody fragments have the requisite number of attachment sites for the desired number of attached groups, such as a linker (L), as described herein. In some aspects, the linkers are attached via a succinimide or hydrolyzed succinimide to the sulfur atoms of cysteine residues of reduced interchain disulfide bonds and/or cysteine residues introduced by genetic engineering. The native form of an antibody is a tetramer and consists of two identical pairs of immunoglobulin chains, each pair having one light chain and one heavy chain. In each pair, the light and heavy chain variable domains (VL and VH) are together primarily responsible for binding to an antigen. The light chain and heavy chain variable domains consist of a framework region interrupted by three hypervariable regions, also called “complementarity determining regions” or “CDRs.” The light chain and heavy chains also contain constant regions that may be recognized by and interact with the immune system. (see, e.g., Janeway et al., 2001, Immuno. Biology, 5th Ed., Garland Publishing, New York). An antibody includes any isotype (e.g., IgG, IgE, IgM, IgD, and IgA) or subclass (e.g., IgG1, IgG2, IgG3, IgG4, IgA1 and IgA2) thereof. The antibody is derivable from any suitable species. In some aspects, the antibody is of human or murine origin, and in some aspects the antibody is a human, humanized or chimeric antibody. Antibodies can be fucosylated to varying extents or afucosylated.

An “intact antibody” is one which comprises an antigen-binding variable region as well as light chain constant domains (C_{L}) and heavy chain constant domains, C_{H}1, C_{H}2, C_{H}3 and C_{H}4, as appropriate for the antibody class. The constant domains are either native sequence constant domains (e.g., human native sequence constant domains) or amino acid sequence variants thereof.

An “antibody fragment” comprises a portion of an intact antibody, comprising the antigen-binding or variable region thereof. Antibody fragments of the present disclosure include at least one cysteine residue (natural or engineered) that provides a site for attachment of a linker and/or linker-drug compound. In some embodiments, an antibody fragment includes Fab, Fab′, or F(ab′)₂.

As used herein the term “engineered cysteine residue” or “eCys residue” refers to a cysteine amino acid or a derivative thereof that is incorporated into an antibody. In those aspects one or more eCys residues can be incorporated into an antibody, and typically, the eCys residues are incorporated into either the heavy chain or the light chain of an antibody. Generally, incorporation of an eCys residue into an antibody is performed by mutagenizing a nucleic acid sequence of a parent antibody to encode for one or more amino acid residues with a cysteine or a derivative thereof. Suitable mutations include replacement of a desired residue in the light or heavy chain of an antibody with a cysteine or a derivative thereof, incorporation of an additional cysteine or a derivative thereof at a desired location in the light or heavy chain of an antibody, as well as adding an additional cysteine or a derivative thereof to the N- and/or C-terminus of a desired heavy or light chain of an amino acid. Further information can be found in U.S. Pat. No. 9,000,130, the contents of which are incorporated herein in its entirety. Derivatives of cysteine (Cys) include but are not limited to beta-2-Cys, beta-3-Cys, homocysteine, and N-methyl cysteine.

In some embodiments, the antibodies of the present disclosure include those having one or more engineered cysteine (eCys) residues. In some embodiments, derivatives of cysteine (Cys) include, but are not limited to beta-2-Cys, beta-3-Cys, homocysteine, and N-methyl cysteine.

In some embodiments, the antibodies of the present disclosure include those having one or more engineered lysine (eLys)residues. In some embodiments, one or more native lysine and/or eLys residues are activated prior to conjugation with a drug-linker intermediate (to form an ADC, as described herein). In some embodiments, the activation comprises contacting the antibody with a compound comprising a succinimydyl ester and a functional group selected from the group consisting of: maleimido, pyridyldisulfidem and iodoacetamido.

As used herein, an “antigen” can be an entity to which an antibody specifically binds.

The terms “specific binding” and “specifically binds” mean that the antibody or antibody fragment thereof will bind, in a selective manner, with its corresponding target antigen and not with a multitude of other antigens. Typically, the antibody or antibody fragment binds with an affinity of at least about 1×10⁻⁷ M, for example, 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely related antigen.

The term “amino acid” as used herein, refers to natural and non-natural, and proteogenic amino acids. Exemplary amino acids include, but are not limited to alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, ornithine, β-alanine, citrulline, serine methyl ether, aspartate methyl ester, glutamate methyl ester, homoserine methyl ether, and N,N-dimethyl lysine.

A “sugar moiety” as used herein, refers to a monosaccharide group, for example, a pyranose or a furanose. A sugar moiety can comprise a hemiacetal or a carboxylic acid (from oxidation of the pendant —CH₂OH group). In some embodiments, the sugar moiety is in the β-D conformation. In some embodiments, the sugar moiety is a glucose, glucuronic acid, or mannose group.

The term “inhibit” or “inhibition of” means to reduce by a measurable amount, or to prevent entirely (e.g., 100% inhibition).

As used herein, TLR7/8 can denote toll-like receptor 7 and toll-like receptor 8, just toll-like receptor, or just toll-like receptor 8. For example, a TLR7/8 ligand can be a TLR7 ligand, a TLR8 ligand, or a bifunctional TLR7 and TLR8 ligand.

A “TLR7/8 agonist” as defined herein includes any compound exhibiting selective TLR7/8 activity. Exemplary TLR7/8 agonists can exhibit activity (EC₅₀) against TLR7/8 of less than about 10 μM, less than about 5 μM, less than about 2 μM, less than about 1 μM, less than about 500 nM, less than about 250 nM, less than about 100 nM, or less than about 10 nM as measured in an assay as described herein. In some embodiments, a TLR7/8 agonist can exhibit selectivity for TLR7 over TLR8 of about 3.5-fold to about 25-fold, for example, about 3.5-fold to about 15-fold, about 10-fold to about 20-fold, about 15-fold to about 25-fold, about 3.5-fold to about 8-fold, about 5-fold to about 12-fold, about 8-fold to about 15-fold, about 12-fold to about 18-fold, about 15-fold to about 20-fold, or about 18-fold to about 25-fold. In some embodiments, a TLR7/8 agonist can exhibit selectivity for TLR8 over TLR7 of about 3.5-fold to about 25-fold, for example, about 3.5-fold to about 15-fold, about 10-fold to about 20-fold, about 15-fold to about 25-fold, about 3.5-fold to about 8-fold, about 5-fold to about 12-fold, about 8-fold to about 15-fold, about 12-fold to about 18-fold, about 15-fold to about 20-fold, or about 18-fold to about 25-fold.

The term “therapeutically effective amount” refers to an amount of an ADC, or a pharmaceutically acceptable salt thereof (as described herein) or a compound (as described herein, e.g. a compound of Formula (IX), or a pharmaceutically acceptable salt thereof), that is effective to treat a disease or disorder in a mammal. In the case of cancer, the therapeutically effective amount of the ADC or the compound provides one or more of the following biological effects: reduction of the number of cancer cells; reduction of tumor size; inhibition of cancer cell infiltration into peripheral organs; inhibition of tumor metastasis; inhibition, to some extent, of tumor growth; and/or relief, to some extent, of one or more of the symptoms associated with the cancer. For cancer therapy, efficacy, in some aspects, is measured by assessing the time to disease progression (TTP) and/or determining the response rate (RR).

Unless otherwise indicated or implied by context, the term “substantial” or “substantially” refers to a majority, i.e., >50% of a population, of a mixture, or a sample, typically more than 50%, 55%, 60%, 65%, 70%, 75%, 80%, 85%, 90%, 91%, 92%, 93%, 94%, 95%, 96%, 97%, 98%, or 99%.

The terms “intracellularly cleaved” and “intracellular cleavage” refer to a metabolic process or reaction occurring inside a cell, in which the cellular machinery acts on the ADC or a fragment thereof, to intracellularly release free drug from the ADC, or other degradant products thereof. The moieties resulting from that metabolic process or reaction are thus intracellular metabolites.

The terms “cancer” and “cancerous” refer to or describe the physiological condition or disorder in mammals that is typically characterized by unregulated cell growth. A “tumor” comprises multiple cancerous cells.

“Subject” as used herein refers to an individual to which an ADC or TLR7/8 agonist, as described herein, is administered. Examples of a “subject” include, but are not limited to, a mammal such as a human, rat, mouse, guinea pig, non-human primate, pig, goat, cow, horse, dog, cat, bird and fowl. Typically, a subject is a rat, mouse, dog, non-human primate, or human. In some aspects, the subject is a human.

The terms “treat” or “treatment,” unless otherwise indicated or implied by context, refer to therapeutic treatment and prophylactic measures to prevent relapse, wherein the object is to inhibit an undesired physiological change or disorder, such as, for example, the development or spread of cancer. For purposes of the present disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” in some aspects also means prolonging survival as compared to expected survival if not receiving treatment.

In the context of cancer, the term “treating” includes any or all of: inhibiting growth of cancer cells or of a tumor; inhibiting replication of cancer cells, lessening of overall tumor burden or decreasing the number of cancer cells, and ameliorating one or more symptoms associated with the disease.

The term “salt,” as used herein, refers to organic or inorganic salts of a compound, such as a TLR7/8 agonist (e.g., a compound of Formula (IX)), Drug Unit (D) (e.g., a compound of any of Formulae (I)-(VIII)), a linker, a drug-linker intermediate (e.g., a compound of Formula (X)), or an ADC, such as those described herein. In some aspects, the compound contains at least one amino group, and accordingly acid addition salts can be formed with the amino group. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A salt may involve the inclusion of another molecule such as an acetate ion, a succinate ion, or other counterion. The counterion may be any organic or inorganic moiety that stabilizes the charge on the parent compound. Furthermore, a salt has one or more than one charged atom in its structure. In instances where there are multiple charged atoms as part of the salt, multiple counter ions can be present. Hence, a salt can have one or more charged atoms and/or one or more counterions. A “pharmaceutically acceptable salt” is one that is suitable for administration to a subject as described herein and in some aspects includes salts as described by P. H. Stahl and C. G. Wermuth, editors, Handbook of Pharmaceutical Salts: Properties, Selection and Use, Weinheim/Zürich:Wiley-VCH/VHCA, 2002, the list for which is specifically incorporated by reference in its entirety. In some embodiments, the ADCs described herein are present in the form of a pharmaceutically acceptable salt. In some embodiments, the compounds described herein are present in the form of a pharmaceutically acceptable salt.

The term “tautomer,” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer.

The term “optionally substituted,” refers to an indicated group being either substituted or unsubstituted.

The term “alkyl” refers to an unsubstituted straight chain or branched, saturated hydrocarbon having the indicated number of carbon atoms (e.g., “C₁-C4 alkyl,” “C₁-C₆ alkyl,” “C₁-C₈ alkyl,” or “C₁-C₁₀” alkyl have from 1 to 4, 1 to 6, 1 to 8, or 1 to 10 carbon atoms, respectively) and is derived by the removal of one hydrogen atom from the parent alkane. Representative straight chain “C₁-C₈ alkyl” groups include, but are not limited to, methyl, ethyl, n-propyl, n-butyl, n-pentyl, n-hexyl, n-heptyl and n-octyl; while branched C₁-C₈ alkyls include, but are not limited to, isopropyl, sec-butyl, isobutyl, tert-butyl, isopentyl, and 2-methylbutyl.

The term “alkylene” refers to a bivalent unsubstituted saturated branched or straight chain hydrocarbon of the stated number of carbon atoms (e.g., a C₁-C₆ alkylene has from 1 to 6 carbon atoms) and having two monovalent centers derived by the removal of two hydrogen atoms from the same or two different carbon atoms of the parent alkane. Alkylene groups can be substituted with 1-6 fluoro groups, for example, on the carbon backbone (as —CHF— or —CF₂—) or on terminal carbons of straight chain or branched alkylenes (such as —CHF₂ or —CF₃). Alkylene groups include but are not limited to: methylene (—CH₂—), ethylene (—CH₂CH₂—), n-propylene (—CH₂CH₂CH₂—), n-propylene (—CH₂CH₂CH₂—), n-butylene (—CH₂CH₂CH₂CH₂—), difluoromethylene (—CF₂—), tetrafluoroethylene (—CF₂CF₂—), and the like.

The term “alkenyl” refers to an unsubstituted straight chain or branched, hydrocarbon having at least one carbon-carbon double bond and the indicated number of carbon atoms (e.g., “C₂-C₈ alkenyl” or “C₂-C₁₀” alkenyl have from 2 to 8 or 2 to 10 carbon atoms, respectively). When the number of carbon atoms is not indicated, the alkenyl group has from 2 to 6 carbon atoms.

The term “alkynyl” refers to an unsubstituted straight chain or branched, hydrocarbon having at least one carbon-carbon triple bond and the indicated number of carbon atoms (e.g., “C₂-C₈ alkynyl” or “C₂-C₁₀” alkynyl have from 2 to 8 or 2 to 10 carbon atoms, respectively). When the number of carbon atoms is not indicated, the alkynyl group has from 2 to 6 carbon atoms.

The term “heteroalkyl” refers to a stable straight or branched chain saturated hydrocarbon having the stated number of total atoms and at least one (e.g., 1 to 15) heteroatom selected from the group consisting of O, N, Si and S. The carbon and heteroatoms of the heteroalkyl group can be oxidized (e.g., to form ketones, N-oxides, sulfones, and the like) and the nitrogen atoms can be quaternized. The heteroatom(s) can be placed at any interior position of the heteroalkyl group and/or at any terminus of the heteroalkyl group, including termini of branched heteroalkyl groups), and/or at the position at which the heteroalkyl group is attached to the remainder of the molecule. Heteroalkyl groups can be substituted with 1-6 fluoro groups, for example, on the carbon backbone (as —CHF— or —CF₂—) or on terminal carbons of straight chain or branched heteroalkyls (such as —CHF₂ or —CF₃). Examples of heteroalkyl groups include, but are not limited to, —CH₂—CH₂—O—CH₃, —CH₂—CH₂—NH—CH₃, —CH₂—CH₂—N(CH₃)₂, —C(═O)—NH—CH₂—CH₂—NH—CH₃, —C(═O)—N(CH₃)—CH₂—CH₂—N(CH₃)₂, —C(═O)—NH—CH₂—CH₂—NH—C(═O)—CH₂—CH₃, —C(═O)—N(CH₃)—CH₂—CH₂—N(CH₃)—C(═O)—CH₂—CH₃, —O—CH₂—CH₂—CH₂—NH(CH₃), —O—CH₂—CH₂—CH₂—N(CH₃)₂, —O—CH₂—CH₂—CH₂—NH—C(═O)—CH₂—CH₃, —O—CH₂—CH₂—CH₂—N(CH₃)—C(═O)—CH₂—CH₃, —CH₂—CH₂—CH₂—NH(CH₃), —O—CH₂—CH₂—CH₂—N(CH₃)₂, —CH₂—CH₂—CH₂—NH—C(═O)—CH₂—CH₃, —CH₂—CH₂—CH₂—N(CH₃)—C(═O)—CH₂—CH₃, —CH₂—S—CH₂—CH₃, —CH₂—CH₂—S(O)—CH₃, —NH—CH₂—CH₂—NH—C(═O)—CH₂—CH₃, —CH₂—CH₂—S(O)₂—CH₃, —CH₂—CH₂—O—CF₃, and —Si(CH₃)₃. Up to two heteroatoms may be consecutive, such as, for example, —CH₂—NH—OCH₃ and —CH₂—O—Si(CH₃)₃. A terminal polyethylene glycol (PEG) moiety is a type of heteroalkyl group.

The term “acyl” refers to an alkyl, haloalkyl, alkenyl, alkynyl, aryl cycloalkyl, heteroaryl, or heterocyclyl group, as defined herein, connected to the remainder of the compound by a C═O (carbonyl) group.

The term “carboxamido” refers to a —C(═O)NRR′ group, wherein R and R′ are independently selected from the group consisting of hydrogen, alkyl, alkenyl, alkynyl, aryl cycloalkyl, heteroaryl, and heterocyclyl, as defined herein.

The term “heteroalkylene” refers to a bivalent unsubstituted straight or branched group derived from heteroalkyl (as defined herein). Examples of heteroalkylene groups include, but are not limited to, —CH₂—CH₂—O—CH₂—, —CH₂—CH₂—O—CF₂—, —CH₂—CH₂—NH—CH₂—, —C(═O)—NH—CH₂—CH₂—NH—CH₂— —C(═O)—N(CH₃)—CH₂—CH₂—N(CH₃)—CH₂—, —C(═O)—NH—CH₂—CH₂—NH—C(═O)—CH₂—CH₂—, —C(═O)—N(CH₃)—CH₂—CH₂—N(CH₃)—C(═O)—CH₂—CH₂—, —O—CH₂—CH₂—CH₂—NH—CH₂—,
—O—CH₂—CH₂—CH₂—N(CH₃)—CH₂—, —O—CH₂—CH₂—CH₂—NH—C(═O)—CH₂—CH₂—, —O—CH₂—CH₂—
CH₂—N(CH₃)—C(═O)—CH₂—CH₂—, —CH₂—CH₂—CH₂—NH—CH₂—, —CH₂—CH₂—CH₂—N(CH₃)—CH₂—, —CH₂—CH₂—CH₂—NH—C(═O)—CH₂—CH₂—, —CH₂—CH₂—CH₂—N(CH₃)—C(═O)—CH₂—CH₂—, —CH₂—CH₂—NH—C(═O)—,
CH₂—CH₂—N(CH₃)—CH₂—, —CH₂—CH₂—N⁺(CH₃)₂—, —NH—CH₂—CH₂(NH₂)—CH₂—, and —NH—CH₂—CH₂(NHCH₃)—CH₂—. A bivalent polyethylene glycol (PEG) moiety is a type of heteroalkylene group.

The term “alkoxy” refers to an alkyl group, as defined herein, which is attached to a molecule via an oxygen atom. For example, alkoxy groups include, but are not limited to methoxy, ethoxy, n-propoxy, iso-propoxy, n-butoxy, sec-butoxy, tert-butoxy, n-pentoxy and n-hexoxy.

The term “alkylthio” refers to an alkyl group, as defined herein, which is attached to a molecule via a sulfur atom. For example, alkythio groups include, but are not limited to thiomethyl, thioethyl, thio-n-propyl, thio-iso-propyl, and the like.

The term “haloalkyl” refers to an unsubstituted straight chain or branched, saturated hydrocarbon having the indicated number of carbon atoms (e.g., “C₁-C4 alkyl,” “C₁-C₆ alkyl,” “C₁-C₈ alkyl,” or “C₁-C₁₀” alkyl have from 1 to 4, 1 to 6, 1 to 8, or 1 to 10 carbon atoms, respectively) wherein at least one hydrogen atom of the alkyl group is replaced by a halogen (e.g., fluoro, chloro, bromo, or iodo). When the number of carbon atoms is not indicated, the haloalkyl group has from 1 to 6 carbon atoms. Representative C₁₋₆ haloalkyl groups include, but are not limited to, trifluoromethyl, 2,2,2-trifluoroethyl, and 1-chloroisopropyl.

The term “haloalkoxy” refers to a haloalkyl group, as defined herein, which is attached to a molecule via an oxygen atom. For example, haloalkoxy groups include, but are not limited to trifluoromethoxy, 2,2,2-trifluoroethoxy, and 1,1,1-trifluoro2-methylpropoxy.

The term “cycloalkyl” refers to a cyclic, saturated or partially unsaturated hydrocarbon having the indicated number of carbon atoms (e.g., “C₃₋₈ cycloalkyl” or “C₃₋₆” cycloalkyl have from 3 to 8 or 3 to 6 carbon atoms, respectively). When the number of carbon atoms is not indicated, the cycloalkyl group has from 3 to 6 carbon atoms. Cycloalkyl groups include bridged, fused, and spiro ring systems, and bridged bicyclic systems where one ring is aromatic and the other is unsaturated. Representative “C₃₋₆ cycloalkyl” groups include, cyclopropyl, cyclobutyl, cyclopentyl, and cyclohexyl.

The term “aryl” refers to an unsubstituted monovalent carbocyclic aromatic hydrocarbon group of 6-10 carbon atoms derived by the removal of one hydrogen atom from a single carbon atom of a parent aromatic ring system. Aryl groups include, but are not limited to, phenyl, naphthyl, anthracenyl, biphenyl, and the like.

The term “heterocycle” refers to a saturated or partially unsaturated ring or a multiple condensed ring system, including bridged, fused, and spiro ring systems. Heterocycles can be described by the total number of atoms in the ring system, for example a 3-10 membered heterocycle has 3 to 10 total ring atoms. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more heterocycles (e.g., decahydronapthyridinyl), carbocycles (e.g., decahydroquinolyl) or aryls. The rings of a multiple condensed ring system can be connected to each other via fused, spiro and bridged bonds when allowed by valency requirements. It is to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g., a nitrogen). Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, and 1,4-benzodioxanyl.

The term “heteroaryl” refers to an aromatic hydrocarbon ring system with at least one heteroatom within a single ring or within a fused ring system, selected from the group consisting of O, N and S. The ring or ring system has 4n+2 electrons in a conjugated π system where all atoms contributing to the conjugated π system are in the same plane. In some embodiments, heteroaryl groups have 5-10 total ring atoms and 1, 2, or 3 heteroatoms (referred to as a “5-10 membered heteroaryl”). Heteroaryl groups include, but are not limited to, imidazole, triazole, thiophene, furan, pyrrole, benzimidazole, pyrazole, pyrazine, pyridine, pyrimidine, and indole.

The term “hydroxyl” refers to an —OH group.

The term “cyano” refers to a —CN group.

The term “carboxy” refers to a —C(═O)OH group.

The term “oxo” refers to a ═O group.

The term “alkanoyl” refers to an alkyl group, as defined herein, connected to the remainder of the molecule by a —C(═O) group. Exemplary alkanoyl groups include, but are not limited to acetyl, n-propanoyl, and n-butanoyl.

The term “alkanoyloxy” refers to an alkyl group, as defined herein, connected to the remainder of the molecule by an —OC(═O) group. Exemplary alkanoyloxy groups include, but are not limited to acetoxy, n-propanoyloxy, and n-butanoyloxy.

The term “alkoxycarbonyl” refers to an alkoxy group, as defined herein, connected to a —C(═O)— group via the oxygen atom of the alkoxy (i.e., an alkyl ester group).

The term “alkoxythiocarbonyl” refers to an alkoxy group, as defined herein, connected to a C(═S)— group via the oxygen atom of the alkoxy (i.e., an alkyl thioester group).

The term “carbamoyl”, as defined herein, refers to —C(═O)—N(R)₂, wherein ‘R’ denotes variable substitution.

The term “amidine”, as defined herein, refers to C(═N)—N(R)₂, wherein ‘R’ denotes variable substitution.

The term “sulfone”, as defined herein, refers to —S(═O)₂—R, wherein ‘R’ denotes variable substitution.

The term “thione”, as defined herein, refers to —C(═S)—R, wherein ‘R’ denotes variable substitution.

The terms “arylalkyl” and “cycloalkylalkyl” refer to an aryl group or a cycloalkyl group (as defined herein) connected to the remainder of the molecule by an alkyl group, as defined herein. Exemplary arylalkyl groups include, but are not limited to benzyl and phenethyl. Exemplary cycloalkylalkyl groups include, but are not limited to cyclopropylmethyl, cyclobutylmethyl, cyclopentylethyl, and cyclohexylethyl.

The term “succinimide” as used as part of an antibody-drug conjugate (ADC) refers to:

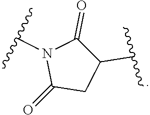 [SMILES: *C1CC(=O)N(*)C1=O]

The term “hydrolyzed succinimide” as used as part of an antibody-drug conjugate (ADC) refers to:

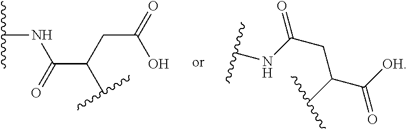 [SMILES: *NC(=O)C(*)CC(=O)O ; "or" ; *NC(=O)CC(*)C(=O)O]

As used herein, the term “hydrolysable group” refers to a moiety which undergoes spontaneous hydrolytic cleavage under specific conditions. For example, a hydrolysable group may be inert in neutral and basic solutions, but may undergo hydrolytic cleavage in days, hours, minutes, or seconds under acidic conditions. In some cases, a hydrolysable group is configured to undergo hydrolytic cleavage in a particular physiological environment, such as blood (e.g., peripheral blood) or oxidative (e.g., lysosomal) or reductive (e.g., cytoplasmic) intracellular compartments. In some cases, a hydrolysable group is configured for catalytic cleavage, for example by enzymes present in a specific organism (e.g., humans) or tissues (e.g., metabolically active tissues such as liver, kidney, or brain). A hydrolysable group can be configured for cleavage by a range of enzymes, or by a specific enzyme. For example, a hydrolysable group can comprise an oligopeptide of the sequence arginine-arginine-valine-arginine, for which human furin may have high cleavage activity. A hydrolysable group can be configured for cleavage within a particular environment, such as endosomes or lysozomes of human cells. In such cases, the hydrolysable group may be stable outside of the environment in which it is configured for cleavage. For example, a hydrolysable group may be stable in circulation within peripheral blood, but hydrolytically cleave upon uptake into a cell. Examples of hydrolysable groups include organophosphates such as phosphate esters, thiophosphates, and dithiophosphates, carbamates, carbonates, thioesters, quaternary amines, ureas, disulfides, organosulfates, diorganosulfates, certain amides and esters, and peptides with protease cleavage sites.

It will be appreciated by those skilled in the art that compounds described herein having a chiral center may exist in and be isolated in optically active and racemic forms.

As used herein, the term “free drug” refers to a biologically active species that is not covalently attached to an antibody. Accordingly, free drug refers to a compound as it exists immediately upon cleavage from the ADC. The release mechanism can be via a cleavable linker in the ADC, or via intracellular conversion or metabolism of the ADC. In some aspects, the free drug will be protonated and/or may exist as a charged moiety. The free drug is a pharmacologically active species which is capable of exerting the desired biological effect. In some embodiments, the pharmacologically active species is the parent drug alone. In some embodiments, the pharmacologically active species is the parent drug bonded to a component or vestige of the ADC (e.g., a component of the linker, succinimide, hydrolyzed succinimide, and/or antibody that has not undergone subsequent intracellular metabolism). In some embodiments, free drug refers to a compound of any one of Formulae (I)-(VIII), or a pharmaceutically acceptable salt thereof, as described herein, for example, wherein one or more of X, Y, W, A, and M are absent. In some embodiments, free drug refers to a compound of Formula (IX). In some embodiments, free drug refers to a compound, or a pharmaceutically acceptable salt thereof, disclosed in U.S. Publ. No. 2017/0217960, which is incorporated by reference in its entirety.

As used herein, the term “Drug Unit” refers to the free drug that is conjugated to an antibody in an ADC, as described herein.

### Antibody Drug Conjugates (ADCs)

Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
each D has the structure of Formula (A):

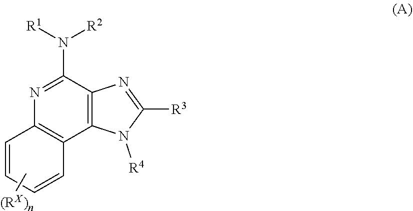

or a pharmaceutically acceptable salt thereof;
wherein:
R¹ is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached, form a 3-6 membered heterocyclyl optionally substituted with 1-3 independently selected C₁-C₆ alkyl;
R³ is (a) the point of covalent attachment to L; or (b) selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) the point of covalent attachment to L; (b) —OR^{C}; or (c) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H;
wherein when R⁴ is (c), the C₁-C₆ alkyl, or a substituent thereof, may be further substituted with the point of covalent attachment to L;
each R^{X} is (a) the point of covalent attachment to L; or (b) independently selected from the group consisting of hydrogen, —C(═O)OR^{F}, —C(═O)NR^{G}R^{H}, —S(O₂)NR^{G}R^{H}, N(R^{I})—C(═O)R^{J}, —N(R^{I})—S(O₂)R^{K}, halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R^{X} is the point of covalent attachment to L;
subscript n is 0, 1, 2, 3, or 4;
each R^{A} and R^{B} is (a) the point of covalent attachment to L, (b) independently selected from the group consisting of hydrogen and C₁-C₆ alkyl; or R^{A} and R^{B} taken together with the nitrogen atom to which they are attached, form a 3-6 membered heterocyclyl optionally substituted with 1-3 independently selected C₁-C₆ alkyl; wherein only one of R^{A} and R^{B} is the point of covalent attachment to L;
R^{C} is (a) the point of covalent attachment to L; or (b) selected from the group consisting of hydrogen, phenyl, and C₁-C₁₀ alkyl optionally substituted with phenyl or 1-3 independently selected halogen;
each R^{D}, R^{E}, R^{G}, and R^{H} are (a) the point of covalent attachment to L; or (b) independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached, form a 3-6 membered heterocyclyl optionally substituted with 1-3 independently selected C₁-C₆ alkyl; wherein only one of R^{D}, R^{E}, R^{G}, and R^{H} is the point of covalent attachment to L;
R^{F} is (a) the point of covalent attachment to L; or (b) selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is (a) the point of covalent attachment to L; or (b) independently selected from the group consisting of hydrogen and C₁-C₆ alkyl; wherein only one of R^{I}, R^{J}, and R^{K} is the point of covalent attachment to L;
wherein only one of R¹, R², R³, R⁴, R^{X}, R^{A}, R^{B}, R^{C}, R^{D}, R^{E}, R^{F}, R^{G}, R^{H}, R^{I}, R^{J} and R^{K} is the point of covalent attachment to L; and
wherein each D has only one point of covalent attachment to L.

In some embodiments, one R^{X} is R⁵ and the remaining R^{X} are R⁶; wherein R⁵ is (a) the point of covalent attachment to L; or (b) selected from the group consisting of hydrogen, —C(═O)OR^{F}, —C(═O)NR^{G}R^{H}, —S(O₂)NR^{G}R^{H}, —N(R^{J})—C(═O)R^{J}, and —N(R¹)—S(O₂)R^{K}; and
each R⁶ is (a) the point of covalent attachment to L; or (b) independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R⁶ is the point of covalent attachment to L. As used herein, the “remaining” R^{X} groups refers to 0 or subscript n-1 R^{X} groups, e.g., 0, 1, 2, or 3 R^{X} groups. Thus, when subscript n is 0, there are zero remaining R^{X} groups; when subscript n is 1, there are also zero remaining R^{X} groups; when subscript n is 2, there is 1 remaining R^{X} group; when subscript n is 3, there are 2 remaining R^{X} groups; when subscript n is 4, there are 3 remaining R^{X} groups.

In some embodiments, one of R⁶ is the point of covalent attachment to L and the other R⁶, if any, are independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}.

In some embodiments, only one of R⁵ and R⁶ is the point of covalent attachment to L. In some embodiments, each R^{x}, if present, is independently selected from the group consisting of hydrogen, —C(═O)OR^{F}, —C(═O)NR^{G}R^{H}, —S(O₂)NR^{G}R^{H}, —N(R^{J})—C(═O)R^{J}, —N(R^{J})—S(O₂)R^{K}, halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}. In some embodiments, one of R^{X} is the point of covalent attachment to L and the other R^{x}, if any, are independently selected from the group consisting of hydrogen, —C(═O)OR^{F}, —C(═O)NR^{G}R^{H}, —S(O₂)NR^{G}R^{H}, —N(R^{J})—C(═O)R^{J}, —N(R^{J})—S(O₂)R^{K}, halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}.

Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
each D is a 1H-imidazo[4,5-c]quinolin-4-amine TLR7/8 agonist.

Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
each D has the structure of Formula (I):

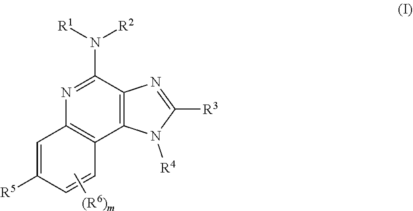

or a pharmaceutically acceptable salt thereof;
wherein:
R¹ is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is (a) the point of covalent attachment to L; or (b) selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) the point of covalent attachment to L; (b) —OR^{C}; or (c) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H;
wherein when R⁴ is (c), the C₁-C₆ alkyl, or a substituent thereof, may be further substituted with the point of covalent attachment to L;
R⁵ is (a) the point of covalent attachment to L; or (b) selected from the group consisting of hydrogen, —C(═O)OR^{F}, —C(═O)NR^{G}R^{H}, —S(O₂)NR^{G}R^{H}, —N(R^{J})—C(═O)R^{J}, and —N(R^{J})—S(O₂)R^{K};
each R⁶ is (a) the point of covalent attachment to L; or (b) independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R⁶ is the point of covalent attachment to L;
subscript m is 0, 1, 2, or 3;
each R^{A} and R^{B} is (a) the point of covalent attachment to L, (b) independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl; wherein only one of R^{A} and R^{B} is the point of covalent attachment to L;
R^{C} is (a) the point of covalent attachment to L; or (b) selected from the group consisting of hydrogen, phenyl, and C₁-C₁₀ alkyl optionally substituted with phenyl or 1-3 independently selected halogen;
each R^{D}, R^{E}, R^{G}, and R^{H} are (a) the point of covalent attachment to L; or (b) independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
wherein only one of R^{D}, R^{E}, R^{G}, and R^{H} is the point of covalent attachment to L;
R^{F} is (a) the point of covalent attachment to L; or (b) selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is (a) the point of covalent attachment to L; or (b) independently selected from the group consisting of hydrogen and C₁-C₆ alkyl; wherein only one of R^{I}, R^{J}, and R^{K} is the point of covalent attachment to L;
wherein only one of R¹, R², R³, R⁴, R⁵, R⁶, R^{A}, R^{B}, R^{C}, R^{D}, R^{E}, R^{F}, R^{G}, R^{H}, R^{I}, R^{J} and R^{K} is the point of covalent attachment to L; and
wherein each D has only one point of covalent attachment to L.

Some embodiments provide an antibody drug conjugate (ADC) having the structure:

Ab-(L-D)ₚ

or a pharmaceutically acceptable salt thereof;
wherein:
Ab is an antibody;
wherein each D is conjugated to a linker (L);
wherein each L is covalently attached to Ab via a sulfur atom of a cysteine residue or an ϵ-amino group of a lysine residue;
subscript p is an integer from 1 to 16;
each D has the structure of Formula (II):

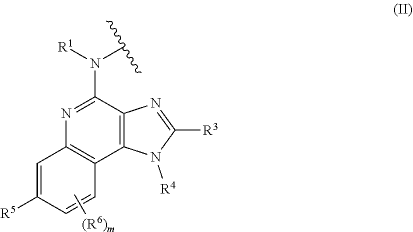

or a pharmaceutically acceptable salt thereof;
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C} or (b) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —C(═O)NR^{G}R^{H}, —S(O₂) NR^{G}R^{H}, —N(R′)—C(═O)R^{J}, and —N(R^{T})—S(O₂)R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; and
each R^{I}, R^{J}, and R^{K} is independently selected from the group consisting of hydrogen and C₁-C₆ alkyl.

Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
each D has the structure of Formula (III):

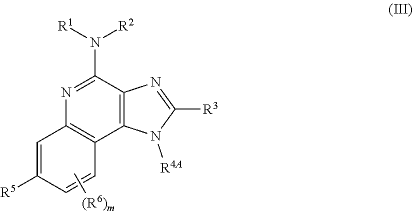

or a pharmaceutically acceptable salt thereof;
   wherein:
   R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
   R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
   R¹ and R², taken together with the nitrogen atom to which they are attached form a 3-6 membered heterocyclyl optionally substituted with 1-3 independently selected C₁-C₆ alkyl;
   R³ is selected from the group consisting of hydrogen, —NR^{A}R^{B}, —C(═O) NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
   R^{4A} is (a) the point of covalent attachment to L or (b) C₁-C₆ alkyl substituted with:
   (i) 1-3 independently selected halogen;
   (ii) —OR^{C};
   (iii) —SR^{C};
   (iv) —NH—S(O₂)R^{C};
   (v) —OC(═O)R^{C};
   (vi) —CO₂H;
   (vii) C₁-C₆ alkoxycarbonyl;
   (viii) —C(═O)NR^{D}R^{E};
   (ix) —NR^{D}R^{E};
   (x) —[N(C₁-C₆ alkyl)R^{D}R^{E}]⁺;
   (xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
   (xii) phenyl substituted with halogen, hydroxyl, C₁-C₆ alkoxy, —C(═O)NR^{D}R^{E} or —CO₂H;
   (xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
   (xiv) 5-10 membered heteroaryl optionally substituted with halogen, —NR^{D}R^{E}, C₁-C₆ alkoxy, —C(═O)NR^{D}R^{E}, or —CO₂H;
   wherein when R^{4A} is (b) the C₁-C₆ alkyl, or a substituent thereof, is further substituted with the point of covalent attachment to L;
   R⁵ is selected from the group consisting of hydrogen, —C(═O)OR^{F}, —C(═O)NR^{G}R^{H}, —S(O₂) NR^{G}R^{H}, —N(R′)—C(═O)R^{J}, and —N(R¹)—S(O₂)R^{K};
   each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
   subscript m is 0, 1, 2, or 3;
   each R^{A} and R^{B} independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
   R^{C} is selected from the group consisting of hydrogen, phenyl, and C₁-C₁₀ alkyl optionally substituted with phenyl or 1-3 independently selected halogen;
   each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
   R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; and
   each R^{I}, R^{J}, and R^{K} is independently selected from the group consisting of hydrogen and C₁-C₆ alkyl.

Some embodiments provide an antibody drug conjugate (ADC) having the structure:

Ab-(L-D)ₚ
or a pharmaceutically acceptable salt thereof;
wherein:
Ab is an antibody;
wherein each D is conjugated to a linker (L);
wherein each L is covalently attached to Ab via a sulfur atom of a cysteine residue or an ϵ-amino group of a lysine residue;
subscript p is an integer from 1 to 16;
   L has the formula -M-(A)ₐ-(W)_{w}—(Y)_{y}—(X)ₓ, wherein:
subscript a is 0 or 1;
subscript y is 0 or 1;
subscript w is 0 or 1;
   subscript x is 0 or 1;
M is a succinimide, a hydrolyzed succinimide, an amide, or a triazole;
   A is a C₂₋₂₀ alkylene optionally substituted with 1-3 R^{a1}; or a 2 to 40 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{a1}R^{e1} C(═O)NR^{d1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
W is from 1-12 amino acids or has the structure:

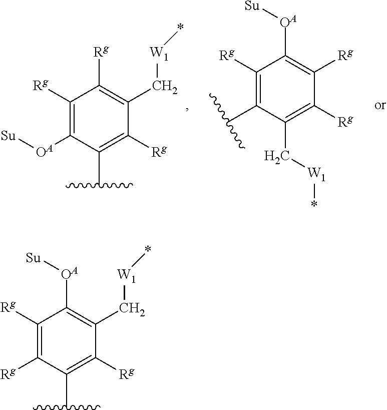

wherein Su is a Sugar moiety;
   —O^{A}— represents the oxygen atom of a glycosidic bond;
   each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
   W¹ is absent, *—C(═O)—O—, or *—O—C(═O)—;
   [symbol] represents covalent attachment to A or M;
* represents covalent attachment to X, Y, or D;
   Y is self-immolative moiety, a non-self-immolative releasable moiety, or a non-cleavable moiety;
   X is a C₁-C₆ alkylene or a 3-6 membered heteroalkylene;
   L is optionally substituted with a PEG Unit from PEG1 to PEG72;
each D has the structure of Formula (II):

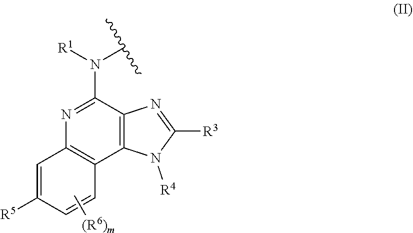

or a pharmaceutically acceptable salt thereof;
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C} or (b) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —C(═O)NR^{G}R^{H}, —S(O₂) NR^{G}R^{H}, —N(R′)—C(═O)R^{J}, and —N(R^{T})—S(O₂)R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; and
each R^{I}, R^{J}, and R^{K} is independently selected from the group consisting of hydrogen and C₁-C₆ alkyl.

Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
   L has the formula -M-(A)ₐ-(W)_{w}—(Y)_{y}—(X)ₓ, wherein:
subscript a is 0 or 1;
subscript y is 0 or 1;
subscript w is 0 or 1;
subscript x is 0 or 1;
M is a succinimide, a hydrolyzed succinimide, an amide, or a triazole;
   A is a C₂₋₂₀ alkylene optionally substituted with 1-3 R^{a1}; or a 2 to 40 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
W is from 1-12 amino acids or has the structure:

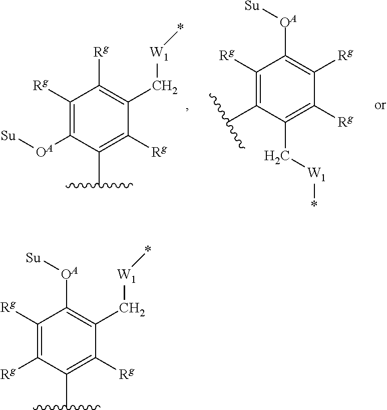

wherein Su is a Sugar moiety;
   —O^{A}— represents the oxygen atom of a glycosidic bond;
   each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
   W¹ is absent, *—C(═O)—O—, or *—O—C(═O)—;
   [symbol] represents covalent attachment to A or M;
* represents covalent attachment to X, Y, or D;
   Y is self-immolative moiety, a non-self-immolative releasable moiety, or a non-cleavable moiety;
   X is a C₁-C₆ alkylene or a 3-6 membered heteroalkylene;
   L is optionally substituted with a PEG Unit from PEG1 to PEG72;
each D has the structure of Formula (III):

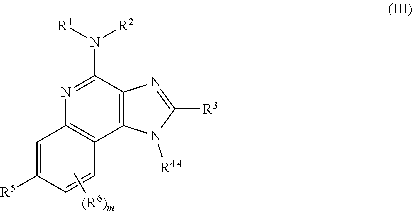

or a pharmaceutically acceptable salt thereof;
wherein:
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B}; R^{4A} is (a) the point of covalent attachment to L or (b) C₁-C₆ alkyl substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
wherein when R^{4A} is (b) the C₁-C₆ alkyl, or a substituent thereof, is further substituted with the point of covalent attachment to L;
R⁵ is selected from the group consisting of hydrogen, —C(═O)OR^{F}, —C(═O)NR^{G}R^{H}, —S(O₂) NR^{G}R^{H}, —N(R′)—C(═O)R^{J}, and —N(R¹)—S(O₂)R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is i independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; and
each R^{I}, R^{J}, and R^{K} is independently selected from the group consisting of hydrogen and C₁-C₆ alkyl.

Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
L has the formula -M-(A)ₐ-(W)_{w}—(Y)_{y}—(X)ₓ, wherein:
subscript a is 0 or 1;
subscript y is 0 or 1;
subscript w is 0 or 1;
subscript x is 0 or 1;
M is a succinimide, a hydrolyzed succinimide, an amide, or a triazole;
   A is a C₂₋₂₀ alkylene optionally substituted with 1-3 R^{a1}; or a 2 to 40 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1} C(═O)NR^{d1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
W is from 1-12 amino acids or has the structure:

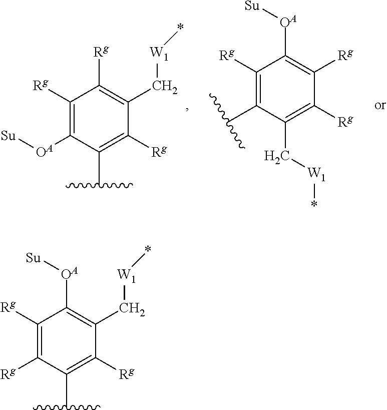

wherein Su is a Sugar moiety;
   —O^{A}— represents the oxygen atom of a glycosidic bond;
   each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
   W¹ is absent, *—C(═O)—O—, or *—O—C(═O)—;
   [symbol] represents covalent attachment to A or M;
* represents covalent attachment to X, Y, or D;
   Y is self-immolative moiety, a non-self-immolative releasable moiety, or a non-cleavable moiety;
   X is a C₁-C₆ alkylene or a 3-6 membered heteroalkylene;
   L is optionally substituted with a PEG Unit from PEG1 to PEG72;
each D has the structure of Formula (IV):

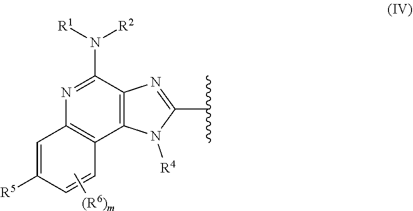

or a pharmaceutically acceptable salt thereof,
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R⁴ is (a) —OR^{C} or (b) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —C(═O)NR^{G}R^{H}, —S(O₂) NR^{G}R^{H}, —N(R′)—C(═O)R^{J}, and —N(R^{T})—S(O₂)R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; and
each R^{I}, R^{J}, and R^{K} is independently selected from the group consisting of hydrogen and C₁-C₆ alkyl.

Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
L has the formula -M-(A)ₐ-(W)_{w}—(Y)_{y}—(X)ₓ, wherein:
subscript a is 0 or 1;
subscript y is 0 or 1;
subscript w is 0 or 1;
subscript x is 0 or 1;
M is a succinimide, a hydrolyzed succinimide, an amide, or a triazole;
   A is a C₂₋₂₀ alkylene optionally substituted with 1-3 R^{a1}; or a 2 to 40 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
W is from 1-12 amino acids or has the structure:

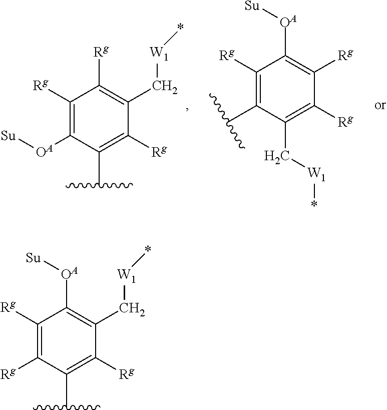

wherein Su is a Sugar moiety;
   —O^{A}— represents the oxygen atom of a glycosidic bond;
   each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
   W¹ is absent, *—C(═O)—O—, or *—O—C(═O)—;
   [symbol] represents covalent attachment to A or M;
* represents covalent attachment to X, Y, or D;
   Y is self-immolative moiety, a non-self-immolative releasable moiety, or a non-cleavable moiety;
   X is a C₁-C₆ alkylene or a 3-6 membered heteroalkylene;
   L is optionally substituted with a PEG Unit from PEG1 to PEG72;
each D has the structure of Formula (V):

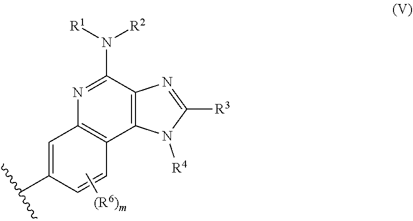

or a pharmaceutically acceptable salt thereof,
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C} or (b) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen; each R^{D} and R^{E} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, together with the nitrogen atom to which they are attached form a 3-6 membered heterocyclyl optionally substituted with 1-3 independently selected C₁-C₆ alkyl. Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
   L has the formula -M-(A)ₐ-(W)_{w}—(Y)_{y}—(X)ₓ, wherein:
subscript a is 0 or 1;
subscript y is 0 or 1;
subscript w is 0 or 1;
   subscript x is 0 or 1;
M is a succinimide, a hydrolyzed succinimide, an amide, or a triazole;
   A is a C₂₋₂₀ alkylene optionally substituted with 1-3 R^{a1}; or a 2 to 40 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{a1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1} C(═O)NR^{a1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
W is from 1-12 amino acids or has the structure:

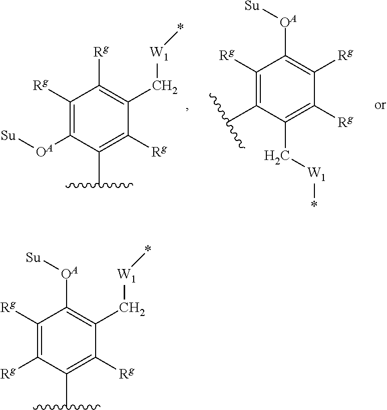

wherein Su is a Sugar moiety;
   —O^{A}— represents the oxygen atom of a glycosidic bond;
   each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
   W¹ is absent, *—C(═O)—O—, or *—O—C(═O)—;
   [symbol] represents covalent attachment to A or M;
* represents covalent attachment to X, Y, or D;
   Y is self-immolative moiety, a non-self-immolative releasable moiety, or a non-cleavable moiety;
   X is a C₁-C₆ alkylene or a 3-6 membered heteroalkylene;
   L is optionally substituted with a PEG Unit from PEG1 to PEG72;
each D has the structure of Formula (VI):

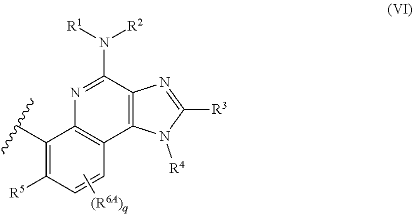

or a pharmaceutically acceptable salt thereof,
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C} or (b) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —C(═O)NR^{G}R^{H}, —S(O₂) NR^{G}R^{H}, —N(R′)—C(═O)R^{J}, and —N(R^{T})—S(O₂)R^{K};
each R^{6A} is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript q is 0, 1, or 2;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; and
each R^{I}, R^{J}, and R^{K} is independently selected from the group consisting of hydrogen and C₁-C₆ alkyl.

Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
L has the formula -M-(A)ₐ-(W)_{w}—(Y)_{y}—(X)ₓ, wherein:
subscript a is 0 or 1;
subscript y is 0 or 1;
subscript w is 0 or 1;
subscript x is 0 or 1;
M is a succinimide, a hydrolyzed succinimide, an amide, or a triazole;
   A is a C₂₋₂₀ alkylene optionally substituted with 1-3 R^{a1}; or a 2 to 40 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{a1}R^{e1} C(═O)NR^{d1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
W is from 1-12 amino acids or has the structure:

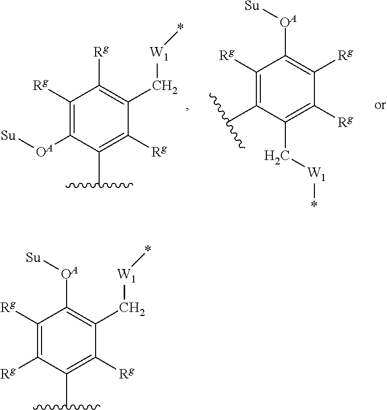

wherein Su is a Sugar moiety;
   —O^{A}— represents the oxygen atom of a glycosidic bond;
   each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
   W¹ is absent, *—C(═O)—O—, or *—O—C(═O)—;
   [symbol] represents covalent attachment to A or M;
* represents covalent attachment to X, Y, or D;
   Y is self-immolative moiety, a non-self-immolative releasable moiety, or a non-cleavable moiety;
   X is a C₁-C₆ alkylene or a 3-6 membered heteroalkylene;
   L is optionally substituted with a PEG Unit from PEG1 to PEG72;
each D has the structure of Formula (VII):

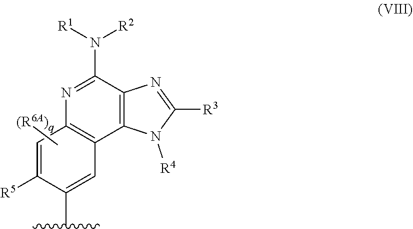

or a pharmaceutically acceptable salt thereof,
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B} C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B}.
R⁴ is (a) —OR^{C} or (b) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —C(═O)NR^{G}R^{H}, —S(O₂) NR^{G}R^{H}, —N(R′)—C(═O)R^{J}, and —N(R¹)—S(O₂)R^{K};
each R^{6A} is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript q is 0, 1, or 2;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; and
each R^{I}, R^{J}, and R^{K} is independently selected from the group consisting of hydrogen and C₁-C₆ alkyl.
Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
   L has the formula -M-(A)ₐ-(W)_{w}—(Y)_{y}—(X)ₓ, wherein:
subscript a is 0 or 1;
subscript y is 0 or 1;
subscript w is 0 or 1;
subscript x is 0 or 1;
M is a succinimide, a hydrolyzed succinimide, an amide, or a triazole;
   A is a C₂₋₂₀ alkylene optionally substituted with 1-3 R^{a1}; or a 2 to 40 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{a1}R^{e1} C(═O)NR^{d1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
W is from 1-12 amino acids or has the structure:

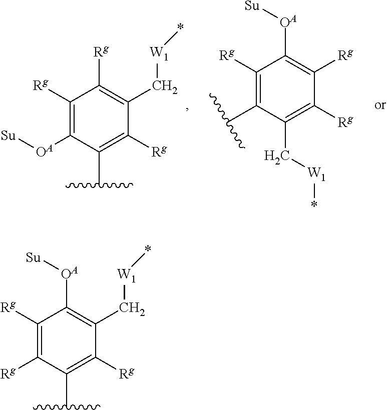

wherein Su is a Sugar moiety;
   —O^{A}— represents the oxygen atom of a glycosidic bond;
   each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
   W¹ is absent, *—C(═O)—O—, or *—O—C(═O)—;
   [symbol] represents covalent attachment to A or M;
represents covalent attachment to X, Y, or D;
   Y is self-immolative moiety, a non-self-immolative releasable moiety, or a non-cleavable moiety;
   X is a C₁-C₆ alkylene or a 3-6 membered heteroalkylene;
   L is optionally substituted with a PEG Unit from PEG1 to PEG72;
each D has the structure of Formula (VIII):

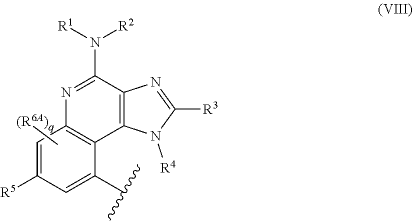

or a pharmaceutically acceptable salt thereof,
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃—C₈ cycloalkyl, phenyl, 5-10 membered heteroaryl, C₁-C₆ alkoxy, C₁-C₆ alkylthio, and —NR^{A}R^{B};
R⁴ is (a) —OR^{C} or (b) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —C(═O)NR^{G}R^{H}, —S(O₂) NR^{G}R^{H}, —N(R′)—C(═O)R^{J}, and —N(R^{T})—S(O₂)R^{K};
each R^{6A} is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript q is 0, 1, or 2;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; and
each R^{I}, R^{J}, and R^{K} is independently selected from the group consisting of hydrogen and C₁-C₆ alkyl.

In some embodiments, R^{D} and R^{E}, or R^{G} and R^{H}, together with the nitrogen atom to which they are attached form a 3-6 membered heterocyclyl optionally substituted with 1-3 independently selected C₁-C₆ alkyl and the other R^{G} and R^{H} or R¹ and R^{E} are independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-. In some embodiments, one of R^{D}, R^{E}, R^{G}, and R^{H} is the point of covalent attachment to L and the other of R^{D}, R^{E}, R^{G}, and R^{H} are independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-. In some embodiments, one of R^{I}, R^{J}, and R^{K} is the point of covalent attachment to L, and the other R^{I}, R^{J}, and R^{K} are independently selected from the group consisting of hydrogen and C₁-C₆ alkyl.

Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
each D has the structure of Formula (A):

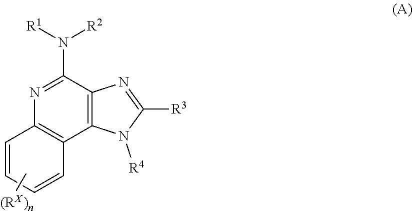

or a pharmaceutically acceptable salt thereof;
   wherein:
   R¹ is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
   R² is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
   R¹ and R², taken together with the nitrogen atom to which they are attached, form a 3-6 membered heterocyclyl optionally substituted with 1-3 independently selected C₁-C₆ alkyl;
   R³ is (a) the point of covalent attachment to L; or (b) selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
   R⁴ is (a) the point of covalent attachment to L¹; (b) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
   (i) 1-3 independently selected halogen;
   (ii) —OR^{C};
   (iii) —SR^{C};
   (iv) —NH—S(O₂)R^{C};
   (v) —OC(═O)R^{C};
   (vi) —CO₂H;
   (vii) C₁-C₆ alkoxycarbonyl;
   (viii) —C(═O)NR^{D}R^{E};
   (ix) —NR^{D}R^{E};
   (x) —[N(C₁-C₆ alkyl)R^{D}R^{E}]⁺;
   (xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
   (xii) phenyl substituted with halogen, hydroxyl, C₁-C₆ alkoxy, —C(═O)NR^{D}R^{E} or —CO₂H;
   (xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
   (xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H;
   wherein when R⁴ is (c), the C₁-C₆ alkyl, or a substituent thereof, may be further substituted with the point of covalent attachment to L;
   each R^{X} is (a) the point of covalent attachment to L; or (b) independently selected from the group consisting of hydrogen, —C(═O)OR^{F}, —C(═O)NR^{G}R^{H}S(O₂)NR^{G}R^{H}, —N(R^{I})—C(═O)R^{J}, —N(R^{T})—S(O₂)R^{K}, halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R^{X} is the point of covalent attachment to L;
   subscript n is 0, 1, 2, 3, or 4;
   each R^{A} and R^{B} is (a) the point of covalent attachment to L, (b) independently selected from the group consisting of hydrogen and C₁-C₆ alkyl; or R^{A} and R^{B} taken together with the nitrogen atom to which they are attached, form a 3-6 membered heterocyclyl optionally substituted with 1-3 independently selected C₁-C₆ alkyl; wherein only one of R^{A} and R^{B} is the point of covalent attachment to L;
   R^{C} is (a) the point of covalent attachment to L; or (b) selected from the group consisting of hydrogen, phenyl, and C₁-C₁₀ alkyl optionally substituted with phenyl or 1-3 independently selected halogen;
   each R^{D}, R^{E}, R^{G}, and R^{H} are (a) the point of covalent attachment to L; or (b) independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached, form a 3-6 membered heterocyclyl optionally substituted with 1-3 independently selected C₁-C₆ alkyl;
   wherein only one of R^{D}, R^{E}, R^{G}, and R^{H} is the point of covalent attachment to L;
   R^{F} is (a) the point of covalent attachment to L; or (b) selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
   each R^{I}, R^{J}, and R^{K} is (a) the point of covalent attachment to L; or (b) independently selected from the group consisting of hydrogen and C₁-C₆ alkyl; wherein only one of R^{I}, R^{J}, and R^{K} is the point of covalent attachment to L;
   wherein only one of R¹, R², R³, R⁴, R^{X}, R^{A}, R^{B}, R^{C}, R^{D}, R^{E}, R^{F}, R^{G}, R^{H}, R^{I}, R^{J} and R^{K} is the point of covalent attachment to L; and
   wherein each D has only one point of covalent attachment to L.

In some embodiments, one R^{X} is R⁵ and the remaining R^{X} are R⁶; wherein R⁵ is (a) the point of covalent attachment to L; or (b) selected from the group consisting of hydrogen, —C(═O)OR^{F}, —NO₂, —CN, —CF₃, —C(═O)NR^{G}R^{H}, —S(O₂)NR^{G}R^{H}, —N(R^{J})—C(═O)R^{J}, —N(R¹)—S(O₂)R^{K}, and SO₃R^{K}; and each R⁶ is (a) the point of covalent attachment to L; or (b) independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R⁶ is the point of covalent attachment to L. As used herein, the “remaining” R^{X} groups refers to 0 or subscript n-1 R^{X} groups, e.g., 0, 1, 2, or 3 R^{X} groups. Thus, when subscript n is 0, there are zero remaining R^{X} groups; when subscript n is 1, there are also zero remaining R^{X} groups; when subscript n is 2, there is 1 remaining R^{X} group; when subscript n is 3, there are 2 remaining R^{X} groups; when subscript n is 4, there are 3 remaining R^{X} groups. In some embodiments, one of R⁶ is the point of covalent attachment to L and the other R⁶, if any, are independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}. In some embodiments, only one of R⁵ and R⁶ is the point of covalent attachment to L. In some embodiments, each R^{x}, if present, is independently selected from the group consisting of hydrogen, —C(═O)OR^{F}, —C(═O)NR^{G}R^{H}, —S(O₂)NR^{G}R^{H}, —N(R^{J})—C(═O)R^{J}, S(O₂)R^{K}, —S(O₃)R^{K}, halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}. In some embodiments, one of R^{X} is the point of covalent attachment to L and the other R^{x}, if any, are independently selected from the group consisting of hydrogen, —C(═O)OR^{F}, —C(═O)NR^{G}R^{H}, —S(O₂)NR^{G}R^{H}, —N(R)—C(═O)R^{J}, —N(R^{J})—S(O₂)R^{K}, halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}.

Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
each D is a 1H-imidazo[4,5-c]quinolin-4-amine TLR7/8 agonist.

Some embodiments provide an antibody drug conjugate (ADC) having the structure:

Ab-(L-D)ₚ
or a pharmaceutically acceptable salt thereof;
   wherein:
   Ab is an antibody;
   each L is a linker;
   wherein each D is conjugated to a linker;
   wherein each L is covalently attached to Ab via a sulfur atom of a cysteine residue or an ϵ-amino group of a lysine residue;
   subscript p is an integer from 1 to 16;
   each D has the structure of Formula (I):

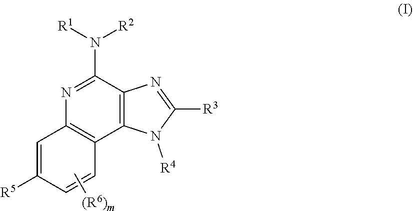

or a pharmaceutically acceptable salt thereof;
wherein:
R¹ is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is (a) the point of covalent attachment to L; or (b) selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) the point of covalent attachment to L¹; (b) —OR^{C}; (c) —S(═O)₂R^{C}; (d) —C(═O)NR^{D}R^{E}; (e) —C(═O)OR^{C}; (f) —C(═O)SR^{C}; (g) —C(═S)R^{C}; (h) —PO₃R^{C}; or (j) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H;
wherein when R⁴ is (j), the C₁-C₆ alkyl, or a substituent thereof, may be further substituted with the point of covalent attachment to L;
R⁵ is (a) the point of covalent attachment to L; or (b) selected from the group consisting of hydrogen, —C(═O)OR^{F}, —NO₂, —CN, —CF₃, —C(═O)NR^{G}R^{H}, —S(O₂)NR^{G}R^{H}, —N(R^{I})—C(═O)R^{J}, —N(R^{I})—S(O₂)R^{K}, and SO₃R^{K};
each R⁶ is (a) the point of covalent attachment to L; or (b) independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R⁶ is the point of covalent attachment to L;
subscript m is 0, 1, 2, or 3;
each R^{A} and R^{B} is (a) the point of covalent attachment to L, (b) independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl; wherein only one of R^{A} and R^{B} is the point of covalent attachment to L;
R^{C} is (a) the point of covalent attachment to L; or (b) selected from the group consisting of hydrogen, phenyl, and C₁-C₁₀ alkyl optionally substituted with phenyl or 1-3 independently selected halogen;
each R^{D}, R^{E}, R^{G}, and R^{H} are (a) the point of covalent attachment to L; or (b) independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
wherein only one of R^{D}, R^{E}, R^{G}, and R^{H} is the point of covalent attachment to L;
R^{F} is (a) the point of covalent attachment to L; or (b) selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is (a) the point of covalent attachment to L; or (b) independently selected from the group consisting of hydrogen and C₁-C₆ alkyl; wherein only one of R^{I}, R^{J}, and R^{K} is the point of covalent attachment to L;
wherein only one of R¹, R², R³, R⁴, R⁵, R⁶, R^{A}, R^{B}, R^{C}, R^{D}, R^{E}, R^{F}, R^{G}, R^{H}, R^{I}, R^{J} and R^{K} is the point of covalent attachment to L; and
wherein each D has only one point of covalent attachment to L.

Some embodiments provide an antibody drug conjugate (ADC) having the structure:

Ab-(L-D)ₚ
or a pharmaceutically acceptable salt thereof;
   wherein:
   Ab is an antibody;
   wherein each D is conjugated to a linker (L);
   wherein each L is covalently attached to Ab via a sulfur atom of a cysteine residue or an ϵ-amino group of a lysine residue;
   subscript p is an integer from 1 to 16;
   each D has the structure of Formula (II):

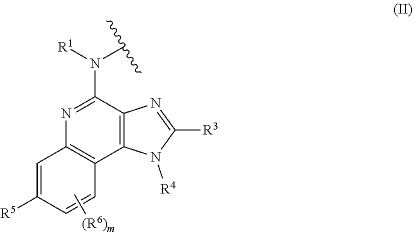

or a pharmaceutically acceptable salt thereof;
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R³ is selected from the group consisting of hydrogen, —NR^{A}R^{B}, —C(═O)NR^{A}R^{B} C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) the point of covalent attachment to L¹; (b) —OR^{C}; (c) —S(═O)₂R^{C}; (d) —C(═O)NR^{D}R^{E}; (e) —C(═O)OR^{C}; (f) —C(═O)SR^{C}; (g) —C(═S)R^{C}; (h) —PO₃R^{C}; or (j) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃ —C(═O)NR^{G}R^{H}, —S(O₂)NR^{G}R^{H}, —N(R′)—C(═O)R^{J}, —N(R^{T})—S(O₂)R^{K}, and SO₃R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
each instance of R¹ and R⁴ is optionally substituted with a solubilizing group selected from the group consisting of phosphoryl, sulfuryl, nitro, C₅-C₉ monosaccharide, C₁₀-C₁₈ disaccharide, and C₁₅-C₂₇ trisaccharide.

Some embodiments provide an antibody drug conjugate (ADC) having the structure:

Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
each D has the structure of Formula (III):

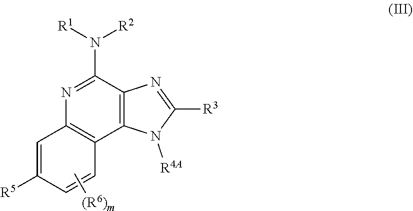

or a pharmaceutically acceptable salt thereof;
   wherein:
   R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
   R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
   R¹ and R², taken together with the nitrogen atom to which they are attached form a 3-6 membered heterocyclyl optionally substituted with 1-3 independently selected C₁-C₆ alkyl;
   R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃—C₈ cycloalkyl, phenyl, 5-10 membered heteroaryl, C₁-C₆ alkoxy, C₁-C₆ alkylthio, and —NR^{A}R^{B};
   R^{4A} is (a) the point of covalent attachment to L or (b) C₁-C₆ alkyl substituted with:
   (i) 1-3 independently selected halogen;
   (ii) —OR^{C};
   (iii) —SR^{C};
   (iv) —NH—S(O₂)R^{C};
   (v) —OC(═O)R^{C};
   (vi) —CO₂H;
   (vii) C₁-C₆ alkoxycarbonyl;
   (viii) —C(═O)NR^{D}R^{E};
   (ix) —NR^{D}R^{E};
   (x) —[N(C₁-C₆ alkyl)R^{D}R^{E}]⁺;
   (xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
   (xii) phenyl substituted with halogen, hydroxyl, C₁-C₆ alkoxy, —C(═O)NR^{D}R^{E} or —CO₂H;
   (xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
   (xiv) 5-10 membered heteroaryl optionally substituted with halogen, —NR^{D}R^{E}, C₁-C₆ alkoxy, —C(═O)NR^{D}R^{E}, or —CO₂H;
   wherein when R^{4A} is (b) the C₁-C₆ alkyl, or a substituent thereof, is further substituted with the point of covalent attachment to L;
   R⁵ is selected from the group consisting of hydrogen, —C(═O)OR^{F}, —NO₂, —CN, —CF₃ —C(═O)NR^{G}R^{H}, —S(O₂)NR^{G}R^{H}, —N(R^{I})—C(═O)R^{J}, —N(R^{I})—S(O₂)R^{K}, and SO₃R^{K};
   each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
   subscript m is 0, 1, 2, or 3;
   each R^{A} and R^{B} independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
   R^{C} is selected from the group consisting of hydrogen, phenyl, and C₁-C₁₀ alkyl optionally substituted with phenyl or 1-3 independently selected halogen;
   each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
   R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
   each R^{I}, R^{J}, and R^{K} is independently selected from the group consisting of hydrogen and C₁-C₆ alkyl; and
   R¹ is optionally substituted with a solubilizing group selected from the group consisting of phosphoryl, sulfuryl, nitro, C₅-C-9 monosaccharide, C₁₀-C₁₈ disaccharide, and C₁₅-C₂₇ trisaccharide.

Some embodiments provide an antibody drug conjugate (ADC) having the structure:

Ab-(L-D)ₚ
or a pharmaceutically acceptable salt thereof;
   wherein:
   Ab is an antibody;
   wherein each D is conjugated to a linker (L);
   wherein each L is covalently attached to Ab via a sulfur atom of a cysteine residue or an ϵ-amino group of a lysine residue;
   subscript p is an integer from 1 to 16;
L has the formula -M-(A)ₐ-(W)_{w}—(Y)_{y}—(X)ₓ, wherein:
   subscript a is 0 or 1;
   subscript y is 0 or 1;
   subscript w is 0 or 1;
subscript x is 0 or 1;
   M is a succinimide, a hydrolyzed succinimide, an amide, or a triazole;
A is a C₂₋₂₀ alkylene optionally substituted with 1-4 R^{a1}; or a 2 to 40 membered heteroalkylene optionally substituted with 1-4 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{a1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1} C(═O)NR^{a1}R^{e1}, —C(═O)(C₁₋₆ alkyl), and —C(═O)O(C₁₋₆ alkyl);
each R^{d1} and R^{e1} are independently hydrogen or C₁₋₃ alkyl;
   W is from 1-12 amino acids or has the structure:

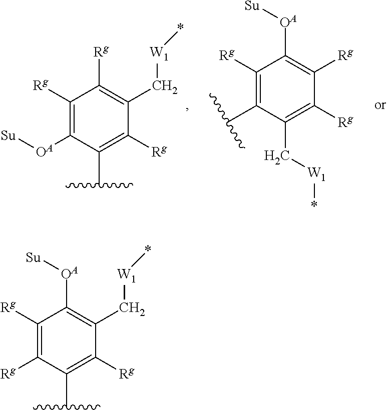

wherein Su is a Sugar moiety;
O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
   * represents covalent attachment to X, Y, or D;
Y is self-immolative moiety, a non-self-immolative releasable moiety, or a non-cleavable moiety;
X is a C₁-C₆ alkylene or a 3-6 membered heteroalkylene;
L is optionally substituted with a PEG Unit from PEG1 to PEG72;
   each D has the structure of Formula (II):

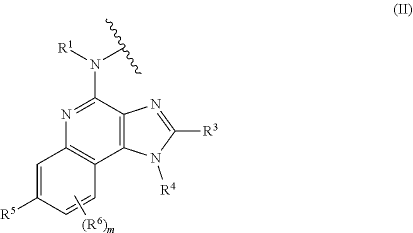

or a pharmaceutically acceptable salt thereof;
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃ —C(═O)NR^{G}R^{H}, —S(O₂)NR^{G}R^{H}, —N(R′)—C(═O)R^{J}, —N(R¹)—S(O₂)R^{K}, and SO₃R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
R⁴ is optionally substituted with a solubilizing group selected from the group consisting of phosphoryl, sulfuryl, nitro, C₅-C-9 monosaccharide, C₁₀-C₁₈ disaccharide, and C₁₅-C₂₇ trisaccharide.

Some embodiments provide an antibody drug conjugate (ADC) having the structure:

Ab-(L-D)ₚ
or a pharmaceutically acceptable salt thereof;
   wherein:
   Ab is an antibody;
   each L is a linker;
   wherein each D is conjugated to a linker;
   wherein each L is covalently attached to Ab via a sulfur atom of a cysteine residue or an ϵ-amino group of a lysine residue;
   subscript p is an integer from 1 to 16;
L has the formula -M-(A)ₐ-(W)_{w}—(Y)_{y}—(X)ₓ, wherein:
   subscript a is 0 or 1;
   subscript y is 0 or 1;
   subscript w is 0 or 1;
subscript x is 0 or 1;
   M is a succinimide, a hydrolyzed succinimide, an amide, a methyl ketone, or a triazole;
A is a C₂₋₂₀ alkylene optionally substituted with 1-4 R^{a1}; or a 2 to 40 membered heteroalkylene optionally substituted with 1-4 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1} C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
   W is from 1-12 amino acids or has the structure:

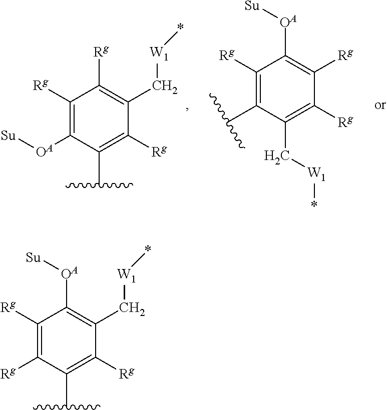

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
   * represents covalent attachment to X, Y, or D;
Y is self-immolative moiety, a non-self-immolative releasable moiety, or a non-cleavable moiety;
X is a C₁-C₆ alkylene or a 3-6 membered heteroalkylene;
L is optionally substituted with a PEG Unit from PEG1 to PEG72;
   each D has the structure of Formula (III):

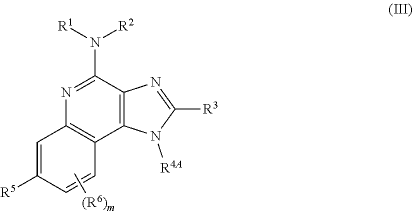

or a pharmaceutically acceptable salt thereof;
wherein:
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R^{4A} is (a) the point of covalent attachment to L or (b) C₁-C₆ alkyl substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
wherein when R^{4A} is (b) the C₁-C₆ alkyl, or a substituent thereof, is further substituted with the point of covalent attachment to L;
R⁵ is selected from the group consisting of hydrogen, —C(═O)OR^{F}, —NO₂, —CN, —CF₃ —C(═O)NR^{G}R^{H}, —S(O₂)NR^{G}R^{H}, —N(R^{I})—C(═O)R^{J}, —N(R^{I})—S(O₂)R^{K}, and SO₃R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is i independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
R¹ is optionally substituted with a solubilizing group selected from the group consisting of phosphoryl, sulfuryl, nitro, C₅-C₉ monosaccharide, C₁₀-C₁₈ disaccharide, and C₁₅-C₂₇ trisaccharide.

Some embodiments provide an antibody drug conjugate (ADC) having the structure:

Ab-(L-D)ₚ
or a pharmaceutically acceptable salt thereof;
   wherein:
   Ab is an antibody;
   each L is a linker;
   wherein each D is conjugated to a linker;
   wherein each L is covalently attached to Ab via a sulfur atom of a cysteine residue or an ϵ-amino group of a lysine residue;
   subscript p is an integer from 1 to 16;
L has the formula -M-(A)ₐ-(W)_{w}—(Y)_{y}—(X)ₓ—, wherein:
   subscript a is 0 or 1;
   subscript y is 0 or 1;
   subscript w is 0 or 1;
subscript x is 0 or 1;
   M is a succinimide, a hydrolyzed succinimide, an amide, a methyl ketone, or a triazole;
A is a C₂₋₂₀ alkylene optionally substituted with 1-4 R^{a1}; or a 2 to 40 membered heteroalkylene optionally substituted with 1-4 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{a1}R^{e1} C(═O)NR^{a1}R^{e1}, —C(═O)(C₁₋₆ alkyl), and —C(═O)O(C₁₋₆ alkyl);
each R^{d1} and R^{e1} are independently hydrogen or C₁₋₃ alkyl;
   W is from 1-12 amino acids or has the structure:

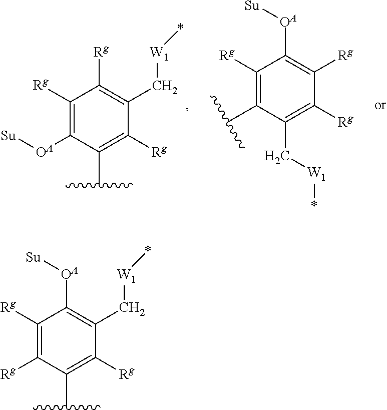

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
   * represents covalent attachment to X, Y, or D;
Y is self-immolative moiety, a non-self-immolative releasable moiety, or a non-cleavable moiety;
X is a C₁-C₆ alkylene or a 3-6 membered heteroalkylene;
L is optionally substituted with a PEG Unit from PEG1 to PEG72;
   each D has the structure of Formula (IV):

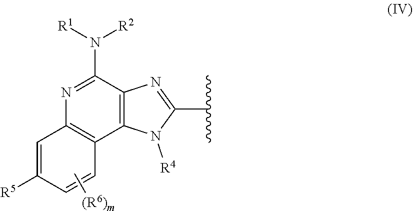

or a pharmaceutically acceptable salt thereof;
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R⁴ is (a) —OR^{C} or (b) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H; R⁵ is selected from the group consisting of hydrogen, —C(═O)OR^{F}, —NO₂, —CN, —CF₃ —C(═O)NR^{G}R^{H}, —S(O₂)NR^{G}R^{H}, —N(R′)—C(═O)R^{J}, —N(R^{T})—S(O₂)R^{K}, and SO₃R^{K}; each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen; each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
R¹ is optionally substituted with a solubilizing group (Sb) selected from the group consisting of phosphoryl, sulfuryl, nitro, C₅-C₉ monosaccharide, C₁₀-C₁₈ disaccharide, and C₁₅-C₂₇ trisaccharide.

Some embodiments provide an antibody drug conjugate (ADC) having the structure:

Ab-(L-D)ₚ
or a pharmaceutically acceptable salt thereof;
   wherein:
   Ab is an antibody;
   each L is a linker;
   wherein each D is conjugated to a linker;
   wherein each L is covalently attached to Ab via a sulfur atom of a cysteine residue or an ϵ-amino group of a lysine residue;
   subscript p is an integer from 1 to 16;
L has the formula -M-(A)ₐ-(W)_{w}—(Y)_{y}—(X)ₓ, wherein:
   subscript a is 0 or 1;
   subscript y is 0 or 1;
   subscript w is 0 or 1;
subscript x is 0 or 1;
M is a succinimide, a hydrolyzed succinimide, an amide, a methyl ketone, or a triazole;
A is a C₂₋₂₀ alkylene optionally substituted with 1-4 R^{a1}; or a 2 to 40 membered heteroalkylene optionally substituted with 1-4 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{a1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{a1}R^{e1}, —C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
   W is from 1-12 amino acids or has the structure:

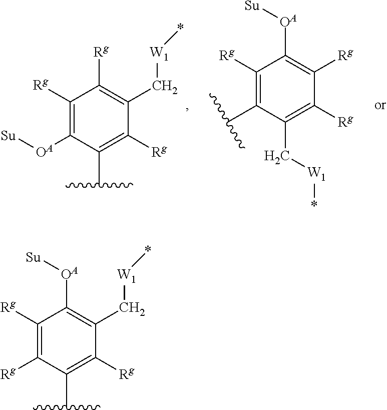

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
   * represents covalent attachment to X, Y, or D;
Y is self-immolative moiety, a non-self-immolative releasable moiety, or a non-cleavable moiety;
X is a C₁-C₆ alkylene or a 3-6 membered heteroalkylene;
L is optionally substituted with a PEG Unit from PEG1 to PEG72;
   each D has the structure of Formula (V):

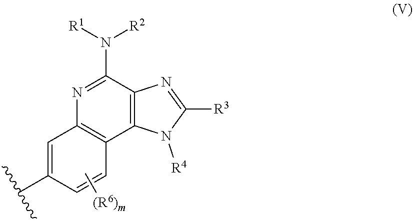

or a pharmaceutically acceptable salt thereof;
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D} and R^{E} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, together with the nitrogen atom to which they are attached form a 3-6 membered heterocyclyl optionally substituted with 1-3 independently selected C₁-C₆ alkyl; and
each instance of R¹ and R⁴ is optionally substituted with a solubilizing group (Sb) selected from the group consisting of phosphoryl, sulfuryl, nitro, C₅-C₉ monosaccharide, C₁₀-C₁₈ disaccharide, and C₁₅-C₂₇ trisaccharide.

Some embodiments provide an antibody drug conjugate (ADC) having the structure:

Ab-(L-D)ₚ
or a pharmaceutically acceptable salt thereof;
   wherein:
   Ab is an antibody;
   each L is a linker;
   wherein each D is conjugated to a linker;
   wherein each L is covalently attached to Ab via a sulfur atom of a cysteine residue or an ϵ-amino group of a lysine residue;
   subscript p is an integer from 1 to 16;
L has the formula -M-(A)ₐ-(W)_{w}—(Y)_{y}—(X)ₓ, wherein:
   subscript a is 0 or 1;
   subscript y is 0 or 1;
   subscript w is 0 or 1;
subscript x is 0 or 1;
   M is a succinimide, a hydrolyzed succinimide, an amide, a methyl ketone, or a triazole;
A is a C₂₋₂₀ alkylene optionally substituted with 1-4 R^{a1}; or a 2 to 40 membered heteroalkylene optionally substituted with 1-4 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1} C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
   W is from 1-12 amino acids or has the structure:

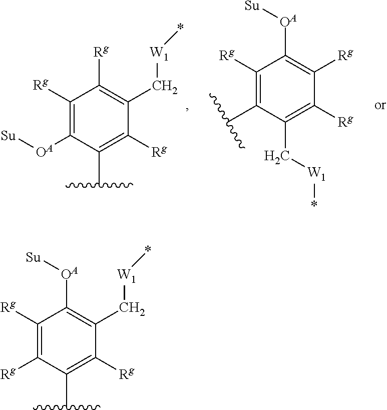

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
   * represents covalent attachment to X, Y, or D;
Y is self-immolative moiety, a non-self-immolative releasable moiety, or a non-cleavable moiety;
X is a C₁-C₆ alkylene or a 3-6 membered heteroalkylene;
L is optionally substituted with a PEG Unit from PEG1 to PEG72;
   each D has the structure of Formula (VI):

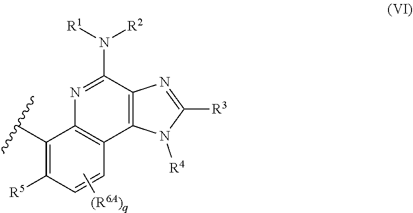

or a pharmaceutically acceptable salt thereof;
wherein
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃ —C(═O)NR^{G}R^{H}, —S(O₂)NR^{G}R^{H}, —N(R^{I})—C(═O)R^{J}, —N(R^{I})—S(O₂)R^{K}, and SO₃R^{K};
each R^{6A} is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript q is 0, 1, or 2;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and each instance of R¹ and R⁴ is optionally substituted with a solubilizing group (Sb) selected from the group consisting of phosphoryl, sulfuryl, nitro, C₅-C₉ monosaccharide, C₁₀-C₁₈ disaccharide, and C₁₅-C₂₇ trisaccharide.

Some embodiments provide an antibody drug conjugate (ADC) having the structure:

Ab-(L-D)ₚ
or a pharmaceutically acceptable salt thereof;
   wherein:
   Ab is an antibody;
   each L is a linker;
   wherein each D is conjugated to a linker;
   wherein each L is covalently attached to Ab via a sulfur atom of a cysteine residue or an ϵ-amino group of a lysine residue;
   subscript p is an integer from 1 to 16;
L has the formula -M-(A)ₐ-(W)_{w}—(Y)_{y}—(X)ₓ, wherein:
   subscript a is 0 or 1;
   subscript y is 0 or 1;
   subscript w is 0 or 1;
subscript x is 0 or 1;
M is a succinimide, a hydrolyzed succinimide, an amide, a methyl ketone, or a triazole;
A is a C₂₋₂₀ alkylene optionally substituted with 1-4 R^{a1}; or a 2 to 40 membered heteroalkylene optionally substituted with 1-4 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{a1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1} C(═O)NR^{a1}R^{e1}, —C(═O)(C₁₋₆ alkyl), and —C(═O)O(C₁₋₆ alkyl);
each R^{d1} and R^{e1} are independently hydrogen or C₁₋₃ alkyl;
   W is from 1-12 amino acids or has the structure:

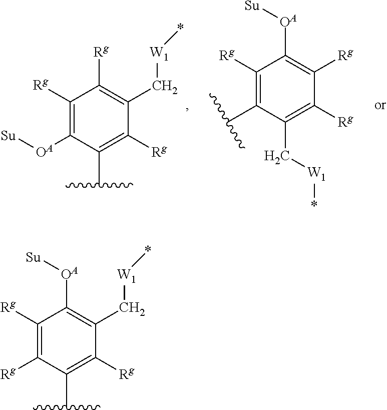

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
   * represents covalent attachment to X, Y, or D;
Y is self-immolative moiety, a non-self-immolative releasable moiety, or a non-cleavable moiety;
X is a C₁-C₆ alkylene or a 3-6 membered heteroalkylene;
L is optionally substituted with a PEG Unit from PEG1 to PEG72;
   each D has the structure of Formula (VII):

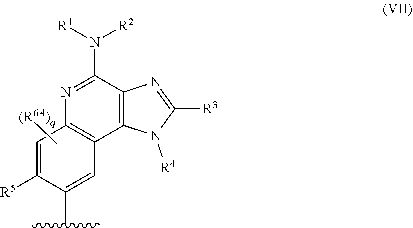

or a pharmaceutically acceptable salt thereof;
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃ —C(═O)NR^{G}R^{H}, —S(O₂)NR^{G}R^{H}, —N(R^{I})—C(═O)R^{J}, —N(R^{I})—S(O₂)R^{K}, and SO₃R^{K};
each R^{6A} is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript q is 0, 1, or 2;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
each instance of R¹ and R⁴ is optionally substituted with a solubilizing group (Sb) selected from the group consisting of phosphoryl, sulfuryl, nitro, C₅-C₉ monosaccharide, C₁₀-C₁₈ disaccharide, and C₁₅-C₂₇ trisaccharide.

Some embodiments provide an antibody drug conjugate (ADC) having the structure:

Ab-(L-D)ₚ
or a pharmaceutically acceptable salt thereof;
   wherein:
   Ab is an antibody;
   each L is a linker;
   wherein each D is conjugated to a linker;
   wherein each L is covalently attached to Ab via a sulfur atom of a cysteine residue or an ϵ-amino group of a lysine residue;
   subscript p is an integer from 1 to 16;
L has the formula -M-(A)ₐ-(W)_{w}—(Y)_{y}—(X)ₓ, wherein:
   subscript a is 0 or 1;
   subscript y is 0 or 1;
   subscript w is 0 or 1;
subscript x is 0 or 1;
   M is a succinimide, a hydrolyzed succinimide, an amide, a methyl ketone, or a triazole;
A is a C₂₋₂₀ alkylene optionally substituted with 1-4 R^{a1}; or a 2 to 40 membered heteroalkylene optionally substituted with 1-4 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{a1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1} —(C₁₋₆ alkylene)-NR^{a1}R^{e1} C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
   W is from 1-12 amino acids or has the structure:

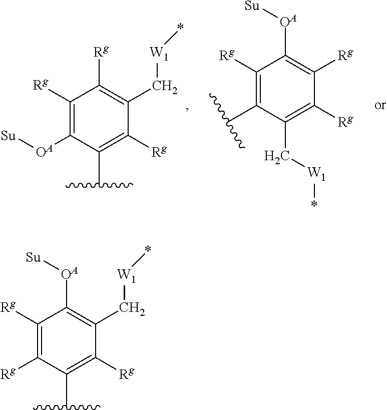

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
   * represents covalent attachment to X, Y, or D;
Y is self-immolative moiety, a non-self-immolative releasable moiety, or a non-cleavable moiety;
X is a C₁-C₆ alkylene or a 3-6 membered heteroalkylene;
L is optionally substituted with a PEG Unit from PEG1 to PEG72;
   each D has the structure of Formula (VIII):

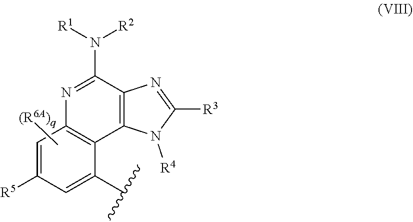

or a pharmaceutically acceptable salt thereof;
wherein:
[symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)2R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]+;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]+, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]+, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃—C(═O)NR^{G}R^{H}, —S(O₂)NR^{G}R^{H}, —N(R′)—C(═O)R, —N(R)—S(O₂)R^{K}, and SO₃R^{K};
each R^{6A} is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; subscript q is 0, 1, or 2;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl; R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
each instance of R¹ and R⁴ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide.

Some embodiments provide an antibody drug conjugate (ADC) having the structure:

Ab-(L-D)p
or a pharmaceutically acceptable salt thereof,
   wherein:
   Ab is an antibody;
   wherein each D is conjugated to a linker (L);
   wherein each L is covalently attached to Ab via a sulfur atom of a cysteine residue or an ϵ-amino group of a lysine residue;
   subscript p is an integer from 1 to 16;
   each D has the structure of Formula (XI):

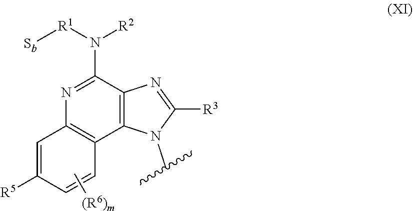

or a pharmaceutically acceptable salt thereof,
wherein
[symbol] represents covalent attachment to L;
R¹ is a hydrolysable group selected from the group consisting of C₁-C₆ alkoxycarbonyl, C₁-C₆ alkoxythiocarbonyl, C₁-C₆ carbamoyl, C₁-C₆ amidine, C₁-C₆ sulfone, and C₁-C₆ thione; wherein each C₁-C₆ alkoxycarbonyl, C₁-C₆ alkoxythiocarbonyl, C₁-C₆ carbamoyl, C₁-C₆ amidine, C₁-C₆ sulfone, and C₁-C₆ thione, is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁵ is selected from the group consisting of hydrogen, —C(═O)OR^{F}, —NO₂, —CN, —CF₃—C(═O)NR^{G}R^{H}, —S(O₂)NR^{G}R^{H}, —N(R′)—C(═O)R, —N(R)—S(O₂)R^{K}, and SO₃R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R¹, R, and R^{K} is independently selected from the group consisting of hydrogen and C₁-C₆ alkyl; and
S_{b} is a solubilizing group selected from the group consisting of phosphoryl, sulfuryl, nitro, C₅-C₉ monosaccharide, C₁₀-C₁₈ disaccharide, and C₁₁₅-C₂₇ trisaccharide.

In some embodiments of Formula (XI), R¹ is a hydrolysable group selected from the group consisting of C₁-C₆ alkoxycarbonyl and C₁-C₆ carbamoyl; wherein each C₁-C₆ alkoxycarbonyl and C₁-C₆ carbamoyl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}. In some embodiments of Formula (XI), R¹ is a C₁-C₆ alkoxycarbonyl optionally substituted with 1-3 substituents independently selected from the group consisting of hydroxyl, C₃-C₈ cycloalkyl, phenyl, 5-10 membered heteroaryl, C₁-C₆ alkoxy, and —NR^{A}R^{B}. In some embodiments of Formula (XI), S_{b} is selected from the group consisting of C₅-C₉ monosaccharide and C₁₀-C₁₈ disaccharide. In some embodiments of Formula (XI), R¹, —S_{b} is

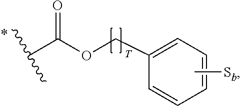

wherein subscript T is 1-6. In some embodiments of Formula (XI), R² is hydrogen or C₁-C₆ alkyl. In some embodiments of Formula (XI), R³ is selected from the group consisting of C₁-C₆ alkyl, C₂-C₆ alkenyl, C₂-C₆ alkynyl, C₃-C₆ cycloalkyl, phenyl, 5-10 membered heteroaryl, and 3-12 membered heterocycle. In some embodiments of Formula (XI), R³ is C₁-C₆ alkyl or C₃-C₆ cycloalkyl. In some embodiments of Formula (XI), R⁵ comprises a pH of at most about 7.0, a dipole moment of at least about 2.0 Debye, or both.

In some embodiments of Formula (XI), R⁵ is selected from the group consisting of —C(═O)OH, —NO₂, —CN, —CF₃, and —S(O₃)H. In some embodiments of Formula (XI), R⁵ is —C(═O)OR^{K}. In some embodiments of Formula (XI), subscript m is 0. In some embodiments of Formula (XI), the linker (L) comprises a cleavable group. In some embodiments of Formula (XI), the cleavable group comprises a glycosidic bond, peptide bond, carbamate, or quaternary amine.

In some embodiments, R^{D} and R^{E}, or R^{G} and R^{H}, together with the nitrogen atom to which they are attached form a 3-6 membered heterocyclyl optionally substituted with 1-3 independently selected C₁-C₆ alkyl and the other R^{G} and R^{H} or R^{D} and R^{E} are independently selected from the group consisting of hydrogen, C₁-C₆ alkyl, C₂-C₆ alkenyl, C₃-C₅ cycloalkyl, C₃-C₈ cycloalkyl(C₁-C₆ alkyl)-, aryl, and aryl(C₁-C₆ alkyl)-. In some embodiments, one of R^{D}, R^{E}, R^{G}, and R^{H} is the point of covalent attachment to L and the other of R^{D}, R^{E}, R^{G}, and R^{H} are independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-. In some embodiments, one of R^{I}, R^{J}, and R^{K} is the point of covalent attachment to L, and the other R^{I}, R^{J}, and R^{K} are independently selected from the group consisting of hydrogen and C₁-C₆ alkyl.

Some embodiments provide an antibody drug conjugate (ADC) having the structure:

Ab [symbol] M-(A)ₐ-(W)_{w}-(Y)_{y}-(X)ₓ-D)ₚ
or a pharmaceutically acceptable salt thereof,
wherein:
Ab is an antibody;
subscript p is an integer from 1 to 16;
each D has the structure selected from the group consisting of:

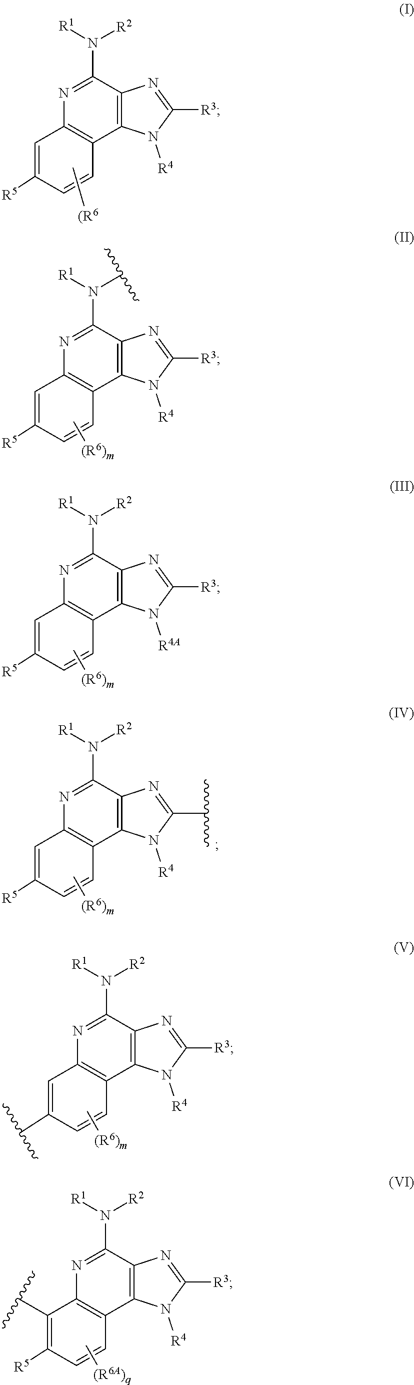

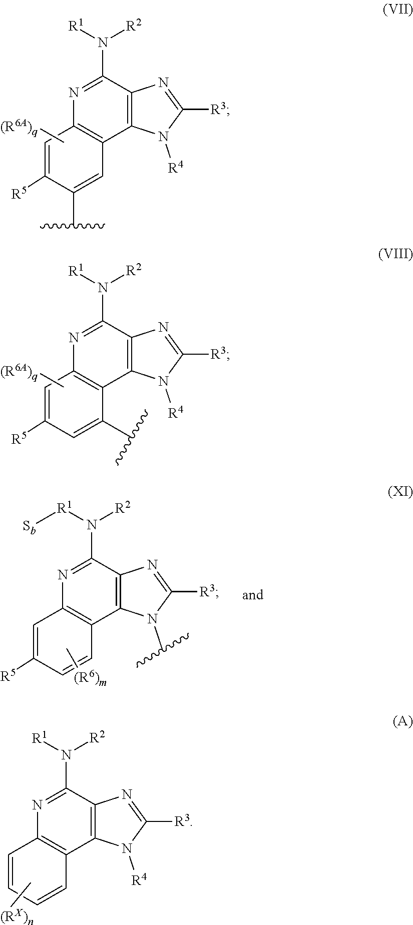

In some embodiments, the ADC is selected from the group consisting of:

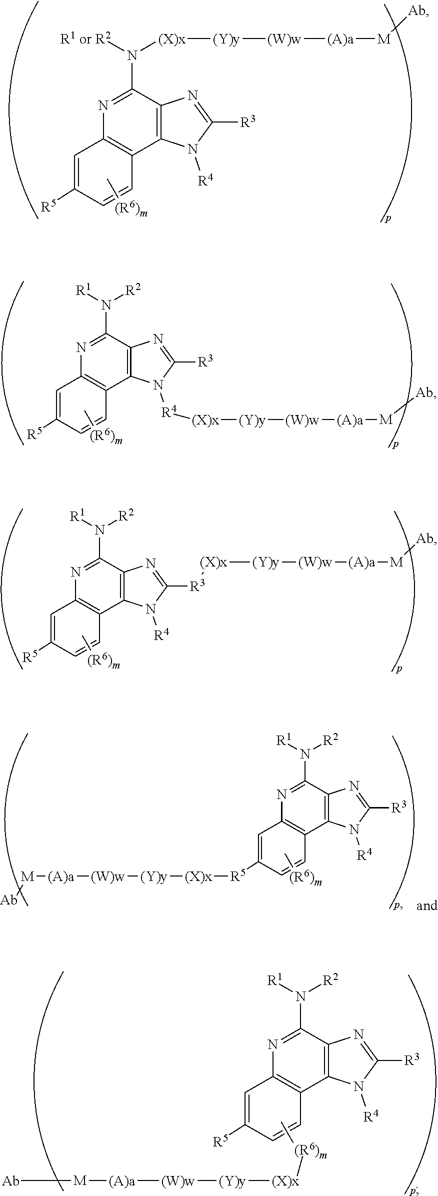

or a pharmaceutically acceptable salt thereof, wherein Ab is an antibody and the remaining variables are as defined herein.

In some embodiments, the ADC is selected from the group consisting of:

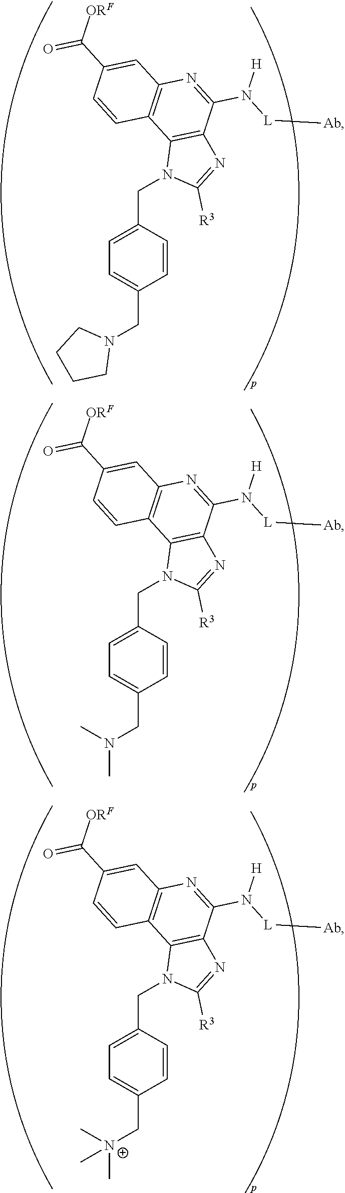

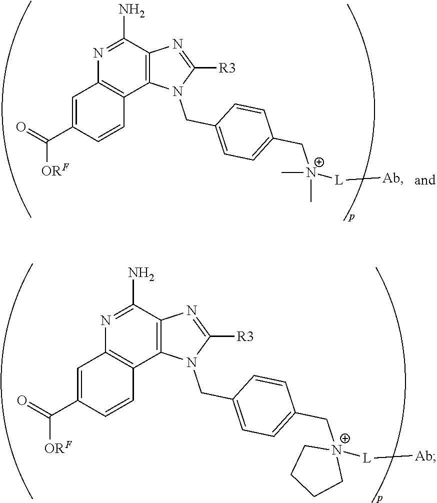

or a pharmaceutically acceptable salt thereof, wherein Ab is an antibody, L is a linker, and R³, R^{F}, and subscript p are as defined herein. In some embodiments, the ADCs described herein are present in the form of a salt. In some embodiments, the salt is a pharmaceutically acceptable salt.

In some embodiments subscript p is an integer from 1 to 8; from 4 to 12; or from 8 to 16. In some embodiments, subscript p is an even number. In some embodiments, subscript p is 2, 4, 6, 8, 10, 12, 14, or 16. In some embodiments, subscript p is 2, 4, 6, or 8. In some embodiments, each L is covalently attached to Ab via a sulfur atom of a cysteine residue. In some embodiments, one or more of the cysteine residues is an engineered cysteine residue. In some embodiments, each cysteine residue is an engineered cysteine residue. In some embodiments, one or more of the cysteine residues is a native cysteine residue. In some embodiments, each cysteine residue is a native cysteine residue. In some embodiments, each sulfur atom is from a cysteine residue from a reduced interchain disulfide bond. In some embodiments, each L is covalently attached to Ab via an ϵ-amino group of a lysine residue.

In some embodiments, the ADC is capable of releasing (i) a component of the linker bound to D; (ii) a component of antibody that has not undergone subsequent intracellular metabolism bound to L-D; and/or (iii) the parent compound D, as the free drug (as defined herein). In some embodiments, the free drug is released at the intended site of action targeted by the antibody. In some embodiments, the free drug is released within the intended site of action targeted by the antibody. In some embodiments, the free drug is capable of binding to a toll-like receptor (TLR). In some embodiments, the binding of the free drug to a TLR exhibits an agonist effect on the TLR. In some embodiments, the binding of the free drug to a TLR exerts an immunostimulatory effect.

### Antibodies

In some embodiments, an antibody is a polyclonal antibody. In some embodiments, an antibody is a monoclonal antibody. In some embodiments, an antibody is chimeric. In some embodiments, an antibody is humanized. In some embodiments, an antibody is an antigen binding fragment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies and is not to be construed as requiring production of the antibody by any particular method.

Useful polyclonal antibodies are heterogeneous populations of antibody molecules derived from the sera of immunized animals. Useful monoclonal antibodies are homogeneous populations of antibodies to a particular antigenic determinant (e.g., a cancer or immune cell antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

In some embodiments, an antibody includes a functionally active fragment, derivative or analog of an antibody that binds specifically to target cells (e.g., cancer cell antigens) or other antibodies bound to cancer cells or matrix. In this regard, “functionally active” means that the fragment, derivative or analog is able to bind specifically to target cells. To determine which CDR sequences bind the antigen, synthetic peptides containing the CDR sequences are typically used in binding assays with the antigen by any binding assay method known in the art (e.g., the Biacore assay) (See, e.g., Kabat et al., 1991, Sequences of Proteins of Immunological Interest, Fifth Edition, National Institute of Health, Bethesda, Md; Kabat E et al., 1980, J Immunology 125(3):961-969).

Additionally, recombinant antibodies, such as chimeric and humanized monoclonal antibodies, comprising both human and non-human portions, which are typically obtained using standard recombinant DNA techniques, are useful antibodies. A chimeric antibody is a molecule in which different portions are derived from different animal species, such as for example, those having a variable region derived from a murine monoclonal and a constant region derived from a human immunoglobulin. See, e.g., U.S. Pat. Nos. 4,816,567; and 4,816,397, which are incorporated herein by reference in their entireties. Humanized antibodies are antibody molecules from non-human species having one or more CDRs from the non-human species and a framework region from a human immunoglobulin molecule. See, e.g., U.S. Pat. No. 5,585,089, which is incorporated herein by reference in its entirety. Such chimeric and humanized monoclonal antibodies can be produced by recombinant DNA techniques known in the art, for example using methods described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; European Patent Publication No. 012 023; Berter et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Cancer. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321: 522-525; Verhoeyan et al., 1988, Science 239:1534; and Beidler et al., 1988, J. Immunol. 141:4053-4060; each of which is incorporated herein by reference in its entirety.

In some embodiments, an antibody is a completely human antibody. In some embodiments, an antibody is produced using transgenic mice that are incapable of expressing endogenous immunoglobulin heavy and light chain genes, but which are capable of expressing human heavy and light chain genes.

In some embodiments, an antibody is an intact or fully-reduced antibody. The term ‘fully-reduced’ is meant to refer to an antibody in which all four inter-chain disulfide linkages have been reduced to provide eight thiols that can be attached to a linker (L).

Attachment to an antibody can be via thioether linkages from native and/or engineered cysteine residues, or from an amino acid residue engineered to participate in a cycloaddition reaction (such as a click reaction) with the corresponding linker intermediate. See, e.g., Maerle, et al., PLOS One 2019: 14(1); e0209860. In some embodiments, an antibody is an intact or fully-reduced antibody, or is an antibody bearing engineered an cysteine group that is modified with a functional group that can participate in, for example, click chemistry or other cycloaddition reactions for attachment of other components of the ADC as described herein (e.g., Diels-Alder reactions or other [3+2] or [4+2] cycloadditions). See, e.g., Agard, et al., J. Am. Chem. Soc. Vol. 126, pp. 15046-15047 (2004); Laughlin, et al., Science, Vol. 320, pp. 664-667 (2008); Beatty, et al., ChemBioChem, Vol. 11, pp. 2092-2095 (2010); and Van Geel, et al., Bioconjug. Chem. Vol. 26, pp. 2233-2242 (2015).

Antibodies that bind specifically to a cancer or immune cell antigen are available commercially or produced by any method known to one of skill in the art such as, e.g., chemical synthesis or recombinant expression techniques. The nucleotide sequences encoding antibodies that bind specifically to a cancer or immune cell antigen are obtainable, e.g., from the GenBank database or similar database, literature publications, or by routine cloning and sequencing.

In some embodiments, the antibody can be used for the treatment of a cancer (e.g., an antibody approved by the FDA and/or EMA). Antibodies that bind specifically to a cancer or immune cell antigen are available commercially or produced by any method known to one of skill in the art such as, e.g., recombinant expression techniques. The nucleotide sequences encoding antibodies that bind specifically to a cancer or immune cell antigen are obtainable, e.g., from the GenBank database or similar database, literature publications, or by routine cloning and sequencing.

In some embodiments, an antibody can be configured to bind to a surface antigen of a cell. The antibody, or a complex comprising the antibody, can be configured to internalize within a cell upon binding to the surface antigen. For example, the antibody or an ADC comprising the antibody can be configured to endocytose upon binding to a surface antigen of a cell. In some embodiments, the antibody (or an ADC comprising the antibody) is configured to internalize within a cancer cell. In some embodiments, the antibody (or an ADC comprising the antibody) is configured to internalize within an immune cell. In some embodiments, the immune cell is a tumor associated macrophage. In some embodiments, the surface antigen is a receptor or a receptor complex (e.g., expressed on lymphocytes). In some embodiments, the receptor or receptor complex comprises an immunoglobulin gene superfamily member, a TNF receptor superfamily member, an integrin, a cytokine receptor, a chemokine receptor, a major histocompatibility protein, a lectin, or a complement control protein or other immune cell expressed surface receptor.

In some embodiments, an antibody is configured to bind specifically to a cancer cell antigen. In some embodiments, an antibody is configured to bind specifically to an immune cell antigen. In some embodiments, the immune cell antigen is a tumor associated macrophage antigen. In some embodiments, an antibody is configured to bind specifically to EphA2. It will be understood that the antibody component in an ADC is an antibody in residue form such that “Ab” in the ADC structures described herein incorporates the structure of the antibody.

Non-limiting examples of antibodies that can be used for treatment of cancer and antibodies that bind specifically to tumor associated antigens are disclosed in Franke, A. E., Sievers, E. L., and Scheinberg, D. A., “Cell surface receptor-targeted therapy of acute myeloid leukemia: a review” Cancer Biother Radiopharm. 2000, 15, 459-76; Murray, J. L., “Monoclonal antibody treatment of solid tumors: a coming of age” Semin Oncol. 2000, 27, 64-70; Breitling, F., and Dubel, S., Recombinant Antibodies, John Wiley, and Sons, New York, 1998, each of which is hereby incorporated by reference in its entirety.

Examples of antibodies that bind to one or more of a cancer cell antigen and an immune cell antigen are provided below.

Non-limiting examples of target antigens and associated antibodies useful for the treatment of cancer and antibodies that bind specifically to cancer cell antigens (also called tumor antigens), include ADAM12 (e.g., Catalog #14139-1-AP); ADAM9 (e.g., IMGC936); AFP (e.g., ThermoFisher Catalog #PA5-25959); AGR2 (e.g., ThermoFisher Catalog #PA5-34517); AKAP-4 (e.g., Catalog #PA5-52230); ALK (e.g., DLX521); ALPP (e.g., Catalog #MA5-15652); ALPPL2 (e.g., Catalog #PA5-22336); AMHR2 (e.g., ThermoFisher Catalog #PA5-13902); androgen receptor (e.g., ThermoFisher Catalog #MA5-13426); ANTXR1 (e.g., Catalog #MA1-91702); ANXA1 (e.g., Catalog #71-3400); ARTN (e.g., ThermoFisher Catalog #PA5-47063); ASCT2 (e.g., idactamab); Axl (e.g., BA3011; tilvestamab); B7-DC (e.g., Catalog #PA5-20344); B7-H3 (e.g., enoblituzumab, omburtamab, MGD009, MGC018, DS-7300); B7-H4 (e.g., Catalog #14-5949-82); B7-H6 (e.g., Catalog #12-6526-42); B7-H7; BAFF-R (e.g., Catalog #14-9117-82); BCMA; BCR-ABL; BMPR2; BORIS; C₅ complement (e.g., BCD-148; CAN106); CA-125; CA19-9 (e.g., AbGn-7; MVT-5873); CA9 (e.g., girentuximab); CALCR (see, e.g., International Publication No. WO 2015077826); CAMPATH-1 (e.g., alemtuzumab; ALLO-647; ANT1034); carcinoembryonic antigen (e.g., arcitumomab; cergutuzumab; amunaleukin; labetuzumab); CCNB1; CD112 (see, e.g., U.S. Publication No. 20100008928); CD115 (e.g., axatilimab; cabiralizumab; emactuzumab); CD123 (e.g., BAY-943; CSL360); CD137 (e.g., ADG106; CTX-471); CD147 (e.g., gavilimomab; metuzumab); CD155 (e.g., U.S. Publication No. 2018/0251548); CD19 (e.g., ALLO-501); CD20 (e.g., divozilimab; ibritumomab tiuxetan); CD24 (see, e.g., U.S. Pat. No. 8,614,301); CD244 (e.g., R&D AF1039); CD247 (e.g., AFM15); CD27 (e.g., varlilumab); CD274 (e.g., adebrelimab; atezolizumab; garivulimab); CD3 (e.g., otelixizumab; visilizumab); CD30 (e.g., iratumumab); CD33 (e.g., lintuzumab; BI 836858; AMG 673); CD352 (e.g., SGN-CD352A); CD37 (e.g., lilotomab; GEN3009); CD38 (e.g., felzartamab; AMG 424); CD3D; CD3E (e.g., foralumab; teplizumab); CD3G; CD45 (e.g., apamistamab); CD47 (e.g., letaplimab; magrolimab); CD48 (e.g., SGN-CD48A); CD5 (e.g., MAT 304; zolimomab aritox); CD70 (e.g., cusatuzumab); CD74 (e.g., milatuzumab); CD79A (see, e.g., International Publication No. WO 2020252110); CD96; CD97; CD-262 (e.g., tigatuzumab); CDCP1 (e.g., RG7287); CDH17 (see, e.g., International Publication No. WO 2018115231); CDH3 (e.g., PCA062); CDH6 (e.g., HKT288); CEACAMI; CEACAM6; CLDN1 (e.g., INSERM anti-Claudin-1); CLDN16; CLDN18.1 (e.g., zolbetuximab); CLDN18.2 (e.g., zolbetuximab); CLDN19; CLDN2 (see, e.g., International Publication No. WO 2018123949); CLEC12A (e.g., tepoditamab); CLPTM1L; CSPG4 (e.g., U.S. Pat. No. 10,822,427); CXCR4 (e.g., ulocuplumab); CYP1B1; DCLK1 (see, e.g., International Publication No. WO 2018222675); DDR1; de2-7 EGFR (e.g., MAb 806); DPEP1; DPEP3; DPP4; DR4 (e.g., mapatumumab); DSG2 (see, e.g., U.S. Pat. No. 10,836,823); EGF; EGFR; endosialin (e.g., ontuxizumab); ENPP1; EPCAM (e.g., adecatumumab); EPHA receptors; EPHA2; ERBB2 (e.g., trastuzumab); ERBB3; ERVMER34-1; ETV6-AML (e.g., Catalog #PA5-81865); FAS; FasL; Fas-related antigen 1; FBP; FGFR1 (e.g., RG7992); FGFR2 (e.g., aprutumab); FGFR3 (e.g., vofatamab); FGFR4 (e.g., MM-161); FLT3 (e.g., 4G8SDIEM); FN; FN1; FOLR1 (e.g., farletuzumab); FSHR; FucGM1 (e.g., BMS-986012); FZD5; FZD8; G250; GAGE; GD2 (e.g., dinutuximab); GD3 (e.g., mitumomab); GITR (e.g., ragifilimab); GloboH; GM2 (e.g., BIW-8962); GM3 (e.g., racotumomab); gp100; GPA33 (e.g., KRN330); GPC3 (e.g., codrituzumab); gpNMB (e.g., glembatumumab); GPR87; GUCY2C (e.g., indusatumab); HAS3; HAVCR2; HLA-E; HLA-F; HLA-G (e.g., TTX-080); HPV E6 E7; hTERT; ICAM1; IDOl; IFNAR1 (e.g., faralimomab); IFNAR2; IL13Ra2; IL1RAP (e.g., nidanilimab); IL-21R (e.g., PF-05230900); IL-5R (e.g., benralizumab); ITGAV (e.g., abituzumab); ITGB6; ITGB8; KISSIR; L1CAM (e.g., JCAR023); LAG-3 (e.g., encelimab); LAMP1; LCK; legumain; LMP2; LY6G6D (e.g., PA5-23303); LY9 (e.g., PA5-95601); LYPD1 (e.g., ThermoFisher Catalog #PA5-26749); MAD-CT-1; MAD-CT-2; MAGEA1 (e.g., Catalog #MA5-11338); MAGEA3 (e.g., ThermoFisher Catalog #60054-1-IG); MAGEA4 (e.g., Catalog #MA5-26117); MAGEC2 (e.g., ThermoFisher Catalog #PA5-64010); MELTF (e.g., ThermoFisher Catalog #H00004241-M04A); MerTk (e.g., DS5MMER, Catalog #12-5751-82); a metalloproteinase; MFSD13A; MICA (e.g., 1E2C₈, Catalog #66384-1-IG); MICB (e.g., Catalog #MA5-29422); Mincle (e.g., OTI2A8, Catalog #TA505101); MLANA (e.g., Catalog #MA5-15237); ML-IAP (e.g., 88C₅₇₀, ThermoFisher Catalog #40958); MSLN (e.g., 5B2, Catalog #MA5-11918); MUC1 (e.g., MH1 (CT2), ThermoFisher Catalog #MA5-11202); MUC5AC (e.g., 45M1, Catalog #MA5-12178); MYCN (e.g., NCM-II 100, ThermoFisher Catalog #MA1-170); NA17; NCAM1 (e.g., ThermoFisher Catalog #MA5-11563); Nectin-4 (e.g., enfortumab); NOX1 (e.g., Catalog #PA5-103220); NT5E (e.g., 7G2, ThermoFisher Catalog #41-0200); NY-BR-1 (e.g., NY-BR-1 No. 2, Catalog #MA5-12645); NY-ESO-1 (e.g., E978m, Catalog #35-6200); OX40 (e.g., ABM193); OY-TES1; p53; p53mutant; PAP; PAX3 (e.g., GT1210, ThermoFisher Catalog #MA5-31583); PAX5; PDGFR-B (e.g., rinucumab); PDPN (e.g., ThermoFisher Catalog #14-5381-82); PLAVl; PMSA; polysialic acid (see, e.g., Watzlawik et al. J Nat Sci. 2015; 1(8):e141); PR1; PROM1 (e.g., Catalog #14-1331-82); PSA (e.g., ThermoFisher Catalog #PA1-38514; Daniels-Wells et al. BMC Cancer 2013; 13:195); PSCA (e.g., AGS-1C₄D4); PSMA (e.g., BAY 2315497); PTK7 (e.g., cofetuzumab); PVRIG; Ras mutant (e.g., Shin et al. Sci Adv. 2020; 6(3):eaay2174); RET (e.g., WO2020210551); RGS5 (e.g., TF-TA503075); RhoC (e.g., ThermoFisher Catalog PA5-77866); ROR1 (e.g., cirmtuzumab); ROR2 (e.g., BA3021); ROS1 (e.g., WO 2019107671); Sarcoma translocation breakpoints; SART3 (e.g., TF 18025-1-AP); Sialyl-Thomsen-nouveau-antigen (e.g., Eavarone et al. PLoS One. 2018; 13(7): e0201314); Siglecs 1-16 (see, e.g., Angata et al. Trends Pharmacol Sci. 2015; 36(10): 645-660); SIRPa (e.g., Catalog #17-1729-42); SIRPg (e.g., PA5-104381); SIT1 (e.g., PA5-53825); SLAMF7 (e.g., elotuzumab); SLC10A2 (e.g., ThermoFisher Catalog #PA5-18990); SLC12A2 (e.g., ThermoFisher Catalog #13884-1-AP); SLC17A2 (e.g., ThermoFisher Catalog #PA5-106752); SLC38A1 (e.g., ThermoFisher Catalog #12039-1-AP); SLC39A5 (e.g., ThermoFisher Catalog #MA5-27260); SLC39A6 (e.g., ladiratuzumab); SLC44A4 (e.g., ASG-5ME); SLC6A15 (e.g., ThermoFisher Catalog #PA5-52586); SLC6A6 (e.g., ThermoFisher Catalog #PA5-53431); SLC7A11 (e.g., ThermoFisher Catalog #PA1-16893); SLC7A5; sLe; SLITRK6 (e.g., sirtratumab); Sperm protein 17 (e.g., BS-5754R); SSX2 (e.g., ThermoFisher Catalog #MA5-24971); survivin (e.g., PA1-16836); TACSTD2 (e.g., PA5-47074); TAG-72 (e.g., MA1-25956); tenascin; TF (e.g., tisotumab); Tie3; TLR2/4/1 (e.g., tomaralimab); TM4SF5 (e.g., 18239-1-AP); TMEM132A (e.g., Catalog #PA5-62524); TMEM40 (e.g., PA5-60636); TMPRSS11D (e.g., PA5-30927); Tn; TNFRSF12 (e.g., BAY-356); TRAIL (e.g., Catalog #12-9927-42); TRAIL 1; TRP-2 (e.g., PA5-52736); ULBP1/2/3/4/5/6 (e.g., PA5-82302); uPAR (e.g., ATN-658); UPK1B (e.g., ThermoFisher Catalog #PA5-56863); UPK2 (e.g., ThermoFisher Catalog #PA5-60318); UPK3B (e.g., ThermoFisher Catalog #PA5-52696); VEGF (e.g., GNR-011); VEGFR2 (e.g., gentuximab); VSIR (e.g., ThermoFisher Catalog #PA5-52493); WT1 (e.g., ThermoFisher Catalog #MA5-32215); and XAGE1 (e.g., ThermoFisher Catalog #PA5-46413).

Non-limiting examples of target antigens and associated antibodies that bind specifically to immune cell antigens include Axl (e.g., BA3011; tilvestamab); B7-1 (e.g., galiximab); B7-2 (e.g., Catalog #12-0862-82); B7-DC (e.g., Catalog #PA5-20344); B7-H3 (e.g., enoblituzumab, omburtamab, MGD009, MGC018, DS-7300); B7-H4 (e.g., Catalog #14-5949-82); B7-H6 (e.g., Catalog #12-6526-42); B7-H7; BAFF-R (e.g., Catalog #14-9117-82); BCMA; C₅ complement (e.g., BCD-148; CAN106); CCR4 (e.g., AT008; mogamulizumab-kpkc); CCR8 (e.g., JTX-1811); CD112 (see, e.g., U.S. Publication No. 20100008928); CD115 (e.g., axatilimab; cabiralizumab; emactuzumab); CD123 (e.g., BAY-943; CSL360); CD137 (e.g., ADG106; CTX-471); CD155 (e.g., U.S. Publication No. 2018/0251548); CD163 (e.g., TBI 304H); CD19 (e.g., ALLO-501); CD2 (e.g., BTI-322; siplizumab); CD20 (e.g., divozilimab; ibritumomab); CD24 (see, e.g., U.S. Pat. No. 8,614,301); CD244 (e.g., R&D AF1039); CD247 (e.g., AFM15); CD25 (e.g., basiliximab); CD27 (e.g., varlilumab); CD274 (e.g., adebrelimab; atezolizumab; garivulimab); CD278 (e.g., feladilimab; vopratelimab); CD28 (e.g., REGN5668); CD3 (e.g., otelixizumab; visilizumab); CD30 (e.g., iratumumab); CD30L (see, e.g., U.S. Pat. No. 9,926,373); CD32 (e.g., mAb 2B6); CD33 (e.g., lintuzumab; BI 836858; AMG 673); CD352 (e.g., SGN-CD352A); CD37 (e.g., lilotomab; GEN3009); CD38 (e.g., felzartamab; AMG 424); CD3D; CD3E (e.g., foralumab; teplizumab); CD3G; CD40 (e.g., dacetuzumab; lucatumumab); CD44 (e.g., RG7356); CD45 (e.g., apamistamab); CD47 (e.g., letaplimab; magrolimab); CD48 (e.g., SGN-CD48A); CD5 (e.g., MAT 304; zolimomab aritox); CD70 (e.g., cusatuzumab); CD74 (e.g., milatuzumab); CD79A (see, e.g., International Publication No. WO 2020252110); CD83 (e.g., CBT004); CD97; CD262 (e.g., tigatuzumab); CLEC12A (e.g., tepoditamab); CTLA4 (e.g., ipilimumab); CXCR4 (e.g., ulocuplumab); DCIR; DCSIGN (see, e.g., International Publication No. WO 2018134389); Dectin1 (see, e.g., U.S. Pat. No. 9,045,542); Dectin2 (e.g., ThermoFisher Catalog #MA5-16250); DR4 (e.g., mapatumumab); endosialin (e.g., ontuxizumab); FasL; FLT3 (e.g., 4G8SDIEM); GITR (e.g., ragifilimab); HAVCR2; HLA-DR; HLA-E; HLA-F; HLA-G (e.g., TTX-080); ICAM1; IDOl; IFNAR1 (e.g., faralimomab); IFNAR2; IL1RAP (e.g., nidanilimab); IL-21R (e.g., PF-05230900); IL-5R (e.g., benralizumab); LAG-3 (e.g., encelimab); LAMP1; LAYN; LCK; LILRB2; LILRB4; MerTk (e.g., DS5MMER, Catalog #12-5751-82); MICA (e.g., 1E2C₈, Catalog #66384-1-IG); MICB (e.g., Catalog #MA5-29422); Mincle (e.g., OTI2A8, Catalog #TA505101); MRCl (e.g., ThermoFisher Catalog #12-2061-82); OX40 (e.g., ABM193); PD-1 (e.g., balstilimab; budigalimab; geptanolimab); PVRIG; Sialyl-Thomsen-nouveau-antigen (e.g., Eavarone et al. PLoS One, 2018; 13(7): e0201314); Siglecs 1-16 (see, e.g., Angata et al. Trends Pharmacol Sci. 2015; 36(10): 645-660);); SIRPa (e.g., Catalog #17-1729-42); SIRPg (e.g., PA5-104381); SIT1 (e.g., PA5-53825); SLAMF7 (e.g., elotuzumab); TIGIT (e.g., etigilimab); TLR2/4/1 (e.g., tomaralimab); Trem2 (e.g., PY314); Tyrol; ULBP1/2/3/4/5/6 (e.g., PA5-82302); uPAR (e.g., ATN-658); and VSIR (e.g., ThermoFisher Catalog #PA5-52493).

Non-limiting examples of target antigens and associated antibodies that bind specifically to stromal cell antigens include FAP (e.g., sibrotuzumab); IFNAR1 (e.g., faralimomab); and IFNAR2.

In some embodiments, the antibody is a non-targeted antibody, for example, non-binding or control antibody.

In some embodiments, an antibody provided herein binds to EphA2. In some embodiments, the antibody comprises CDR-H1, CDR-H2, CDR-H3, CDR-L1, CDR-L2, and CDR-L3 comprising the amino acid sequences of SEQ ID NOs: 1, 2, 3, 4, 5, and 6, respectively. In some embodiments, the antibody comprises a CDR-H1 comprising an amino acid sequence that is at least 80% identical to the amino acid sequence of SEQ ID NO: 1. In some embodiments, the antibody comprises a CDR-H2 comprising an amino acid sequence that is at least 88% or at least 94% identical to the amino acid sequence of SEQ ID NO: 2. In some embodiments, the antibody comprises a CDR-H3 comprising an amino acid sequence that is at least 89% or at least 94% identical to the amino acid sequence of SEQ ID NO: 3. In some embodiments, the antibody comprises a CDR-L1 comprising an amino acid sequence that is at least 90% identical to the amino acid sequence of SEQ ID NO: 4. In some embodiments, the antibody comprises a CDR-L2 comprising an amino acid sequence that is at least 85% identical to the amino acid sequence of SEQ ID NO: 5. In some embodiments, the antibody comprises a CDR-L3 comprising an amino acid sequence that is at least 88% identical to the amino acid sequence of SEQ ID NO: 6. In some embodiments, the anti-EphA2 antibody comprises a heavy chain variable region comprising an amino acid sequence that is at least 95%, at least 96%, at least 97%, at least 98%, at least 99%, or 100% identical to the amino acid sequence of SEQ ID NO: 7 and a light chain variable region comprising an amino acid sequence that is at least 95% at least 96%, at least 97%, at least 98%, at least 99%, or 100% identical to the amino acid sequence of SEQ ID NO: 8. In some embodiments, the anti-EphA2 antibody comprises a heavy chain comprising the amino acid sequence of SEQ ID NO: 9 or SEQ ID NO: 10 and a light chain comprising the amino acid sequence of SEQ ID NO: 11. In some embodiments, the anti-EphA2 antibody comprises a heavy chain comprising the amino acid sequence of SEQ ID NO: 12 or SEQ ID NO: 13 and a light chain comprising the amino acid sequence of SEQ ID NO: 14. In some embodiments, the anti-EphA2 antibody comprises a heavy chain comprising the amino acid sequence of SEQ ID NO: 15 or SEQ ID NO: 16 and a light chain comprising the amino acid sequence of SEQ ID NO: 17. In some embodiments, the antibody is h1C₁ or 1C₁. SEQ ID NO: 1-17 are provided in TABLE 1 below.

TABLE 1

| SEQ | | |
|---|---|---|
| ID NO | Description | Sequence |
| 1 | h1C1 | HYMMA |
| | CDR-H1 | |
| 2 | h1C1 | RIGPSGGPTHYADSVKG |
| | CDR-H2 | |
| 3 | h1C1 | YDSGYDYVAVAGPAEYFQH |
| | CDR-H3 | |
| 4 | h1C1 | RASQSISTWLA |
| | CDR-L1 | |
| 5 | h1C1 | KASNLHT |
| | CDR-L2 | |
| 6 | h1C1 | QQYNSYSRT |
| | CDR-L3 | |
| 7 | h1C1 VH | EVQLLESGGGLVQPGGSLRLSCAASGFTFSHYMMAWVRQAPGKGL |
| | | EWVSRIGPSGGPTHYADSVKGRFTISRDNSKNTLYLQMNSLRAEDT |
| | | AVYYCAGYDSGYDYVAVAGPAEYFQHWGQGTLVTVSS |
| 8 | h1C1 VL | DIQMTQSPSSLSASVGDRVTITCRASQSISTWLAWYQQKPGKAPKLL |
| | | IYKASNLHTGVPSRFSGSGSGTEFSLTISGLQPDDFATYYCQQYNSYS |
| | | RTFGQGTKVEIK |
| 9 | h1C1 HC | EVQLLESGGGLVQPGGSLRLSCAASGFTFSHYMMAWVRQAPGKGL |
| | | EWVSRIGPSGGPTHYADSVKGRFTISRDNSKNTLYLQMNSLRAEDT |
| | | AVYYCAGYDSGYDYVAVAGPAEYFQHWGQGTLVTVSSASTKGPS |
| | | VFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 10 | h1C1 HC | EVQLLESGGGLVQPGGSLRLSCAASGFTFSHYMMAWVRQAPGKGL |
| | v2 | EWVSRIGPSGGPTHYADSVKGRFTISRDNSKNTLYLQMNSLRAEDT |
| | | AVYYCAGYDSGYDYVAVAGPAEYFQHWGQGTLVTVSSASTKGPS |
| | | VFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 11 | h1C1 LC | DIQMTQSPSSLSASVGDRVTITCRASQSISTWLAWYQQKPGKAPKLL |
| | | IYKASNLHTGVPSRFSGSGSGTEFSLTISGLQPDDFATYYCQQYNSYS |
| | | RTFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEKH |
| | | KVYACEVTHQGLSSPVTKSFNRGEC |
| 12 | h1C1 | EVQLLESGGGLVQPGGSLRLSCAASGFTFSHYMMAWVRQAPGKGL |
| | mIgG2a | EWVSRIGPSGGPTHYADSVKGRFTISRDNSKNTLYLQMNSLRAEDT |
| | HC | AVYYCAGYDSGYDYVAVAGPAEYFQHWGQGTLVTVSSAKTTAPS |
| | | VYPLAPVCGDTTGSSVTLGCLVKGYFPEPVTLTWNSGSLSSGVHTFP |
| | | AVLQSDLYTLSSSVTVTSSTWPSQSITCNVAHPASSTKVDKKIEPRGP |
| | | TIKPCPPCKCPAPNLLGGPSVFIFPPKIKDVLMISLSPIVTCVVVDVSE |
| | | DDPDVQISWFVNNVEVHTAQTQTHREDYNSTLRVVSALPIQHQDW |
| | | MSGKEFKCKVNNKDLPAPIERTISKPKGSVRAPQVYVLPPPEEEMTK |
| | | KQVTLTCMVTDFMPEDIYVEWTNNGKTELNYKNTEPVLDSDGSYF |
| | | MYSKLRVEKKNWVERNSYSCSVVHEGLHNHHTTKSFSRTPGK |
| 13 | h1C1 | EVQLLESGGGLVQPGGSLRLSCAASGFTFSHYMMAWVRQAPGKGL |
| | mIgG2a | EWVSRIGPSGGPTHYADSVKGRFTISRDNSKNTLYLQMNSLRAEDT |
| | HC v2 | AVYYCAGYDSGYDYVAVAGPAEYFQHWGQGTLVTVSSAKTTAPS |
| | | VYPLAPVCGDTTGSSVTLGCLVKGYFPEPVTLTWNSGSLSSGVHTFP |
| | | AVLQSDLYTLSSSVTVTSSTWPSQSITCNVAHPASSTKVDKKIEPRGP |
| | | TIKPCPPCKCPAPNLLGGPSVFIFPPKIKDVLMISLSPIVTCVVVDVSE |
| | | DDPDVQISWFVNNVEVHTAQTQTHREDYNSTLRVVSALPIQHQDW |
| | | MSGKEFKCKVNNKDLPAPIERTISKPKGSVRAPQVYVLPPPEEEMTK |
| | | KQVTLTCMVTDFMPEDIYVEWTNNGKTELNYKNTEPVLDSDGSYF |
| | | MYSKLRVEKKNWVERNSYSCSVVHEGLHNHHTTKSFSRTPG |
| 14 | h1C1 | DIQMTQSPSSLSASVGDRVTITCRASQSISTWLAWYQQKPGKAPKLL |
| | mIgG2a | IYKASNLHTGVPSRFSGSGSGTEFSLTISGLQPDDFATYYCQQYNSYS |
| | LC | RTFGQGTKVEIKRADAAPTVSIFPPSSEQLTSGGASVVCFLNNFYPK |
| | | DINVKWKIDGSERQNGVLNSWTDQDSKDSTYSMSSTLTLTKDEYER |
| | | HNSYTCEATHKTSTSPIVKSFNRNEC |
| 15 | h1C1 | EVQLLESGGGLVQPGGSLRLSCAASGFTFSHYMMAWVRQAPGKGL |
| | mIgG2a | EWVSRIGPSGGPTHYADSVKGRFTISRDNSKNTLYLQMNSLRAEDT |
| | LALAPG | AVYYCAGYDSGYDYVAVAGPAEYFQHWGQGTLVTVSSAKTTAPS |
| | HC | VYPLAPVCGDTTGSSVTLGCLVKGYFPEPVTLTWNSGSLSSGVHTFP |
| | | AVLQSDLYTLSSSVTVTSSTWPSQSITCNVAHPASSTKVDKKIEPRGP |
| | | TIKPCPPCKCPAPNAAGGPSVFIFPPKIKDVLMISLSPIVTCVVVDVSE |
| | | DDPDVQISWFVNNVEVHTAQTQTHREDYNSTLRVVSALPIQHQDW |
| | | MSGKEFKCKVNNKDLGAPIERTISKPKGSVRAPQVYVLPPPEEEMT |
| | | KKQVTLTCMVTDFMPEDIYVEWTNNGKTELNYKNTEPVLDSDGSY |
| | | FMYSKLRVEKKNWVERNSYSCSVVHEGLHNHHTTKSFSRTPGK |
| 16 | h1C1 | EVQLLESGGGLVQPGGSLRLSCAASGFTFSHYMMAWVRQAPGKGL |
| | mIgG2a | EWVSRIGPSGGPTHYADSVKGRFTISRDNSKNTLYLQMNSLRAEDT |
| | LALAPG | AVYYCAGYDSGYDYVAVAGPAEYFQHWGQGTLVTVSSAKTTAPS |
| | HC v2 | VYPLAPVCGDTTGSSVTLGCLVKGYFPEPVTLTWNSGSLSSGVHTFP |
| | | AVLQSDLYTLSSSVTVTSSTWPSQSITCNVAHPASSTKVDKKIEPRGP |
| | | TIKPCPPCKCPAPNAAGGPSVFIFPPKIKDVLMISLSPIVTCVVVDVSE |
| | | DDPDVQISWFVNNVEVHTAQTQTHREDYNSTLRVVSALPIQHQDW |
| | | MSGKEFKCKVNNKDLGAPIERTISKPKGSVRAPQVYVLPPPEEEMT |
| | | KKQVTLTCMVTDFMPEDIYVEWTNNGKTELNYKNTEPVLDSDGSY |
| | | FMYSKLRVEKKNWVERNSYSCSVVHEGLHNHHTTKSFSRTPG |
| 17 | h1C1 | DIQMTQSPSSLSASVGDRVTITCRASQSISTWLAWYQQKPGKAPKLL |
| | mIgG2a | IYKASNLHTGVPSRFSGSGSGTEFSLTISGLQPDDFATYYCQQYNSYS |
| | LALAPG | RTFGQGTKVEIKRADAAPTVSIFPPSSEQLTSGGASVVCFLNNFYPK |
| | LC | DINVKWKIDGSERQNGVLNSWTDQDSKDSTYSMSSTLTLTKDEYER |
| | | HNSYTCEATHKTSTSPIVKSFNRNEC |

### Linkers

As described herein, linkers (L) are optional groups that can connect D with Ab.

In some embodiments, the linker (L) comprises a cleavable group. The cleavable group can be configured for cleavage by particular enzymes or under specific physiological conditions. When coupled to targeting antibodies which direct coupling to or uptake by specific cells or tissues, such linkers (L) can ensure location-specific payload release. For example, the cleavable group can be configured for cleavage at low pH, such as that typically present in a tumor microenvironment; or in an oxidizing environment, such as those of lysosomes and some endosomes. Examples of such cleavable groups can include ortho-esters, ketals, acetals, hydrazones, imines, and maleic acid amides.

The cleavable group can be configured for enzymatic cleavage. When the enzymes for such cleavage are localized within specific tissues, cells, or sub-cellular compartments, the cleavable group can exhibit location specific cleavage, thereby preventing payload release outside of desired locations. Examples of such cleavable groups include protease and hydrolase cleavage sites. In some cases, the cleavable group includes a cleavable glycosidic group. In some cases, the cleavable glycosidic group comprises β-D-glucuronide, β-D-galactose, β-D-glucose, β-D-xylose, hexamaltose, β-L-gulose, β-L-allose, β-mannose-6-phosphate, β-L-fucose, α-E-mannose, β-D-fucose, 6-deoxy-β-D-glucose, β-mannose-6-phosphate, lactose, maltose, cellobiose, gentiobiose, maltotriose, β-D-GlcNAc, and β-D-GalNAc. For example, the cleavable group can comprise β-glucuronidase or α-mannosidase-cleavage sites cleavable by lysosomal β-glucuronidases or α-mannosidases, thereby rendering the linker (L) inert prior to lysosomal uptake and cleavable subsequent to lysosomal uptake. In some cases, the cleavable group comprises an enzymatically cleavable glycosidic bond, peptide bond, carbamate, or quaternary amine. In some cases, the enzyme for such cleavage is associated with a cancer cell, such as extracellular cathepsin.

The linker (L) can be tuned for tissue, cell, or sub-cellular localization. In some cases, the linker (L) is lipophilic, thereby promoting endocytic uptake when in proximity of a target cell. For example, in certain embodiments disclosed herein, the linker (L) comprises polyethylene glycol (PEG), non-charged and non-polar peptides, and/or other membrane-permeable groups.

In some embodiments, the linker (L) has the formula -M-(A)ₐ-(W)_{w}—(Y)_{y}—(X)ₓ—,
wherein:
M is a succinimide, a hydrolyzed succinimide, an amide, or a triazole;
subscript x is 0 or 1;
X is a C₁-C₆ alkylene or a 3-6 membered heteroalkylene;
A is a C₂₋₂₀ alkylene optionally substituted with 1-3 R^{a1}; or a 2 to 40 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{d1} and R¹ are independently hydrogen or C₁₋₃ alkyl;
subscript a is 0 or 1;
W is from 1-12 amino acids or has the structure:

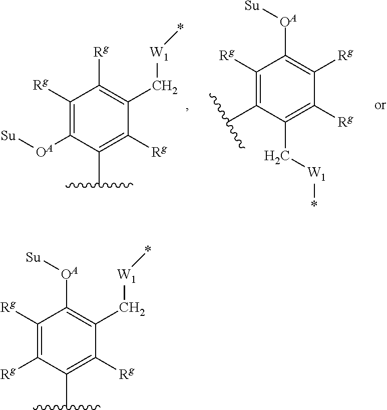

wherein Su is a Sugar moiety;
   —O^{A}— represents the oxygen atom of a glycosidic bond;
   each R^{g} is independently hydrogen, halogen, C₁-C₆ alkoxy, —N(C₁-C₆ alkyl)2, —NHC(═O)(C₁-C₆ alkyl), —CN, —CF₃, acyl, carboxamido, C₁-C₆ alkyl, or —NO₂;
   W₁ is absent, *—C(═O)—O—, or *—O—C(═O)—;
   [symbol] represents covalent attachment to A or M;
   represents covalent attachment to Y, X, or D;
subscript w is 0 or 1;
Y is a self-immolative moiety, a non-self-immolative releasable moiety, or a non-cleavable moiety; and
subscript y is 0 or 1.

In some embodiments, the linker (L) has the formula -M-(A)ₐ-(W)_{w}—(Y)_{y}—(X)ₓ—,
wherein:
M is a succinimide, a hydrolyzed succinimide, an amide, a methyl ketone, or a triazole;
subscript x is 0 or 1;
X is a C₁-C₆ alkylene or a 3-6 membered heteroalkylene;
A is a C₂₋₂₀ alkylene optionally substituted with 1-4 R^{a1}; or a 2 to 40 membered heteroalkylene optionally substituted with 1-4 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{d1} and R¹ are independently hydrogen or C₁₋₃ alkyl;
subscript a is 0 or 1;
W is from 1-12 amino acids or has the structure:

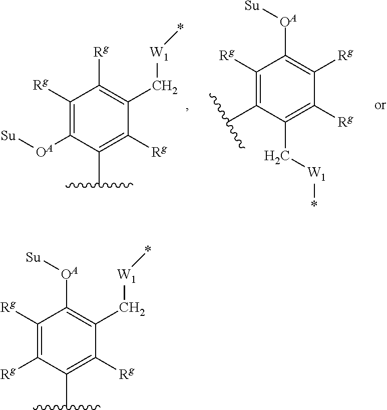

wherein Su is a Sugar moiety;
   —O^{A}— represents the oxygen atom of a glycosidic bond;
   each R^{g} is independently hydrogen, halogen, C₁-C₆ alkoxy, —N(C₁-C₆ alkyl)2, —NHC(═O)(C₁-C₆ alkyl), —CN, —CF₃, acyl, carboxamido, C₁-C₆ alkyl, or —NO₂;
   W₁ is absent, *—C(═O)—O—, or *—O—C(═O)—;
   [symbol] represents covalent attachment to A or M;
   represents covalent attachment to Y, X, or D;
subscript w is 0 or 1;
Y is a self-immolative moiety, a non-self-immolative releasable moiety, or a non-cleavable moiety; and
subscript y is 0 or 1.

In some embodiments, —O^{A}— represents the oxygen atom of a glycosidic bond. In some embodiments, the glycosidic bond provides a β-glucuronidase or a α-mannosidase-cleavage site. In some embodiments, the β-glucuronidase-cleavage site is cleavable by human lysosomal β-glucuronidase. In some embodiments, the α-mannosidase-cleavage site is cleavable by human lysosomal α-mannosidase.

In some embodiments, subscript x is 0. In some embodiments, subscript x is 1. In some embodiments, subscript a is 0. In some embodiments, subscript a is 1. In some embodiments, subscript w is 0. In some embodiments, subscript w is 1. In some embodiments, subscript y is 0. In some embodiments, subscript y is 1. In some embodiments, subscripts a+y+w=1. In some embodiments, subscripts a+y+w=2. In some embodiments, subscripts a+y+w=3. In some embodiments, subscripts a+y+w=0. In some embodiments, subscripts x+a+y+w=0 (i.e., the linker (L) is M).

In some embodiments, X is a C₁-C₆ alkylene. In some embodiments, X is a C₁-C₃ alkylene. In some embodiments, X is a 3-4 membered heteroalkylene. In some embodiments, X is *—CH₂—N(CH₂CH₃)—, wherein the * represents covalent attachment to D.

In some embodiments, A is a C₂₋₂₀ alkylene optionally substituted with 1-4 R^{a1}. In some embodiments, A is a C₂₋₁₀ alkylene optionally substituted with 1-4 R^{a1}. In some embodiments, A is a C₄₋₁₀ alkylene optionally substituted with 1-4 R^{a1}. In some embodiments, A is a C₂₋₂₀ alkylene substituted with one R^{a1}. In some embodiments, A is a C₂-10 alkylene substituted with one R^{a1}. In some embodiments, A is a C₂₋₁₀ alkylene substituted with one R^{a1}.

In some embodiments, each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —C(═O)NR^{d1}R^{e1}, —C(═O)(C₁₋₆ alkyl), and —C(═O)O(C₁₋₆ alkyl). In some embodiments, each R^{a1} is C₁₋₆ alkyl. In some embodiments, each R^{a1} is C₁₋₆ haloalkyl. In some embodiments, each R^{a1} is C₁₋₆ alkoxy. In some embodiments, each R^{a1} is C₁₋₆ haloalkoxy. In some embodiments, each R^{a1} is halogen. In some embodiments, each R^{a1} is —OH. In some embodiments, each R^{a1} is ═O. In some embodiments, each R^{a1} is —NR^{d1}R^{e1}. In some embodiments, each R^{a1} is —(C₁₋₆ alkylene)-NR^{d1}R^{e1}. In some embodiments, each R^{a1} is —C(═O)NR^{d1}R^{e1}. In some embodiments, each R^{a1} is —C(═O)(C₁₋₆ alkyl). In some embodiments, each R^{a1} is —C(═O)O(C₁₋₆ alkyl). In some embodiments, one occurrence of R^{a1} is —NR^{d1}R^{e1}. In some embodiments, one occurrence of R^{a1} is —(C₁₋₆ alkylene)-NR^{d1}R^{e1}. In some embodiments, one occurrence of R^{a1} is —(C₁₋₂ alkylene)-NR^{d1}R^{e1}. In some embodiments, A is a C₂₋₂₀ alkylene substituted with 1 or 2 R^{a1}, each of which is ═O.

In some embodiments, R^{d1} and R^{e1} are independently hydrogen or C₁₋₃ alkyl. In some embodiments, one of R^{d1} and R^{e1} is hydrogen, and the other of R^{d1} and R^{e1} is C₁₋₃ alkyl. In some embodiments, R^{d1} and R^{e1} are both hydrogen or C₁₋₃ alkyl. In some embodiments, R^{d1} and R^{e1} are both C₁₋₃ alkyl. In some embodiments, R^{d1} and R^{e1} are both methyl.

In some embodiments, A is a C₂₋₂₀ alkylene. In some embodiments, A is a C₂₋₁₀ alkylene. In some embodiments, A is a C₂₋₁₀ alkylene. In some embodiments, A is a C₂₋₆ alkylene. In some embodiments, A is a C₄₋₁₀ alkylene.

In some embodiments, A is a 2 to 40 membered heteroalkylene optionally substituted with 1-4 R^{b1}. In some embodiments, A is a 2 to 20 membered heteroalkylene optionally substituted with 1-4 R^{b1}. In some embodiments, A is a 2 to 12 membered heteroalkylene optionally substituted with 1-4 R^{b1}. In some embodiments, A is a 4 to 12 membered heteroalkylene optionally substituted with 1-4 R^{b1}. In some embodiments, A is a 4 to 8 membered heteroalkylene optionally substituted with 1-4 R^{b1}. In some embodiments, A is a 2 to 40 membered heteroalkylene substituted with one R^{b1}. In some embodiments, A is a 2 to 20 membered heteroalkylene substituted with one R^{b1}. In some embodiments, A is a 2 to 12 membered heteroalkylene substituted with one R^{b1}. In some embodiments, A is a 4 to 12 membered heteroalkylene substituted with one R^{b1}. In some embodiments, A is a 4 to 8 membered heteroalkylene substituted with one R^{b1}.

In some embodiments, each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl). In some embodiments, each R^{b1} is C₁₋₆ alkyl. In some embodiments, each R^{b1} is C₁₋₆ haloalkyl. In some embodiments, each R^{b1} is C₁₋₆ alkoxy. In some embodiments, each R^{b1} is C₁₋₆ haloalkoxy. In some embodiments, each R^{b1} is halogen. In some embodiments, each R^{b1} is —OH. In some embodiments, each R^{b1} is —NR^{d1}R^{e1}. In some embodiments, each R^{b1} is —(C₁₋₆ alkylene)-NR^{d1}R^{e1}. In some embodiments, each R^{b1} is C(═O)NR^{d1}R^{e1}. In some embodiments, each R^{b1} is —C(═O)(C₁₋₆ alkyl). In some embodiments, each R^{b1} is —C(═O)O(C₁₋₆ alkyl). In some embodiments, one occurrence of R^{b1} is —NR^{d1}R^{e1}. In some embodiments, one occurrence of R^{b1} is —(C₁₋₆ alkylene)-NR^{d1}R^{e1}. In some embodiments, one occurrence of R^{b1} is —(C₁₋₂ alkylene)-NR^{d1}R^{e1}.

In some embodiments, R^{d1} and R^{e1} are independently hydrogen or C₁₋₃ alkyl. In some embodiments, one of R^{d1} and R¹ is hydrogen, and the other of R^{d1} and R¹ is C₁₋₃ alkyl. In some embodiments, R^{d1} and R^{e1} are both hydrogen or C₁₋₃ alkyl. In some embodiments, R^{d1} and R^{e1} are both C₁₋₃ alkyl. In some embodiments, R^{d1} and R^{e1} are both methyl.

In some embodiments, A is a 2 to 40 membered heteroalkylene. In some embodiments, A is a 2 to 20 membered heteroalkylene. In some embodiments, A is a 2 to 12 membered heteroalkylene. In some embodiments, A is a 4 to 12 membered heteroalkylene. In some embodiments, A is a 4 to 8 membered heteroalkylene. In some embodiments, A is selected from the group consisting of:

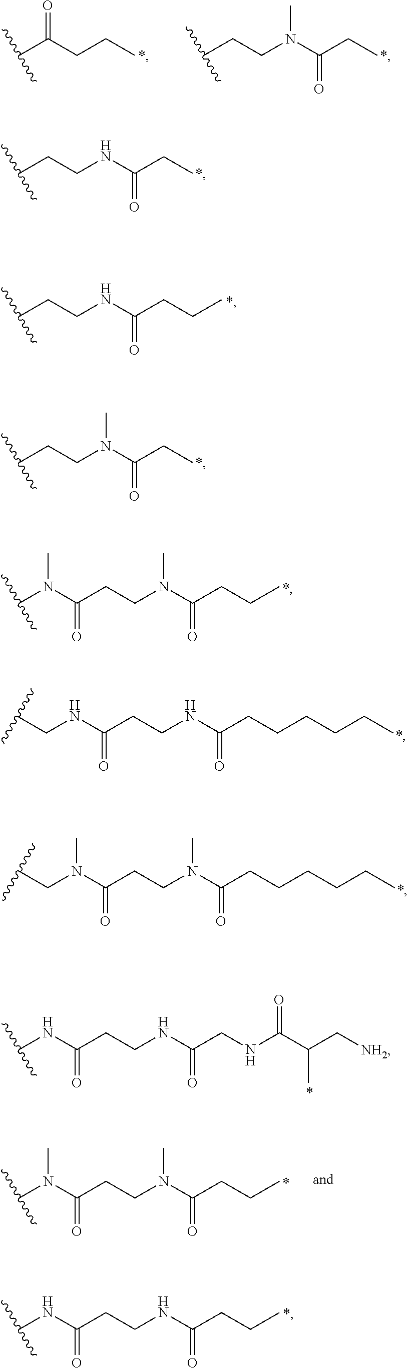

wherein [symbol] represents covalent attachment to W, Y, X, or D, and * represents covalent linkage to M. In some embodiments, M is a succinimide. In some embodiments, M is a hydrolyzed succinimide. It will be understood that a hydrolyzed succinimide may exist in two regioisomeric form(s). Those forms are exemplified below for hydrolysis of M, wherein the structures representing the regioisomers from that hydrolysis are formula M′ and M″; wherein wavy line a indicates the point of covalent attachment to the antibody, and wavy line b indicates the point of covalent attachment to A.

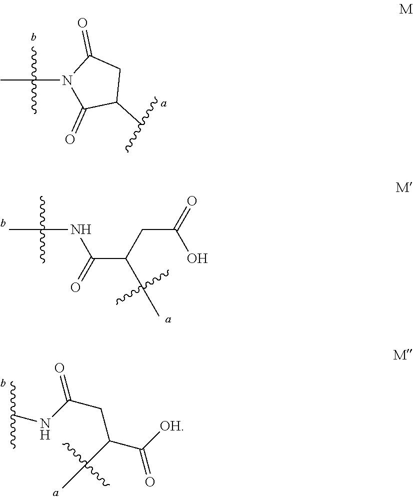 [SMILES: *C1CC(=O)N(*)C1=O ; *NC(=O)C(*)CC(=O)O ; *NC(=O)CC(*)C(=O)O]

In some embodiments, M′ is

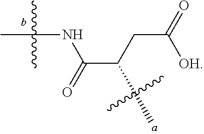 [SMILES: *NC(=O)[C@@H](C)CC(=O)O]

In some embodiments, M′ is

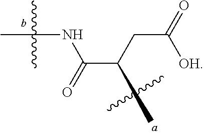 [SMILES: *NC(=O)[C@H](C)CC(=O)O]

In some embodiments, M″ is

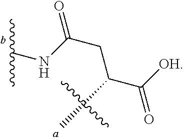 [SMILES: *NC(=O)C[C@@H](C)C(=O)O]

In some embodiments, M″ is

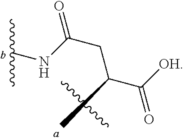 [SMILES: *NC(=O)C[C@H](C)C(=O)O]

In some embodiments, M is a triazole. In some embodiments, M is an amide. In some embodiments, A is a PEG4 to PEG12. In some embodiments, A is a PEG4 to PEG8. Representative A groups include, but are not limited to:

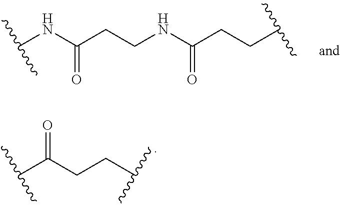 [SMILES: *CCC(=O)NCCC(=O)N* ; *CCC(*)=O]

In some embodiments, M is a methylketone.

In some embodiments, subscript w is 0. In some embodiments subscript w is 1.

In some embodiments, W is a single amino acid. In some embodiments, W is a single natural amino acid. In some embodiments, W is a peptide including from 2-12 amino acids, wherein each amino acid is independently a natural or unnatural amino acid. In some embodiments, each amino acid is independently a natural amino acid. In some embodiments, W is a dipeptide. In some embodiments, W is a tripeptide. In some embodiments, W is a tetrapeptide. In some embodiments, W is a pentapeptide. In some embodiments, W is a hexapeptide. In some embodiments, W is 7, 8, 9, 10, 11, or 12 amino acids. In some embodiments, each amino acid of W is independently selected from the group consisting of valine, alanine, β-alanine, glycine, lysine, leucine, phenylalanine, proline, aspartic acid, serine, glutamic acid, homoserine methyl ether, aspartate methyl ester, N,N-dimethyl lysine, arginine, valine-alanine, valine-citrulline, phenylalanine-lysine, and citrulline. In some embodiments, W is an aspartic acid. In some embodiments, W is a lysine. In some embodiments, W is a glycine. In some embodiments, W is an alanine. In some embodiments, W is aspartate methyl ester. In some embodiments, W is a N,N-dimethyl lysine. In some embodiments, W is a homoserine methyl ether. In some embodiments, W is a serine. In some embodiments, W is a valine-alanine.

In some embodiments, W is from 1-12 amino acids; and the bond between W and the X^{B} or between W and Y is enzymatically cleavable by a tumor-associated protease. In some embodiments, the tumor-associated protease is a cathepsin. In some embodiments, the tumor-associated protease is cathepsin B, C, or D.

In some embodiments, W has the structure of:

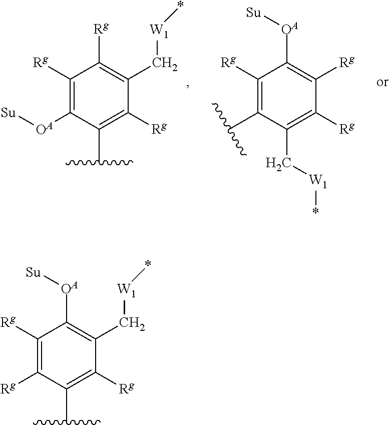

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)2,
—NHC(═O)(C₁-C₆ alkyl), —CN, —CF₃, acyl, carboxamido, C₁-C₆ alkyl, or —NO₂;
W₁ is absent, *—C(═O)—O—, or *—O—C(═O)—;
[symbol] represents covalent attachment to A or M; and
the * represents covalent attachment to Y, X, or D.

In some embodiments, —O^{A}— represents the oxygen atom of a glycosidic bond. In some embodiments, the glycosidic bond provides a β-glucuronidase or a α-mannosidase-cleavage site. In some embodiments, the β-glucuronidase or a α-mannosidase-cleavage site is cleavable by human lysosomal β-glucuronidase or by human lysosomal α-mannosidase.

In some embodiments, W is

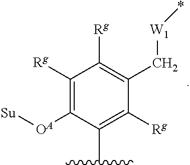

In some embodiments, W is

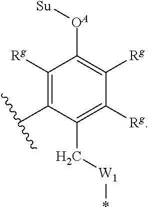

In some embodiments, W is

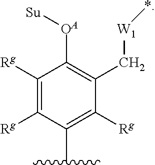

In some embodiments, each R^{g} is hydrogen. In some embodiments, one R^{g} is hydrogen, and the remaining R^{g} are independently halogen, C₁-C₆ alkoxy, —N(C₁-C₆ alkyl)2, —NHC(═O)(C₁-C₆ alkyl), —CN, —CF₃, acyl, carboxamido, C₁-C₆ alkyl, or —NO₂. In some embodiments, two R^{g} are hydrogen, and the remaining R^{g} is halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂.

In some embodiments, one R^{g} is halogen, C₁-C₆ alkoxy, —N(C₁-C₆ alkyl)2, —NHC(═O)(C₁-C₆ alkyl), —CN, —CF₃, acyl, carboxamido, C₁-C₆ alkyl, or —NO₂, and the other R^{g} are hydrogen.

In some embodiments, O^{A}—Su is charged neutral at physiological pH. In some embodiments, O^{A}—Su is mannose. In some embodiments, O^{A}-Su is

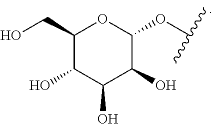 [SMILES: *O[C@H]1O[C@H](CO)[C@@H](O)[C@H](O)[C@@H]1O]

In some embodiments, O^{A}—Su comprises a carboxylate moiety. In some embodiments, O^{A}—Su is glucuronic acid. In some embodiments, O^{A}-Su is

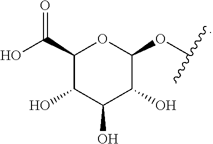 [SMILES: *O[C@@H]1O[C@H](C(=O)O)[C@@H](O)[C@H](O)[C@H]1O]

In some embodiments, W is

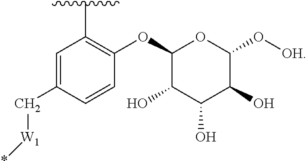

In some embodiments, W is

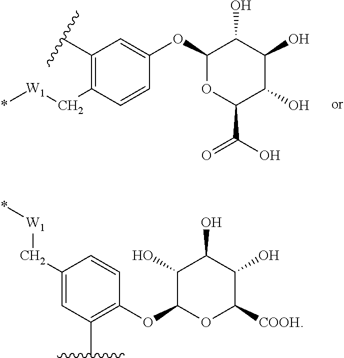

In some embodiments, W is

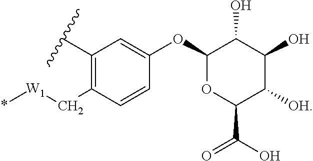

In some embodiments, W is

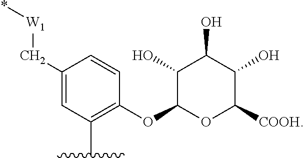

In some embodiments W₁ is absent. In some embodiments, W₁ is *—C(═O)—O—. In some embodiments, W₁ is absent or *—O—C(═O)—. In some embodiments, W₁ is *—O—C(═O)—.

In some embodiments, the linker comprises a cleavable unit. In some embodiments, W is a Cleavable Unit. In some embodiments, W is a Peptide Cleavable Unit. In some embodiments, W is a Glucuronide Unit.

In some embodiments, subscript a is 0.

In some embodiments, subscript y is 0. In some embodiments subscript y is 1.

In some embodiments, Y is a self-immolative moiety, a non-self-immolative releasable moiety, or a non-cleavable moiety. In some embodiments, Y is a self-immolative moiety or a non-self-immolative releasable moiety. In some embodiments, Y is a self-immolative moiety. In some embodiments, Y is a non-self-immolative moiety.

A non-self-immolative moiety is one which requires enzymatic cleavage, and in which part or all of the group remains bound to the Drug Unit after cleavage from the ADC, thereby forming free drug. Examples of a non-self-immolative moiety include, but are not limited to: -glycine- and -glycine-glycine. When an ADC having Y is -glycine- or -glycine-glycine-undergoes enzymatic cleavage (for example, via a cancer-cell-associated protease or a lymphocyte-associated protease), the Drug Unit is cleaved from the ADC such that the free drug includes the glycine or glycine-glycine group from Y. In some embodiments, an independent hydrolysis reaction takes place within, or in proximity to, the target cell, further cleaving the glycine or glycine-glycine group from the free drug. For example, an ADC with a non-self-immolative linker with a PAB optionally substituted with 1-4 substituents independently selected from halogen, cyano, and nitro, can undergo enzymatic cleavage of the linker (for example, via a cancer-cell-associated protease or a lymphocyte-associated protease), releasing a free drug which includes the optionally substituted PAB. This compound may further undergo 1,6-elimination of the PAB, removing any portion of Y from the free drug. See, e.g., Told et al., 2002, J. Org. Chem. 67:1866-1872. In some embodiments, enzymatic cleavage of the non-self-immolative moiety, as described herein, and does not result in any further hydrolysis step(s).

A self-immolative moiety refers to a bifunctional chemical moiety that is capable of covalently linking together two spaced chemical moieties into a normally stable tripartite molecule. The self-immolative moiety will spontaneously separate from the second chemical moiety if its bond to the first moiety is cleaved. For example, a self-immolative moiety includes a p-aminobenzyl alcohol (PAB) optionally substituted with one or more halogen, C₁-C₆ alkoxy, —N(C₁-C₆ alkyl)2, —NHC(═O)(C₁-C₆ alkyl), —CN, —CF₃, acyl, carboxamido, C₁-C₆ alkyl, or —NO₂. Other examples of self-immolative moieties include, but are not limited to, aromatic compounds that are electronically similar to the PAB group such as 2-aminoimidazol-5-methanol derivatives (see, e.g., Hay et al., 1999, Bioorg. Med. Chem. Lett. 9:2237), ortho or para-aminobenzylacetals, substituted and unsubstituted 4-aminobutyric acid amides (see, e.g., Rodrigues et al., 1995, Chemistry Biology 2:223), appropriately substituted bicyclo[2.2.1] and bicyclo[2.2.2] ring systems (see, e.g., Storm et al., 1972, J. Amer. Chem. Soc. 94:5815), 2-aminophenylpropionic acid amides (see, e.g., Amsberry et al., 1990, J. Org. Chem. 55:5867), and elimination of amine-containing drugs that are substituted at the α-position of glycine (see, e.g., Kingsbury et al., 1984, J. Med. Chem. 27:1447).

In some embodiments, Y is a p-aminobenzyl alcohol (PAB) optionally substituted with 1-4 substituents independently selected from halogen, C₁-C₆ alkoxy, —N(C₁-C₆ alkyl)2, —NHC(═O)(C₁-C₆ alkyl), —CN, —CF₃, acyl, carboxamido, C₁-C₆ alkyl, or —NO₂. In some embodiments, Y is an unsubstituted p-aminobenzyl alcohol (PAB).

In some embodiments, Y is a para-aminobenzyloxy-carbonyl (PABC) group optionally substituted with a sugar moiety. In some embodiments, Y is -glycine- or -glycine-glycine-. In some embodiments, Y is a branched bis(hydroxymethyl)styrene (BHMS) unit, which is capable of incorporating (and releasing) multiple Drug Units.

In some embodiments, -M-(A)ₐ-(W)_{w}—(Y)_{y}—(X)ₓ— is a non-self-immolative releasable linker, which provides release of the free drug once the ADC has been internalized into the target cell. In some embodiments, -M-(A)ₐ-(W)_{w}—(Y)_{y}—(X)ₓ— is a releasable linker, which provides release of the free drug with, or in the vicinity, of targeted cells. In some embodiments, releasable linkers possess a recognition site, such as a peptide cleavage site, sugar cleavage site, or disulfide cleavage site. In some embodiments, each releasable linker is a di-peptide. In some embodiments, each releasable linker is a disulfide. In some embodiments, each releasable linker is a hydrazone. In some embodiments, each releasable linker is independently selected from the group consisting of Val-Cit-, -Phe-Lys-, and -Val-Ala-. In some embodiments, each releasable linker, when bound to a succinimide or hydrolyzed succinimide, is independently selected from the group consisting of succinimido-caproyl (mc), succinimido-caproyl-valine-citrulline (sc-vc), succinimido-caproyl-valine-citrulline-paraaminobenzyloxycarbonyl (sc-vc-PABC), and SDPr-vc (where “S” refers to succinimido).

In some embodiments, -M-(A)ₐ-(W)_{w}—(Y)_{y}—(X)ₓ— comprises a non-cleavable linker. Non-cleavable linkers are known in the art and can be adapted for use with the ADCs described herein as the “Y” group. A non-cleavable linker is capable of linking a Drug Unit to an antibody in a generally stable and covalent manner and is substantially resistant to cleavage, such as acid-induced cleavage, light-induced cleavage, peptidase- or esterase-induced cleavage, and disulfide bond cleavage. The free drug can be released from the ADCs containing non-cleavable linkers via alternative mechanisms, such as proteolytic antibody degradation. In some embodiments, the Drug Unit can exert a biological effect as a part of the ADC (i.e., while still conjugated to the antibody via a linker).

Reagents that form non-cleavable linker-maleimide and non-cleavable linker-succinimide compounds are known in the art and can adapted for use herein. Exemplary reagents comprise a maleimido or haloacetyl-based moiety, such as 6-maleimidocaproic acid N-hydroxy succinimide ester (MCC), N-succinimidyl 4-(maleimidomethyl)cyclohexanecarboxylate (SMCC), N-succinimidyl-4-(N-maleimidomethyl)-cyclohexane-1-carboxy-(6-amidocaproate) (LC-SMCC), maleimidoundecanoic acid N-succinimidyl ester (KMUA), γ-maleimidobutyric acid N-succinimidyl ester (GMBS), c-maleimidocaproic acid N-hydroxysuccinimide ester (EMCS), m-maleimidobenzoyl-N-hydroxysuccinimide ester (MBS), N-(α-maleimidoacetoxy)-succinimide ester [AMAS], succinimidyl-6-(β-maleimidopropionamido)hexanoate (SMPH), N-succinimidyl-4-(p-maleimidophenyl)-butyrate (SMPB), and N-(p-maleimidophenyl)isocyanate (PMPI), N-succinimidyl-4-(iodoacetyl)-aminobenzoate (STAB), N-succinimidyl iodoacetate (SIA), N-succinimidyl bromoacetate (SBA) and N-succinimidyl 3-(bromoacetamido)propionate (SBAP). Additional “A-M” groups for use in the ADCs described herein can be found, for example, in U.S. Pat. No. 8,142,784, incorporated herein by reference in its entirety.

In some embodiments, Y is

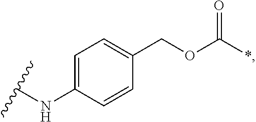

wherein [symbol] represents connection to W, A, or M; and the * represents connection to X or D, in the ADCs described herein.

In some embodiments, -M-(A)ₐ-(W)_{w}—(Y)_{y}—(X)ₓ—, comprises a non-releasable linker, wherein the free drug is released after the ADC has been internalized into the target cell and degraded, liberating the free drug.

In some embodiments, subscript x is 0; subscript y is 0; subscript w is 1; subscript a is 1; and M is a succinimide or a hydrolyzed succinimide.

In some embodiments, subscript x is 0; subscript y is 0; subscript w is 1; subscript a is 1; M is a succinimide or a hydrolyzed succinimide; and W has the structure of:

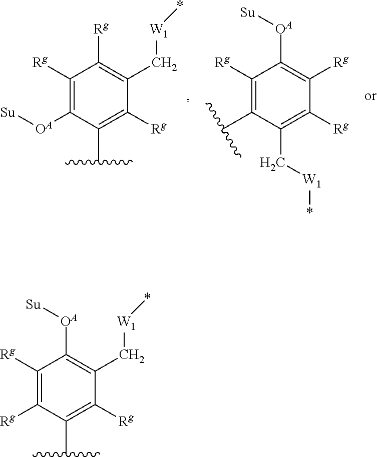

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W₁ is absent;
[symbol] represents covalent attachment to A; and
the * represents covalent attachment to D.

In some embodiments, subscript x is 0; subscript y is 0; subscript w is 1; subscript a is 1; M is a succinimide or a hydrolyzed succinimide; and W has the structure of:

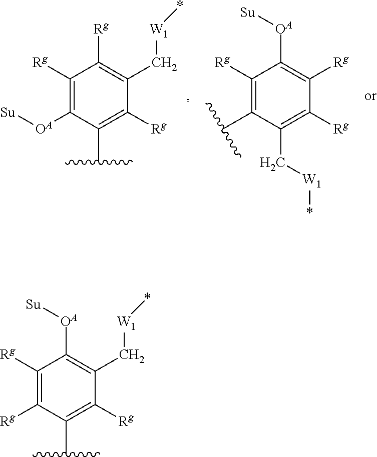

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W₁ is *—C(═O)—O—;
[symbol] represents covalent attachment to A; and
the * represents covalent attachment to D.

In some embodiments, subscript x is 0; subscript y is 1; subscript w is 1; subscript a is 1; and M is a succinimide or a hydrolyzed succinimide. In some embodiments, Y is a PAB group and W is a dipeptide.

In the linkers described herein, A, when present, is covalently attached to M; Y, when present, is attached to X, when present; and M is attached to Ab.

In some embodiments, the linker (L) is substituted with a polyethylene glycol moiety selected from the group consisting of PEG2 to PEG20. In some embodiments, L is substituted with a polyethylene glycol moiety selected from the group consisting of PEG2, PEG4, PEG6, PEG8, PEG10, PEG12, PEG16, and PEG20. In some embodiments, L is not substituted with a polyethylene glycol moiety selected from the group consisting of PEG2 to PEG20.

In some embodiments, A is substituted with a polyethylene glycol moiety selected from the group consisting of PEG2 to PEG20. In some embodiments, W is substituted with a polyethylene glycol moiety selected from the group consisting of PEG2 to PEG20. In some embodiments, Y is substituted with a polyethylene glycol moiety selected from the group consisting of PEG2 to PEG20. In some embodiments, X is substituted with a polyethylene glycol moiety selected from the group consisting of PEG2 to PEG20. In some embodiments, the linker (L) is substituted with one polyethylene glycol moiety. In some embodiments, the linker (L) is substituted with 2 or 3 independently selected polyethylene glycol moieties.

Polydisperse PEGs, monodisperse PEGs and discrete PEGs can be used to make the ADCs and intermediates thereof. Polydisperse PEGs are a heterogeneous mixture of sizes and molecular weights whereas monodisperse PEGs are typically purified from heterogeneous mixtures and therefore provide a single chain length and molecular weight. Discrete PEGs are synthesized in step-wise fashion and not via a polymerization process. Discrete PEGs provide a single molecule with defined and specified chain length. The number of —CH₂CH₂O-subunits of a PEG Unit ranges, for example, from 8 to 24 or from 12 to 24, referred to as PEG8 to PEG24 and PEG12 to PEG24, respectively.

The PEG moieties provided herein, which are also referred to as PEG Units, comprise one or multiple polyethylene glycol chains. The polyethylene glycol chains are linked together, for example, in a linear, branched or star shaped configuration. Typically, at least one of the polyethylene glycol chains of a PEG Unit is derivatized at one end for covalent attachment to an appropriate site on a component of the ADC (e.g., L). Exemplary attachments to ADCs are by means of non-conditionally cleavable linkages or via conditionally cleavable linkages. Exemplary attachments are via amide linkage, ether linkages, ester linkages, hydrazone linkages, oxime linkages, disulfide linkages, peptide linkages or triazole linkages. In some embodiments, attachment to ADC is by means of a non-conditionally cleavable linkage. In some embodiments, attachment to the ADC is not via an ester linkage, hydrazone linkage, oxime linkage, or disulfide linkage. In some embodiments, attachment to the ADC is not via a hydrazone linkage.

A conditionally cleavable linkage refers to a linkage that is not substantially sensitive to cleavage while circulating in plasma but is sensitive to cleavage in an intracellular or intratumoral environment. A non-conditionally cleavable linkage is one that is not substantially sensitive to cleavage in any biologically relevant environment in a subject that is administered the ADC. Chemical hydrolysis of a hydrazone, reduction of a disulfide bond, and enzymatic cleavage of a peptide bond or glycosidic bond of a Glucuronide Unit as described by WO 2007/011968 (which is incorporated by reference in its entirety) are examples of conditionally cleavable linkages.

In some embodiments, the PEG Unit is directly attached to the ADC (or an intermediate thereof) at L. In those embodiments, the other terminus (or termini) of the PEG Unit is free and untethered (i.e., not covalently attached), and in some embodiments, is a methoxy, carboxylic acid, alcohol or other suitable functional group. The methoxy, carboxylic acid, alcohol or other suitable functional group acts as a cap for the terminal polyethylene glycol subunit of the PEG Unit. By untethered, it is meant that the PEG Unit will not be covalently attached at that untethered site to a Drug Unit, to an antibody, or to a linking component to a Drug Unit and/or an antibody. Such an arrangement can allow a PEG Unit of sufficient length to assume a parallel orientation with respect to the drug in conjugated form, i.e., as a Drug Unit (D). For those embodiments in which the PEG Unit comprises more than one polyethylene glycol chain, the multiple polyethylene glycol chains are independently chosen, e.g., are the same or different chemical moieties (e.g., polyethylene glycol chains of different molecular weight or number of —CH₂CH₂O— subunits). A PEG Unit having multiple polyethylene glycol chains is attached to the ADC at a single attachment site. The skilled artisan will understand that the PEG Unit, in addition to comprising repeating polyethylene glycol subunits, may also contain non-PEG material (e.g., to facilitate coupling of multiple polyethylene glycol chains to each other or to facilitate coupling to the ADC). Non-PEG material refers to the atoms in the PEG Unit that are not part of the repeating —CH₂CH₂O— subunits. In some embodiments provided herein, the PEG Unit comprises two monomeric polyethylene glycol chains attached to each other via non-PEG elements. In other embodiments provided herein, the PEG Unit comprises two linear polyethylene glycol chains attached to a central core that is attached to the ADC (i.e., the PEG Unit itself is branched).

There are a number of PEG attachment methods available to those skilled in the art: see, for example: Goodson, et al. (1990) Bio Technology 8:343 (PEGylation of interleukin-2 at its glycosylation site after site-directed mutagenesis); EP 0 401 384 (coupling PEG to G-CSF); Malik, et al., (1992) Exp. Hematol. 20:1028-1035 (PEGylation of GM-CSF using tresyl chloride); ACT Pub. No. WO 90/12874 (PEGylation of erythropoietin containing a recombinantly introduced cysteine residue using a cysteine-specific mPEG derivative); U.S. Pat. No. 5,757,078 (PEGylation of EPO peptides); U.S. Pat. No. 5,672,662 (Poly(ethylene glycol) and related polymers monosubstituted with propionic or butanoic acids and functional derivatives thereof for biotechnical applications); U.S. Pat. No. 6,077,939 (PEGylation of an N-terminal α-carbon of a peptide); Veronese et al., (1985) Appl. Biochem. Bioechnol 11:141-142 (PEGylation of an N-terminal α-carbon of a peptide with PEG-nitrophenylcarbonate (“PEG-NPC”) or PEG-trichlorophenylcarbonate); and Veronese (2001) Biomaterials 22:405-417 (Review article on peptide and protein PEGylation).

For example, a PEG Unit may be covalently bound to an amino acid residue via reactive groups of a polyethylene glycol-containing compound and the amino acid residue. Reactive groups of the amino acid residue include those that are reactive to an activated PEG molecule (e.g., a free amino or carboxyl group). For example, N-terminal amino acid residues and lysine (K) residues have a free amino group; and C-terminal amino acid residues have a free carboxyl group. Thiol groups (e.g., as found on cysteine residues) are also useful as a reactive group for forming a covalent attachment to a PEG. In addition, enzyme-assisted methods for introducing activated groups (e.g., hydrazide, aldehyde, and aromatic-amino groups) specifically at the C-terminus of a polypeptide have been described. See Schwarz, et al. (1990) Methods Enzymol. 184:160; Rose, et al. (1991) Bioconjugate Chem. 2:154; and Gaertner, et al. (1994) J. Biol. Chem. 269: 7224.

In some embodiments, a polyethylene glycol-containing compound forms a covalent attachment to an amino group using methoxylated PEG (“mPEG”) having different reactive moieties. Non-limiting examples of such reactive moieties include succinimidyl succinate (SS), succinimidyl carbonate (SC), mPEG-imidate, para-nitrophenylcarbonate (NPC), succinimidyl propionate (SPA), and cyanuric chloride. Non-limiting examples of such mPEGs include mPEG-succinimidyl succinate (mPEG-SS), mPEG₂-succinimidyl succinate (mPEG₂-SS); mPEG-succinimidyl carbonate (mPEG-SC), mPEG₂-succinimidyl carbonate (mPEG₂-SC); mPEG-imidate, mPEG-para-nitrophenylcarbonate (mPEG-NPC), mPEG-imidate; mPEG₂-para-nitrophenylcarbonate (mPEG₂-NPC); mPEG-succinimidyl propionate (mPEG-SPA); mPEG₂-succinimidyl propionate (mPEG-SPA); mPEG-N-hydroxy-succinimide (mPEG-NHS); mPEG₂-N-hydroxy-succinimide (mPEG₂-NHS); mPEG-cyanuric chloride; mPEG₂-cyanuric chloride; mPEG₂-Lysinol-NPC, and mPEG₂-Lys-NHS.

In some instances, at least one of the polyethylene glycol chains that make up the PEG is functionalized to provide covalent attachment to the ADC. Functionalization of the polyethylene glycol-containing compound that is the precursor to the PEG includes, for example, via an amine, thiol, NHS ester, maleimide, alkyne, azide, carbonyl, or other functional group. In some embodiments, the PEG further comprises non-PEG material (i.e., material not comprised of —CH₂CH₂O—) that provides coupling to the ADC or in constructing the polyethylene glycol-containing compound or PEG facilitates coupling of two or more polyethylene glycol chains.

In some embodiments, the presence of the PEG Unit in an ADC is capable of having two potential impacts upon the pharmacokinetics of the resulting ADC. One impact is a decrease in clearance (and consequent increase in exposure) that arises from the reduction in non-specific interactions induced by the exposed hydrophobic elements of the Drug Unit. The second impact is a decrease in volume and rate of distribution that sometimes arises from the increase in the molecular weight of the ADC. Increasing the number of polyethylene glycol subunits increases the hydrodynamic radius of a conjugate, typically resulting in decreased diffusivity. In turn, decreased diffusivity typically diminishes the ability of the ADC to penetrate into a tumor. See Schmidt and Wittrup, Mol Cancer Ther 2009; 8:2861-2871. Because of these two competing pharmacokinetic effects, it can be desirable to use a PEG Unit that is sufficiently large to decrease the ADC clearance thus increasing plasma exposure, but not so large as to greatly diminish its diffusivity to an extent that it interferes with the ability of the ADC to reach the intended target cell population. See, e.g., Examples 1, 18, and 21 of US 2016/0310612, which is incorporated by reference herein (e.g., for methodology for selecting an optimal size of a PEG Unit for a particular Drug Unit, Linker, and/or drug-linker compound).

In some embodiments, the PEG Unit comprises one or more linear polyethylene glycol chains each having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In some embodiments, the PEG comprises a combined total of at least 8 subunits, at least 10 subunits, or at least 12 subunits. In some such embodiments, the PEG comprises no more than a combined total of about 72 subunits. In some such embodiments, the PEG comprises no more than a combined total of about 36 subunits. In some embodiments, the PEG comprises about 8 to about 24 subunits (referred to as PEG8 to PEG24).

In some embodiments, the PEG Unit comprises a combined total of from 2 to 72, 2 to 60, 2 to 48, 2 to 36 or 2 to 24 subunits, from 3 to 72, 3 to 60, 3 to 48, 3 to 36 or 3 to 24 subunits, from 4 to 72, 8 to 60, 4 to 48, 4 to 36 or 4 to 24 subunits, from 5 to 72, 5 to 60, 5 to 48, 5 to 36 or 5 to 24 subunits, from 6 to 72, 6 to 60, 6 to 48, 6 to 36 or 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits. In some embodiments, the PEG Unit comprises a combined total of from 2 to 24 subunits, 2 to 16 subunits, 2 to 12 subunits, 2 to 8 subunits, or 2 to 6 subunits.

Illustrative linear PEGs that can be used in any of the embodiments provided herein are as follows:

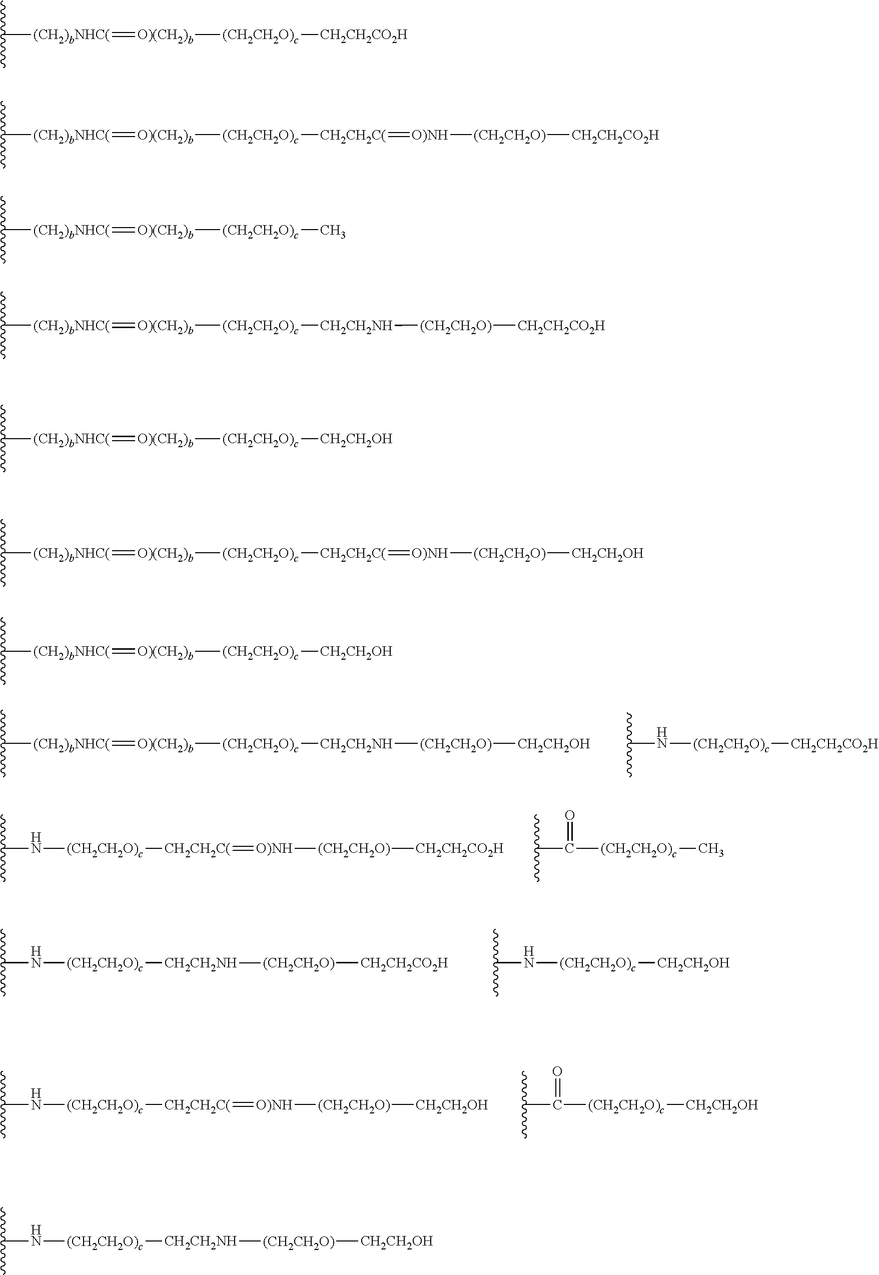

wherein the wavy line indicates the site of attachment to the ADC; each subscript b is independently selected from the group consisting of 2 to 12; and each subscript c is independently selected from the group consisting of 1 to 72, 8 to 72, 10 to 72, 12 to 72, 6 to 24, or 8 to 24. In some embodiments, each subscript b is 2 to 6. In some embodiments, each subscript c is about 2, about 4, about 8, about 12, or about 24.

As described herein, the PEG Unit can be selected such that it improves clearance of the resultant ADC but does not significantly impact the ability of the ADC to penetrate into the tumor.

In some embodiments, the PEG is from about 300 daltons to about 5 kilodaltons; from about 300 daltons to about 4 kilodaltons; from about 300 daltons to about 3 kilodaltons; from about 300 daltons to about 2 kilodaltons; from about 300 daltons to about 1 kilodalton; or any value in between. In some embodiments, the PEG has at least 8, 10 or 12 subunits. In some embodiments, the PEG Unit is PEG8 to PEG72, for example, PEG8, PEG10, PEG12, PEG16, PEG20, PEG24, PEG28, PEG32, PEG36, PEG48, or PEG72.

In some embodiments, apart from the PEGylation of the ADC, there are no other PEG subunits present in the ADC (i.e., no PEG subunits are present as part of any of the other components of the conjugates and linkers provided herein, such as A and X^{B}). In some embodiments, apart from the PEG, there are no more than 8, no more than 7, no more than 6, no more than 5, no more than 4, no more than 3, no more than 2 or no more than 1 other polyethylene glycol (—CH₂CH₂O—) subunits present in the ADC, or intermediate thereof (i.e., no more than 8, 7, 6, 5, 4, 3, 2, or 1 other polyethylene glycol subunits in other components of the ADCs (or intermediates thereof) provided herein).

It will be appreciated that when referring to polyethylene glycol subunits of a PEG Unit, and depending on context, the number of subunits can represent an average number, e.g., when referring to a population of ADCs or intermediates thereto and/or using polydisperse PEGs.

### Compounds of Formulae (A), (I)-(VIII), and (XI)

In some embodiments, each Drug Unit (D), as described herein, is a compound of any one of Formulae (A), (I)-(VIII), or (XI), as described herein. In some embodiments, each Drug Unit (D) is selected from a compound disclosed in U.S. Publ. No. 2017/0217960, which is incorporated by reference in its entirety, wherein the compound is further substituted with a covalent attachment to L.

In some embodiments, each D has the structure of Formula (A):

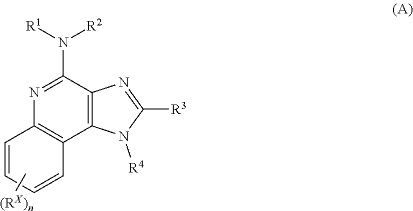

or a pharmaceutically acceptable salt thereof, wherein: R¹ is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached, form a 3-6 membered heterocyclyl optionally substituted with 1-3 independently selected C₁-C₆ alkyl;
R³ is (a) the point of covalent attachment to L; or (b) selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) the point of covalent attachment to L¹; (b) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]+;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]+, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]+, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H;
wherein when R⁴ is (c), the C₁-C₆ alkyl, or a substituent thereof, may be further substituted with the point of covalent attachment to L;
each R^{X} is (a) the point of covalent attachment to L; or (b) independently selected from the group consisting of hydrogen, —C(═O)OR^{F}, —C(═O)NR^{G}R^{H}, —S(O₂)NR^{G}R^{H}, —N(R)—C(═O)R^{J}, —N(R′)—S(O₂)R^{K}, halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R^{X} is the point of covalent attachment to L;
subscript n is 0, 1, 2, 3, or 4;
each R^{A} and R^{B} is (a) the point of covalent attachment to L, (b) independently selected from the group consisting of hydrogen and C₁-C₆ alkyl; or R^{A} and R^{B} taken together with the nitrogen atom to which they are attached, form a 3-6 membered heterocyclyl optionally substituted with 1-3 independently selected C₁-C₆ alkyl; wherein only one of R^{A} and R^{B} is the point of covalent attachment to L;
R^{C} is (a) the point of covalent attachment to L; or (b) selected from the group consisting of hydrogen, phenyl, and C₁-C₁₀ alkyl optionally substituted with phenyl or 1-3 independently selected halogen;
each R^{D}, R^{E}, R^{G}, and R^{H} are (a) the point of covalent attachment to L; or (b) independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached, form a 3-6 membered heterocyclyl optionally substituted with 1-3 independently selected C₁-C₆ alkyl; wherein only one of R^{D}, R^{E}, R^{G}, and R^{H} is the point of covalent attachment to L;
R^{F} is (a) the point of covalent attachment to L; or (b) selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is (a) the point of covalent attachment to L; or (b) independently selected from the group consisting of hydrogen and C₁-C₆ alkyl; wherein only one of R^{I}, R^{J}, and R^{K} is the point of covalent attachment to L;
wherein only one of R¹, R², R³, R⁴, R^{X}, R^{A}, R^{B}, R^{C}, R^{D}, R^{E}, R^{F}, R^{G}, R^{H}, R^{I}, R^{J}, and R^{K} is the point of covalent attachment to L; and
   wherein each D has only one point of covalent attachment to L.

In some embodiments, each D has the structure of Formula (I):

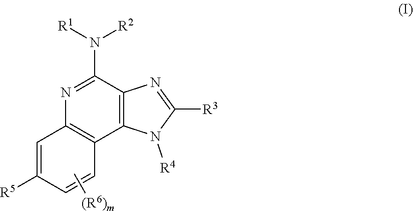

or a pharmaceutically acceptable salt thereof, wherein: R¹ is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is (a) the point of covalent attachment to L; or (b) selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) the point of covalent attachment to L¹; (b) —OR^{C}; (c) —S(═O)₂R^{C}; (d) —C(═O)NR^{D}R^{E}; (e) —C(═O)OR^{C}; (f) —C(═O)SR^{C}; (g) —C(═S)R^{C}; (h) —PO₃R^{C}; or (j) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]+;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]+, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]+, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H;
wherein when R⁴ is (j), the C₁-C₆ alkyl, or a substituent thereof, may be further substituted with the point of covalent attachment to L;
R⁵ is (a) the point of covalent attachment to L; or (b) selected from the group consisting of hydrogen, —C(═O)OR^{F}, —NO₂, —CN, —CF₃—C(═O)NR^{G}R^{H}, —S(O₂)NR^{G}R^{H}, —N(R¹)—C(═O)R^{J}, —N(R′)—S(O₂)R^{K}, and SO₃R^{K};
each R⁶ is (a) the point of covalent attachment to L; or (b) independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R⁶ is the point of covalent attachment to L;
subscript m is 0, 1, 2, or 3;
each R^{A} and R^{B} is (a) the point of covalent attachment to L, (b) independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl; wherein only one of R^{A} and R^{B} is the point of covalent attachment to L;
R^{C} is (a) the point of covalent attachment to L; or (b) selected from the group consisting of hydrogen, phenyl, and C₁-C₁₀ alkyl optionally substituted with phenyl or 1-3 independently selected halogen;
each R^{D}, R^{E}, R^{G}, and R^{H} are (a) the point of covalent attachment to L; or (b) independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl; wherein only one of R^{D}, R^{E}, R^{G}, and R^{H} is the point of covalent attachment to L;
R^{F} is (a) the point of covalent attachment to L; or (b) selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is (a) the point of covalent attachment to L; or (b) independently selected from the group consisting of hydrogen and C₁-C₆ alkyl; wherein only one of R^{I}, R^{J}, and R^{K} is the point of covalent attachment to L;
wherein only one of R¹, R², R³, R⁴, R⁵, R⁶, R^{A}, R^{B}, R^{C}, R^{D}, R^{E}, R^{F}, R^{G}, R^{H}, R^{I}, R^{J}, and R^{K} is the point of covalent attachment to L; and wherein each D has only one point of covalent attachment to L.

In some embodiments, each D has the structure of Formula (II):

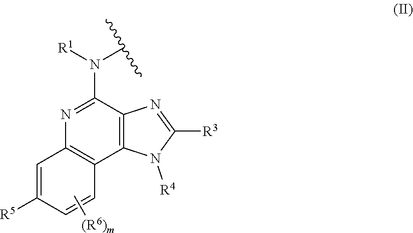

or a pharmaceutically acceptable salt thereof, wherein-˜ represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃—C₈ cycloalkyl, phenyl, 5-10 membered heteroaryl, C₁-C₆ alkoxy, C₁-C₆ alkylthio, and —NR^{A}R^{B};
R⁴ is (a) the point of covalent attachment to L¹; (b) —OR^{C}; (c) —S(═O)₂R^{C}; (d) —C(═O)NR^{D}R^{E}; (e) —C(═O)OR^{C}; (f) —C(═O)SR^{C}; (g) —C(═S)R^{C}; (h) —PO₃R^{C}; or (j) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]+;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃—C(═O)NR^{G}R^{H}, —S(O₂)NR^{G}R^{H}, —N(R′)—C(═O)R, —N(R)—S(O₂)R^{K}, and SO₃R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
each instance of R¹ and R⁴ is optionally substituted with a solubilizing group selected from the group consisting of phosphoryl, sulfuryl, nitro, C₅-C₉ monosaccharide, C₁₀-C₁₈ disaccharide, and C₁₅-C₂₇ trisaccharide.

In some embodiments, each D has the structure of Formula (III):

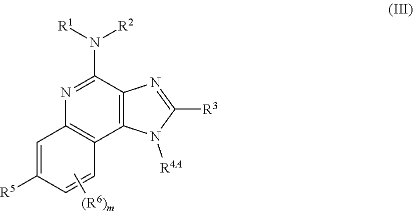

or a pharmaceutically acceptable salt thereof, wherein: R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R^{4A} is (a) the point of covalent attachment to L or (b) C₁-C₆ alkyl substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
wherein when R^{4A} is (b) the C₁-C₆ alkyl, or a substituent thereof, is further substituted with the point of covalent attachment to L;
R⁵ is selected from the group consisting of hydrogen, —C(═O)OR^{F}, —NO₂, —CN, —CF₃—C(═O)NR^{G}R^{H}, —S(O₂)NR^{G}R^{H}, —N(R′)—C(═O)R, —N(R)—S(O₂)R^{K}, and SO₃R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
R¹ is optionally substituted with a solubilizing group selected from the group consisting of phosphoryl, sulfuryl, nitro, C₅-C-9 monosaccharide, C₁₀-C₁₈ disaccharide, and C₁₅-C₂₇ trisaccharide.

In some embodiments, each D has the structure of Formula (IV):

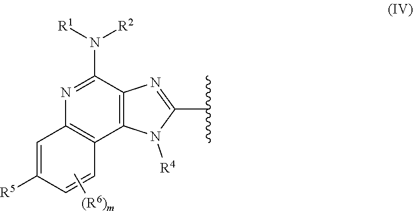

or a pharmaceutically acceptable salt thereof, wherein: [symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R⁴ is (a) —OR^{C} or (b) C₁-C₆ alkyl optionally substituted with:
(i) 1-3 independently selected halogen;
(ii) —OR^{C}.
(iii) —SR^{C}.
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃—C(═O)NR^{G}R^{H}, —S(O₂)NR^{G}R^{H}, —N(R′)—C(═O)R, —N(R)—S(O₂)R^{K}, and SO₃R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
R¹ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide.

In some embodiments, each D has the structure of Formula (V):

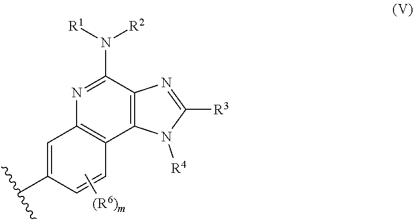

or a pharmaceutically acceptable salt thereof, wherein: [symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D} and R^{E} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, together with the nitrogen atom to which they are attached form a 3-6 membered heterocyclyl optionally substituted with 1-3 independently selected C₁-C₆ alkyl; and
   each instance of R¹ and R⁴ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide.

In some embodiments, each D has the structure of Formula (VI):

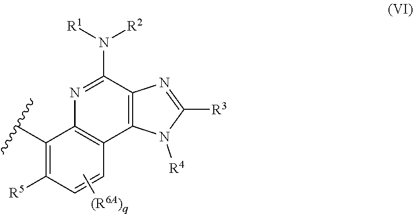

or a pharmaceutically acceptable salt thereof, wherein: [symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃—C(═O)NR^{G}R^{H}, —S(O₂)NR^{G}R^{H}, —N(R′)—C(═O)R, —N(R)—S(O₂)R^{K}, and SO₃R^{K};
each R^{6A} is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript q is 0, 1, or 2;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
each instance of R¹ and R⁴ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide.

In some embodiments, each D has the structure of Formula (VII):

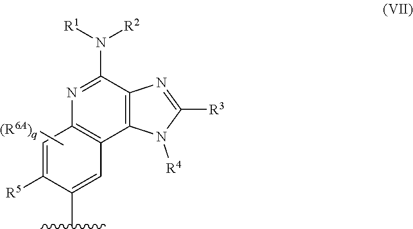

or a pharmaceutically acceptable salt thereof, wherein: [symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃—C(═O)NR^{G}R^{H}, —S(O₂)NR^{G}R^{H}, —N(R′)—C(═O)R, —N(R)—S(O₂)R^{K}, and SO₃R^{K};
each R^{6A} is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript q is 0, 1, or 2;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
each instance of R¹ and R⁴ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide.

In some embodiments, each D has the structure of Formula (VIII):

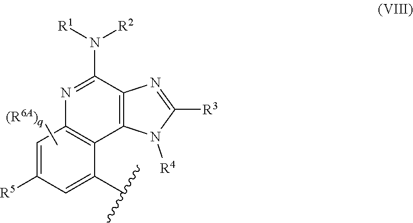

or a pharmaceutically acceptable salt thereof, wherein: [symbol] represents covalent attachment to L;
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃—C(═O)NR^{G}R^{H}, —S(O₂)NR^{G}R^{H}, —N(R′)—C(═O)R, —N(R)—S(O₂)R^{K}, and SO₃R^{K};
each R^{6A} is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript q is 0, 1, or 2;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
each instance of R¹ and R⁴ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide.

In some embodiments, each D has the structure of Formula (XI):

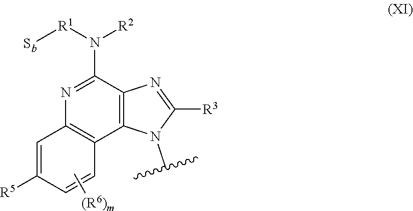

or a pharmaceutically acceptable salt thereof, wherein: [symbol] represents covalent attachment to L;
R¹ is a hydrolysable group selected from the group consisting of C₁-C₆ alkoxycarbonyl, C₁-C₆ alkoxythiocarbonyl, C₁-C₆ carbamoyl, C₁-C₆ amidine, C₁-C₆ sulfone, and C₁-C₆ thione; wherein each C₁-C₆ alkoxycarbonyl, C₁-C₆ alkoxythiocarbonyl, C₁-C₆ carbamoyl, C₁-C₆ amidine, C₁-C₆ sulfone, and C₁-C₆ thione, is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁵ is selected from the group consisting of hydrogen, —C(═O)OR^{F}, —NO₂, —CN, —CF₃—C(═O)NR^{G}R^{H}, —S(O₂)NR^{G}R^{H}, —N(R′)—C(═O)R, —N(R)—S(O₂)R^{K}, and SO₃R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
   S_{b} is a solubilizing group selected from the group consisting of phosphoryl, sulfuryl, nitro, C₅-C₉ monosaccharide, C₁₀-C₁₈ disaccharide, and C₁₁₅-C₂₇ trisaccharide.

In some embodiments, the compounds described herein are present in the form of a salt. In some embodiments, the salt is a pharmaceutically acceptable salt. In some embodiments of Formulae (A), or (I), R¹, R², R³, R⁴, or R⁵ is the point of covalent attachment to the linker. In some embodiments of Formulae (A) or (I), R¹, R², or R⁴ is the point of covalent attachment to the linker. In some embodiments of Formulae (A) or (I), R¹ is the point of covalent attachment to the linker. In some embodiments of Formulae (A) or (I), R² is the point of covalent attachment to the linker. In some embodiments of Formulae (A) or (I), R³ is the point of covalent attachment to the linker. In some embodiments of Formulae (A) or (I), R⁴ is the point of covalent attachment to the linker. In some embodiments of Formulae (A) or (I), the C₁-C₆ alkyl of R⁴, or a substituent thereof, is the point of covalent attachment to the linker. In this context, the “substituent thereof” refers to when R⁴ is a substituted C₁-C₆ alkyl, the point of covalent attachment to the linker can be via the substituent group, or via the C₁-C₆ alkyl group. In some embodiments of Formulae (A) or (I), a substituent of the C₁-C₆ alkyl of R⁴ is the point of covalent attachment to the linker. In some embodiments of Formulae (A) or (I), one of R^{A} and R^{B} is the point of covalent attachment to the linker. In some embodiments of Formulae (A) or (I), R^{A} is the point of covalent attachment to the linker. In some embodiments of Formulae (A) or (I), R^{B} is the point of covalent attachment to the linker. In some embodiments of Formulae (A) or (I), R^{C} is the point of covalent attachment to the linker. In some embodiments of Formulae (A) or (I), one of R^{D}, R^{E}, R^{G}, and R^{H} is the point of covalent attachment to the linker. In some embodiments of Formulae (A) or (I), R^{D} is the point of covalent attachment to the linker. In some embodiments of Formulae (A) or (I), R^{E} is the point of covalent attachment to the linker. In some embodiments of Formulae (A) or (I), R^{G} is the point of covalent attachment to the linker. In some embodiments of Formulae (A) or (I), R^{H} is the point of covalent attachment to the linker. In some embodiments of Formulae (A) or (I), R^{F} is the point of covalent attachment to the linker. In some embodiments of Formulae (A) or (I), one of R¹, R, and R^{K} is the point of covalent attachment to the linker. In some embodiments of Formulae (A) or (I), R is the point of covalent attachment to the linker. In some embodiments of v R^{J} is the point of covalent attachment to the linker. In some embodiments of Formulae (A) or (I), R^{K} is the point of covalent attachment to the linker. In some embodiments of Formula (A), R^{X} is the point of covalent attachment to the linker. In some embodiments of Formula (I), R⁵ is the point of covalent attachment to the linker. In some embodiments of Formula (I), one R⁶ is the point of covalent attachment to the linker.

Some embodiments of Formulae (A), (I)-(VIII), or (XI), D is in prodrug form. In some such embodiments of Formulae (A), (I)-(VIII), or (XI), R¹ is selected from the group consisting of C₁-C₆ alkoxycarbonyl, C₁-C₆ alkoxythiocarbonyl, C₁-C₆ carbamoyl, C₁-C₆ amidine, C₁-C₆ sulfone, and C₁-C₆ thione. In some cases of Formulae (A), (I)-(VIII), or (XI), R¹ in prodrug form is selected from the group consisting of C₁-C₆ alkoxycarbonyl, C₁-C₆ alkoxythiocarbonyl, and C₁-C₆ carbamoyl. In some cases, R¹ of the compound of Formulae (A), (I)-(VIII), or (XI), in prodrug form is C₁-C₆ alkoxycarbonyl.

In some embodiments of Formulae (A), (I)-(VIII), or (XI), R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₅ cycloalkyl, phenyl, 5-10 membered heteroaryl, C₁-C₆ alkoxy, C₁-C₆ alkylthio, and —NR^{A}R^{B}.

In some embodiments of Formulae (A), (I)-(VIII), or (XI), R¹ is selected from the group consisting of hydrogen, C₁-C₆ alkyl, C₂-C₆ alkenyl, C₂-C₆ alkynyl, C₁-C₆ alkoxycarbonyl, C₁-C₆ alkoxythiocarbonyl, C₁-C₆ carbamoyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkoxycarbonyl, C₁-C₆ alkoxythiocarbonyl, C₁-C₆ carbamoyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₅ cycloalkyl, phenyl, 5-10 membered heteroaryl, C₁-C₆ alkoxy, C₁-C₆ alkylthio, and —NR^{A}R^{B}.

In some embodiments of Formulae (A), (I)-(VIII), or (XI), R¹ is selected from the group consisting of hydrogen, C₁-C₆ alkyl, C₂-C₆ alkenyl, C₁-C₆ alkoxycarbonyl, C₁-C₆ carbamoyl, C₃-C₆ cycloalkyl, phenyl, and 5-10 membered heteroaryl; wherein each C₁-C₆ alkyl, C₂-C₆ alkenyl, C₁-C₆ alkoxycarbonyl, C₁-C₆ carbamoyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₅ cycloalkyl, phenyl, 5-10 membered heteroaryl, C₁-C₆ alkoxy, C₁-C₆ alkylthio, and —NR^{A}R^{B} In some embodiments of Formulae (A), (I)-(VIII), or (XI), R¹ is selected from the group consisting of C₁-C₆ alkoxycarbonyl and C₁-C₆ carbamoyl; wherein each C₁-C₆ alkoxycarbonyl and C₁-C₆ carbamoyl is optionally substituted with 1-3 substituents independently selected from the group consisting of hydroxyl, halogen, sulfhydryl, cyano, oxiranyl, C₃-C₅ cycloalkyl, phenyl, 5-10 membered heteroaryl, C₁-C₆ alkoxy, C₁-C₆ alkylthio, and —NR^{A}R^{B}.

In some embodiments of Formulae (A), (I)-(VIII), or (XI), R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}.

In some embodiments of Formulae (A), (I)-(VIII), or (XI), R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with one substituent selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}.

In some embodiments of Formulae (A), (I)-(VIII), or (XI), R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein the C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is unsubstituted.

In some embodiments of Formulae (A), (I)-(VIII), or (XI), R¹ is selected from the group consisting of hydrogen and C₁-C₆ alkyl. In some embodiments of Formulae (A), (I)-(VIII), or (XI), R¹ is hydrogen. In some embodiments of Formulae (A), (I)-(VIII), or (XI), R¹ is an unsubstituted C₁-C₆ alkyl. In some embodiments of Formulae (A), (I)-(VIII), or (XI), R¹ is methyl.

In some embodiments of Formulae (A), (I)-(VIII), or (XI), R¹ is a hydrolysable group selected from the group consisting of C₁-C₆ alkoxycarbonyl, C₁-C₆ alkoxythiocarbonyl, C₁-C₆ carbamoyl, C₁-C₆ amidine, C₁-C₆ sulfone, and C₁-C₆ thione; wherein each C₁-C₆ alkoxycarbonyl, C₁-C₆ alkoxythiocarbonyl, C₁-C₆ carbamoyl, C₁-C₆ amidine, C₁-C₆ sulfone, and C₁-C₆ thione is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}. In some such cases, R¹ may be hydrolysable enzymatically or under physiological conditions. In some embodiments of Formulae (A), (I)-(VIII), or (XI), R¹ is a hydrolysable group selected from the group consisting of C₁-C₆ alkoxycarbonyl and C₁-C₆ carbamoyl; wherein each C₁-C₆ alkoxycarbonyl and C₁-C₆ carbamoyl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}. In some embodiments of Formulae (A), (I)-(VIII), or (XI), R¹ is a hydrolysable group selected from the group consisting of C₁-C₆ alkoxycarbonyl and C₁-C₆ carbamoyl; wherein each C₁-C₆ alkoxycarbonyl and C₁-C₆ carbamoyl is optionally substituted with 1-3 substituents independently selected from the group consisting of hydroxyl, halogen, C₃-C₈ cycloalkyl, phenyl, 5-10 membered heteroaryl, C₁-C₆ alkoxy, and —NR^{A}R^{B}. In some embodiments of Formulae (A), (I)-(VIII), or (XI), R¹ is a hydrolysable group selected from the group consisting of C₁-C₃ alkoxycarbonyl and C₁-C₃ carbamoyl; wherein each C₁-C₆ alkoxycarbonyl and C₁-C₆ carbamoyl is optionally substituted with 1-3 substituents independently selected from the group consisting of hydroxyl, halogen, C₃-C₈ cycloalkyl, phenyl, 5-10 membered heteroaryl, C₁-C₆ alkoxy, and —NR^{A}R^{B}. In some embodiments of Formulae (A), (I)-(VIII), or (XI), R¹ is a hydrolysable group selected from the group consisting of C₁-C₃ alkoxycarbonyl and C₁-C₃ carbamoyl; wherein each C₁-C₆ alkoxycarbonyl and C₁-C₆ carbamoyl is optionally substituted with 1-3 substituents independently selected from the group consisting of C₃-C₈ cycloalkyl, phenyl, and 5-10 membered heteroaryl. In some embodiments of Formulae (A), (I)-(VIII), or (XI), R¹ is a hydrolysable C₁-C₃ alkoxycarbonyl optionally substituted with 1 substituent selected from the group consisting of C₃-C₈ cycloalkyl, phenyl, and 5-10 membered heteroaryl.

In some embodiments of Formulae (A), (I), (III)-(VIII), or (XI), R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}. In some embodiments of Formulae (A), (I), (III)-(VIII), or (XI), R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with one substituent selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —N^{A}R^{B}. In some embodiments of Formulae (A), (I), (III)-(VIII), or (XI), R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein the C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl are unsubstituted. In some embodiments of Formulae (A), (I), (III)-(VIII), or (XI), R² is selected from the group consisting of hydrogen and unsubstituted C₁-C₆ alkyl. In some embodiments of Formulae (A), (I), (III)-(VIII), or (XI), R² is hydrogen. In some embodiments of Formulae (A), (I), (III)-(VIII), or (XI), R² is unsubstituted C₁-C₆ alkyl. In some embodiments of Formulae (A), (I), (III)-(VIII), or (XI), R² is methyl. In some embodiments of Formulae (A), (I), (III)-(VIII), or (XI), R¹ and R² are both hydrogen. In some embodiments of Formulae (A), (I), (III)-(VIII), or (XI), R¹ and R² are both unsubstituted C₁-C₆ alkyl. In some embodiments of Formulae (A), (I), (III)-(VIII), or (XI), R¹ and R² are both methyl. In some embodiments of Formulae (A), (I), (III)-(VIII), or (XI), R¹ and R², taken together with the nitrogen atom to which they are attached, form a 3-6 membered heterocyclyl optionally substituted with 1-3 independently selected C₁-C₆ alkyl. In some embodiments of Formulae (A), (I), (III)-(VIII), or (XI), R¹ and R², taken together with the nitrogen atom to which they are attached, form a 3-6 membered heterocyclyl substituted with 1-3 independently selected C₁-C₆ alkyl. In some embodiments of Formulae (A), (I), (III)-(VIII), or (XI), R¹ and R², taken together with the nitrogen atom to which they are attached, form an unsubstituted 3-6 membered heterocyclyl.

In some embodiments of Formulae (A), (I)-(III), (V)-(VIII), or (XI), R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with one substituent selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B}.

In some embodiments of Formulae (A), (I)-(III), (V)-(VIII), or (XI), R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein the C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃—C₆ cycloalkyl, phenyl, 5-10 membered heteroaryl, and 3-12 membered heterocycle are unsubstituted.

In some embodiments of Formulae (A), (I)-(III), (V)-(VIII), or (XI), R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, and C₁-C₆ alkanoyloxy; wherein the C₁-C₆ alkyl, C₂-C₆ alkenyl, C₂-C₆ alkynyl, C₁-C₆ alkanoyl, C₁-C₆ alkoxycarbonyl, and C₁-C₆ alkanoyloxy are unsubstituted. In some embodiments of Formulae (A), (I)-(III), (V)-(VIII), or (XI), R³ is selected from the group consisting of hydrogen, unsubstituted C₁-C₆ alkyl, unsubstituted C₂-C₆ alkenyl, and unsubstituted C₂-C₆ alkynyl. In some embodiments of Formulae (A), (I)-(III), (V)-(VIII), or (XI), R³ is an unsubstituted C₁-C₆ alkyl. In some embodiments of Formulae (A), (I)-(III), (V)-(VIII), or (XI), R³ is an unsubstituted C₃-C₆ alkyl. In some embodiments of Formulae (A), (I)-(III), (V)-(VIII), or (XI), R³ is n-butyl. In some embodiments of Formulae (A), (I)-(III), (V)-(VIII), or (XI), R³ is C₁-C₆ alkyl substituted with C₁-C₆ alkoxy. In some embodiments of Formulae (A), (I)-(III), (V)-(VIII), or (XI), R³ is C₁-C₆ alkyl substituted with hydroxyl.

In some embodiments of Formulae (A), (I), (II), or (IV)-(VIII), R⁴ is C₁-C₆ alkyl optionally substituted with:
(i) 1-3 independently selected halogen;
(ii) —OR^{C}.
(iii) —SR^{C}.
(iv) —NH—S(O₂) R^{C};
(v) —OC(═O) R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl) R^{D}R^{E}]⁺.
(xi) -(phenyl)C₁-C₆ alkyl, wherein its C₁-C₆ alkyl is substituted with 5-10 membered heteroaryl, —NR^{D}R^{E}, —[N(C₁-C₆ alkyl) R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H.

In some embodiments of Formulae (A), (I), (II), or (IV)-(VIII), R⁴ is C₁-C₆ alkyl substituted with:
(i) 1-3 independently selected halogen;
(ii) —OR^{C}.
(iii) —SR^{C}.
(iv) —NH—S(O₂) R^{C};
(v) —OC(═O) R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl) R^{D}R^{E}]⁺.
(xi) -(phenyl)C₁-C₆ alkyl, wherein its C₁-C₆ alkyl is substituted with 5-10 membered heteroaryl, —NR^{D}R^{E}, —[N(C₁-C₆ alkyl) R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H.

In some embodiments of Formulae (A), (I), (II), or (IV)-(VIII), R⁴ is C₁-C₆ alkyl substituted with:
(ix) —NR^{D}R^{E};
(x) —[N(C₁-C₆ alkyl) R^{D}R^{E}]⁺.
(xi) -(phenyl)C₁-C₆ alkyl, wherein its C₁-C₆ alkyl is substituted with 5-10 membered heteroaryl, —NR^{D}R^{E}, —[N(C₁-C₆ alkyl) R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H.

In some embodiments of Formulae (A), (I), (II), or (IV)-(VIII), R⁴ is an optionally substituted C₁-C₆ alkyl having a least one substituent that is the point of covalent attachment to L, wherein the at least one substituent is one of (ii)-(xiv), as described herein, and wherein the optional substituent(s), if any are selected from the group consisting of (i)-(xiv), as described herein. In some embodiments of Formulae (A), (I), (II), or (IV)-(VIII), R⁴ is C₁-C₆ alkyl substituted with —OR^{c}. In some embodiments of Formulae (A), (I), (II), or (IV)-(VIII), R⁴ is C₁-C₆ alkyl substituted with —NR^{D}R^{E}. In some embodiments of Formulae (A), (I), (II), or (IV)-(VIII), R⁴ is C₁-C₆ alkyl substituted with —[N(C₁-C₆ alkyl) R^{D}R^{E}]⁺. In some embodiments of Formulae (A), (I), (II), or (IV)-(VIII), R⁴ is C₁-C₆ alkyl substituted with -(phenyl)C₁-C₆ alkyl, wherein the C₁-C₆ alkyl of the -(phenyl)C₁-C₆ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formulae (A), (I), (II), or (IV)-(VIII), R⁴ is C₁-C₃ alkyl substituted with -(phenyl)C₁-C₃ alkyl, wherein the C₁-C₃ alkyl of the -(phenyl)C₁-C₃ alkyl is substituted with 5-10 membered heteroaryl, —NR^{D}R^{E}, —[N(C₁-C₆ alkyl) R^{D}R^{E}]⁺, or 1-3 independently selected halogen.

In some embodiments of Formulae (A), (I), (II), or (IV)-(VIII), R⁴ is C₁-C₃ alkyl substituted with -(phenyl)C₁-C₃ alkyl, wherein the C₁-C₃ alkyl of the -(phenyl)C₁-C₃ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formulae (A), (I), (II), or (IV)-(VIII), R⁴ is —CH₂-(phenyl)-(C₁-C₂ alkyl), wherein the C₁-C₂ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formulae (A), (I), (II), or (IV)-(VIII), R⁴ is —CH₂-(phenyl)-(C₁-C₂ alkyl), wherein the C₁-C₂ alkyl is substituted with —NR^{D}R^{E} or —[N(C₁-C₆ alkyl) R^{D}R^{E}]⁺. In some embodiments of Formulae (A), (I), (II), or (IV)-(VIII), R⁴ is —CH₂-(phenyl)-(C₁-C₂ alkyl), wherein the C₁-C₂ alkyl is substituted with —NR^{D}R^{E}. In some embodiments of Formulae (A), (I), (II), or (IV)-(VIII), R⁴ is —CH₂-(phenyl)-(C₁-C₂ alkyl), wherein the C₁-C₂ alkyl is substituted with —[N(C₁-C₆ alkyl) R^{D}R^{E}]⁺. In some embodiments of Formulae (A), (I), (II), or (IV)-(VIII), R⁴ is C₁-C₆ alkyl substituted with phenyl substituted with halogen, hydroxyl, C₁-C₆ alkoxy, —C(═O)NR^{D}R^{E} or —CO₂H. In some embodiments of Formulae (A), (I), (II), or (IV)-(VIII), R⁴ is —CH₂-phenyl, wherein the phenyl is substituted with halogen, hydroxyl, C₁-C₆ alkoxy, —C(═O)NR^{D}R^{E}, or —CO₂H. In some embodiments of Formulae (A), (I), (II), or (IV)-(VIII), R⁴ is C₁-C₆ alkyl substituted with -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen. In some embodiments of Formulae (A), (I), (II), or (IV)-(VIII), R⁴ is C₁-C₃ alkyl substituted with -(5-10 membered heteroaryl)C₁-C₃ alkyl, wherein the C₁-C₃ alkyl of the -(5-10 membered heteroaryl)C₁-C₃ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formulae (A), (I), (II), or (IV)-(VIII), the C₁-C₃ alkyl of the -(5-6 membered heteroaryl)C₁-C₃ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formulae (A), (I), (II), or (IV)-(VIII), R⁴ is —CH₂-(5-6 membered heteroaryl)-(C₁-C₂ alkyl), wherein the C₁-C₂ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formulae (A), (I), (II), or (IV)-(VIII), R⁴ is —CH₂-(5-6 membered heteroaryl)-(C₁-C₂ alkyl), wherein the C₁-C₂ alkyl is substituted with —NR^{D}R^{E} or —[N(C₁-C₆ alkyl) R^{D}R^{E}]⁺. In some embodiments of Formulae (A), (I), (II), or (IV)-(VIII), R⁴ is —CH₂-(5-6 membered heteroaryl)-(C₁-C₂ alkyl), wherein the C₁-C₂ alkyl is substituted with —NR^{D}R^{E}. In some embodiments of Formulae (A), (I), (II), or (IV)-(VIII), R⁴ is —CH₂-(5-6 membered heteroaryl)-(C₁-C₂ alkyl), wherein the C₁-C₂ alkyl is substituted with —[N(C₁-C₆ alkyl) R^{D}R^{E}]⁺. In some embodiments of Formulae (A), (I), (II), or (IV)-(VIII), the 5-6 membered heteroaryl of R⁴ is pyridinyl, pyrimidinyl, or pyrazinyl. In some embodiments of Formulae (A), (I), (II), or (IV)-(VIII), R⁴ is C₁-C₆ alkyl substituted with 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H. In some embodiments of Formulae (A), (I), (II), or (IV)-(VIII), R⁴ is C₁-C₃ alkyl substituted with 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H. In some embodiments of Formulae (A), (I), (II), or (IV)-(VIII), R⁴ is —CH₂-(5-10 membered heteroaryl), wherein the 5-10 membered heteroaryl is optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H. In some embodiments of Formulae (A), (I), (II), or (IV)-(VIII), R⁴ is —CH₂-(5-6 membered heteroaryl), wherein the 5-6 membered heteroaryl is optionally substituted with halogen, -NR^{D}R^{E}, C₁-C₆ alkoxy, —C(═O)NR^{D}R^{E}, —SR^{C}, (C₁-C₆)alkoxycarbonyl, or —CO₂H. In some embodiments of Formulae (A), (I), (II), or (IV)-(VIII), the 5-6 membered heteroaryl of R⁴ is pyridinyl, pyrimidinyl, or pyrazinyl. In some embodiments of Formulae (A), (I), (II), or (IV)-(VIII), R⁴ is —OR^{C}. In some embodiments of Formulae (A), (I), (II), or (IV)-(VIII), R⁴ is an unsubstituted C₁-C₆ alkyl. In some embodiments of Formulae (A), (I), (II), or (IV)-(VIII), R⁴ is

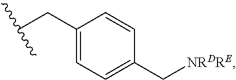

wherein [symbol] represent covalent attachment to the remainder of Formulae (A), (I), (II), or (IV)-(VIII). In some embodiments of Formulae (A), (I), (II), or (IV)-(VIII), R⁴ is

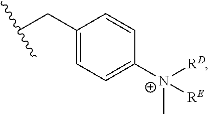

wherein [symbol] represent covalent attachment to the remainder of Formulae (A), (I), (II), or (IV)-(VIII), In some embodiments of Formula (III), R^{4A} is the point of covalent attachment to L.

In some embodiments of Formulae (A), (I), (II), or (IV)-(VIII), R⁴ is (a) the point of covalent attachment to L¹; (b) —OR^{C}; (c) —S(═O)₂R^{C}; (d) —C(═O)NR^{D}R^{E}; (e) —C(═O)OR^{C}; (f) —C(═O)SR^{C}; (g) —C(═S)R^{C}; (h) —PO₃R^{C}; or (j) C₁-C₆ alkyl optionally substituted with a group selected from the group consisting of (i)-(xiv), as described herein. In some embodiments of Formulae (A), (I), (II), or (IV)-(VIII), R⁴ is (a) the point of covalent attachment to L¹; (d) —C(═O)NR^{D}R^{E}; (e) —C(═O)OR^{C}; or (j) C₁-C₆ alkyl optionally substituted with a group selected from the group consisting of (i)-(xiv), as described herein. In some embodiments of Formulae (A), (I), (II), or (IV)-(VIII), R⁴ is (a) the point of covalent attachment to L¹; (d) —C(═O)NR^{D}R^{E}; or (e) —C(═O)OR^{C}. In some embodiments of Formulae (A), (I), (II), or (IV)-(VIII), R⁴ is (a) the point of covalent attachment to L¹; or (d) —C(═O)NR^{D}R^{E}. In some embodiments of Formulae (A), (I), (II), or (IV)-(VIII), R⁴ is —C(═O)NR^{D}R^{E}. In some embodiments of Formulae (A), (I), (II), or (IV)-(VIII), R⁴ is —C(═O)NR^{D}R^{E}, wherein R^{D} and R^{E} are not points of covalent attachment to L.

In some embodiments of Formula (III), R^{4A} is C₁-C₆ alkyl substituted with:
(i) 1-3 independently selected halogen;
(ii) —OR^{C}.
(iii) —SR^{C}.
(iv) —NH—S(O₂) R^{C};
(v) —OC(═O) R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl) R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl) R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H.

In some embodiments of Formula (III), R^{4A} is C₁-C₆ alkyl substituted with: (vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl) R^{D}R^{E}]⁺.
(xi) -(phenyl)C₁-C₆ alkyl, wherein its C₁-C₆ alkyl is substituted with 5-10 membered heteroaryl, —NR^{D}R^{E}, —[N(C₁-C₆ alkyl) R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H.

In some embodiments of Formula (III), R^{4A} is C₁-C₆ alkyl substituted with: (ix) —NR^{D}R^{E};
(x) —[N(C₁-C₆ alkyl) R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl) R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H.

In some embodiments of Formula (III), R^{4A} is C₁-C₆ alkyl substituted with —NR^{D}R^{E} In some embodiments of Formula (III), R^{4A} is C₁-C₆ alkyl substituted with —[N(C₁-C₆ alkyl) R^{D}R^{E}]⁺. In some embodiments of Formula (III), R^{4A} is C₁-C₆ alkyl substituted with -(phenyl)C₁-C₆ alkyl, wherein the C₁-C₆ alkyl of the -(phenyl)C₁-C₆ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formula (III), R^{4A} is C₁-C₃ alkyl substituted with -(phenyl)C₁-C₃ alkyl, wherein the C₁-C₃ alkyl of the -(phenyl)C₁-C₃ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formula (III), R^{4A} is C₁-C₃ alkyl substituted with -(phenyl)C₁-C₃ alkyl, wherein the C₁-C₃ alkyl of the -(phenyl)C₁-C₃ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formula (III), R^{4A} is —CH₂-(phenyl)-(C₁-C₂ alkyl), wherein the C₁-C₂ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formula (III), R^{4A} is —CH₂-(phenyl)-(C₁-C₂ alkyl), wherein the C₁-C₂ alkyl is substituted with —NR^{D}R^{E} or —[N(C₁-C₆ alkyl) R^{D}R^{E}]⁺. In some embodiments of Formula (III), R^{4A} is-CH₂-(phenyl)-(C₁-C₂ alkyl), wherein the C₁-C₂ alkyl is substituted with —NR^{D}R^{E}. In some embodiments of Formula (III), R^{4A} is-CH₂-(phenyl)-(C₁-C₂ alkyl), wherein the C₁-C₂ alkyl is substituted with —[N(C₁-C₆ alkyl) R^{D}R^{E}]⁺. In some embodiments of Formula (III), R^{4A} is C₁-C₆ alkyl substituted with phenyl substituted with halogen, hydroxyl, C₁-C₆ alkoxy, —C(═O)NR^{D}R^{E} or —CO₂H. In some embodiments of Formula (III), R^{4A} is —CH₂-phenyl, wherein the phenyl is substituted with halogen, hydroxyl, C₁-C₆ alkoxy, —C(═O)NR^{D}R^{E}, or —CO₂H. In some embodiments of Formula (III), R^{4A} is C₁-C₆ alkyl substituted with -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen. In some embodiments of Formula (III), R^{4A} is C₁-C₃ alkyl substituted with -(5-10 membered heteroaryl)C₁-C₃ alkyl, wherein the C₁-C₃ alkyl of the -(5-10 membered heteroaryl)C₁-C₃ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formula (III), R^{4A} is C₁-C₃ alkyl substituted with -(5-6 membered heteroaryl)C₁-C₃ alkyl, wherein the C₁-C₃ alkyl of the -(5-6 membered heteroaryl)C₁-C₃ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formula (III), R^{4A} is —CH₂-(5-6 membered heteroaryl)-(C₁-C₂ alkyl), wherein the C₁-C₂ alkyl is substituted with 5-10 membered heteroaryl, —NR^{D}R^{E}, —[N(C₁-C₆ alkyl) R^{D}R^{E}]⁺, or 1-3 independently selected halogen. In some embodiments of Formula (III), R^{4A} is —CH₂-(5-6 membered heteroaryl)-(C₁-C₂ alkyl), wherein the C₁-C₂ alkyl is substituted with —NR^{D}R^{E} or —[N(C₁-C₆ alkyl) R^{D}R^{E}]⁺. In some embodiments of Formula (III), R^{4A} is —CH₂-(5-6 membered heteroaryl)-(C₁-C₂ alkyl), wherein the C₁-C₂ alkyl is substituted with —NR^{D}R^{E}. In some embodiments of Formula (III), R^{4A} is —CH₂-(5-6 membered heteroaryl)-(C₁-C₂ alkyl), wherein the C₁-C₂ alkyl is substituted with —[N(C₁-C₆ alkyl) R^{D}R^{E}]⁺. In some embodiments of Formula (III), the 5-6 membered heteroaryl of R^{4A} is pyridinyl, pyrimidinyl, or pyrazinyl. In some embodiments of Formula (III), R^{4A} is C₁-C₆ alkyl substituted with 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H. In some embodiments of Formula (III), R^{4A} is C₁-C₃ alkyl substituted with 5-10 membered heteroaryl optionally substituted with halogen, —NR^{D}R^{E} C₁-C₆ alkoxy, —C(═O)NR^{D}R^{E}, —SR^{C}, (C₁-C₆)alkoxycarbonyl, or —CO₂H. In some embodiments of Formula (III), R^{4A} is —CH₂-(5-10 membered heteroaryl), wherein the 5-10 membered heteroaryl is optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H. In some embodiments of Formula (III), R^{4A} is —CH₂-(5-6 membered heteroaryl), wherein the 5-6 membered heteroaryl is optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H. In some embodiments of Formula (III), the 5-6 membered heteroaryl of R^{4A} is pyridinyl, pyrimidinyl, or pyrazinyl. In some embodiments of Formula (III), R^{4A} is

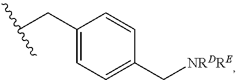

wherein [symbol] represent covalent attachment to the remainder of Formulae (III). In some embodiments of Formula (III), R^{4A} is

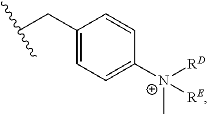

wherein [symbol] represent covalent attachment to the remainder of Formulae (III).

In some embodiments of Formula (A), R^{X} is selected from the group consisting of —C(═O)OR^{F}, —C(═O)NR^{G}R^{H}, —S(O₂)NR^{G}R^{H}, —N(R′)—C(═O) R^{J}, and —N(R′)—S(O₂) R^{K}. In some embodiments of Formula (A), R^{X} is —C(═O)OR^{F}.

In some embodiments of Formulae (A), (I)-(IV), (VI-VIII), or (XI), R⁵ is selected from the group consisting of hydrogen, —C(═O)OR^{F}, —C(═O)NR^{G}R^{H}, —S(O₂)NR^{G}R^{H}, —N(R)—C(═O)R^{J}, —N(R′)- and S(O₂)R^{K}. In some embodiments of Formulae (A), (I)-(IV), (VI-VIII), or (XI), R⁵ is selected from the group consisting of hydrogen, —C(═O)OR^{F}, —C(═O)NR^{G}R^{H}, —N(R¹)—C(═O)R, and S(O₂)R^{K}. In some embodiments of Formulae (I)-(IV), (VI-VIII), (XI)-(IV), (XVI)-(XVIII), or (XI), R⁵ is —C(═O)OR^{F}. In some embodiments of Formulae (I)-(IV), (VI-VIII), (XI)-(XIV), (XVI)-(XVIII), or (XI), R⁵ is —C(═O)OH or —C(═O)—O-Me.

In some embodiments disclosed herein, R⁵ is acidic, negatively charged, and/or highly polar (e.g., comprises a dipole moment of at least about 2.0 Debye). A surprising observation disclosed herein (e.g., as demonstrated in Example 37) is that C₇ imidazoquinoline functionalization (R⁵) can enhance toll-like receptor activation. It is contemplated that the importance of this functionalization in certain imidazoquinolines may have been previously missed due to its tendency to decrease cellular uptake, potentially masking its potency when applied as a free drug. However, when used in tandem with an effective targeting and uptake system, such as an antibody as disclosed herein, imidazoquinolines with negative or highly polar C₇ functionalizations can affect enhanced TLR7/8 responses. Without being limited by theory, it is posited that TLR7/8 binding may position the imidazoquinoline C₇ proximal to charged or polar protic residues, enabling strong hydrogen bonding interactions which enhance binding strength and agonistic behavior.

Accordingly, in certain embodiments of Formulae (A), (I)-(IV), (VI-VIII), or (XI), R⁵ is negatively charged under physiological conditions and/or highly polar (e.g., comprises a dipole moment of at least about 2.0 Debye). In some embodiments of Formulae (A), (I)-(IV), (VI-VIII), or (XI), R⁵ comprises a pKa of at most about 7.0. In some embodiments of Formulae (A), (I)-(IV), (VI-VIII), or (XI), R⁵ comprises a pKa of at most about 6.0. In some embodiments of Formulae (A), (I)-(IV), (VI-VIII), or (XI), R⁵ comprises a pKa of at most about 5.0. In some embodiments of Formulae (A), (I)-(IV), (VI-VIII), or (XI), R⁵ comprises a pKa of at most about 4.0. In some embodiments of Formulae (A), (I)-(IV), (VI-VIII), or (XI), R⁵ comprises a pKa of at most about 3.0. In some embodiments of Formulae (A), (I)-(IV), (VI-VIII), or (XI), R⁵ comprises a pKa of at most about 2.0. In some embodiments of Formulae (A), (I)-(IV), (VI-VIII), or (XI), R⁵ comprises a dipole moment of at least about 2.0 Debye (e.g., as calculated with density functional theory). In some embodiments of Formulae (A), (I)-(IV), (VI-VIII), or (XI), R⁵ comprises a dipole moment of at least about 2.5 Debye. In some embodiments of Formulae (A), (I)-(IV), (VI-VIII), or (XI), R⁵ comprises a dipole moment of at least about 3.0 Debye. In some embodiments of Formulae (A), (I)-(IV), (VI-VIII), or (XI), R⁵ comprises a pka of at most about 7.0 or a dipole moment of at least 2.0 Debye.

In some embodiments of Formulae (A), (I)-(IV), (VI-VIII), or (XI), R⁵ is selected from the group consisting of —C(═O)OH, —NO₂, —CN, —CF₃, and —S(O₃)H. In some embodiments of Formulae (A), (I)-(IV), (VI-VIII), or (XI), R⁵ is selected from the group consisting of —C(═O)OH and —S(O₃)H. In some embodiments Formulae (A), (I)-(IV), (VI-VIII), or (XI), R⁵ is —C(═O)OH.

In some embodiments of Formulae (A), (I)-(IV), (VI)-(VIII), or (XI), R^{F} is selected from the group consisting of trifluoromethyl, C₂-C₆ alkenyl, C₂-C₆ alkynyl, C₃-C₅ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl. In some embodiments of Formulae (A), (I)-(IV), (VI)-(VIII), or (XI), R^{F} is selected from the group consisting of trifluoromethyl, C₂-C₆ alkenyl, C₂-C₆ alkynyl, C₃-C₈ cycloalkyl, aryl, aryl(C₁-C₆ alkyl)-, and unsubstituted C₁-C₆ alkyl. In some embodiments of Formulae (A), (I)-(IV), (VI)-(VIII), or (XI), R^{F} is selected from the group consisting of C₂-C₆ alkenyl, C₂-C₆ alkynyl, and unsubstituted C₁-C₆ alkyl. In some embodiments Formulae (A), (I)-(IV), (VI)-(VIII), or (XI), R^{F} is C₁-C₆ alkyl. In some embodiments of Formulae (A), (I)-(IV), (VI)-(VIII), or (XI), R^{F} is methyl. In some embodiments of Formulae (A), (I)-(IV), (VI)-(VIII), or (XI), R^{F} is hydrogen.

In some embodiments of Formulae (A), (I)-(IV), (VI)-(VIII), or (XI), R⁵ is —C(═O)NR^{G}R^{H}. In some embodiments of Formulae (A), (I)-(IV), (VI)-(VIII), or (XI), R⁵ is —C(═O)OH. In some embodiments of Formulae (A), (I)-(IV), (VI)-(VIII), or (XI), R⁵ is —S(O₂)NR^{G}R^{H}. In some embodiments of Formulae (A), (I)-(IV), (VI)-(VIII), or (XI), R⁵ is —C(═O)NR^{G}R^{H}. In some embodiments of Formulae (A), (I)-(IV), (VI)-(VIII), or (XI), R⁵ is —S(O₂) NR^{G}R^{H}.

In some embodiments of Formulae (A), (I)-(IV), (VI)-(VIII), or (XI), each R^{G} and R^{H} are independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-. In some embodiments of Formulae (A), (I)-(IV), (VI)-(VIII), or (XI), each R^{G} and R^{H} are independently selected from the group consisting of hydrogen and C₁-C₆ alkyl. In some embodiments of Formulae (A), (I)-(IV), (VI)-(VIII), or (XI), R^{G} and R^{H}, together with the nitrogen atom to which they are attached, form a 3-6 membered heterocyclyl optionally substituted with 1-3 independently selected C₁-C₆ alkyl. In some embodiments of Formulae (A), (I)-(IV), (VI)-(VIII), or (XI), R¹ and R are independently selected from the group consisting of hydrogen and C₁-C₆ alkyl.

In some embodiments of Formula (A), R^{X} is —N(R)—S(O₂) R^{K}. In some embodiments of Formula (A), R^{X} is hydrogen.

In some embodiments of Formulae (A), (I)-(IV), (VI-VIII), or (XI), R⁵ is —N(R¹)—S(O₂)R^{K}. In some embodiments of Formulae (A), (I)-(IV), (VI-VIII), or (XI), R⁵ is hydrogen.

In some embodiments of Formulae (A), (I)-(IV), (VI-VIII), or (XI), R¹ and R^{K} are independently selected from the group consisting of hydrogen and C₁-C₆ alkyl.

In some embodiments of Formulae (A), (I)-(IV), (VI)-(VIII), or (XI), R⁵ is selected from the group consisting of —C(═O)OR^{F}, —CN, —CF₃—C(═O)NR^{G}R^{H}, and —N(R′)—C(═O)R^{J}. In some embodiments of Formulae (A), (I)-(IV), (VI)-(VIII), or (XI), R⁵ is selected from the group consisting of —C(═O)OR^{F}, —CN, —CF₃, —C(═O)NR^{G}R^{H}, and —N(R′)—C(═O)R^{J} In some embodiments of Formula (A), each R^{X} is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}. In some embodiments of Formula (A), each R^{X} is independently selected from the group consisting of halogen, hydroxyl, nitro, and cyano. In some embodiments of Formula (A), subscript n is 0. In some embodiments of Formula (A), subscript n is 1. In some embodiments of Formula (A), subscript n is 2. In some embodiments of Formula (A), subscript n is 3. In some embodiments of Formula (A), subscript n is 4.

In some embodiments of Formulae (A), (I)-(V), or (XI), each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}. In some embodiments of Formulae (A), (I)-(V), or (XI), each R⁶ is independently selected from the group consisting of halogen, hydroxyl, nitro, and cyano. In some embodiments of Formulae (A), (I)-(V), or (XI), subscript m is 0. In some embodiments of Formulae (A), (I)-(V), or (XI), subscript m is 1. In some embodiments of Formulae (A), (I)-(V), or (XI), subscript m is 2. In some embodiments of Formulae (A), (I)-(V), or (XI), subscript m is 3.

In some embodiments of Formulae (VI)-(VIII), each R^{6A} is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}. In some embodiments of Formulae (VI)-(VIII), each R^{6A} is independently selected from the group consisting of halogen, hydroxyl, nitro, and cyano. In some embodiments of Formulae (VI)-(VIII), subscript q is 0. In some embodiments of Formulae (VI)-(VIII), subscript q is 1. In some embodiments of Formulae (VI)-(VIII), subscript q is 2.

In some embodiments of Formulae (A), (I)-(VIII), or (XI), each R^{A} and R^{B} is independently selected from the group consisting of hydrogen and C₁-C₆ alkyl. In some embodiments of Formulae (A), (I)-(VIII), or (XI), each R^{A} and R^{B} is hydrogen. In some embodiments of Formulae (A), (I)-(VIII), or (XI), each R^{A} and R^{B} is an independently selected C₁-C₆ alkyl. In some embodiments of Formulae (A), (I)-(VIII), or (XI), one of R^{A} and R^{B} is hydrogen and the other of R^{A} and R^{B} is C₁-C₆ alkyl. In some embodiments, one of R^{A} and R^{B} is the point of covalent attachment to L and the other of R^{A} and R^{B} is hydrogen or C₁-C₆ alkyl. In some embodiments of Formulae (A), (I)-(VIII), or (XI), R^{C} is selected from the group consisting of hydrogen, phenyl, and C₁-C₁₀ alkyl optionally substituted with phenyl or 1-3 independently selected halogen.

In some embodiments of Formulae (A), (I)-(VIII), or (XI), R^{C} is selected from the group consisting of hydrogen, phenyl, and C₁-C₁₀ alkyl. In some embodiments of Formulae (A), (I)-(VIII), or (XI), R^{C} is selected from the group consisting of hydrogen and C₁-C₁₀ alkyl. In some embodiments of Formulae (A), (I)-(VIII), or (XI), R^{C} is hydrogen. In some embodiments of Formulae (A), (I)-(VIII), or (XI), R^{C} is C₁-C₁₀ alkyl. In some embodiments of Formulae (A), (I)-(VIII), or (XI), each R^{D} and R^{E} are independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl).

In some embodiments of Formulae (A), (I)-(VIII), or (XI), each R^{D} and R^{E} are independently selected from the group consisting of hydrogen and C₁-C₆ alkyl. In some embodiments of Formulae (A), (I)-(VIII), or (XI), R^{D} and R^{E}, together with the nitrogen atom to which they are attached, form a 3-6 membered heterocyclyl optionally substituted with 1-3 independently selected C₁-C₆ alkyl. In some embodiments of Formulae (A), (I)-(VIII), or (XI), R^{D} and R^{E}, together with the nitrogen atom to which they are attached which is also the point of covalent attachment to L, form a quaternary amine.

In some embodiments of Formulae (A), (I), or (III), R^{A}, R^{B}, R^{C}, R^{D}, and R^{E} are not points of covalent attachment to L. In some embodiments of Formulae (A), (I), or (III), R^{A}R^{B}, R^{C}, R^{D}, and R^{E} on R¹, R² and R⁴ are not points of covalent attachment to L. In some embodiments of Formulae (A), (I), or (III), R^{A}, R^{B}, R^{C}, R^{D}, and R^{E} on R¹ and R⁴ are not points of covalent attachment to L. In some embodiments of Formulae (A), (I), or (III), R^{A}R^{B}, R^{C}, R^{D}, and R^{E} on R¹ are not points of covalent attachment to L. In some embodiments of Formulae (A), (I), or (III), R^{A}, R^{B}, R^{C}, R^{D}, and R^{E} on R⁴ are not points of covalent attachment to L.

In some embodiments of Formulae (A) and (I)-(X), R¹ is not substituted with a solubilizing group (S_{b}). In some embodiments of Formulae (A) and (I)-(X), R⁴ is not substituted with a solubilizing group (S_{b}). In some embodiments of Formulae (A) and (I)-(X), R¹ and R⁴ are not substituted with solubilizing groups (S_{b}). In some embodiments of Formulae (A) and (I)-(X), only one of R¹ and R⁴ is substituted with a solubilizing group (S_{b}). In some embodiments of Formulae (A), (I), or (III), R¹ is not substituted with a solubilizing group if R¹ is a point of covalent attachment to L. In some embodiments of Formulae (A), (I), or (III), R⁴ is not substituted with a solubilizing group (S_{b}) if R¹ is a point of covalent attachment to L.

In some embodiments of Formulae (A), (I)-(VIII), or (XI), the solubilizing groups (S_{b}) are selected from the group consisting of C₅-C₉ monosaccharide and C₁₀-C₁₈ disaccharide. In some embodiments of Formulae (A), (I)-(VIII), or (XI), the C₅-C₉ monosaccharide is selected from the group consisting of glucose, galactose, fructose, fucose, mannose, xylose, xylitol, arabinose, rhamnose, ribose, sialic acid, sorbose, sorbitol, mannitol, tagatose. In some embodiments of Formulae (A), (I)-(VIII), or (XI), the C₁₀-C₁₈ disaccharide is selected from the group consisting of isomaltose, isomaltulose, gentiobiose, kojibiose, lactose, nigerose, laminaribiose, maltose, maltulose, mannobiose, melibiulose, rutinulose, sialic acid dimers, sophorose, sucrose, trehalose, turanose, and xylobiose. In some embodiments of Formulae (A), (I)-(VIII), or (XI), the C₁₅-C₂₇ trisaccharide is selected from the group consisting of isomaltotriose, kestose, nigerotriose, maltotriose, melezitose, maltotriulose, raffinose, and sialic acid trimers. In some embodiments of Formulae (A), (I)-(VIII), or (XI), the solubilizing group (S_{b}) is a C₅-C₆ monosaccharide. In some embodiments of Formulae (A), (I)-(VIII), or (XI), the solubilizing group (S_{b}) is a C₆ monosaccharide.

In some embodiments of Formula (XI), S_{b} is a solubilizing group selected from the group consisting of C₅-C₉ monosaccharide, C₁₀-C₁₈ disaccharide, and C₁₅-C₂₇ trisaccharide. In some embodiments of Formula (XI), S_{b} is a solubilizing group selected from the group consisting of C₅-C₉ monosaccharide and C₁₀-C₁₈ disaccharide. In some embodiments of Formula (XI), S_{b} is a C₅-C₉ monosaccharide. In some embodiments of Formula (XI), S_{b} is a C₆ monosaccharide. In some embodiments of Formula (XI), R¹ is C₁-C₆ alkoxycarbonyl or C₁-C₆ carbamoyl. In some embodiments of Formula (XI), R¹ is C₁-C₃ alkoxycarbonyl or C₁-C₃ carbamoyl. In some embodiments of Formula (XI), R¹ is C₁-C₃ alkoxycarbonyl or C₁-C₃ carbamoyl substituted with phenyl or 5-10 membered heteroaryl. In some embodiments of Formula (XI), R¹ is C₁-C₃ alkoxycarbonyl substituted with phenyl or 5-10 membered heteroaryl.

In some embodiments of Formula (XI), R¹—S_{b} is

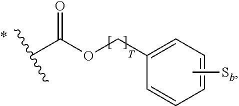

wherein subscript T is 1-6, and [symbol] indicates a covalent attachment to the remainder of Formula (XI).

In some embodiments of Formula (XI), R¹—S_{b} is

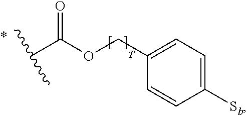

wherein subscript T is 1-6, and [symbol] indicates a covalent attachment to the remainder of Formula (XI). In some embodiments, subscript T is 1-3. In some embodiments, subscript T is 1.

### Methods of Making Compounds of the Present Disclosure

Further embodiments of the present disclosure provide methods of preparing compounds of Formulae (A) and (I)-(XI) and are illustrated by the following procedures in which the meanings of the generic radicals are as given above unless otherwise qualified. Certain compounds of Formulae (A) and (I)-(XI) can be useful as intermediates for preparing other compounds of Formulae (A) and (I)-(XI). In cases where compounds are sufficiently basic or acidic, a pharmaceutically acceptable salt of a compound of Formulae (A) and (I)-(XI) can be useful as an intermediate for isolating or purifying a compound of Formulae (A) and (I)-(XI).

General synthetic approaches toward imidazoquinolines (e.g., as shown in Formulae (A) and (I)-(XI)) are provided in Bioorg. Med. Chem. Lett. 59 (2022) 128548 and Molbank 2021, 2021, M1305, which are herein incorporated by reference. As such syntheses can be limited in their abilities to produce versatile functional group patterns on imidazoquinoline cores, some embodiments provided herein provide improved synthetic routes toward such imidazoquinoline structures as shown in Formulae (A) and (I)-(XI).

In certain embodiments, substituted imidazoquinoline compounds Formula (IX), can be synthesized according to SCHEME 70 below:

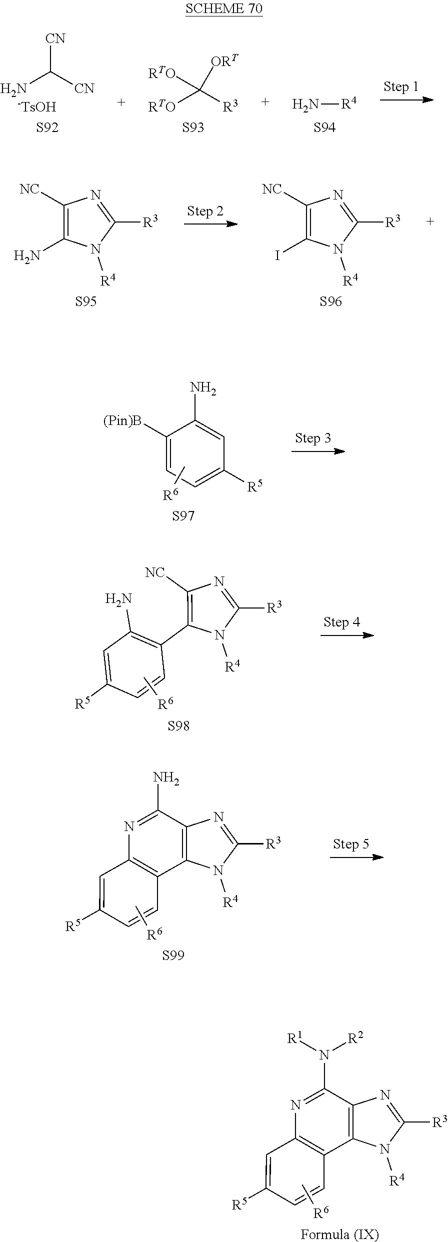

wherein:
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; (xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃—C(═O)NR^{G}R^{H}, —S(O₂)NR^{G}R^{H}, —N(R′)—C(═O)R, —N(R)—S(O₂)R^{K}, and SO₃R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl;
when R^{F} is trifluoromethyl, C₂-C₆ alkenyl, C₂-C₆ alkynyl, C₃-C₈ cycloalkyl, aryl, aryl(C₁-C₆ alkyl)-, or C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; R⁴ is —[N(C₁-C₆alkyl)R^{D}R^{E}]⁺; -(phenyl)C₁-C₆ alkyl, wherein its C₁-C₆ alkyl is substituted with —[N(C₁-C₆ alkyl)R^{D}R^{E}]⁺; or -(5-10 membered heteroaryl)C₁-C₆ alkyl, wherein its C₁-C₆ alkyl is substituted with —[N(C₁-C₆ alkyl)R^{D}R^{E}]⁺; and
each instance of R¹ and R⁴ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide.

Such a procedure can begin with a condensation (Step 1) between an aminomalontrile S92, and orthoester containing R³, S93, and an amine containing R⁴, S94, yielding a 4-cyano-5-imidazole intermediate S95 bearing R³ and R⁴. Step 1 can be performed under mild reflux conditions (e.g., 40-50° C.) in basic organic solvent, such as DCM with a tertiary amine. In Step 2, activation of S95 and conversion to S96 can be achieved with a Sandmeyer Reaction. In Step 3, S97 can then be combined with an R⁵ and R⁶ substituted 2-amino phenylboronic acid S98 through a cross-coupling step to form S99. A compound of Formula (IX) can then be generated via acid catalyzed cyclization of S99 (Step 4). In an optional subsequent step (Step 5), the imidazoquinoline 4-amino group can be substituted with R^{I} and/or R².

In many cases, a compound of Formula (IX) can be converted to a compound of Formula (X). As outlined in SCHEMES 71A-F below, a linker intermediate (L¹) can be coupled to R¹ or R², R³, R⁴, R⁵, R⁶, or the imidazoquinoline C4 amine of Formula (IX). Such a step can comprise site selective nucleophilic substitution by any of R¹-R⁶, or the imidazoquinoline C4 amine. The reagent nucleophilic substitution can include a linker intermediate (L¹) coupled to a carbonate (e.g., pentafluorophenyl carbonate (S100)), a carbamate (e.g., tosyl carbamate), a urea, a thiocarbonate, a thiocarbamate, an alkylbromide, an alkyl iodide, or an iodoketone. In some cases, the reagent for nucleophilic substitution comprises the linker intermediate (L¹) coupled to a carbonate or a carbamate. In some cases, the reagent for nucleophilic substitution comprises the linker intermediate (L¹) coupled to a carbonate. In some cases, the reagent for nucleophilic substitution comprises the linker intermediate (L¹) coupled to a pentafluorophenyl carbonate. In some cases, an amine, a thiol, or an enol of R¹-R⁶ couples to the nucleophilic substitution reagent (e.g., S100). In some cases, an amine of R¹-R⁶ couples to the nucleophilic substitution reagent. In some cases, the imidazoquinoline C4 amine is protected prior to R¹, R², R³, R⁴, R⁵, or R⁶ coupling to the nucleophilic substitution reagent.

SCHEME 71A depicts coupling between a representative reagent for nucleophilic substitution (S100) and R¹ of Formula (IX) to form Formula (X). While reaction between R² of Formula (IX) and S100 is not shown, such a reaction can be achieved in an analogous manner. SCHEME 71B depicts coupling between a representative reagent for nucleophilic substitution (S100) and R³ of Formula (IX) to form Formula (X). SCHEME 71C depicts coupling between a representative reagent for nucleophilic substitution (S100) and R⁴ of Formula (IX) to form Formula (X). SCHEME 71D depicts coupling between a representative reagent for nucleophilic substitution (S100) and R⁵ of Formula (IX) to form Formula (X). SCHEME 71E depicts coupling between a representative reagent for nucleophilic substitution (S100) and R⁶ of Formula (IX) to form Formula (X). SCHEME 71G depicts coupling between a representative reagent for nucleophilic substitution (S100) and the imidazoquinoline C4 amine of Formula (IX) to form Formula (X).

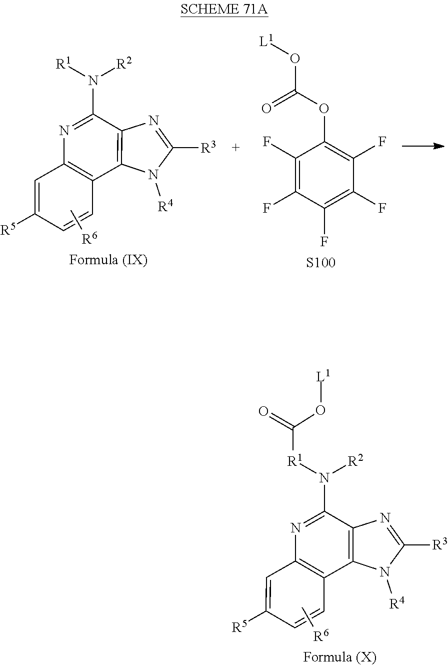

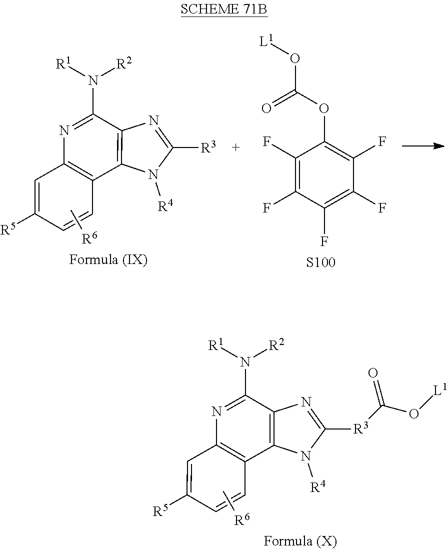

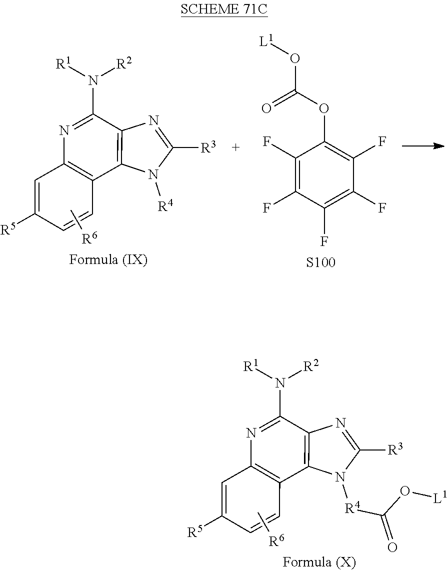

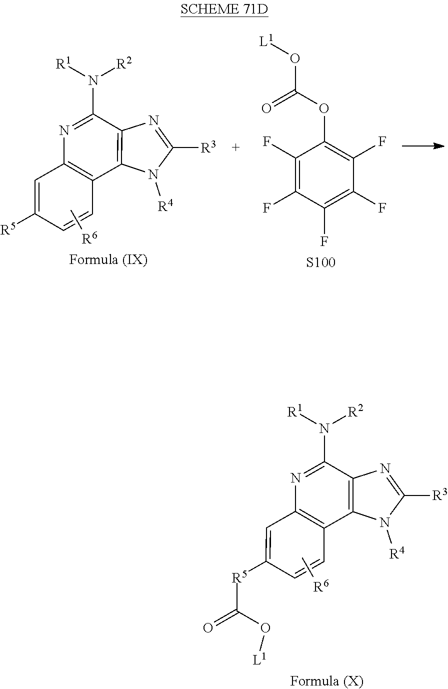

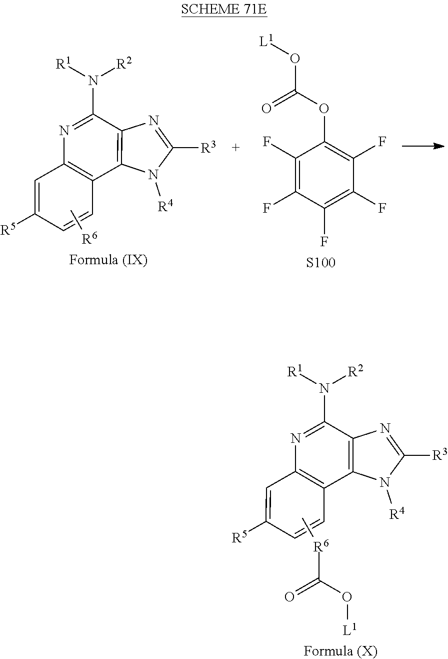

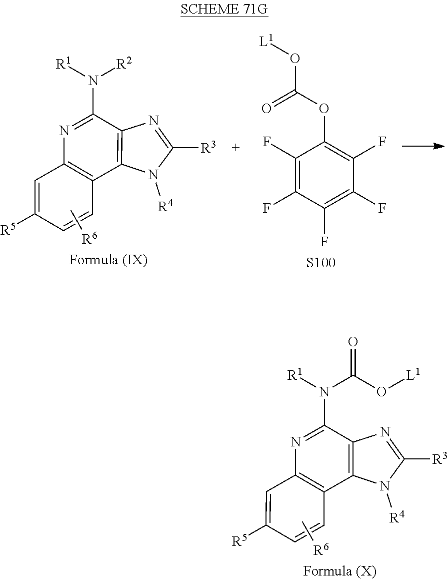

Alternative nucleophilic substitution schemes for forming Formula (X) by attaching a linker intermediate (L¹) to Formula (IX) are provided in SCHEMES 72A-F. As outlined in these schemes, R¹, R², R³, R⁴, R⁵, R⁶, or an imidazoquinoline amine (e.g., N1 or a C4 amine) can couple to an sp3 carbon of S101 through nucleophilic substitution, thereby displacing leaving group X_{L}. Examples of leaving groups (X_{L}) suitable for this step include chlorine, bromine, iodine, hydroxyl, nitrates, phosphates, alkoxides, phenoxides, and tosylates. In some cases, R¹, R², R³, R⁴, R⁵, or R⁶ of Formula (IX) couple through an amine. In some cases, the amine is a tertiary amine. In some cases, the amine comprises the formula —N(Me)₂. In some cases, reaction of the R¹, R², R³, R⁴, R⁵, or R⁶ amine with S101 forms a tertiary amine.

SCHEME 72A depicts coupling between a S101 and R¹ of Formula (IX) to form Formula (X). While reaction between R² of Formula (IX) and S101 is not shown, such a reaction can be achieved in an analogous manner. SCHEME 72B depicts coupling between S101 and R³ of Formula (IX) to form Formula (X). SCHEME 72C depicts coupling between S101 and R⁴ of Formula (IX) to form Formula (X). SCHEME 72D depicts coupling between S101 and R⁵ of Formula (IX) to form Formula (X). SCHEME 72E depicts coupling between S101 and R⁶ of Formula (IX) to form Formula (X). SCHEME 72G depicts coupling between S101 and the imidazoquinoline C4 amine of Formula (IX) to form Formula (X).

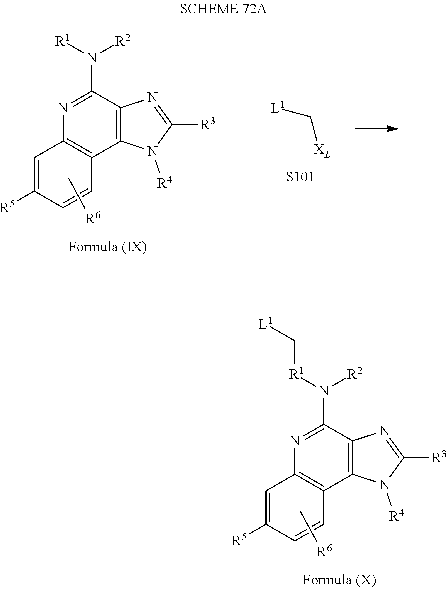

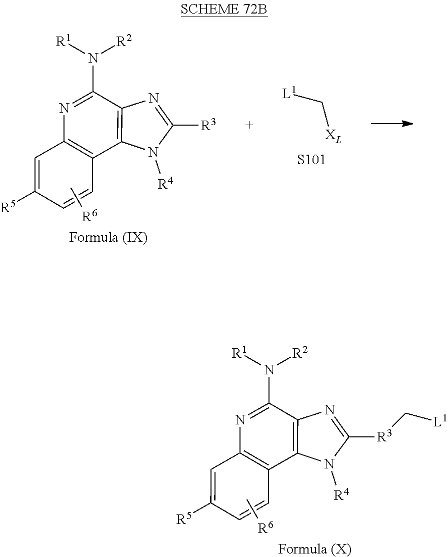

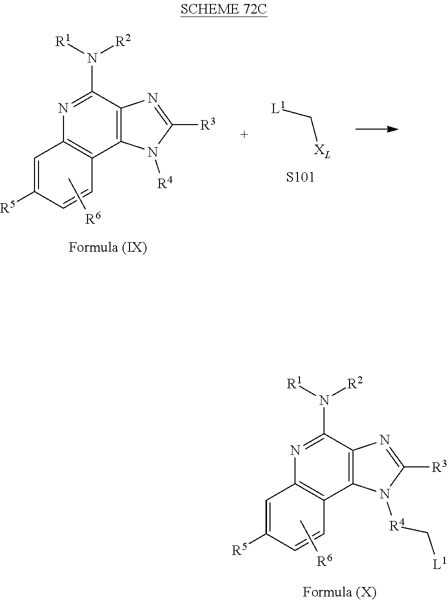

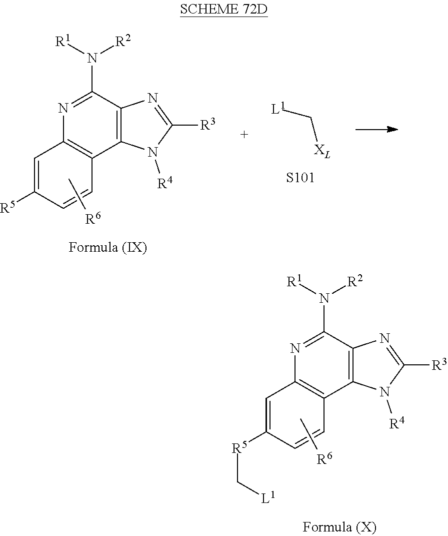

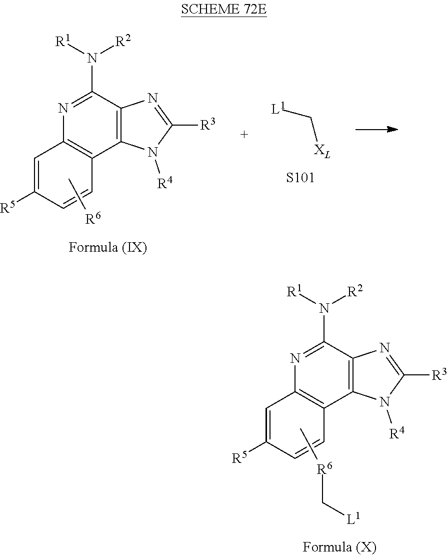

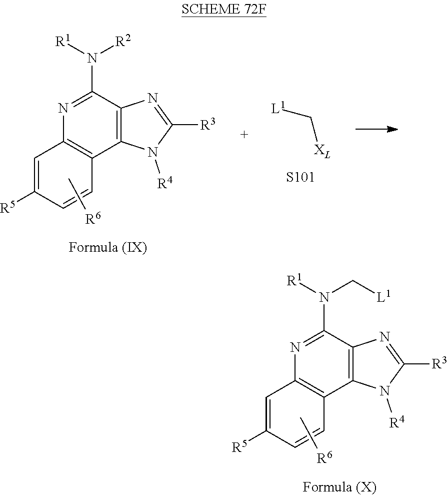

Formula (X) can be coupled to a biomolecule via the linker intermediate (L¹) to form any of Formulae (A), (I)-(VIII), or (XI). While SCHEME 73 depicts the formation of Formula (II) with a C4 amine-coupled linker, this scheme can be generalized for all of Formulae (A), (I)-(VIII), or (XI). The linker intermediate (L¹) can have the formula M¹-(A)ₐ-(W)_{w}—(Y)_{y}—(X)ₓ—; wherein:
M¹ comprises a functional group that will react with a protein (e.g., an antibody) to form a covalent bond;
subscripts a, w, y, and x are each independently 0 or 1; wherein the sum of subscripts a, w, y, and x is greater than or equal to 1; and
A, W, Y, X, are as defined for the linker (L).

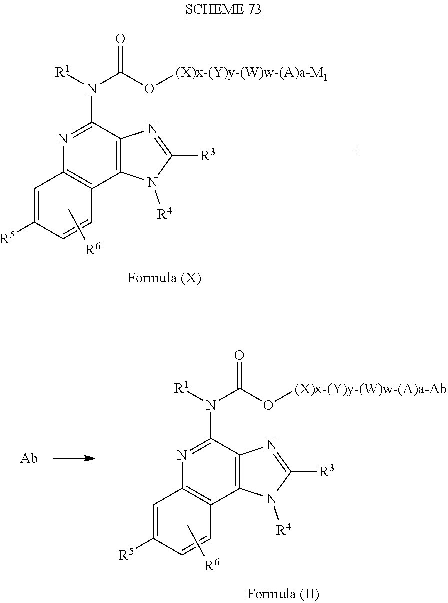

In some embodiments, M^{I} comprises a functional group that will react with an antibody to form a covalent bond (the Ab-M bond). In some embodiments, M^{I} is selected from the group consisting of maleimido, azido, C₁-C₆ alkynyl, cycloalkynyl optionally substituted with 1 or 2 fluoro (e.g., cyclooctynyl or DIFO), sulfhydryl, succinimidyl esters (e.g., N-hydroxysuccinimidyl (NHS) or sulfo-NHS esters), 4-nitrophenyl esters, pentafluorophenyl esters, tetrafluorophenyl esters, anhydrides, acid chlorides, sulfonyl chlorides, isocyanates, isothiocyanates, alpha-haloketones, alpha-O-sulfonate (e.g., mesyl or tosyl) ketones, alkyl hydrazines, hydrazides, and hydroxylamines. In some embodiments, M¹ is selected from the group consisting of maleimido, azido, C₁-C₆ alkynyl, cycloalkynyl optionally substituted with 1 or 2 fluoro (e.g., cyclooctynyl or DIFO), sulfhydryl, and succinimidyl esters.

In some embodiments, M¹ is configured to react with lysyl amines. In some embodiments, M¹ is configured to react with cysteine thiols. In some embodiments, M¹ is configured to react with lysyl amines and cysteine thiols. In some embodiments, M¹ is not reactive towards lysyl amines or cysteine thiols. In such cases, M¹ can be configured to couple with functionalized protein residues, for example with an alkyne or azide for click chemistry mediated coupling.

### Methods of Using Compounds of the Present Disclosure

In some embodiments, the ADCs described herein, or pharmaceutically acceptable salts thereof, are used to deliver the conjugated drug to a target tissue, cancer site, or cell. Without being bound by theory, in some embodiments, an ADC associates with an antigen on the surface of a target cell or in proximity to a target tissue, cancer site, or cell (e.g., an exosome surface protein in a tumor microenvironment), thereby localizing the ADC to the target tissue, cancer site, or cell.

In certain embodiments, an ADC as disclosed herein can elicit cancer-site specific immunostimulation. Cancer cells often generate immunosuppressive microenvironments, preventing recognition, immune cell activation, and cytotoxic activities that would otherwise remediate the cancer. In particular, many cancers generate localized concentrations of immune checkpoint inhibitors which can diminish the responsiveness of immune cells (e.g., T-cells), and in some cases actively recruit and transform them to promote further tumor growth and block responses from other immune cells. Accordingly, localized immune activation at cancer sites, as provided by certain ADCs of the present disclosure, can be critical for successful therapy.

In certain embodiments, an ADC of the present disclosure targets a cancer cell. In certain embodiments, the ADC is configured to internalize within the cancer cell (e.g., undergo endocytosis). The ADC can comprise a linker which is configured for cleavage inside of the cancer cell, but inert outside of the cancer cell. The ADC can comprise a drug (e.g., a compound comprising Formula (IX)) with low uptake efficiency but high potency, for example certain imidazoquinolines with negative C₇ functionalization.

In certain embodiments, the ADC is configured to bind to or near a cancer cell without undergoing cellular uptake. In such cases, the ADC can be configured to release a drug unit outside of the cancer cell. For example, the ADC can comprise a linker which undergoes cleavage in high pH cancer microenvironments, but which is otherwise stable during circulation. Alternatively or additionally thereto, the ADC can comprise a linker with a cleavage sequence for a protease overexpressed by the cancer cell, for example a serine protease overexpressed by a prostate cancer cell.

Alternatively or in addition thereto, an ADC can be configured to undergo transcytosis upon binding to a target cell or tissue. Such an ADC can target a receptor or comprise a structure (e.g., an IgA immunoglobulin) which facilitates transport across a cell barrier. For example, upon binding to a receptor on a tumor associated macrophage (TAM), such an ADC may transcytose, thereby passing through a passivating TAM layer and reaching underlying cancer cells.

In some embodiments, an ADC targets a surface antigen of a cell. The ADC can be configured to localize to the cell (e.g., bind to the cell and not internalize within the cell, or bind to the cell to increase uptake into the cell). In some such cases, the ADC can release its drug payload in proximity to the cell. For example, the ADC can comprise a linker with a peptide portion that is cleavable by a protease excreted by the cell. The ADC can also be configured to internalize within the cell. For example, the ADC may endocytose within the cell upon binding to the surface antigen.

In certain cases, the surface antigen is associated with a cancer cell. In certain cases, the surface antigen is associated with a cancer cell and is not associated with an immune cell. In certain cases, the surface antigen is associated with an immune cell. In certain cases, the surface antigen is associated with an immune cell and is not associated with a cancer cell. In certain cases, the surface antigen is associated with a cancer cell and an immune cell. In certain cases, the immune cell is a tumor associated macrophage.

ADC internalization can enable drug localization to otherwise inaccessible targets. As many signalling pathways are primarily intracellular (e.g., the majority of signalling proteins and signal transduction events occur inside of a cell), signal modulation often requires effective drug internalization and subcellular localization. As many drugs exhibit poor cellular uptake and non-specific subcellular localization, many targets remain inaccessible for drug targeting. Archetypal examples of such targets are TLR7 and TLR8, whose endosomal localizations are inaccessible to many treatments.

Certain ADCs disclosed herein overcome this challenge by mediating uptake and subcellular localization to deliver high payload concentrations at select target sites. In some cases, ADC binding to a cell surface antigen mediates uptake into the cell. In specific cases, an ADC is configured to endocytose into a target cell, thereby accessing endosomally compartmentalized species, such as TLR7 and TLR8. In some cases, a linker (L) of an ADC is configured to undergo cleavage subsequent to internalizing within the target cell. In some cases, the linker (L) of an ADC is configured to undergo cleavage within a specific subcellular space. For example, a linker (L) of an ADC can comprise a peptide with a cleavage sequence specific for a lysosomal protease.

Some embodiments provide a method of treating a viral or bacterial infection in a subject in need thereof, comprising administering to the subject a therapeutically effective amount of an ADC described herein, or a pharmaceutically acceptable salt thereof.

Some embodiments provide a method of treating viral or bacterial infection in a subject in need thereof, comprising administering to the subject a therapeutically effective amount of a composition comprising an ADC described herein, or a pharmaceutically acceptable salt thereof.

Some embodiments provide a method of inducing an anti-viral or anti-bacterial immune response in a subject in need thereof, comprising administering to the subject a therapeutically effective amount of a composition comprising an ADC described herein, or a pharmaceutically acceptable salt thereof.

Some embodiments provide a method of inducing an anti-viral or anti-bacterial immune response in a subject in need thereof, comprising administering to the subject a therapeutically effective amount of an ADC described herein, or a pharmaceutically acceptable salt thereof.

Some embodiments provide a method of treating cancer in a subject in need thereof, comprising administering to the subject a therapeutically effective amount of an ADC described herein, or a pharmaceutically acceptable salt thereof.

Some embodiments provide a method of treating cancer in a subject in need thereof, comprising administering to the subject a therapeutically effective amount of a composition comprising an ADC described herein, or a pharmaceutically acceptable salt thereof.

Some embodiments provide a method of inducing an anti-tumor immune response in a subject in need thereof, comprising administering to the subject a therapeutically effective amount of a composition comprising an ADC described herein, or a pharmaceutically acceptable salt thereof.

Some embodiments provide a method of inducing an anti-tumor immune response in a subject in need thereof, comprising administering to the subject a therapeutically effective amount of an ADC described herein, or a pharmaceutically acceptable salt thereof.

Some embodiments provide a method of treating cancer in a subject in need thereof, comprising administering to the subject a therapeutically effective amount of an ADC as described herein, or a pharmaceutically acceptable salt thereof, in combination with another anticancer therapy (e.g., surgery and radiation therapy) and/or anticancer agent (e.g., an immunotherapy such as nivolumab or pembrolizumab). The ADCs described herein can be administered to the subject before, during, or after administration of the anticancer therapy and/or anticancer agent and/or surgery. In some embodiments, the ADCs described herein can be administered to the subject following treatment with radiation and/or after surgery.

Some embodiments provide a method for delaying or preventing acquired resistance to an anticancer agent, comprising administering to the subject a therapeutically effective amount of an ADC as described herein, or a pharmaceutically acceptable salt thereof, to a patient at risk for developing or having acquired resistance to an anticancer agent. In some embodiments, the patient is administered a dose of the anticancer agent (e.g., at substantially the same time as a dose of an ADC as described herein, or a pharmaceutically acceptable salt thereof is administered to the patient).

Some embodiments provide a method of delaying and/or preventing development of cancer resistant to an anticancer agent in a subject, comprising administering to the subject a therapeutically effective amount of an ADC as described herein, or a pharmaceutically acceptable salt thereof, before, during, or after administration of a therapeutically effective amount of the anticancer agent.

The ADCs described herein are useful for inhibiting the multiplication of a cancer cell, causing apoptosis in a cancer cell, for increasing phagocytosis of a cancer cell, and/or for treating cancer in a subject in need thereof. The ADCs can be used accordingly in a variety of settings for the treatment of cancers. The ADCs can be used to deliver a drug to a cancer cell. Without being bound by theory, in some embodiments, the antibody of an ADC binds to or associates with a cancer-cell-associated antigen. The antigen can be attached to a cancer cell or can be an extracellular matrix protein associated with the cancer cell. The drug can be released in proximity to the cancer cell, thus recruiting/activating immune cells to attack the cancer cell. In some embodiments, the Drug Unit is cleaved from the ADC outside the cancer cell. In some embodiments, the Drug Unit remains attached to the antibody bound to the antigen.

In some embodiments, the antibody binds to the cancer cell. In some embodiments, the antibody binds to a cancer cell antigen which is on the surface of the cancer cell. In some embodiments, the antibody binds to a cancer cell antigen which is an extracellular matrix protein associated with the tumor cell or cancer cell. In some embodiments, the antibody of an ADC binds to or associates with a cancer-associated cell or an antigen on a cancer-associated cell. In some embodiments, the cancer-associated cell is a stromal cell in a tumor, for example, a cancer-associated fibroblast (CAF).

In some embodiments, the antibody of an ADC binds to or associates with an immune cell or an immune-cell-associated antigen. The antigen can be attached to an immune cell or can be an extracellular matrix protein associated with the immune cell. The drug can be released in proximity to the immune cell, thus recruiting/activating the immune cell to attack a cancer cell. In some embodiments, the Drug Unit is cleaved from the ADC outside the immune cell. In some embodiments, the Drug Unit remains attached to the antibody bound to the antigen. In some embodiments, the immune cell is a lymphocyte, an antigen-presenting cell, a natural killer (NK) cell, a neutrophil, an eosinophil, a basophil, a mast cell, an innate lymphoid cell, or a combination of any of the foregoing. In some embodiments, the immune cell is selected from the group consisting of B cells, plasma cells, T cells, NKT cells, gamma delta T cells, monocytes, macrophages, dendritic cells, natural killer (NK) cells, neutrophils, eosinophils, basophils, mast cells, and a combination of any of the foregoing.

The specificity of the antibody for a particular cancer cell can be important for determining those tumors or cancers that are most effectively treated. For example, ADCs that target a cancer cell antigen present on hematopoietic cancer cells in some embodiments treat hematologic malignancies. In some embodiments, ADCs target a cancer cell antigen present on abnormal cells of solid tumors for treating such solid tumors. In some embodiments an ADC are directed against abnormal cells of hematopoietic cancers such as, for example, lymphomas (Hodgkin Lymphoma and Non-Hodgkin Lymphomas) and leukemias.

Cancers, including, but not limited to, a tumor, metastasis, or other disease or disorder characterized by abnormal cells that are characterized by uncontrolled cell growth in some embodiments are treated or inhibited by administration of an ADC.

In some embodiments, the subject has previously undergone treatment for the cancer. In some embodiments, the prior treatment is surgery, radiation therapy, administration of one or more anticancer agents, or a combination of any of the foregoing.

In any of the methods described herein, the cancer is selected from the group consisting of: adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, esophagus and stomach carcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, kidney carcinoma, leukemia lymphocytic, leukemia lymphocytic chronic, liver cholangiocarcinoma, liver hepatocellular carcinoma, liver carcinoma, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, upper aerodigestive tract squamous cell carcinoma, upper aerodigestive tract carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, lymphoma, non-Hodgkins, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), large intestine/colon carcinoma, large intestine adenocarcinoma, soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epitheliod cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, lymphoid neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma.

In some embodiments, the subject is concurrently administered one or more additional anticancer agents with the ADCs described herein, or a pharmaceutically acceptable salt thereof. In some embodiments, the subject is concurrently receiving radiation therapy with the ADCs described herein, or a pharmaceutically acceptable salt thereof. In some embodiments, the subject is administered one or more additional anticancer agents after administration of the ADCs described herein, or a pharmaceutically acceptable salt thereof. In some embodiments, the subject receives radiation therapy after administration of the ADCs described herein, or a pharmaceutically acceptable salt thereof.

In some embodiments, the subject has discontinued a prior therapy, for example, due to unacceptable or unbearable side effects, wherein the prior therapy was too toxic, or wherein the subject developed resistance to the prior therapy.

Some embodiments provide a method for delaying or preventing a disease or disorder, comprising administering to the subject a therapeutically effective amount of an ADC as described herein, or a pharmaceutically acceptable salt thereof, and a vaccine against the disease or disorder, to a patient at risk for developing the disease or disorder. In some embodiments, the disease or disorder is cancer, as described herein. In some embodiments, the disease or disorder is a viral pathogen. In some embodiments, the vaccine is administered subcutaneously. In some embodiments, the vaccine is administered intramuscularly. In some embodiments, the ADC and the vaccine are administered via the same route (for example, the ADC and the vaccine are both administered subcutaneously). In some embodiments, the ADC, or a pharmaceutically acceptable salt thereof, and the vaccine are administered via different routes. In some embodiments, the vaccine and the ADC, or a pharmaceutically acceptable salt thereof, are provided in a single formulation. In some embodiments, the vaccine and the ADC, or a pharmaceutically acceptable salt thereof, are provided in separate formulations.

In some embodiments, the ADCs described herein are present in the form of a salt. In some embodiments, the salt is a pharmaceutically acceptable salt.

### Pharmaceutical Compositions of the Present Disclosure and Methods of Use Thereof

Some embodiments provide a composition comprising a distribution of ADCs, as described herein. In some embodiments, the composition comprises a distribution of ADCs, as described herein and at least one pharmaceutically acceptable carrier. In some embodiments, the route of administration is parenteral. Parenteral administration includes subcutaneous injections, intravenous, intramuscular, intrasternal injection or infusion techniques. In some embodiments, the compositions are administered parenterally. In one of those embodiments, the ADCs are administered intravenously. Administration is typically through any convenient route, for example by infusion or bolus injection.

Compositions of an ADC can be formulated so as to allow the ADC to be bioavailable upon administration of the composition to a subject. Compositions can be in the form of one or more injectable dosage units.

Materials used in preparing the compositions can be non-toxic in the amounts used. It will be evident to those of ordinary skill in the art that the optimal dosage of the active ingredient(s) in the composition will depend on a variety of factors. Relevant factors include, without limitation, the type of animal (e.g., human), the particular form of the compound, the manner of administration, and the composition employed.

In some embodiments, the ADC composition is a solid, for example, as a lyophilized powder, suitable for reconstitution into a liquid prior to administration. In some embodiments, the ADC composition is a liquid composition, such as a solution or a suspension. A liquid composition or suspension is useful for delivery by injection and a lyophilized solid is suitable for reconstitution as a liquid or suspension using a diluent suitable for injection. In a composition administered by injection, one or more of a surfactant, preservative, wetting agent, dispersing agent, suspending agent, buffer, stabilizer and isotonic agent is typically included.

In some embodiments, the liquid compositions, whether they are solutions, suspensions or other like form, can also include one or more of the following: sterile diluents such as water for injection, saline solution, physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. A parenteral composition is typically enclosed in ampoule, a disposable syringe or a multiple-dose vial made of glass, plastic or other material. In some embodiments, the sterile diluent comprises physiological saline. In some embodiments, the sterile diluent is physiological saline. In some embodiments, the composition described herein are liquid injectable compositions that are sterile.

The amount of the ADC that is effective in the treatment of a particular disorder or condition will depend on the nature of the disorder or condition, which is usually determined by standard clinical techniques. In addition, in vitro or in vivo assays are sometimes employed to help identify optimal dosage ranges. The precise dose to be employed in the compositions will also depend on the route of parenteral administration, and the seriousness of the disease or disorder, and should be decided according to the judgment of the practitioner and each subject's circumstances.

In some embodiments, the compositions comprise an effective amount of an ADC such that a suitable dosage will be obtained. Typically, this amount is at least about 0.01% of the ADC by weight of the composition.

In some embodiments, the compositions dosage of an ADC administered to a subject is from about 0.01 mg/kg to about 100 mg/kg, from about 1 to about 100 mg of a per kg or from about 0.1 to about 25 mg/kg of the subject's body weight. In some embodiments, the dosage administered to a subject is about 0.01 mg/kg to about 15 mg/kg of the subject's body weight. In some embodiments, the dosage administered to a subject is about 0.1 mg/kg to about 15 mg/kg of the subject's body weight. In some embodiments, the dosage administered to a subject is about 0.1 mg/kg to about 20 mg/kg of the subject's body weight. In some embodiments, the dosage administered is about 0.1 mg/kg to about 5 mg/kg or about 0.1 mg/kg to about 10 mg/kg of the subject's body weight. In some embodiments, the dosage administered is about 1 mg/kg to about 15 mg/kg of the subject's body weight. In some embodiments, the dosage administered is about 1 mg/kg to about 10 mg/kg of the subject's body weight. In some embodiments, the dosage administered is about 0.1 to about 4 mg/kg, about 0.1 to about 3.2 mg/kg, or about 0.1 to about 2.7 mg/kg of the subject's body weight over a treatment cycle.

The term “carrier” refers to a diluent, adjuvant or excipient, with which a compound is administered. Such pharmaceutical carriers are liquids. Water is an exemplary carrier when the compounds are administered intravenously. Saline solutions and aqueous dextrose and glycerol solutions are also useful as liquid carriers for injectable solutions. Suitable pharmaceutical carriers also include glycerol, propylene, glycol, or ethanol. The present compositions, if desired, will in some embodiments also contain minor amounts of wetting or emulsifying agents, and/or pH buffering agents.

In some embodiments, the ADCs are formulated in accordance with routine procedures as a composition adapted for intravenous administration to animals, particularly human beings. Typically, the carriers or vehicles for intravenous administration are sterile isotonic aqueous buffer solutions. In some embodiments, the composition further comprises a local anesthetic, such as lignocaine, to ease pain at the site of the injection. In some embodiments, the ADC and the remainder of the formulation are supplied either separately or mixed together in unit dosage form, for example, as a dry lyophilized powder or water free concentrate in a hermetically sealed container such as an ampoule or sachette indicating the quantity of active agent. Where an ADC is to be administered by infusion, it is sometimes dispensed, for example, with an infusion bottle containing sterile pharmaceutical grade water or saline. Where the ADCs are administered by injection, an ampoule of sterile water for injection or saline is typically provided so that the ingredients can be mixed prior to administration.

The compositions are generally formulated as sterile, substantially isotonic and in full compliance with all Good Manufacturing Practice (GMP) regulations of the U.S. Food and Drug Administration.

### Compounds of Formula (IX)

Some embodiments provide a compound of Formula (IX):

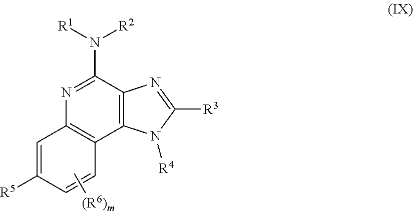

or a pharmaceutically acceptable salt thereof,
wherein:
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C} or (b) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]+, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is —C(═O)OR^{F};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl; and
when R^{F} is trifluoromethyl, C₂-C₆ alkenyl, C₂-C₆ alkynyl, C₃-C₈ cycloalkyl, aryl, aryl(C₁-C₆ alkyl)-, or C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; R⁴ is —[N(C₁-C₆alkyl)R^{D}R^{E}]⁺; -(phenyl)C₁-C₆ alkyl, wherein its C₁-C₆ alkyl is substituted with —[N(C₁-C₆ alkyl)R^{D}R^{E}]⁺; or -(5-10 membered heteroaryl)C₁-C₆ alkyl, wherein its C₁-C₆ alkyl is substituted with —[N(C₁-C₆ alkyl)R^{D}R^{E}]⁺.

Some embodiments provide a compound of Formula (IX):

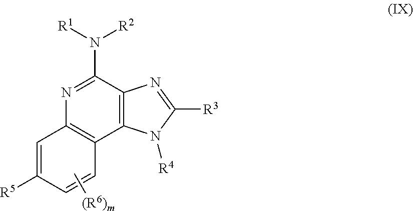

or a pharmaceutically acceptable salt thereof,
wherein:
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is selected from the group consisting of hydrogen, —C(═O)OR^{F}, —NO₂, —CN, —CF₃—C(═O)NR^{G}R^{H}, —S(O₂)NR^{G}R^{H}, —N(R′)—C(═O)R, —N(R)—S(O₂)R^{K}, and SO₃R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is independently selected from the group consisting of hydrogen and C₁-C₆ alkyl;
when R^{F} is trifluoromethyl, C₂-C₆ alkenyl, C₂-C₆ alkynyl, C₃-C₈ cycloalkyl, aryl, aryl(C₁-C₆ alkyl)-, or C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; R⁴ is —[N(C₁-C₆alkyl)R^{D}R^{E}]⁺; -(phenyl)C₁-C₆ alkyl, wherein its C₁-C₆ alkyl is substituted with —[N(C₁-C₆ alkyl)R^{D}R^{E}]⁺; or -(5-10 membered heteroaryl)C₁-C₆ alkyl, wherein its C₁-C₆ alkyl is substituted with —[N(C₁-C₆ alkyl)R^{D}R^{E}]⁺; and
each instance of R¹ and R⁴ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide.

In some embodiments, the free drug, as described herein, is a compound of Formula (IX). In some embodiments, the free drug, as described herein, comprises a compound of Formula (IX).

In some embodiments, the free drug, as described herein, comprises a compound of Formula (IX) in prodrug form. In some cases, R¹ of the compound of Formula (IX) in prodrug form is selected from the group consisting of C₁-C₆ alkoxycarbonyl, C₁-C₆ alkoxythiocarbonyl, C₁-C₆ carbamoyl, C₁-C₆ amidine, C₁-C₆ sulfone, and C₁-C₆ thione. In some cases, R¹ of the compound of Formula (IX) in prodrug form is selected from the group consisting of C₁-C₆ alkoxycarbonyl, C₁-C₆ alkoxythiocarbonyl, and C₁-C₆ carbamoyl. In some cases, R¹ of the compound of Formula (IX) in prodrug form is C₁-C₆ alkoxycarbonyl.

In some embodiments of Formula (IX), R¹ is selected from the group consisting of hydrogen, C₁-C₆ alkyl, C₂-C₆ alkenyl, C₂-C₆ alkynyl, C₁-C₆ alkoxycarbonyl, C₁-C₆ alkoxythiocarbonyl, C₁-C₆ carbamoyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkoxycarbonyl, C₁-C₆ alkoxythiocarbonyl, C₁-C₆ carbamoyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}.

In some embodiments of Formula (IX), R¹ is selected from the group consisting of hydrogen, C₁-C₆ alkyl, C₂-C₆ alkenyl, C₁-C₆ alkoxycarbonyl, C₁-C₆ carbamoyl, C₃-C₆ cycloalkyl, phenyl, and 5-10 membered heteroaryl; wherein each C₁-C₆ alkyl, C₂-C₆ alkenyl, C₁-C₆ alkoxycarbonyl, C₁-C₆ carbamoyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}. In some embodiments of Formula (IX), R¹ is selected from the group consisting of C₁-C₆ alkoxycarbonyl and C₁-C₆ carbamoyl; wherein each C₁-C₆ alkoxycarbonyl and C₁-C₆ carbamoyl is optionally substituted with 1-3 substituents independently selected from the group consisting of hydroxyl, halogen, sulfhydryl, cyano, oxiranyl, C₃-C₅ cycloalkyl, phenyl, 5-10 membered heteroaryl, C₁-C₆ alkoxy, C₁-C₆ alkylthio, and —NR^{A}R^{B}.

In some embodiments, the compound of Formula (IX) has the structure of Formula (IX-A):

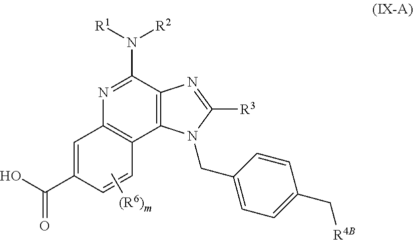

or a pharmaceutically acceptable salt thereof,
wherein:
R¹ is selected from the group consisting of hydrogen and C₁-C₆ alkyl;
R² is selected from the group consisting of hydrogen and C₁-C₆ alkyl; or
R¹ and R², taken together with the nitrogen atom to which they are attached, form a 3-6 membered heterocyclyl optionally substituted with 1-3 independently selected C₁-C₆ alkyl;
R³ is selected from the group consisting of hydrogen and C₁-C₆ alkyl optionally substituted with 1-3 substituents independently selected from the group consisting of hydroxyl, halogen, sulfhydryl, cyano, oxo, oxiranyl, C₁-C₆ alkoxy, and C₁-C₆ alkylthio;
R^{4B} is selected from the group consisting of 5-10 membered heteroaryl, —NR^{D}R^{E}, and —[N(C₁-C₆ alkyl)R^{D}R^{E}]⁺;
R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, and C₁-C₆ haloalkoxy;
subscript m is 0 or 1; and
R^{D} and R^{E} are independently selected from the group consisting of hydrogen, C₁-C₆ alkyl, C₂-C₆ alkenyl, C₃-C₅ cycloalkyl, C₃-C₅ cycloalkyl(C₁-C₆ alkyl)-, aryl, and aryl(C₁-C₆ alkyl)-; or R^{D} and R^{E}, together with the nitrogen atom to which they are attached, form a 3-6 membered heterocyclyl optionally substituted with 1-3 independently selected C₁-C₆ alkyl.

In some embodiments, the compound of Formula (IX) has the structure of Formula (IX-B):

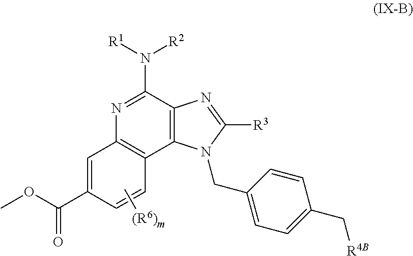

or a pharmaceutically acceptable salt thereof,
wherein:
R¹ is selected from the group consisting of hydrogen and C₁-C₆ alkyl;
R² is selected from the group consisting of hydrogen and C₁-C₆ alkyl; or
R¹ and R², taken together with the nitrogen atom to which they are attached, form a 3-6 membered heterocyclyl optionally substituted with 1-3 independently selected C₁-C₆ alkyl;
R³ is selected from the group consisting of hydrogen and C₁-C₆ alkyl optionally substituted with 1-3 substituents independently selected from the group consisting of hydroxyl, halogen, sulfhydryl, cyano, oxo, oxiranyl, C₁-C₆ alkoxy, and C₁-C₆ alkylthio;
R^{4B} is selected from the group consisting of 5-10 membered heteroaryl, —NR^{D}R^{E}, and —[N(C₁-C₆ alkyl)R^{D}R^{E}]⁺;
R⁶ selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, and C₁-C₆ haloalkoxy;
subscript m is 0 or 1; and
R^{D} and R^{E} are independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, together with the nitrogen atom to which they are attached, form a 3-6 membered heterocyclyl optionally substituted with 1-3 independently selected C₁-C₆ alkyl.

In some embodiments of Formula (IX), R¹ and R² are each independently selected C₁-C₆ alkyl. In some embodiments of Formula (IX), R¹ and R² are both methyl. In some embodiments of Formula (IX), one of R¹ and R² is hydrogen and the other of R¹ and R² is C₁-C₆ alkyl. In some embodiments of Formula (IX), one of R¹ and R² is hydrogen and the other of R¹ and R² is methyl. In some embodiments of Formula (IX), R¹ and R² are both hydrogen. In some embodiments of Formula (IX), R¹ is selected from the group consisting of hydrogen, C₁-C₆ alkyl, C₂-C₆ alkenyl, C₁-C₆ alkoxycarbonyl, C₁-C₆ carbamoyl, C₃-C₆ cycloalkyl, phenyl, and 5-10 membered heteroaryl; wherein each C₁-C₆ alkyl, C₂-C₆ alkenyl, C₁-C₆ alkoxycarbonyl, C₁-C₆ carbamoyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}. In some embodiments of Formula (IX), R¹ is selected from the group consisting of C₁-C₆ alkoxycarbonyl and C₁-C₆ carbamoyl; wherein each C₁-C₆ alkoxycarbonyl and C₁-C₆ carbamoyl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}.

In some embodiments of Formula (IX), R¹ and R², taken together with the nitrogen atom to which they are attached, form a 3-6 membered heterocyclyl optionally substituted with 1-3 independently selected C₁-C₆ alkyl.

In some embodiments of Formula (IX), R³ is C₁-C₆ alkyl optionally substituted with 1-3 substituents independently selected from the group consisting of hydroxyl, halogen, sulfhydryl, cyano, oxo, oxiranyl, C₁-C₆ alkoxy, and C₁-C₆ alkylthio. In some embodiments of Formula (IX), R³ is C₁-C₆ alkyl substituted with one substituent selected from the group consisting of hydroxyl, halogen, sulfhydryl, cyano, oxo, oxiranyl, C₁-C₆ alkoxy, and C₁-C₆ alkylthio. In some embodiments of Formula (IX), R³ is C₁-C₆ alkyl substituted with one substituent selected from the group consisting of hydroxyl and C₁-C₆ alkoxy. In some embodiments of Formula (IX), R³ is an unsubstituted C₁-C₆ alkyl. In some embodiments of Formula (IX), R³ is n-butyl. In some embodiments of Formula (IX), R³ is hydrogen.

In some embodiments of Formula (IX), R⁴ is —S(═O)₂R^{C}; —C(═O)NR^{D}R^{E}; —C(═O)OR^{C}; —C(═O)SR^{C}; or C₁-C₆ alkyl optionally substituted with a group selected from the group consisting of (i)-(xiv), as described herein. In some embodiments of Formula (IX), R⁴ is —C(═O)NR^{D}R^{E}; —C(═O)OR^{c}; or C₁-C₆ alkyl optionally substituted with a group selected from the group consisting of (i)-(xiv), as described herein. In some embodiments of Formula (IX), R⁴ is —C(═O)NR^{D}R^{E} or —C(═O)OR^{C}. In some embodiments of Formula (IX), R⁴ is —C(═O)NR^{D}R^{E}.

In some embodiments of Formula (IX), R⁵ is selected from the group consisting of —C(═O)OH, —NO₂, —CN, —CF₃, and —S(O₃)H. In some embodiments of Formula (IX), R⁵ is selected from the group consisting of —C(═O)OH and —S(O₃)H. In some embodiments Formula (IX), R⁵ is —C(═O)OH.

In some embodiments of Formula (IX), R¹ is not substituted with a solubilizing group (S_{b}). In some embodiments of Formula (IX), R⁴ is not substituted with a solubilizing group (S_{b}). In some embodiments of Formula (IX), R¹ and R⁴ are not substituted with solubilizing groups (S_{b}). In some embodiments of Formula (IX), only one of R¹ and R⁴ is substituted with a solubilizing group (S_{b}). In some embodiments of Formula (IX), R¹ is not substituted with a solubilizing group if R¹ is a point of covalent attachment to L. In some embodiments of Formula (IX), R⁴ is not substituted with a solubilizing group (S_{b}) if R¹ is a point of covalent attachment to L.

In some embodiments of Formulae (IX-A) and (IX-B), R^{4B} is -NR^{D}R^{E}.

In some embodiments of Formulae (IX-A) and (IX-B), R^{4B} is —[N(C₁-C₆ alkyl)R^{D}R^{E}]⁺.

In some embodiments of Formulae (IX-A) and (IX-B), R^{D} and R^{E} are independently selected from the group consisting of hydrogen, C₁-C₆ alkyl, C₂-C₆ alkenyl, C₃-C₅ cycloalkyl, C₃-C₈ cycloalkyl(C₁-C₆ alkyl)-, aryl, and aryl(C₁-C₆ alkyl)-. In some embodiments of Formulae (IX-A) and (IX-B), R^{D} and R^{E} are independently selected from the group consisting of hydrogen, C₁-C₆ alkyl, and C₂-C₆ alkenyl.

In some embodiments of Formulae (IX-A) and (IX-B), R^{D} and R^{E} are each independently selected C₁-C₆ alkyl. In some embodiments of Formulae (IX-A) and (IX-B), R^{D} and R^{E} are both methyl. In some embodiments of Formulae (IX-A) and (IX-B), one of R^{D} and R^{E} is hydrogen and the other of R^{D} and R^{E} is C₁-C₆ alkyl. In some embodiments of Formulae (IX-A) and (IX-B), one of R^{D} and R^{E} is hydrogen and the other of R^{D} and R^{E} is methyl.

In some embodiments of Formulae (IX-A) and (IX-B), R^{D} and R^{E} are both hydrogen.

In some embodiments of Formulae (IX-A) and (IX-B), R^{D} and R^{E}, together with the nitrogen atom to which they are attached, form a 3-6 membered heterocyclyl optionally substituted with 1-3 independently selected C₁-C₆ alkyl.

In some embodiments of Formulae (IX-A) and (IX-B), R^{4B} is 5-10 membered heteroaryl.

In some embodiments of Formula (IX), subscript m is 1. In some embodiments of Formula (IX), subscript m is 0.

In some embodiments of Formula (IX), R⁶ is selected from the group consisting of halogen, hydroxyl, nitro, cyano, C₁-C₆ alkyl, C₁-C₆ alkoxy, C₁-C₆ haloalkyl, and C₁-C₆ haloalkoxy. In some embodiments of Formula (IX), R⁶ is selected from the group consisting of halogen, hydroxyl, nitro, and cyano.

In some embodiments, the compound of Formula (IX) is selected from the compounds shown in TABLE 2, or a pharmaceutically acceptable salt thereof. In some embodiments, the compounds of Formula (IX) described herein are present in the form of a salt. In some embodiments, the salt is a pharmaceutically acceptable salt.

TABLE 2

| Cmpd No. | Structure |
|---|---|
| Resiquimod | 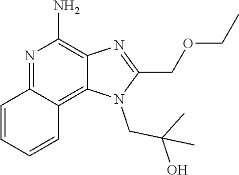 [SMILES: CCOCc1nc2c(N)nc3ccccc3c2n1CC(C)(C)O] |
| Gardiquimod | 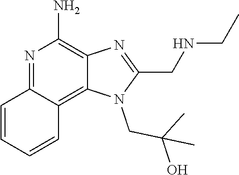 [SMILES: CCNCc1nc2c(N)nc3ccccc3c2n1CC(C)(C)O] |
| S5a | 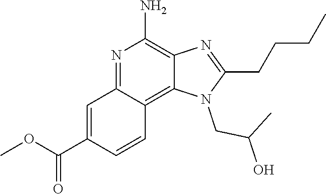 [SMILES: CCCCc1nc2c(N)nc3cc(C(=O)OC)ccc3c2n1CC(C)O] |
| S5b | 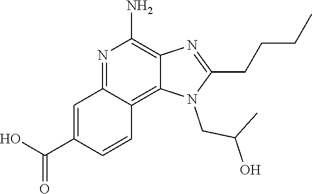 [SMILES: CCCCc1nc2c(N)nc3cc(C(=O)O)ccc3c2n1CC(C)O] |
| S8a | 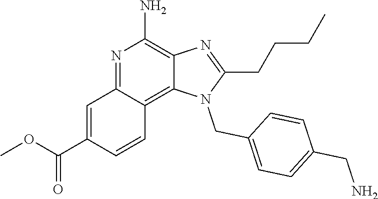 [SMILES: CCCCc1nc2c(N)nc3cc(C(=O)OC)ccc3c2n1Cc1ccc(CN)cc1] |
| S8b | 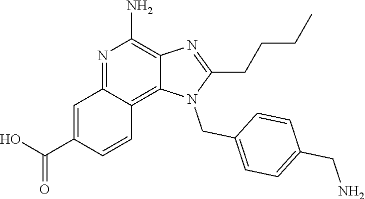 [SMILES: CCCCc1nc2c(N)nc3cc(C(=O)O)ccc3c2n1Cc1ccc(CN)cc1] |
| S11a | 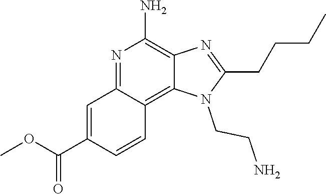 [SMILES: CCCCc1nc2c(N)nc3cc(C(=O)OC)ccc3c2n1CCN] |
| S11b | 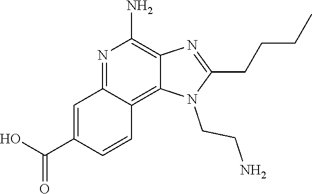 [SMILES: CCCCc1nc2c(N)nc3cc(C(=O)O)ccc3c2n1CCN] |
| S14a | 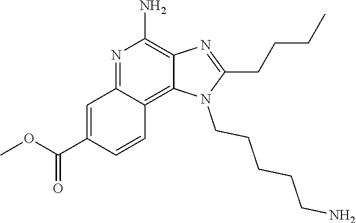 [SMILES: CCCCc1nc2c(N)nc3cc(C(=O)OC)ccc3c2n1CCCCCN] |
| S14b | 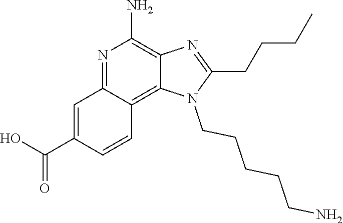 [SMILES: CCCCc1nc2c(N)nc3cc(C(=O)O)ccc3c2n1CCCCCN] |
| S18a | 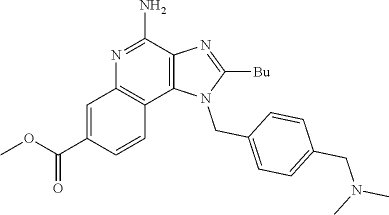 |
| S18b | 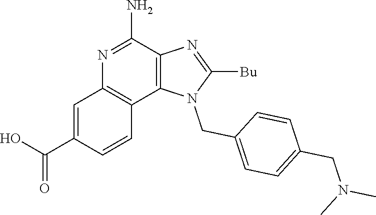 |
| S63a | 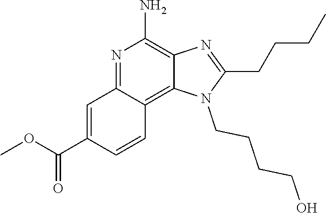 [SMILES: CCCCc1nc2c(N)nc3cc(C(=O)OC)ccc3c2n1CCCCO] |
| S63b | 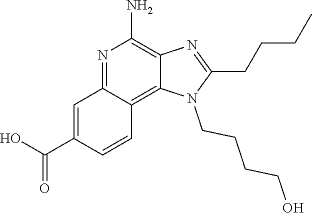 [SMILES: CCCCc1nc2c(N)nc3cc(C(=O)O)ccc3c2n1CCCCO] |
| S65a | 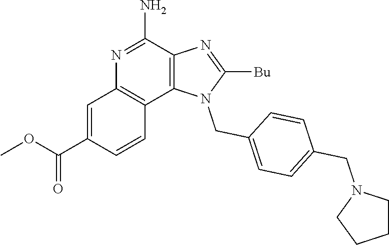 |
| S65b | 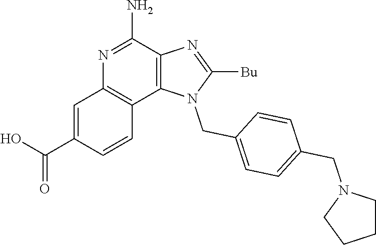 |
| S72a | 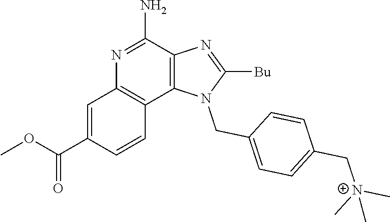 |
| S72b | 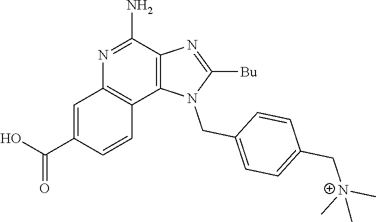 |
| S73a | 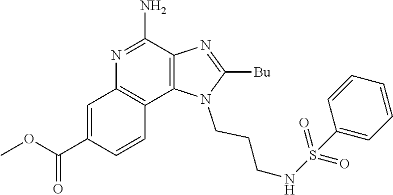 |
| S73b | 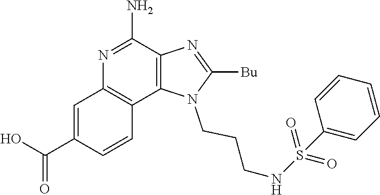 |
| S74a | 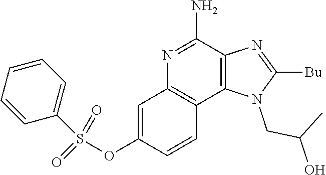 |
| S75a | 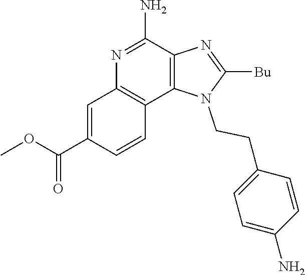 |
| S75b | 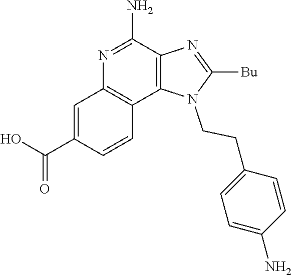 |
| S76a | 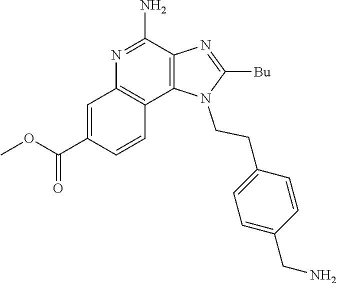 |
| S76b | 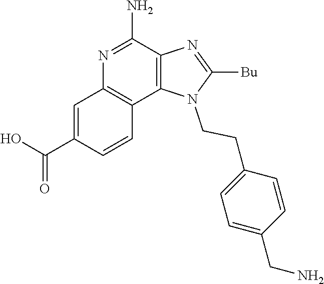 |
| S77a | 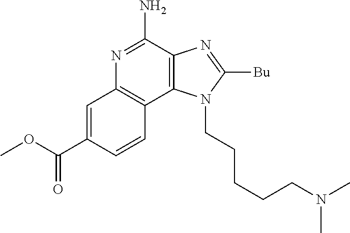 |
| S77b | 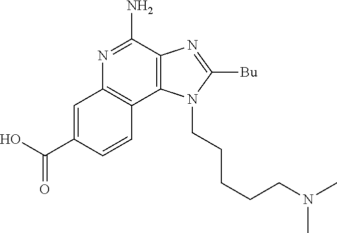 |
| S78a | 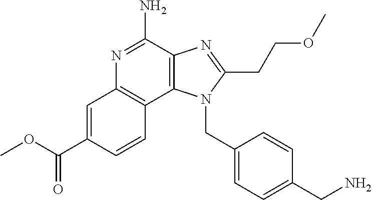 [SMILES: COCCc1nc2c(N)nc3cc(C(=O)OC)ccc3c2n1Cc1ccc(CN)cc1] |
| S78b | 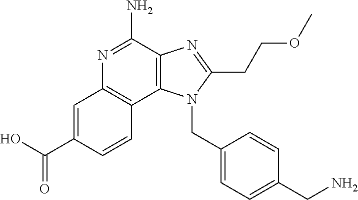 [SMILES: COCCc1nc2c(N)nc3cc(C(=O)O)ccc3c2n1Cc1ccc(CN)cc1] |
| S79a | 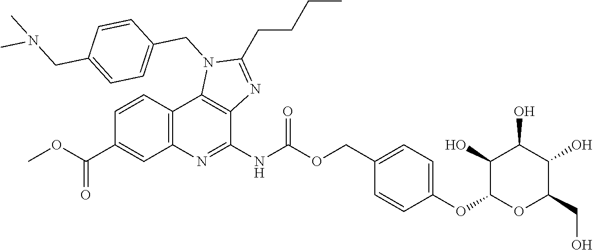 [SMILES: CCCCc1nc2c(NC(=O)OCc3ccc(O[C@H]4O[C@H](CO)[C@@H](O)[C@H](O)[C@@H]4O)cc3)nc3cc(C(=O)OC)ccc3c2n1Cc1ccc(CN(C)C)cc1] |
| S79b | 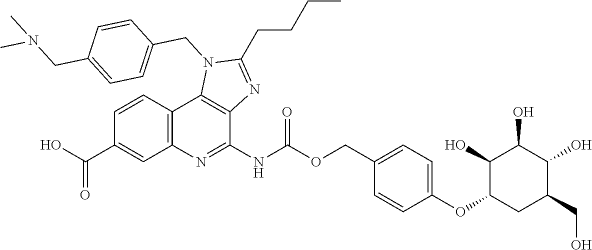 [SMILES: CCCCc1nc2c(NC(=O)OCc3ccc(O[C@H]4C[C@H](CO)[C@@H](O)[C@H](O)[C@@H]4O)cc3)nc3cc(C(=O)O)ccc3c2n1Cc1ccc(CN(C)C)cc1] |
| S80a | 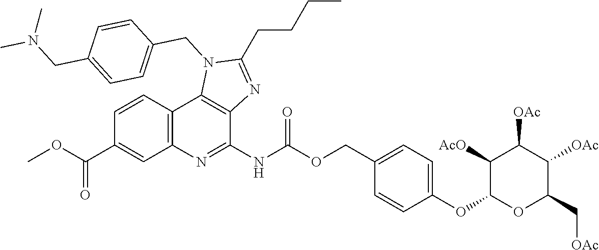 [SMILES: CCCCc1nc2c(NC(=O)OCc3ccc(O[C@H]4O[C@H](COC(C)=O)[C@@H](OC(C)=O)[C@H](OC(C)=O)[C@@H]4OC(C)=O)cc3)nc3cc(C(=O)OC)ccc3c2n1Cc1ccc(CN(C)C)cc1] |
| S80b | 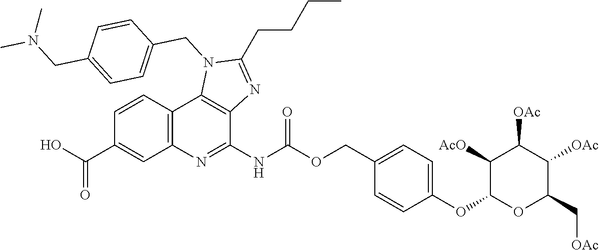 [SMILES: CCCCc1nc2c(NC(=O)OCc3ccc(O[C@H]4O[C@H](COC(C)=O)[C@@H](OC(C)=O)[C@H](OC(C)=O)[C@@H]4OC(C)=O)cc3)nc3cc(C(=O)O)ccc3c2n1Cc1ccc(CN(C)C)cc1] |
| S81a | 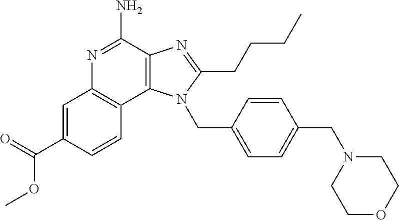 [SMILES: CCCCc1nc2c(N)nc3cc(C(=O)OC)ccc3c2n1Cc1ccc(CN2CCOCC2)cc1] |
| S81b | 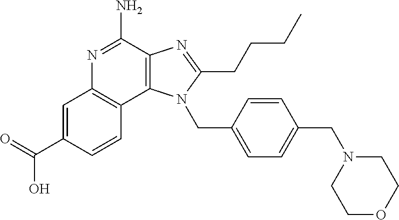 [SMILES: CCCCc1nc2c(N)nc3cc(C(=O)O)ccc3c2n1Cc1ccc(CN2CCOCC2)cc1] |
| S82a | 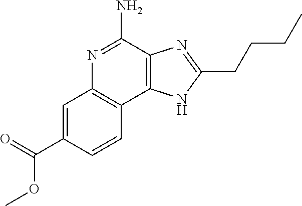 [SMILES: CCCCc1nc2c(N)nc3cc(C(=O)OC)ccc3c2[nH]1] |
| S82b | 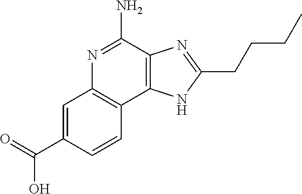 [SMILES: CCCCc1nc2c(N)nc3cc(C(=O)O)ccc3c2[nH]1] |
| S83a | 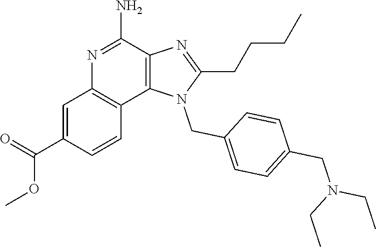 [SMILES: CCCCc1nc2c(N)nc3cc(C(=O)OC)ccc3c2n1Cc1ccc(CN(CC)CC)cc1] |
| S83b | 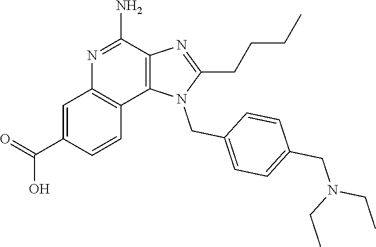 [SMILES: CCCCc1nc2c(N)nc3cc(C(=O)O)ccc3c2n1Cc1ccc(CN(CC)CC)cc1] |
| S84a | 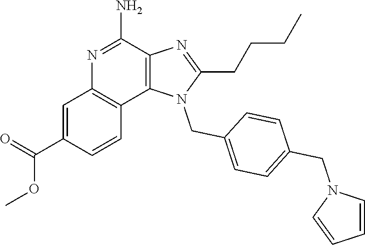 [SMILES: CCCCc1nc2c(N)nc3cc(C(=O)OC)ccc3c2n1Cc1ccc(Cn2cccc2)cc1] |
| S84b | 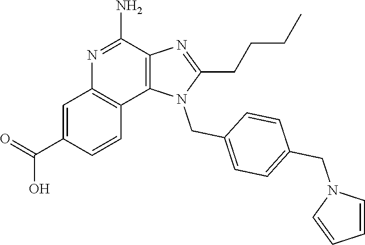 [SMILES: CCCCc1nc2c(N)nc3cc(C(=O)O)ccc3c2n1Cc1ccc(Cn2cccc2)cc1] |
| S85a | 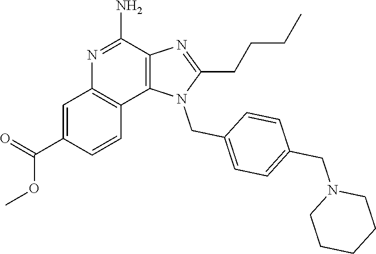 [SMILES: CCCCc1nc2c(N)nc3cc(C(=O)OC)ccc3c2n1Cc1ccc(CN2CCCCC2)cc1] |
| S85b | 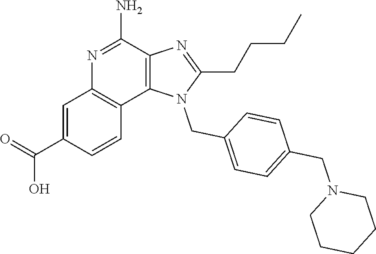 [SMILES: CCCCc1nc2c(N)nc3cc(C(=O)O)ccc3c2n1Cc1ccc(CN2CCCCC2)cc1] |

### Methods of Use of Compounds of Formula (IX)

Some embodiments provide a method of treating cancer in a subject in need thereof, comprising administering to the subject a therapeutically effective amount a compound of Formula (IX), or a pharmaceutically acceptable salt thereof.

Some embodiments provide a method of treating a viral or bacterial infection in a subject in need thereof, comprising administering to the subject a therapeutically effective amount of a compound of Formula (IX), or a pharmaceutically acceptable salt thereof.

Some embodiments provide a method of inducing an anti-viral or anti-bacterial immune response in a subject in need thereof, comprising administering to the subject a therapeutically effective amount a compound of Formula (IX), or a pharmaceutically acceptable salt thereof.

Some embodiments provide a method of inducing an anti-tumor immune response in a subject in need thereof, comprising administering to the subject a therapeutically effective amount a compound of Formula (IX), or a pharmaceutically acceptable salt thereof.

Some embodiments provide a method of treating cancer in a subject in need thereof, comprising administering to the subject a therapeutically effective amount a compound of Formula (IX), or a pharmaceutically acceptable salt thereof, in combination with another anticancer therapy (e.g., surgery and radiation therapy) and/or anticancer agent (e.g., an immunotherapy such as nivolumab or pembrolizumab). Compounds of Formula (IX) can be administered to the subject before, during, or after administration of the anticancer therapy and/or anticancer agent. In some embodiments, the compounds of Formula (IX) described herein can be administered to the subject following treatment with radiation and/or after surgery.

Some embodiments provide a method for delaying or preventing acquired resistance to an anticancer agent, comprising administering to the subject a therapeutically effective amount a compound of Formula (IX), or a pharmaceutically acceptable salt thereof, to a patient at risk for developing or having acquired resistance to an anticancer agent. In some embodiments, the patient is administered a dose of the anticancer agent (e.g., at substantially the same time as a dose of the compound of Formula (IX), or a pharmaceutically acceptable salt thereof is administered to the patient).

Some embodiments provide a method of delaying and/or preventing development of cancer resistant to an anticancer agent in a subject, comprising administering to the subject a therapeutically effective amount a compound of Formula (IX), or a pharmaceutically acceptable salt thereof, before, during, or after administration of a therapeutically effective amount of the anticancer agent.

Compounds of Formula (IX) are useful for inhibiting the multiplication of a cancer cell, causing apoptosis in a cancer cell, for increasing phagocytosis of a cancer cell, and/or for treating cancer in a subject in need thereof. In some embodiments, the cancer is as described herein. In some embodiments, the subject has previously undergone treatment for the cancer. In some embodiments, the prior treatment is surgery, radiation therapy, administration of one or more anticancer agents, or a combination of any of the foregoing. In some embodiments, the subject has discontinued a prior therapy, for example, due to unacceptable or unbearable side effects, wherein the prior therapy was too toxic, or wherein the subject developed resistance to the prior therapy.

Some embodiments provide a method for delaying or preventing a disease or disorder, comprising administering to the subject a therapeutically effective amount of a compound of Formula (IX), or a pharmaceutically acceptable salt thereof, and a vaccine against the disease or disorder, to a patient at risk for developing the disease or disorder. In some embodiments, the disease or disorder is cancer, as described herein. In some embodiments, the disease or disorder is a viral pathogen. In some embodiments, the vaccine is administered subcutaneously. In some embodiments, the vaccine is administered intramuscularly. In some embodiments, the compound of Formula (IX), or a pharmaceutically acceptable salt thereof, and the vaccine are administered via the same route (for example, the compound of Formula (IX), or a pharmaceutically acceptable salt thereof, and the vaccine are both administered subcutaneously). In some embodiments, the compound of Formula (IX), or a pharmaceutically acceptable salt thereof, and the vaccine are administered via different routes. In some embodiments, the vaccine and the compound of Formula (IX), or a pharmaceutically acceptable salt thereof, are provided in a single formulation. In some embodiments, the vaccine and the compound of Formula (IX), or a pharmaceutically acceptable salt thereof, are provided in separate formulations.

In some embodiments, the compounds of Formula (IX) described herein are present in the form of a salt. In some embodiments, the salt is a pharmaceutically acceptable salt.

In some embodiments, the compounds of Formula (IX) and ADCs of Formulae (A), (I)-(VIII), and (XI) are provided as prodrugs. In some such embodiments, the prodrug comprises a functional group configured to hydrolytically cleave under specific physiological conditions (e.g., the low pH of a tumor microenvironment) or in the presence of hydrolytic enzymes (e.g., in the presence of human hydrolases or proteases associated with a target tissue or cell). In some embodiments of Formulae (A), (I)-(VIII), and (XI), R¹, R⁴, or R¹ and R⁴ comprise a hydrolysable group. The hydrolysable group can be configured for cleavage within or in proximity to a target tissue or cell. Prior to cleavage, the hydrolysable group can prevent the drug unit from binding to its target (e.g., TLR7/8), thereby diminishing off-target activity and enhancing specificity for the target.

### Compositions and Methods of Administration of Compounds of Formula (IX)

Some embodiments provide a composition comprising a compound of Formula (IX), or a pharmaceutically acceptable salt thereof, and one or more excipients, as described herein. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral.

Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. In making the compositions provided herein, the active ingredient is typically mixed with an excipient, diluted by an excipient or enclosed within such an excipient in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Suitable excipients are known in the art. Descriptions of some of these excipients can be found in The Handbook of Pharmaceutical Excipients, published by the American Pharmaceutical Association and the Pharmaceutical Society of Great Britain.

The compositions comprising a compound of Formula (IX), or a pharmaceutically acceptable salt thereof, can be formulated in a unit dosage form, each dosage containing from about 5 to about 1,000 mg (1 g), more usually about 100 mg to about 500 mg, of the active ingredient. The term “unit dosage form” refers to physically discrete units suitable as unitary dosages for human subjects and other subjects, each unit containing a predetermined quantity of active material (i.e., a compound of Formula (IX), or a pharmaceutically acceptable salt thereof) calculated to produce the desired therapeutic effect, in association with a suitable pharmaceutical excipient.

An effective amount of the active material (i.e., a compound of Formula (IX), or a pharmaceutically acceptable salt of any of the foregoing) is ordinarily supplied at a dosage level of from about 0.01 mg/kg to about 1000 mg/kg of body weight per day, or any range therein. Preferably, the range is from about 0.05 to about 500 mg/kg of body weight per day, or any range therein. More preferably, from about 0.1 to about 250 mg/kg of body weight per day, or any range therein. More preferably, from about 0.1 to about 100 mg/kg of body weight per day, or any range therein. In an example, the range can be from about 0.1 to about 50.0 mg/kg of body weight per day, or any amount or range therein. In another example, the range can be from about 0.01 to about 15.0 mg/kg of body weight per day, or any range therein. In yet another example, the range can be from about 0.05 to about 7.5 mg/kg of body weight per day, or any amount to range therein. In yet another example, the range can be from about 0.1 to about 5.0 mg/kg of body weight per day, or any amount to range therein. Pharmaceutical compositions comprising a compound of Formula (IX), or a pharmaceutically acceptable salt of any of the foregoing, can be administered on a regimen of 1 to 4 times per day or in a single daily dose.

### Drug-Linker Intermediates (L¹-D)

Some embodiments provide a compound having the formula L¹-D, or a pharmaceutically acceptable salt thereof, wherein:
L¹ is a linker intermediate; and
D has the structure of Formula (X):

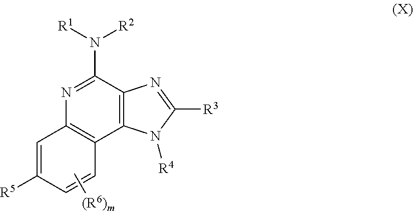

or a pharmaceutically acceptable salt thereof;
   wherein:
   L¹ is a linker intermediate;
   R¹ is (a) the point of covalent attachment to L¹; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
   R² is (a) the point of covalent attachment to L¹; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
   R¹ and R², taken together with the nitrogen atom to which they are attached form a 3-6 membered heterocyclyl optionally substituted with 1-3 independently selected C₁-C₆ alkyl;
   R³ is (a) the point of covalent attachment to L¹; or (b) selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
   R⁴ is (a) the point of covalent attachment to L¹; (b) —OR^{C}; or (c) C₁-C₆ alkyl optionally substituted with:
   (i) 1-3 independently selected halogen;
   (ii) —OR^{C};
   (iii) —SR^{C};
   (iv) —NH—S(O₂)R^{C};
   (v) —OC(═O)R^{C};
   (vi) —CO₂H;
   (vii) C₁-C₆ alkoxycarbonyl;
   (viii) —C(═O)NR^{D}R^{E};
   (ix) —NR^{D}R^{E};
   (x) —[N(C₁-C₆ alkyl)R^{D}R^{E}]⁺;
   (xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
   (xii) phenyl substituted with halogen, hydroxyl, C₁-C₆ alkoxy, —C(═O)NR^{D}R^{E} or —CO₂H;
   (xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
   (xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H;
   R⁵ is (a) the point of covalent attachment to L¹; or (b) selected from the group consisting of hydrogen, —C(═O)OR^{F}, —C(═O)NR^{G}R^{H}, —S(O₂)NR^{G}R^{H}, —N(R′)—C(═O)R^{J} and —N(R¹)—S(O₂)R^{K};
   each R⁶ is (a) the point of covalent attachment to L¹; or (b) independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R⁶ is the point of covalent attachment to L¹;
   wherein when R⁴ is (c), the C₁-C₆ alkyl, or a substituent thereof, may be further substituted with the point of covalent attachment to L¹;
   subscript m is 0, 1, 2, or 3;
   each R^{A} and R^{B} is (a) the point of covalent attachment to L¹, (b) independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl; wherein only one of R^{A} and R^{B} is the point of covalent attachment to L¹;
   R^{C} is (a) the point of covalent attachment to L¹; or (b) selected from the group consisting of hydrogen, phenyl, and C₁-C₁₀ alkyl optionally substituted with phenyl or 1-3 independently selected halogen;
   each R^{D}, R^{E}, R^{G}, and R^{H} are (a) the point of covalent attachment to L¹; or (b) independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl; wherein only one of R^{D}, R^{E}, R^{G}, and R^{H} is the point of covalent attachment to L¹;
   R^{F} is (a) the point of covalent attachment to L¹; or (b) selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
   each R^{I}, R^{J}, and R^{K} is (a) the point of covalent attachment to L¹; or (b) independently selected from the group consisting of hydrogen and C₁-C₆ alkyl; wherein only one of R^{I}, R^{J}, and R^{K} is the point of covalent attachment to L¹;
   wherein only one of R¹, R², R³, R⁴, R⁵, R^{A}, R⁶, R^{B}, R^{C}, R^{D}, R^{E}, R^{F}, R^{G}, R^{H}, R^{I}, R^{J} and R^{K} is the point of covalent attachment to L¹; and
wherein Formula (X) has only one point of covalent attachment to L¹.

Some embodiments provide a compound having the formula L¹-D, or a pharmaceutically acceptable salt thereof, wherein:
L¹ is a linker intermediate; and
D has the structure of Formula (X):

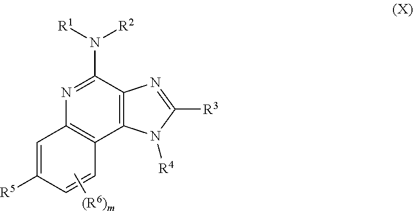

or a pharmaceutically acceptable salt thereof;
wherein:
L¹ is a linker intermediate;
R¹ is (a) the point of covalent attachment to L¹; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is (a) the point of covalent attachment to L¹; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is (a) the point of covalent attachment to L¹; or (b) selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) the point of covalent attachment to L¹; (b) —OR⁵; (c) —S(═O)₂R^{C}; (d) —C(═O)NR^{D}R^{E}; (e) —C(═O)OR^{C}; (f) —C(═O)SR^{C}; (g) —C(═S)R^{C}; (h) —PO₃R^{C}; or (j) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H;
R⁵ is (a) the point of covalent attachment to L¹; or (b) selected from the group consisting of hydrogen, —C(═O)OR^{F}, —NO₂, —CN, —CF₃, —C(═O)NR^{G}R^{H}, —S(O₂)NR^{G}R^{H}, —N(R′)—C(═O)R^{J}, —N(R¹)—S(O₂)R^{K}, and SO₃R^{K};
each R⁶ is (a) the point of covalent attachment to L¹; or (b) independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R⁶ is the point of covalent attachment to L¹;
wherein when R⁴ is (c), the C₁-C₆ alkyl, or a substituent thereof, may be further substituted with the point of covalent attachment to L¹;
subscript m is 0, 1, 2, or 3;
each R^{A} and R^{B} is (a) the point of covalent attachment to L¹, (b) independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl; wherein only one of R^{A} and R^{B} is the point of covalent attachment to L¹;
R^{C} is (a) the point of covalent attachment to L¹; or (b) selected from the group consisting of hydrogen, phenyl, and C₁-C₁₀ alkyl optionally substituted with phenyl or 1-3 independently selected halogen;
each R^{D}, R^{E}, R^{G}, and R^{H} are (a) the point of covalent attachment to L¹; or (b) independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl; wherein only one of R^{D}, R^{E}, R^{G}, and R^{H} is the point of covalent attachment to L¹;
R^{F} is (a) the point of covalent attachment to L¹; or (b) selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl; each R^{I}, R^{J}, and R^{K} is (a) the point of covalent attachment to L¹; or (b) independently selected from the group consisting of hydrogen and C₁-C₆ alkyl; wherein only one of R^{I}, R^{J}, and R^{K} is the point of covalent attachment to L¹;
each instance of R¹ and R⁴ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide;
wherein only one of R¹, R², R³, R⁴, R⁵, R^{A}, R⁶, R^{B}, R^{C}, R^{D}, R^{E}, R^{F}, R^{G}, R^{H}, R^{I}, R^{J}, and R^{K} is the point of covalent attachment to L¹; and wherein Formula (X) has only one point of covalent attachment to L¹.

In some embodiments, the drug-linker intermediate compounds of Formula (X) is used to prepare the ADCs described herein.

Each of R¹, R², R³, R⁴, R⁵, R^{A}, R⁶, R^{B}, R^{C}, R^{D}, R^{E}, R^{F}, R^{G}, R^{H}, R^{I}, R^{J}, and R^{K}, in compounds of Formula (X) are as described herein with respect to the compounds of Formulae (I)-(IX), with the exception that covalent attachment to L in those compounds corresponds to covalent attachment to L¹ in compounds of Formula (X).

In some embodiments, the linker intermediate (L¹) has the formula M¹-(A)ₐ-(W)_{w}—(Y)_{y}—(X)ₓ—; wherein A, W, Y, X, are as defined for the linker (L); and wherein subscripts a, w, y, and x are each independently 0 or 1; wherein the sum of subscripts a, w, y, and x is greater than or equal to 1.

In some embodiments, M¹ comprises a functional group that will react with an antibody to form a covalent bond (the Ab-M bond). In some embodiments, M¹ is selected from the group consisting of maleimido, azido, C₁-C₆ alkynyl, cycloalkynyl optionally substituted with 1 or 2 fluoro (e.g., cyclooctynyl or DIFO), sulfhydryl, succinimidyl esters (e.g., N-hydroxysuccinimidyl (NHS) or sulfo-NHS esters), 4-nitrophenyl esters, pentafluorophenyl esters, tetrafluorophenyl esters, anhydrides, acid chlorides, sulfonyl chlorides, isocyanates, isothiocyanates, alpha-haloketones, alpha-O-sulfonate (e.g., mesyl or tosyl) ketones, alkyl hydrazines, hydrazides, hydroxylamines, and iodoketones. In some embodiments, M¹ is selected from the group consisting of maleimido, azido, C₁-C₆ alkynyl, cycloalkynyl optionally substituted with 1 or 2 fluoro (e.g., cyclooctynyl or DIFO), sulfhydryl, succinimidyl esters (e.g., N-hydroxysuccinimidyl (NHS) or sulfo-NHS esters), 4-nitrophenyl esters, pentafluorophenyl esters, tetrafluorophenyl esters, anhydrides, acid chlorides, sulfonyl chlorides, isocyanates, isothiocyanates, alpha-haloketones, alpha-O-sulfonate (e.g., mesyl or tosyl) ketones, alkyl hydrazines, hydrazides, and hydroxylamines. In some embodiments, M¹ is selected from the group consisting of maleimido, azido, C₁-C₆ alkynyl, cycloalkynyl optionally substituted with 1 or 2 fluoro (e.g., cyclooctynyl or DIFO), sulfhydryl, succinimidyl esters. Additional examples of functional groups that will react with an antibody to form an a covalent bond are described in PCT Publication No. WO2016/040684, which is hereby incorporated by reference in its entirety.

In some embodiments, M¹ is

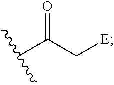

wherein [symbol] indicates the covalent bond to the remainder of L¹ (e.g., A, W, Y, or X); and wherein E is halogen or —O(SO₂)-E′; wherein E′ is alkyl, aryl, or aryl substituted with alkyl, as described herein (e.g., tosyl or mesyl).

In some embodiments, M¹ is

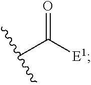

wherein [symbol] indicates the covalent bond to the remainder of L¹(e.g., A, W, Y, or X); wherein E¹ is halogen, —O—N-succinimide, —O-(aryl), wherein the aryl is substituted with nitro, 4 or 5 fluoro, —OC(═O)—O(C₁-C₆ alkyl), or —OC(═O)—O(aryl).

In some embodiments, M¹ is

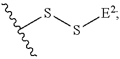

wherein [symbol] indicates the covalent bond to the remainder of L¹(e.g., A, W, Y, or X); wherein E² is aryl or heteroaryl, as described herein.

In some embodiments, M¹ is

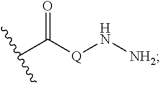

wherein [symbol] indicates the covalent bond to the remainder of L¹(e.g., A, W, Y, or X); and wherein Q is a bond or C₁-C₁₀ alkylene.

In some embodiments, M¹ is

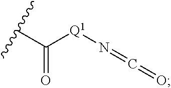

wherein [symbol] indicates the covalent bond to the remainder of L¹ (e.g., A, W, Y, or X); and wherein Q¹ is C₁-C₁₀ alkylene.

In some embodiments, M¹ is

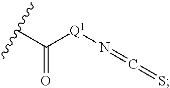

wherein [symbol] indicates the covalent bond to the remainder of L¹ (e.g., A, W, Y, or X); and wherein Q¹ is C₁-C₁₀ alkylene.

In some embodiments, M¹ is

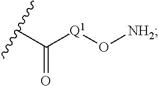

wherein [symbol] indicates the covalent bond to the remainder of L¹ (e.g., A, W, Y, or X); and wherein Q¹ is C₁-C₁₀ alkylene.

In some embodiments, M¹ is

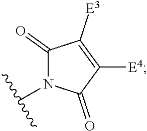

wherein [symbol] indicates the covalent bond to the remainder of L¹ (e.g., A, W, Y, or X); and wherein E³ and E⁴ are independently selected from the group consisting of hydrogen, halogen, C₁-C₆ alkyl, and —O(SO₂)-E⁵; wherein E⁵ is alkyl, aryl, or aryl substituted with alkyl, as described herein (e.g., tosyl or mesyl).

In some embodiments, M¹ is

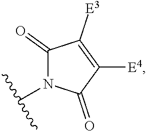

and E³ and E⁴ are both hydrogen. As such, in some embodiments M¹ is

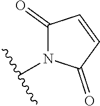 [SMILES: *N1C(=O)C=CC1=O]

(maleimido). In some embodiments, M¹ is maleimido.

In some embodiments, L¹-D has the structure:

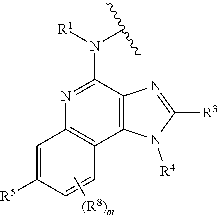

or a pharmaceutically acceptable salt thereof; wherein [symbol] represents covalent attachment to L¹.

In some embodiments, L¹-D has the structure:

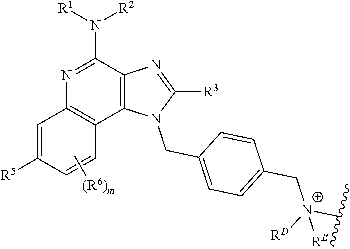

or a pharmaceutically acceptable salt thereof; wherein [symbol] represents covalent attachment to L¹.

In some embodiments, R⁵ is —C(═O)OR^{F}. In some embodiments, R^{F} is C₁-C₆ alkyl. In some embodiments, R^{F} is methyl. In some embodiments, R^{F} is hydrogen.

Some embodiments of Formula (X), D is in prodrug form. In some cases, R¹ of the compound of Formula (X) in prodrug form is selected from the group consisting of C₁-C₆ alkoxycarbonyl, C₁-C₆ alkoxythiocarbonyl, C₁-C₆ carbamoyl, C₁-C₆ amidine, C₁-C₆ sulfone, and C₁-C₆ thione. In some cases, R¹ of the compound of Formula (X) in prodrug form is selected from the group consisting of C₁-C₆ alkoxycarbonyl, C₁-C₆ alkoxythiocarbonyl, and C₁-C₆ carbamoyl. In some cases, R¹ of the compound of Formula (X) in prodrug form is C₁-C₆ alkoxycarbonyl.

In some embodiments of Formula (X), R¹ is selected from the group consisting of hydrogen, C₁-C₆ alkyl, C₂-C₆ alkenyl, C₂-C₆ alkynyl, C₁-C₆ alkoxycarbonyl, C₁-C₆ alkoxythiocarbonyl, C₁-C₆ carbamoyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkoxycarbonyl, C₁-C₆ alkoxythiocarbonyl, C₁-C₆ carbamoyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}.

In some embodiments of Formula (X), R¹ is selected from the group consisting of hydrogen, C₁-C₆ alkyl, C₂-C₆ alkenyl, C₁-C₆ alkoxycarbonyl, C₁-C₆ carbamoyl, C₃-C₆ cycloalkyl, phenyl, and 5-10 membered heteroaryl; wherein each C₁-C₆ alkyl, C₂-C₆ alkenyl, C₁-C₆ alkoxycarbonyl, C₁-C₆ carbamoyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}. In some embodiments of Formula (X), R¹ is selected from the group consisting of C₁-C₆ alkoxycarbonyl and C₁-C₆ carbamoyl; wherein each C₁-C₆ alkoxycarbonyl and C₁-C₆ carbamoyl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}.

In some embodiments of Formula (X), R⁴ is —S(═O)₂R^{C}; —C(═O)NR^{D}R^{E}; —C(═O)OR^{C}; —C(═O)SR^{c}; or C₁-C₆ alkyl optionally substituted with a group selected from the group consisting of (i)-(xiv), as described herein. In some embodiments of Formula (X), R⁴ is —C(═O)NR^{D}R^{E}; —C(═O)OR^{C}; or C₁-C₆ alkyl optionally substituted with a group selected from the group consisting of (i)-(xiv), as described herein. In some embodiments of Formula (X), R⁴ is —C(═O)NR^{D}R^{E} or —C(═O)OR^{c}. In some embodiments of Formula (X), R⁴ is —C(═O)NR^{D}R^{E}.

In some embodiments of Formula (X), R⁵ is selected from the group consisting of —C(═O)OH, —NO₂, —CN, —CF₃, and —S(O₃)H. In some embodiments of Formula (X), R⁵ is selected from the group consisting of —C(═O)OH and —S(O₃)H. In some embodiments Formula (X), R⁵ is —C(═O)OH.

In some embodiments of Formula (X), R¹ is not substituted with a solubilizing group (S_{b}). In some embodiments of Formula (X), R⁴ is not substituted with a solubilizing group (S_{b}).

In some embodiments of Formula (X), R¹ and R⁴ are not substituted with solubilizing groups (S_{b}). In some embodiments of Formula (X), only one of R¹ and R⁴ is substituted with a solubilizing group (S_{b}). In some embodiments of Formula (X), R¹ is not substituted with a solubilizing group if R¹ is a point of covalent attachment to L. In some embodiments of Formula (X), R⁴ is not substituted with a solubilizing group (S_{b}) if R¹ is a point of covalent attachment to L.

In some embodiments, subscript m is 0. In some embodiments, R³ is C₁-C₆ alkyl. In some embodiments, R³ is n-butyl. In some embodiments, R¹ is hydrogen or C₁-C₆ alkyl. In some embodiments, R¹ is hydrogen or methyl. In some embodiments, R¹ is hydrogen. In some embodiments, R² is hydrogen or C₁-C₆ alkyl. In some embodiments, R² is hydrogen or methyl.

In some embodiments, R² is hydrogen. In some embodiments, R^{D} and R^{E} are independently C₁-C₆ alkyl. In some embodiments, R^{D} and R^{E} are both methyl. In some embodiments, RP and R^{E}, together with the nitrogen atom to which they are attached form a 3-6 membered heterocyclyl optionally substituted with 1-3 independently selected C₁-C₆ alkyl. In some embodiments, R^{D} and R^{E}, together with the nitrogen atom to which they are attached form an unsubstituted 3-6 membered heterocyclyl. In some embodiments, the compound of L¹-D is selected from the compounds shown in TABLE 3, or a pharmaceutically acceptable salt thereof.

TABLE 3

| Cmpd | |
|---|---|
| No. | Structure |
| 1 | 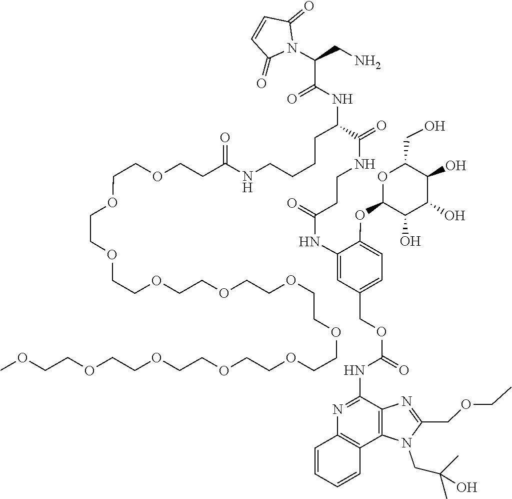 [SMILES: CCOCc1nc2c(NC(=O)OCc3ccc(O[C@H]4O[C@H](CO)[C@@H](O)[C@H](O)[C@@H]4O)c(NC(=O)CCNC(=O)[C@H](CCCCNC(=O)CCOCCOCCOCCOCCOCCOCCOCCOCCOCCOCCOCCOC)NC(=O)[C@H](CN)N4C(=O)C=CC4=O)c3)nc3ccccc3c2n1CC(C)(C)O] |
| 2 | 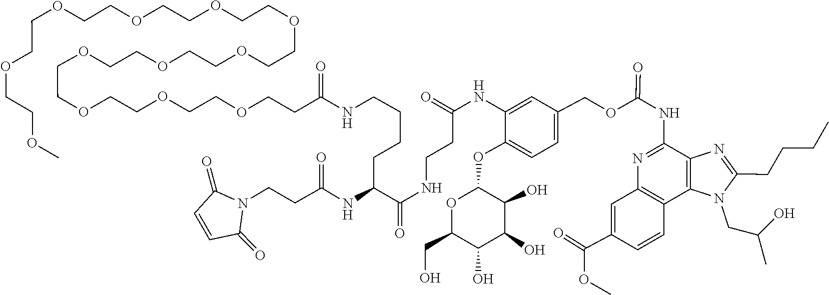 [SMILES: CCCCc1nc2c(NC(=O)OCc3ccc(O[C@H]4O[C@H](CO)[C@@H](O)[C@H](O)[C@@H]4O)c(NC(=O)CCNC(=O)[C@H](CCCCNC(=O)CCOCCOCCOCCOCCOCCOCCOCCOCCOCCOCCOCCOC)NC(=O)CCN4C(=O)C=CC4=O)c3)nc3cc(C(=O)OC)ccc3c2n1CC(C)O] |
| 3 | 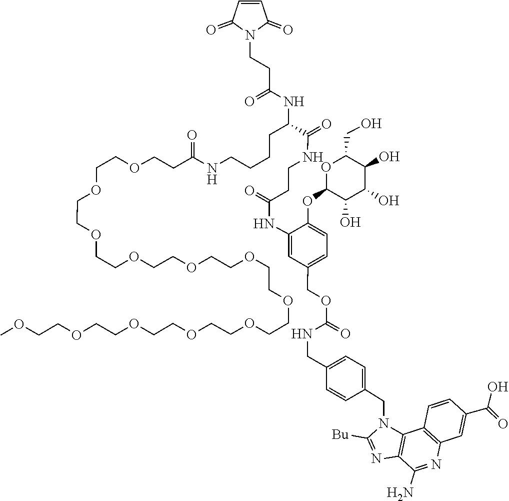 |
| 4 | 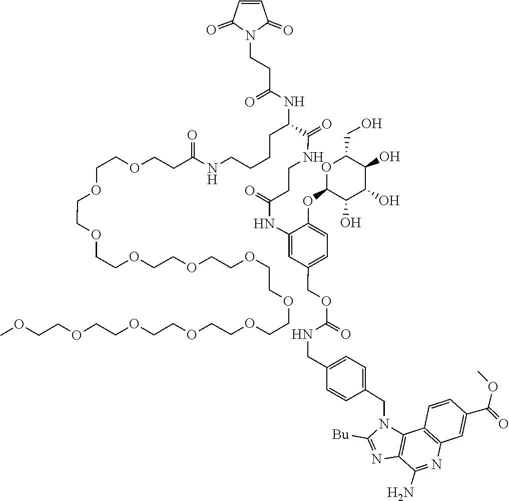 |
| 5 | 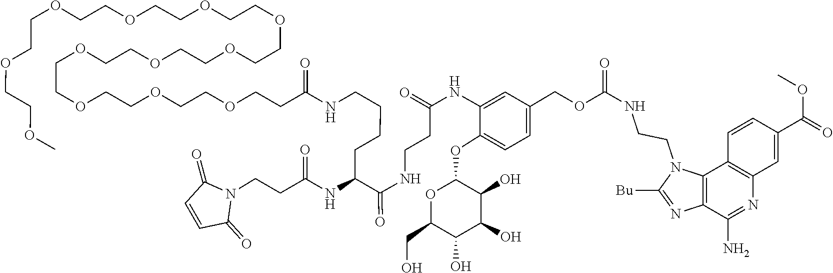 |
| 6 | 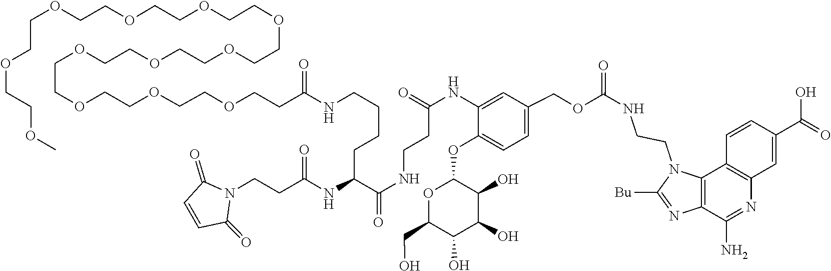 |
| 7 | 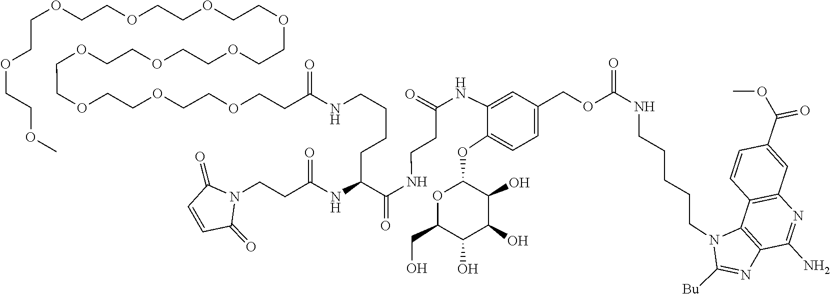 |
| 8 | 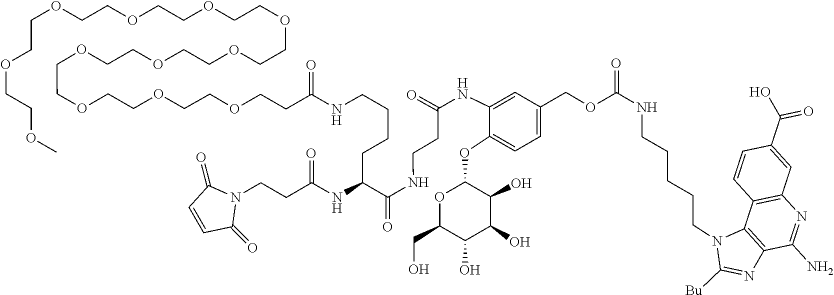 |
| 9 | 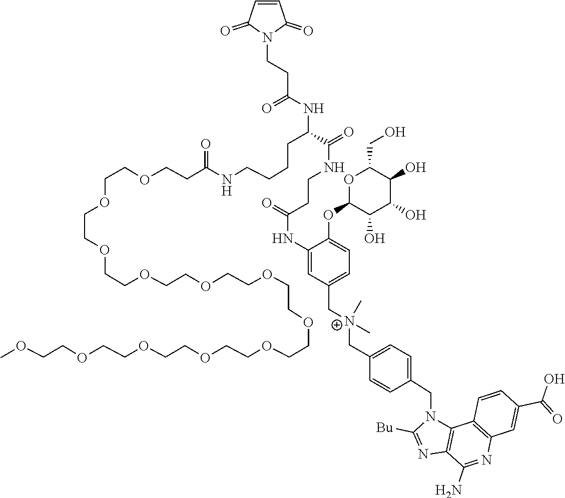 |
| 10 | 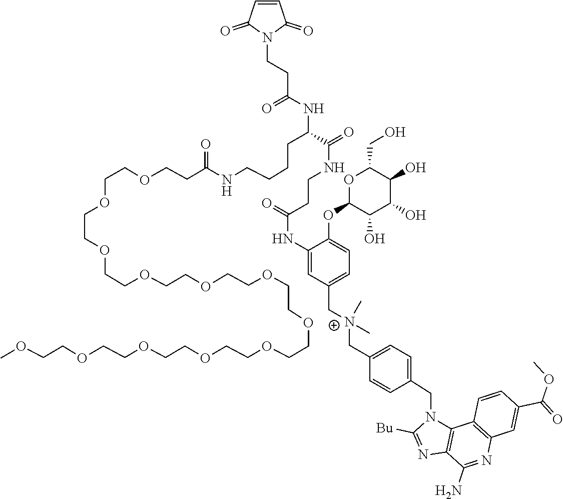 |
| 11 | 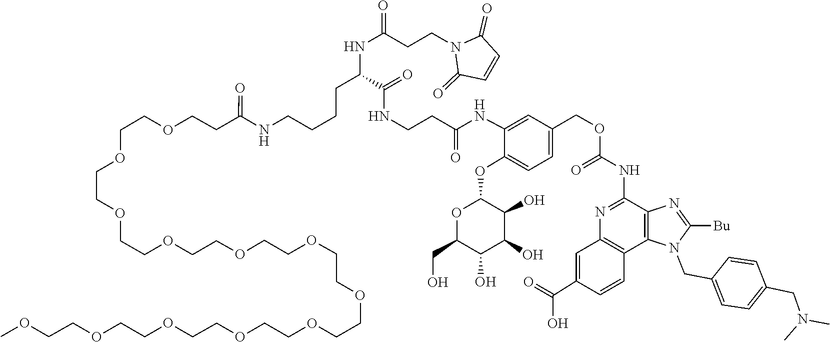 |
| 12 | 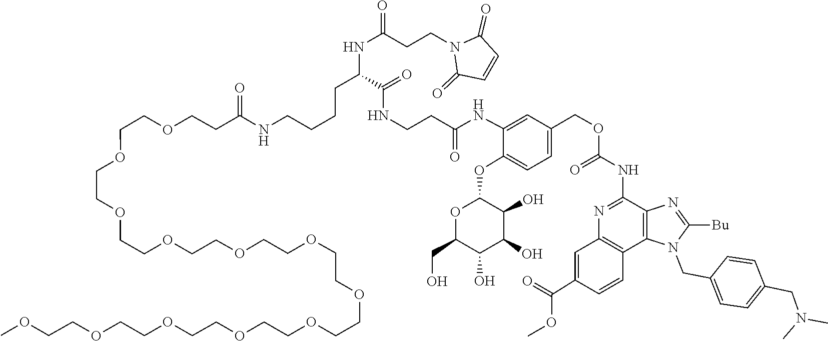 |
| 13 | 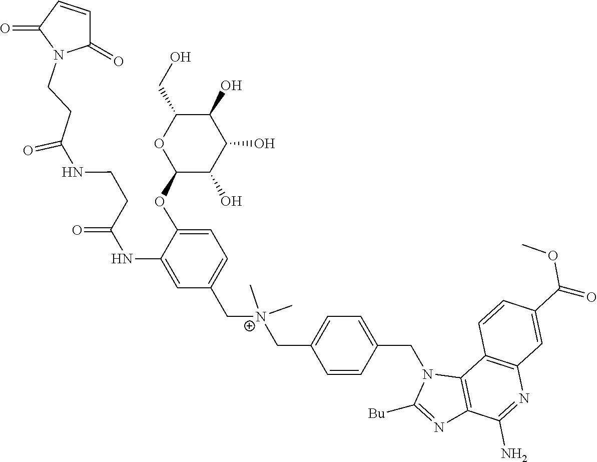 |
| 14 | 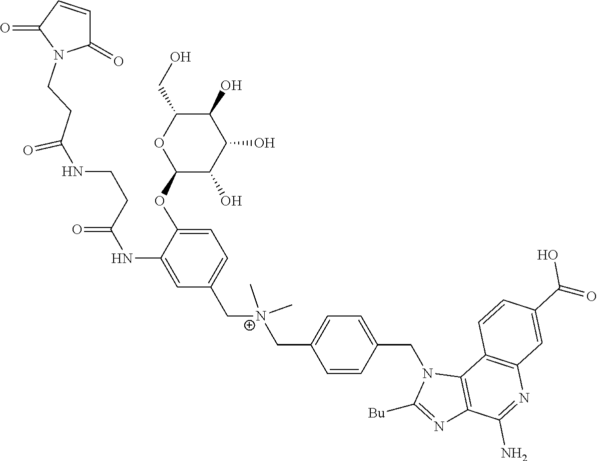 |
| 15 | 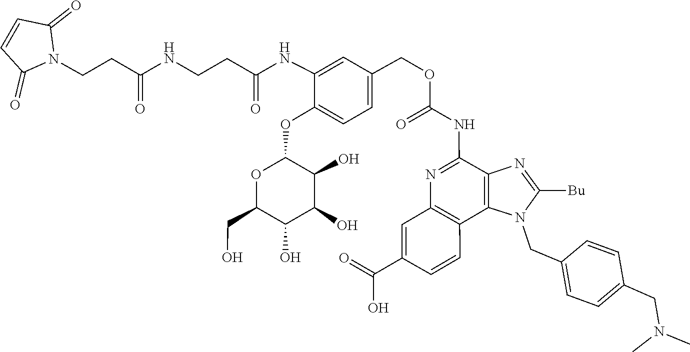 |
| 16 | 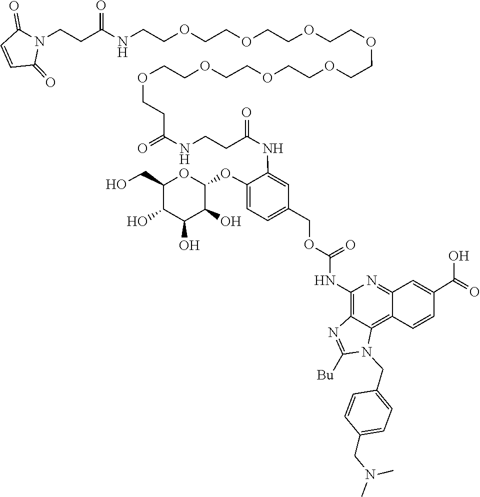 |
| 17 | 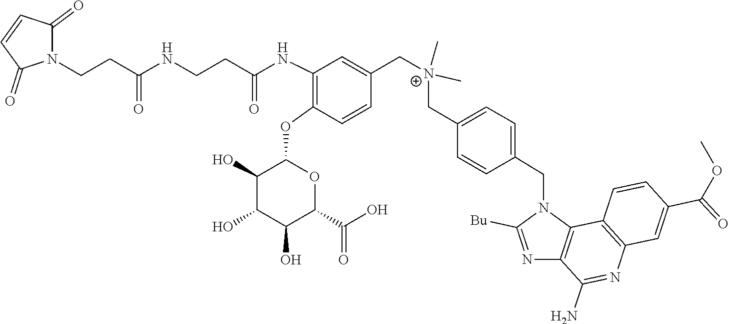 |
| 18 | 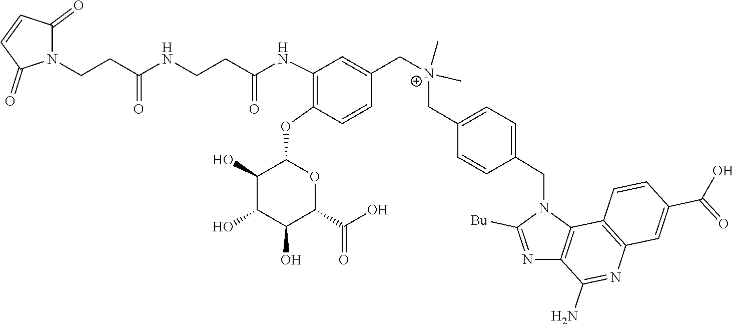 |
| 19 | 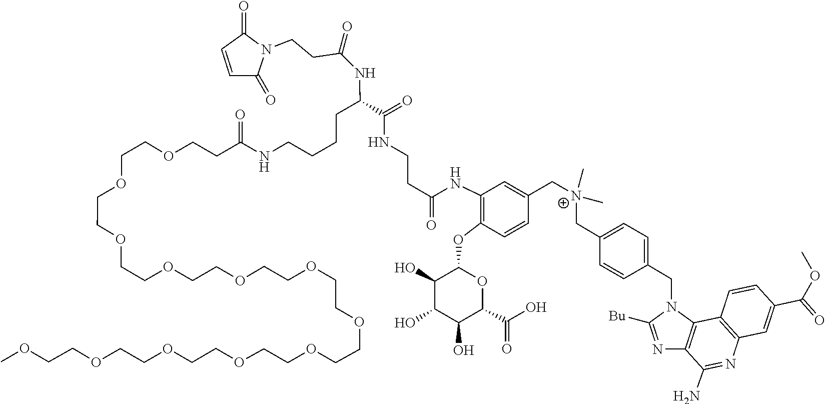 |
| 20 | 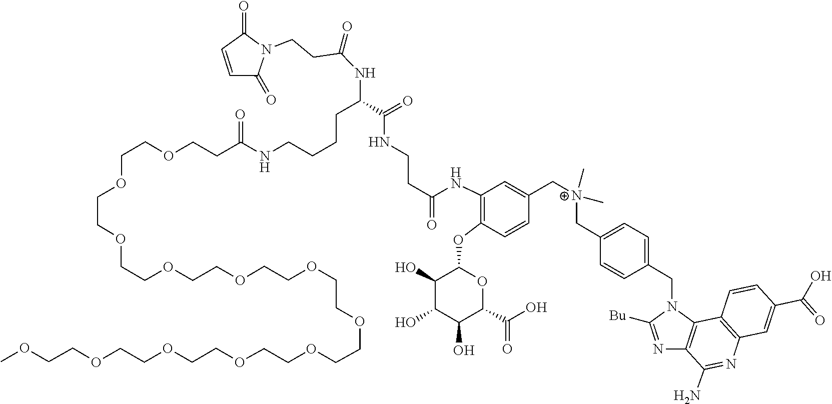 |
| 21 | 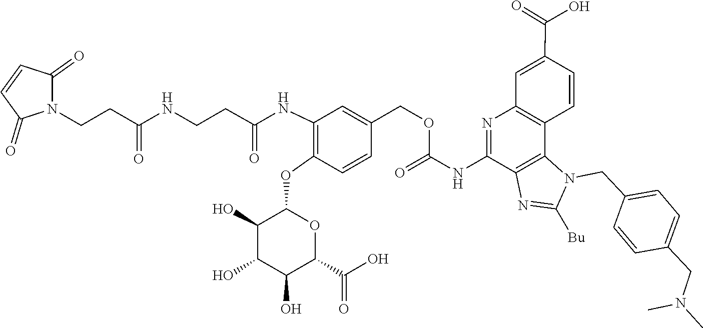 |
| 22 | 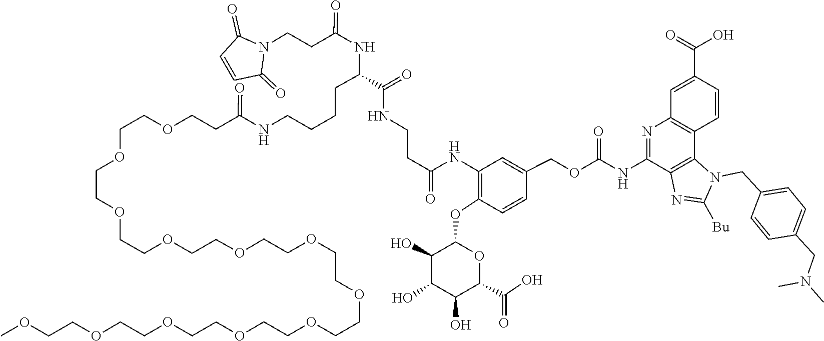 |
| 23 | 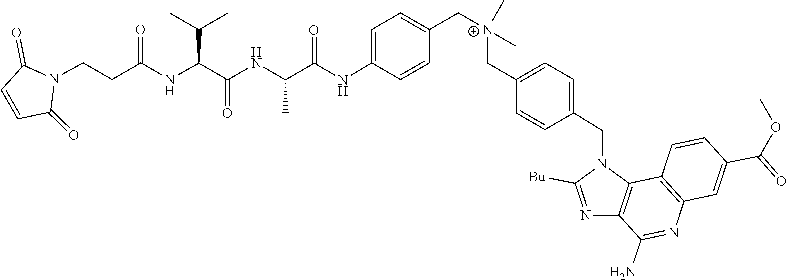 |
| 24 | 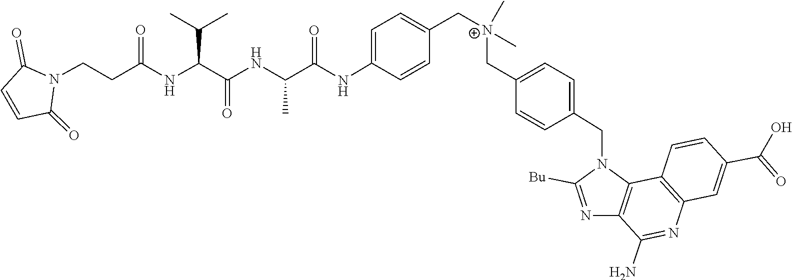 |
| 25 | 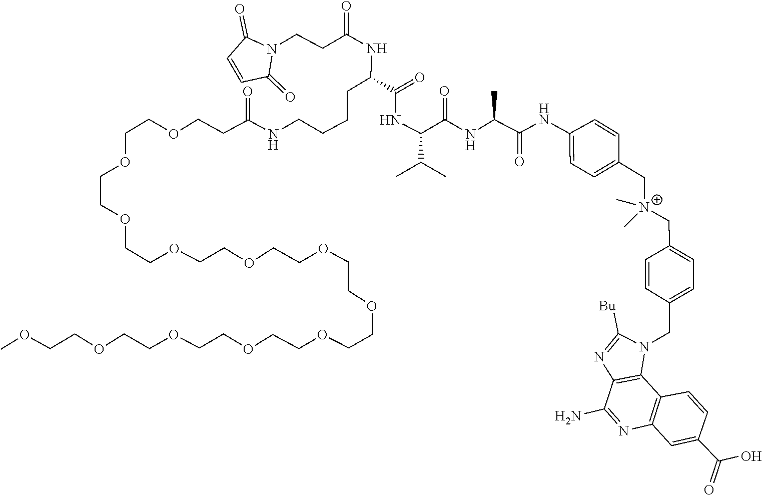 |
| 26 | 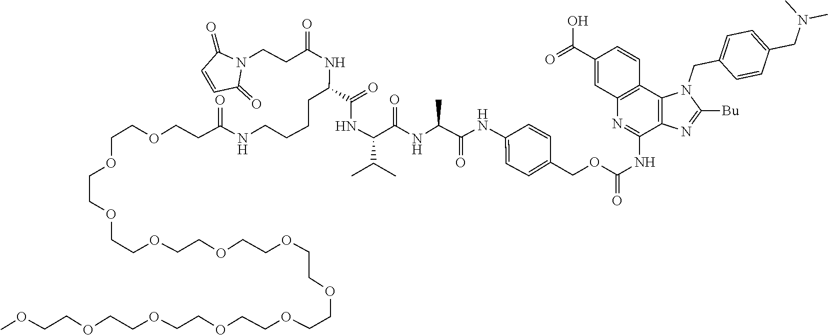 |
| 27 | 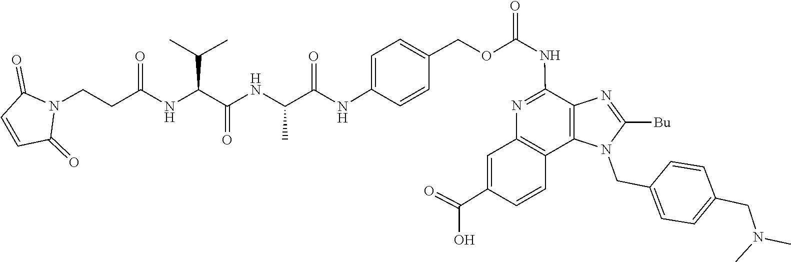 |
| 28 | 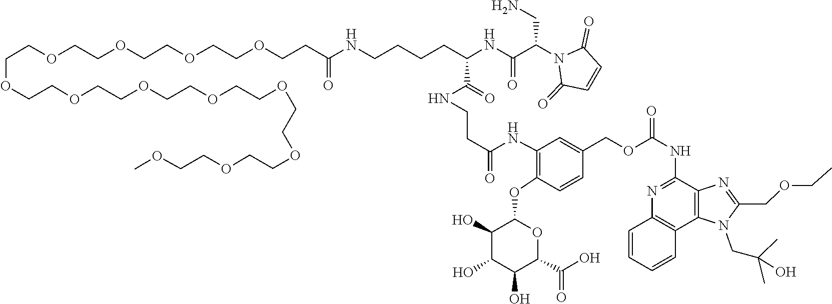 [SMILES: CCOCc1nc2c(NC(=O)OCc3ccc(O[C@@H]4O[C@H](C(=O)O)[C@@H](O)[C@H](O)[C@H]4O)c(NC(=O)CCNC(=O)[C@H](CCCCNC(=O)CCOCCOCCOCCOCCOCCOCCOCCOCCOCCOCCOCCOC)NC(=O)[C@H](CN)N4C(=O)C=CC4=O)c3)nc3ccccc3c2n1CC(C)(C)O] |
| 29 | 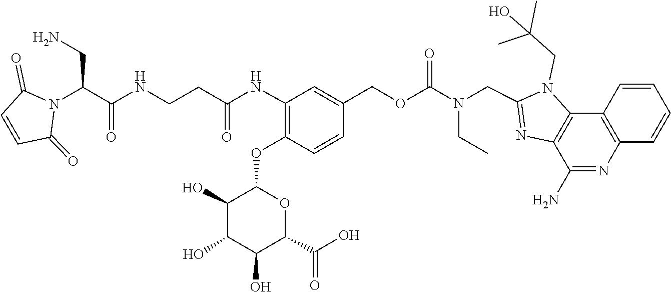 [SMILES: CCN(Cc1nc2c(N)nc3ccccc3c2n1CC(C)(C)O)C(=O)OCc1ccc(O[C@@H]2O[C@H](C(=O)O)[C@@H](O)[C@H](O)[C@H]2O)c(NC(=O)CCNC(=O)[C@H](CN)N2C(=O)C=CC2=O)c1] |
| 30 | 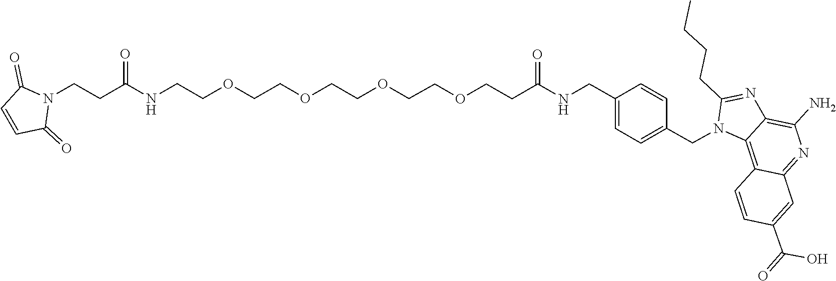 [SMILES: CCCCc1nc2c(N)nc3cc(C(=O)O)ccc3c2n1Cc1ccc(CNC(=O)CCOCCOCCOCCOCCNC(=O)CCN2C(=O)C=CC2=O)cc1] |
| 31 | 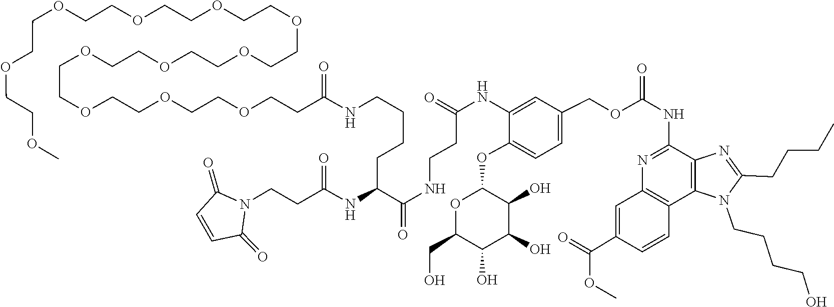 [SMILES: CCCCc1nc2c(NC(=O)OCc3ccc(O[C@H]4O[C@H](CO)[C@@H](O)[C@H](O)[C@@H]4O)c(NC(=O)CCNC(=O)[C@H](CCCCNC(=O)CCOCCOCCOCCOCCOCCOCCOCCOCCOCCOCCOCCOC)NC(=O)CCN4C(=O)C=CC4=O)c3)nc3cc(C(=O)OC)ccc3c2n1CCCCO] |
| 32 | 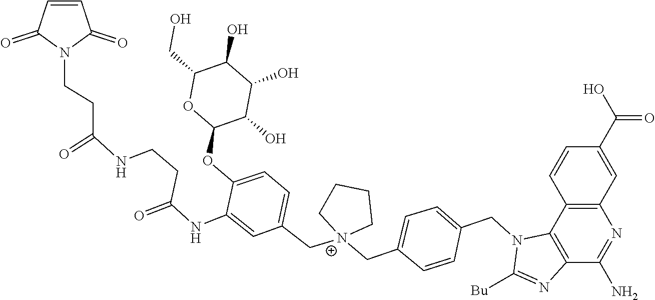 |
| 33 | 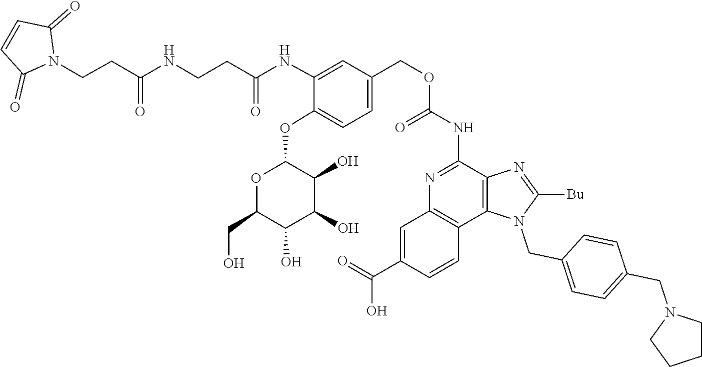 |
| 34 | 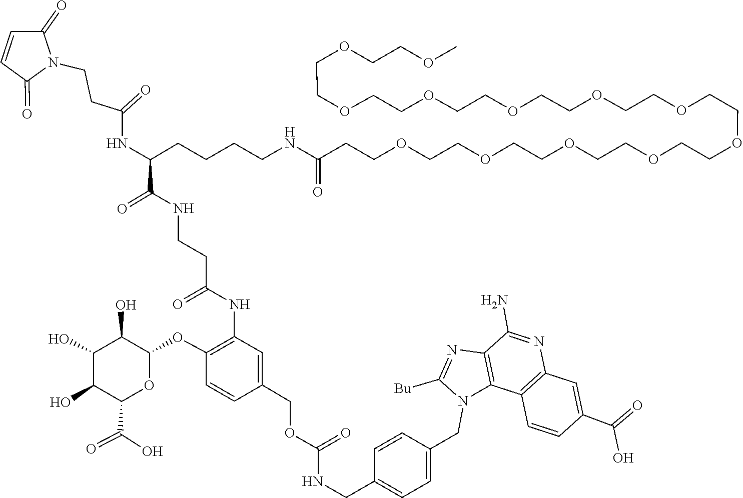 |
| 35 | 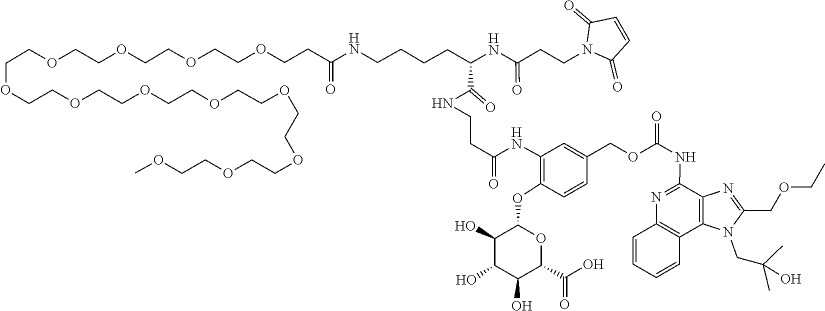 [SMILES: CCOCc1nc2c(NC(=O)OCc3ccc(O[C@@H]4O[C@H](C(=O)O)[C@@H](O)[C@H](O)[C@H]4O)c(NC(=O)CCNC(=O)[C@H](CCCCNC(=O)CCOCCOCCOCCOCCOCCOCCOCCOCCOCCOCCOCCOC)NC(=O)CCN4C(=O)C=CC4=O)c3)nc3ccccc3c2n1CC(C)(C)O] |
| 36 | 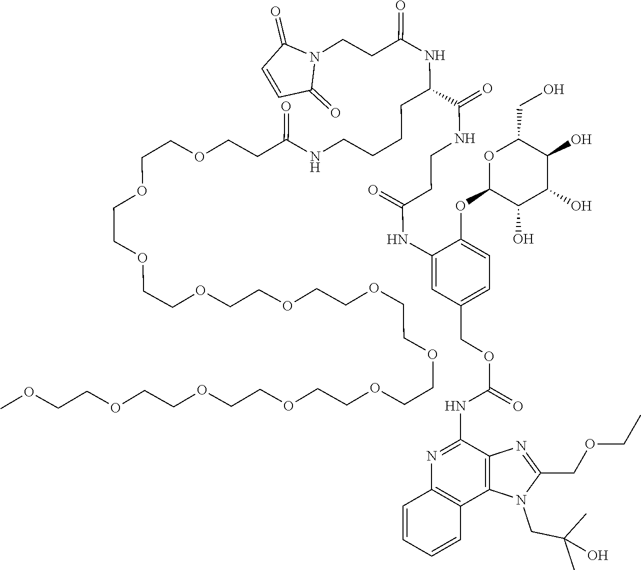 [SMILES: CCOCc1nc2c(NC(=O)OCc3ccc(O[C@H]4O[C@H](CO)[C@@H](O)[C@H](O)[C@@H]4O)c(NC(=O)CCNC(=O)[C@H](CCCCNC(=O)CCOCCOCCOCCOCCOCCOCCOCCOCCOCCOCCOCCOC)NC(=O)CCN4C(=O)C=CC4=O)c3)nc3ccccc3c2n1CC(C)(C)O] |
| 37 | 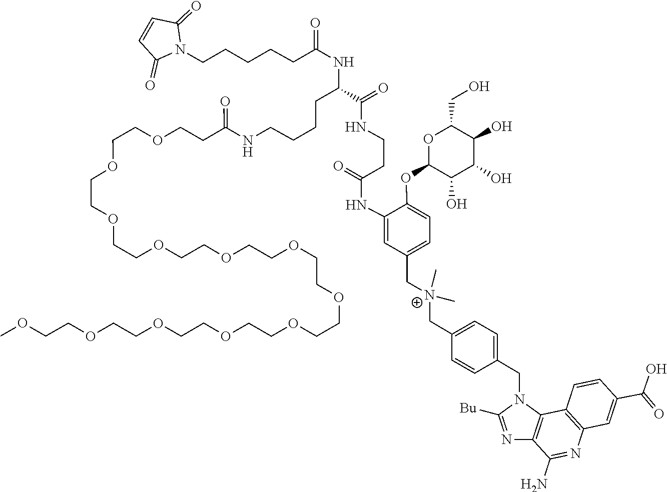 |
| 38 | 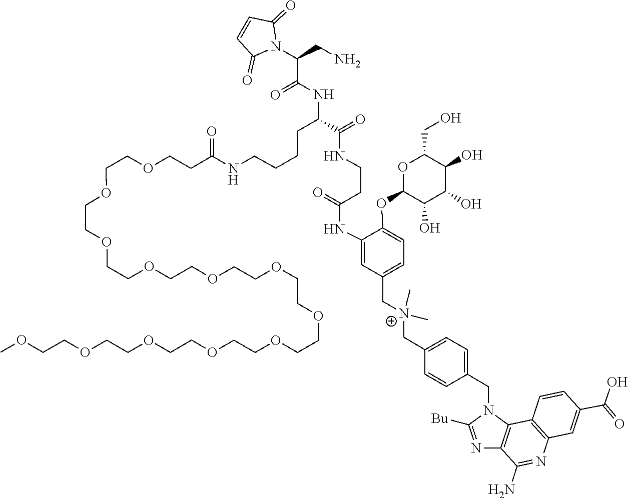 |
| 39 | 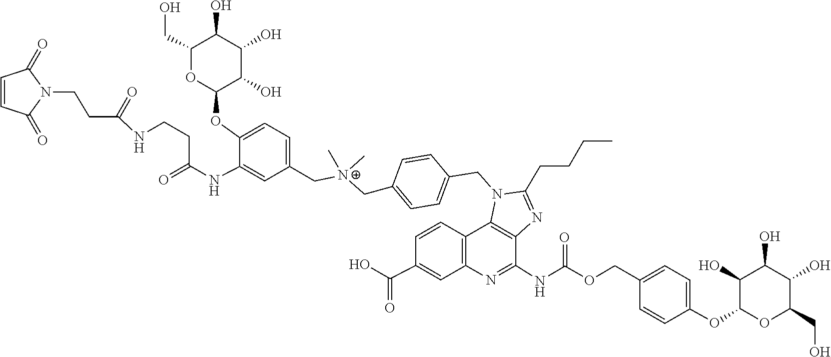 [SMILES: CCCCc1nc2c(NC(=O)OCc3ccc(O[C@H]4O[C@H](CO)[C@@H](O)[C@H](O)[C@@H]4O)cc3)nc3cc(C(=O)O)ccc3c2n1Cc1ccc(C[N+](C)(C)Cc2ccc(O[C@H]3O[C@H](CO)[C@@H](O)[C@H](O)[C@@H]3O)c(NC(=O)CCNC(=O)CCN3C(=O)C=CC3=O)c2)cc1] |
| 40 | 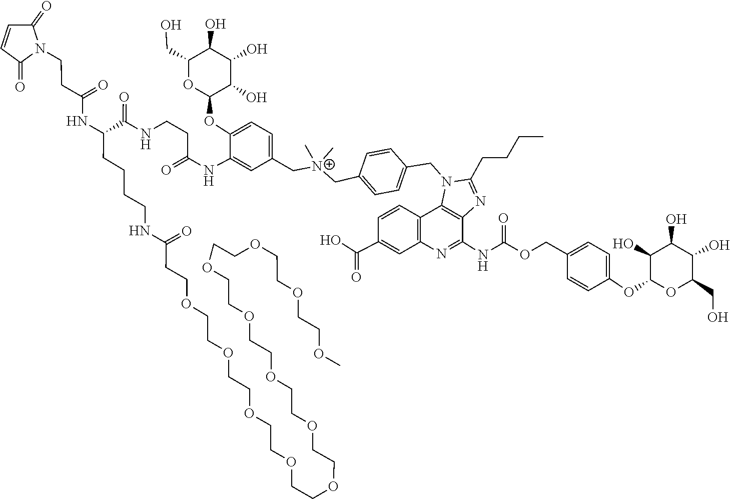 [SMILES: CCCCc1nc2c(NC(=O)OCc3ccc(O[C@H]4O[C@H](CO)[C@@H](O)[C@H](O)[C@@H]4O)cc3)nc3cc(C(=O)O)ccc3c2n1Cc1ccc(C[N+](C)(C)Cc2ccc(O[C@H]3O[C@H](CO)[C@@H](O)[C@H](O)[C@@H]3O)c(NC(=O)CCNC(=O)[C@H](CCCCNC(=O)CCOCCOCCOCCOCCOCCOCCOCCOCCOCCOCCOCCOC)NC(=O)CCN3C(=O)C=CC3=O)c2)cc1] |

In some embodiment, the compounds of Formula L¹D described herein are present in the form of a salt. In some embodiments, the salt is a pharmaceutically acceptable salt.

### EXAMPLES

### General Methods:

All commercially available anhydrous solvents and reagents were used without further purification. Preparative high-performance liquid chromatography (HPLC) was carried out on a Waters 2545 solvent delivery system configured with a Waters 2998 photodiode array (PDA) detector. Unless otherwise specified, products were purified using either Method A or Method B.

Method A: Reversed phase HPLC (RP-HPLC) using Phenomenex Synergi C₁₂ columns (10-50 mm in diameter, 250 mm in length, 4 m, 80 A), eluting with 0.05% (v/v) trifluoroacetic acid (TFA) in water (solvent A) and 0.05% (v/v) trifluoroacetic acid (TFA) in acetonitrile (MeCN) (solvent B); consisted of linear gradients of solvent A to solvent B, ramping from 5 to 10% aqueous solvent B to 95% solvent B; flow rate was varied from 4.6 mL/min to 60 mL/min depending on column diameter.

Method B: Normal phase Biotage Isolera system with Biotage Sfar silica columns, eluting with either hexane or dichloromethane (DCM) as Solvent A and ethyl acetate (EtOAc) or methanol (MeOH) as solvent B. The normal phase purification methods generally consisted of linear gradients of solvent A to solvent B, ramping from 0% solvent B to 100% solvent B; flow rate was varied depending on column diameter.

LC-MS analyses were performed on either Waters Acquity SDS system (Method C) or Waters Acquity QSM system (Method D) with the following parameters: Method C: Column=Waters CORTECS UPLC C18 1.6 μm, 2.1×50 mm column; 0.1% (v/v) formic acid (FA) in water (solvent A) and 0.1% (v/v) FA in MeCN (solvent B) as mobile phases. Elution gradients are listed as follows: 3% B to 60% B over 1.7 min, 60% B to 95% B over 0.3 min, 95% B back to 3% B (initial condition) over 0.5 min and equilibrate for 0.3 min; flow rate was set at 0.5 mL/min.

Method D: Column=Waters CORTECS UPLC C18 1.6 μm, 2.1×50 mm column; 0.1% (v/v) formic acid (FA) in water (solvent A) and 0.1% (v/v) FA in MeCN (solvent B) as mobile phases. Elution gradients are listed as follows: 3% B to 60% B over 1.7 min, 60% B to 97% B over 0.4 min, hold at 97% for 0.4 min, 97% B back to 3% B (initial condition) over 0.1 min and equilibrate for 0.1 min; the flow rate was set at 0.6 mL/min.

Method E: Column=Waters CORTECS UPLC C18 1.6 μm, 2.1×50 mm column; 0.1% (v/v) formic acid (FA) in water (solvent A) and 0.1% (v/v) FA in MeCN (solvent B) as mobile phases. Elution gradients are listed as follows: 3% B to 60% B over 1.1 min, 60% B to 97% B over 0.4 min, hold at 97% for 1.0 min, 97% B back to 3% B (initial condition) over 0.1 min and equilibrate for 0.1 min; the flow rate was set at 0.6 mL/min.

### General Procedures for the Preparation of ADCs

In some embodiments, ADCs were prepared as described previously (Methods Enzymol. 2012, 502, 123-138). Briefly, conjugates were prepared by partial or full reduction of the antibody inter-chain disulfide bonds using various amounts of tris(2-carboxyethyl)phosphine (TCEP) according to the targeted DAR (drug-to-antibody ratio). In the case of DAR4, TCEP was added at approximately 2.2 molar equivalents relative to the antibody (TCEP:antibody) to a pre-warmed (37° C.) antibody stock solution in phosphate buffered saline, (PBS,Gibco, PN 10010023) and 1 M EDTA. The reduction reaction mixture was incubated at 37° C. for approximately 60 minutes. Conjugation of the partially-reduced antibody with maleimide drug-linker was carried out by adding 6 molar equivalents of the drug-linker as a DMSO stock solution. Additional DMSO was added as necessary to achieve a final reaction concentration of 10% (v/v) DMSO to keep the drug-linker remain in solution during the conjugation reaction. The conjugation reaction was allowed to proceed for 30 minutes at room temperature or until all available antibody cysteine thiols had been alkylated by drug-linker as indicated by reversed-phase HPLC (Method G). Removal of excess drug-linker was achieved by incubating the reaction mixture with 100% molar excess QuadraSil® MP resin (Millipore Sigma, PN 679526) for 30 minutes at room temperature. Buffer exchange into formulation buffer (PBS, Gibco, PN 10010023) was achieved by gel filtration chromatography using a prepacked PD-10 column (GE Life Sciences, PN 17043501) according to manufacturer's instructions. Further removal of residual drug-linker was achieved by repeated diafiltration (5-10 times) of the reaction mixture containing the ADCs in formulation buffer using a 30 kilodalton molecular weight cutoff centrifugal filter (Millipore Sigma, PN Z717185), until there was no detectable free drug-linker remaining, as indicated by HPLC analysis (Method K).

### General Procedures for the Characterization of ADCs

ADCs were characterized using the following methods:

Method I: Size-exclusion chromatography (SEC) was performed with a Waters ACQUITY UPLC system and an Acquity UPLC Protein BEH SEC Column, (200 Å, 1.7 μm, 4.6×150 mm, PN: 186005225). The mobile phase used was 7.5% isopropanol in 92.5% aqueous (25 mM sodium phosphate, 350 mM NaCl, pH 6.8), v/v. Elution was performed isocratically at a flow rate of 0.4 mL/min at ambient temperature.

Method J: Reversed-phase chromatography (RP-HPLC) was performed on a Waters 2695 HPLC system and an Agilent PLRP-S column (1000 Å, 8 μm 50×2.1 mm, PN: PL1912-1802). ADCs were treated with 10 mM DTT to reduce disulfide bonds prior to analysis. Sample elution was done using Mobile Phase A (0.05% (v/v) TFA in water) and Mobile Phase B (0.01% (v/v) TFA in MeCN) with a gradient of 25-44% B over 12.5 minutes at 80° C. The drug-to-antibody ratio (DAR) was calculated based on the integrated peak area measured at UV 280 nm.

### Calculation of Molar Ratios

The average drug loading per antibody light-chain (MR_{DLC}) or antibody heavy-chain (MR_{DHC}) was calculated using the equations below:

$${MR}_{D L C} = \frac{\sum ( L C \% {area}_{n} \times {MR}_{n} )}{100}$$

where MR_{DLC}=average drug-to-light chain ration
   LC % areaₙ=% area of the nth loaded light chain species
      % areas based on light chain peaks only
   MRₙ=drug-to-antibody ratio of the nth loaded species
AND

$${MR}_{D H C} = \frac{\sum ( H C \% {area}_{n} \times {MR}_{n} )}{100}$$

where MR_{DHC}=average drug-to-heavy chain ratio
   HC % areaₙ=% area of the nth loaded heavy chain species
      % areas based on heavy chain peaks only
MRₙ=drug-to-antibody ratio of the nth loaded species

The average drug loading per antibody (MR_{D}) was calculated using the equation below:

MR_{D}=2×(MR_{DLC}+MR_{DHC})
where MR_{D}=average drug-to-antibody ratio
   MR_{DLC}=average drug-to-light chain ratio
   MR_{DHC}=average drug-to-heavy chain ratio.

Method K: Residual unconjugated drug linker was measured on a Waters ACQUITY UPLC system using an ACQUITY UPLC BEH C₁₈ Column (130 Å, 1.7 μm, 2.1 mm×50 mm, PN: 186002350). ADC samples were treated with 2× volumes of ice-cold MeOH to induce precipitation and pelleted by centrifugation. The supernatant, containing any residual, unconjugated drug-linker, was injected onto the system. Sample elution was done using Mobile Phase A (0.05% (v/v) TFA in Water) and Mobile Phase B (0.01% TFA (v/v) in MeCN) with a gradient of 1-95% B over 2 minutes at 50° C. Detection was performed at 215 nm and quantitation of the residual drug-linker compound was achieved using an external standard of the corresponding linke

### Example 1

### Synthesis of a PEGylated Drug Linker

Synthesis of 3-((S)-44-((S)-3-amino-2-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4S,5S,6R)-3,4,5-trihydroxy-6-(hydroxymethyl)tetrahydro-2H-pyran-2-yl)oxy)benzyl (2-(ethoxymethyl)-1-(2-hydroxy-2-methylpropyl)-1H-imidazo[4,5-c]quinolin-4-yl)carbamate (S1). A general scheme for linker (S1) synthesis is provided in SCHEME 1 below.

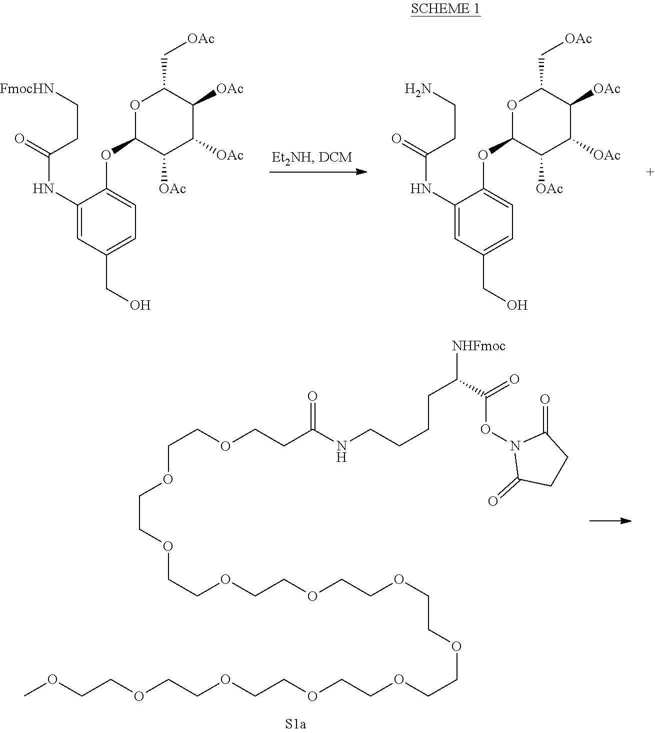

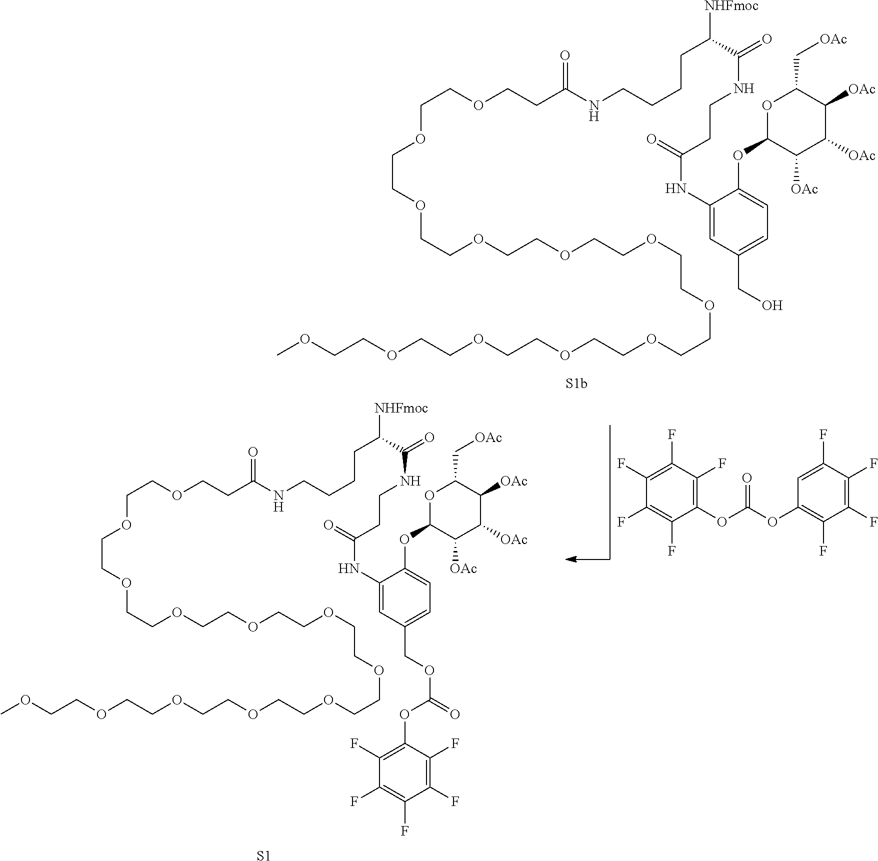

Synthesis of (9H-fluoren-9-yl)methyl (S)-(45-(((2,5-dioxocyclopentyl)oxy)amino)-38,45-dioxo-2,5,8,11,14,17,20,23,26,29,32,35-dodecaoxa-39-azapentatetracontan-44-yl)carbamate compound with methane (1:1) (Sla). A general synthesis of intermediate Sla for generating linker Si is provided in SCHEME 2 below. Briefly, Fmoc-Lys-OH (Sigma-Aldrich, 1.34 g, 3.65 mmol) and PEG12-NHS ester (BroadPharm, 2.5 g, 3.65 mmol) were dissolved in dimethylformamide (DMF, 4 mL), followed by the addition of N,N-diisopropylethylamine (DIPEA, 1.27 ml, 7.29 mmol). The reaction mixture was stirred at room temperature for 6 h, after which solvents were removed in vacuo. The crude reaction mixture was purified by NP-Biotage (Method B) to provide Fmoc-LysPEG12-OH (2.99 g, 03.18 mmol, 87.3%) as colorless liquid. LCMS: m/z [M+H]⁺=939.51 (theoretical); 939.46 (observed). HPLC retention time=1.83 min (Method C). Fmoc-LysPEG12-OH (890 mg, 0.92 mmol) and N-hydroxysuccinimide (Sigma-Aldrich, 212 mg, 1.84 mmol) was dissolved in tetrahydrofuran (THF, (10 mL), followed by the addition of diisopropylc carbodiimide (DIC, Sigma-Aldrich, 288 μL, 1.84 mmol). After stirring for 3 h at room temperature, the solids were filtered off and solvents were removed in vacuo. The crude product was purified by NP-Biotage (Method B) to give intermediate Sla (843 mg, 0.81 mmol, 88.4%) as colorless liquid. LCMS: m/z [M+H]⁺=1036.52 (theoretical); 1036.66 (observed). HPLC retention time=1.85 min (Method C).

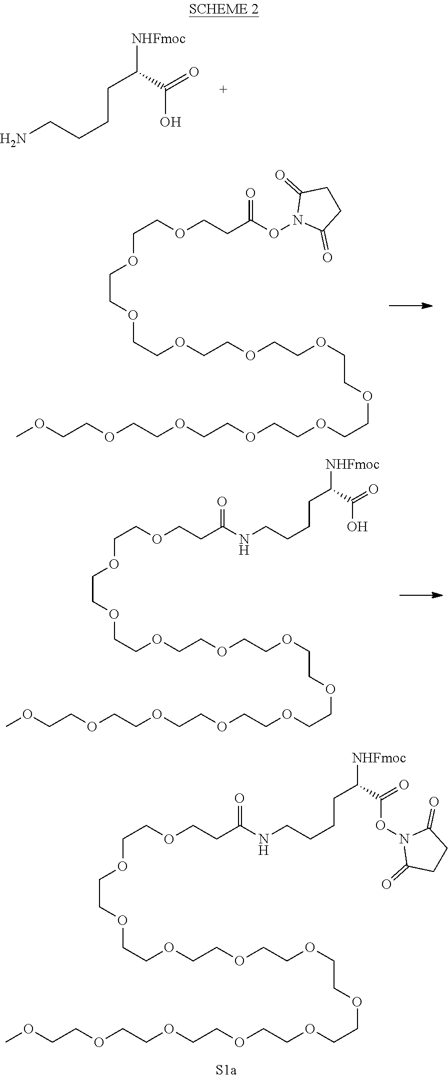

Synthesis of (2R,3S,4S,5R,6R)-2-(2-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-((((perfluorophenoxy)carbonyl)oxy)methyl)phenoxy)-6-(acetoxymethyl)tetrahydro-2H-pyran-3,4,5-triyl triacetate (S1) from Sla. Fmoc-mann-PAB-OH (Wuxi AppTech, 2.2 g (2.85 mmol) was dissolved in a 5:1 (v/v) mixture of dichloromethane/diethylamine (DCM/Et₂NH, 20 mL) and the resulting reaction mixture was stirred at room temperature for 2 h. Solvents were removed after 2 h and the crude product was used directly in the next step without purification. LCMS: m/z [M+H]⁺=541.20 (theoretical); 541.41 (observed). HPLC retention time=1.50 min (Method C). NH₂-mann-PAB-OH (1.54 g, 2.85 mmol) and Sla (3.54 g, 3.42 mmol) were dissolved in DMA (20 mL), followed by the addition of N,N-diisopropylethylamine (DIPEA, 1.5 ml, 8.55 mmol) and the reaction mixture was stirred at room temperature for 2 h, after which solvents were removed in vacuo. The crude reaction mixture was purified by NP-Biotage (Method B) to provide S1b (3.21 g, 2.2 mmol, 77.1%) as colorless liquid. LCMS: m/z [M+H]⁺=1461.69 (theoretical); 1461.66 (observed). HPLC retention time=1.80 min (Method C). Fmoc-Compound S1b (460 mg, 0.315 mmol) and Bis(pentafluorophenyl) carbonate (Sigma-Aldrich, 186 mg, 0.472 mmol) were dissolved in dimethylacetamide (DMA, 2 mL) followed by the addition of DIPEA (0.164 ml, 0.944 mmol). The reaction mixture was stirred at room temperature for 6 h after which solvents were removed in vacuo. The crude reaction mixture was purified by NP-Biotage (Method B) to provide S1 (301 mg, 0.18 mmol, 57.2%) as colorless sticky liquid. LCMS: m/z [M+H]⁺=1671.67 (theoretical); 1671.60 (observed). HPLC retention time=2.17 min (Method C).

### Example 2

### Synthesis of a Drug-Linker Conjugate

This example covers synthesis of a complex with a toll-like receptor 7 and toll like receptor 8 (TLR7/8) agonist coupled to a linker by a C4 amine of its imidazoquinoline core and capable of coupling to a protein. The TLR7/8 agonist is unfunctionalized at its imidazoquinoline C₇ position.

Synthesis of (2R,3S,4S,5R,6R)-2-(2-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-((((2-(ethoxymethyl)-1-(2-hydroxy-2-methylpropyl)-1H-imidazo[4,5-c]quinolin-4-yl)carbamoyl)oxy)methyl)phenoxy)-6-(acetoxymethyl)tetrahydro-2H-pyran-3,4,5-triyl triacetate (S2). Intermediate Si (49.3 mg, 0.029 mmol), resiquimod (Asta Tech, 7.1 mg, 0.023 mmol), 1-Hydroxybenzotriazole (HOBt, 1.53 mg, 0.011 mmol), and DIPEA (11.9 μl, 0.068 mmol) were dissolved in DMA and the reaction mixture was stirred at 30° C. for 14 h. After 14 h, the reaction mixture was diluted with dimethylsulfoxide (DMSO)/water and purified by RP-HPLC (Method A) to give intermediate S2 (20.1 mg, 0.011 mmol, 39.5%) as TFA salt, white solid. LCMS: m/z [M+H]⁺=1801.84 (theoretical); 1801.77 (observed); HPLC retention time=1.77 min (Method C). Synthesis of 3-((S)-44-amino-38,45-dioxo-2,5,8,11,14,17,20,23,26,29,32,35-dodecaoxa-39,46-diazanonatetracontan-49-amido)-4-(((2R,3 S,4S,5S,6R)-3,4,5-trihydroxy-6-(hydroxymethyl)tetrahydro-2H-pyran-2-yl)oxy)benzyl (2-(ethoxymethyl)-1-(2-hydroxy-2-methylpropyl)-1H-imidazo[4,5-c]quinolin-4-yl)carbamate (S3). A synthesis of S3 using intermediate S2 is outlined in SCHEME 3 below. Intermediate S2 (20.1 mg, 0.011 mmol) was dissolved in dichloromethane (DCM, 0.5 mL) at rom temperature (RT) followed by the addition of dimethylamine (Et₂NH, 0.25 mL). The reaction mixture was stirred at RT for 45 min. After 45 min, solvents were removed in vacuo and the crude reaction mixture was re-dissolved in methanol (MeOH, 0.8 mL) at 0° C. and a sodium methoxide (NaOMe) solution (0.5 M in MeOH, 0.178 mL, 0.089 mmol) was added. The resulting reaction mixture was stirred at 0° C. for 90 min, upon which, glacial acetic acid (AcOH, 6.32 uL, 0.112 mmol) was added to neutralize the reaction mixture. The reaction mixture was diluted with water/DMSO and purified by preparative RP-HPLC (Method A) to give intermediate S3 (9.7 mg, 0.006 mmol, 57.0% yield) as TFA salt, white solid. LCMS: m/z [M+H]⁺=1411.7 (theoretical); 1411.99 (observed). HPLC retention time=1.17 min (Method C).

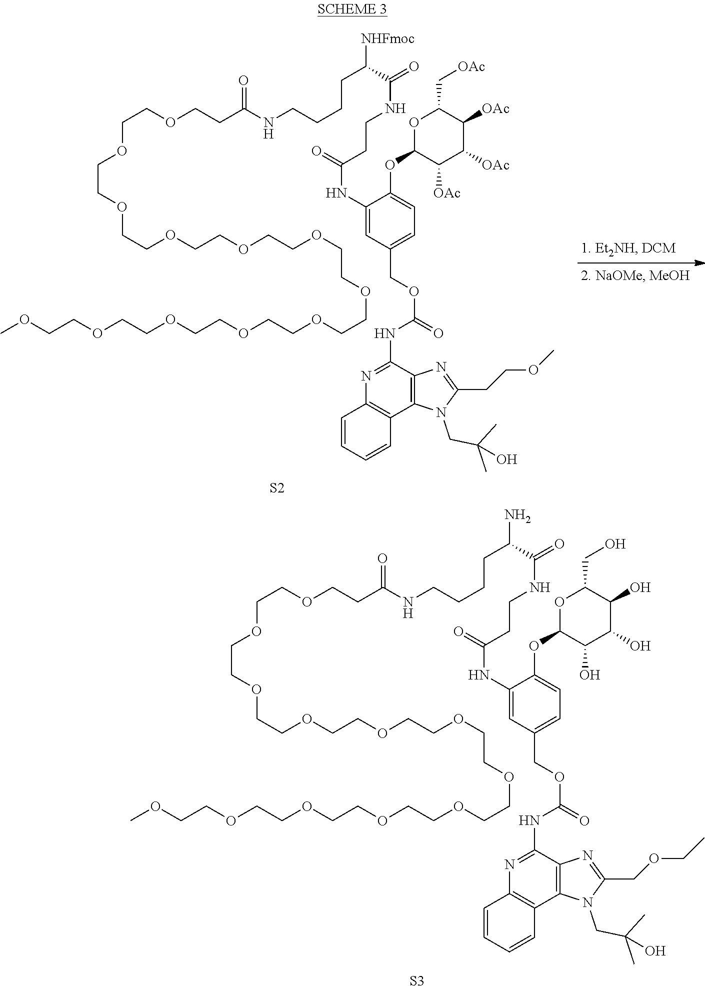

Synthesis of 3-((S)-44-((S)-3-amino-2-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4S,5S,6R)-3,4,5-trihydroxy-6-(hydroxymethyl)tetrahydro-2H-pyran-2-yl)oxy)benzyl (2-(ethoxymethyl)-1-(2-hydroxy-2-methylpropyl)-1H-imidazo[4,5-c]quinolin-4-yl)carbamate (1). Intermediate S4 was prepared according to the procedures as described in Lyon, et al. Nat. Biotechnol. Vol. 32, 1059-1062 (2014). Intermediate S3 (6.9 mg, 0.005 mmol) and 2,5-dioxopyrrolidin-1-yl (S)-3-((tert-butoxycarbonyl)amino)-2-(2,5-dioxo-2,5-dihydro-1H-pyrrol-1-yl)propanoate (S4, (2.07 mg, 0.006 mmol) were dissolved in anhydrous DMF (0.5 mL) and DIPEA (2.36 μL, 0.014 mmol) at room temperature. The reaction mixture was stirred at room temperature for 9 h. After 9 h, solvents were removed in vacuo and the crude reaction mixture was re-dissolved in DCM/TFA (10:1 (v/v), 0.5 mL total) and the reaction mixture was stirred at room temperature for another 30 min. Solvents were then removed and the crude reaction mixture was diluted with DMSO/water and purified by preparative RP-HPLC (Method A) to give Compound 1 (3.44 mg, 0.002 mmol, 45.3% yield) as TFA salt, white solid. LCMS: m/z [M+H]⁺=1577.77 (theoretical); 1577.83 (observed). HPLC retention time=1.29 min (Method C).

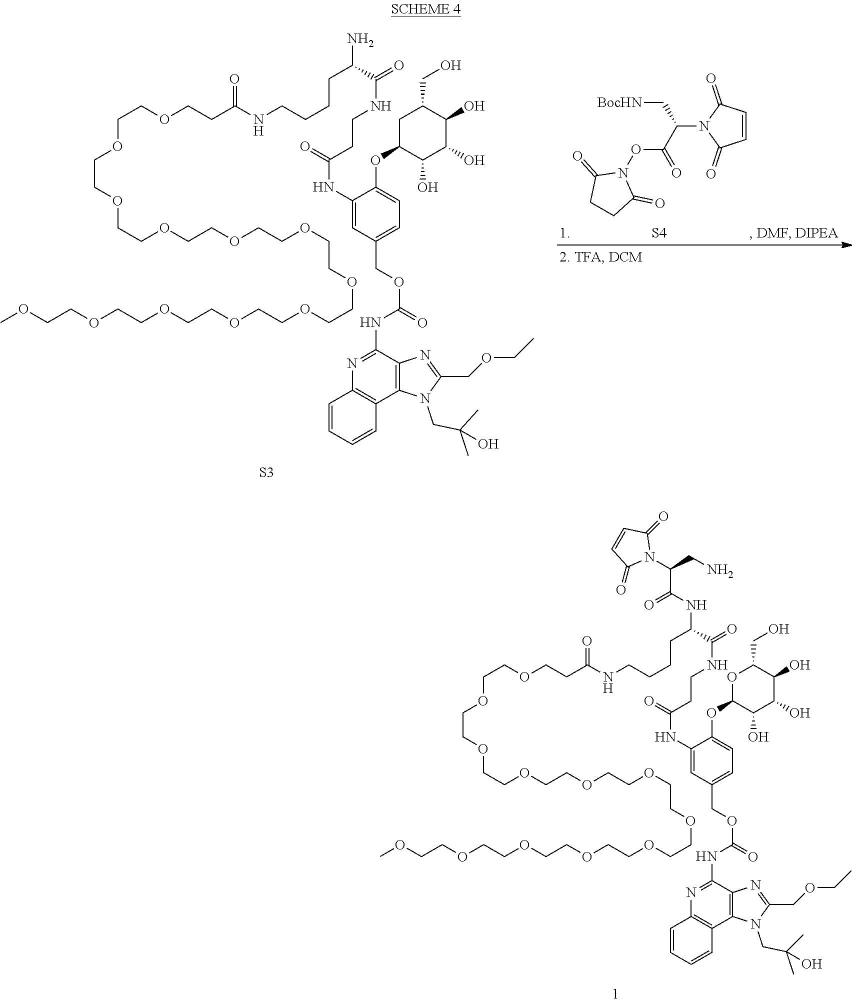

### Example 3

### Synthesis of a Drug-Linker Conjugate

This example covers synthesis of a complex with a toll-like receptor 7 and toll like receptor 8 (TLR7/8) agonist coupled to a linker by a C4 amine of its imidazoquinoline core and capable of coupling to a protein. The TLR7/8 agonist contains methyl ester functionalization at its imidazoquinoline C₇ position.

Synthesis of methyl 2-butyl-4-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-1-(2-hydroxypropyl)-1H-imidazo[4,5-c]quinoline-7-carboxylate (2). SCHEME 5 summarizes a synthesis of a TLR7/8 agonist, linker conjugate with a maleimide group configured to bind to basic protein residues.

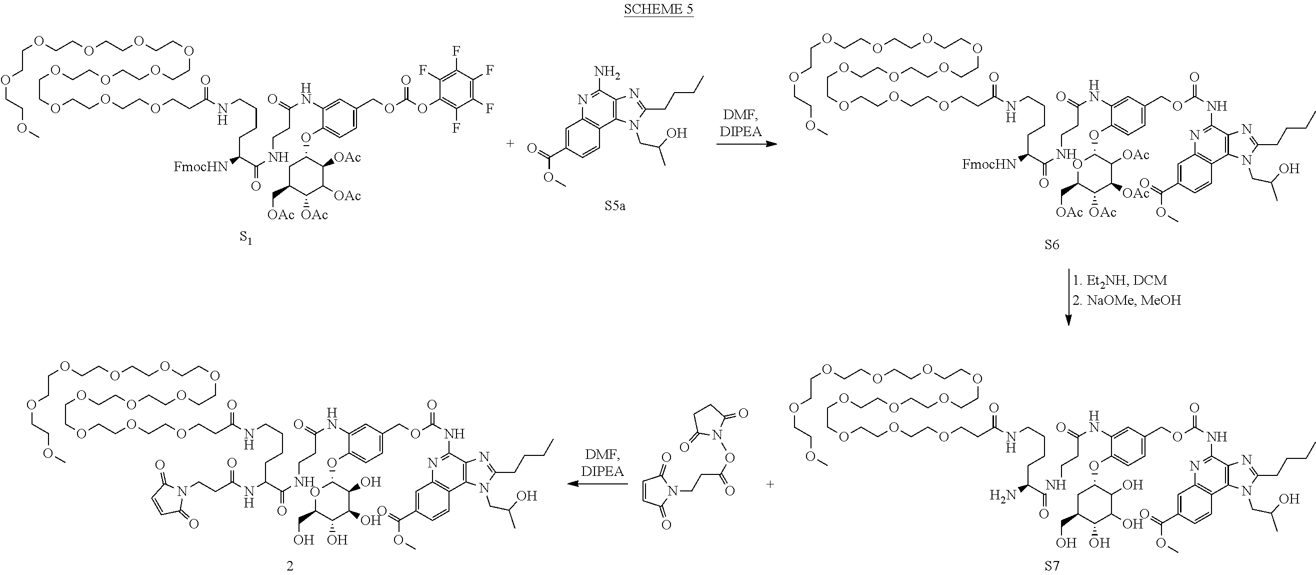

Compound S5a was prepared according to the procedures as described in Larson, et al. ACS Med. Chem. Lett. Vol. 8, No. 11, 1148-1152 (2017).

Synthesis of (2R,3S,4S,5R,6R)-2-(2-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-((((2-butyl-1-(2-hydroxypropyl)-7-(methoxycarbonyl)-1H-imidazo[4,5-c]quinolin-4-yl)carbamoyl)oxy)methyl)phenoxy)-6-(acetoxymethyl)tetrahydro-2H-pyran-3,4,5-triyl triacetate (S6). Compound S6 was synthesized using similar procedures as those used to prepare compound S2, and was obtained as TFA salt, white solid. LCMS: m/z [M+H]⁺=1843.85 (theoretical); 1843.94 (observed). HPLC retention time=1.84 min (Method C).

Synthesis of methyl 4-((((3-((S)-44-amino-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-2-butyl-1-(2-hydroxypropyl)-1H-imidazo[4,5-c]quinoline-7-carboxylate (S7). Compound S7 was synthesized using similar procedures as those used to prepare compound S3, and was obtained as TFA salt, white solid. LCMS: m/z [M+H]⁺=1453.74 (theoretical); 1453.86 (observed). HPLC retention time=1.41 min (Method C).

Synthesis of methyl 2-butyl-4-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4S,5S,6R)-3,4,5-trihydroxy-6-(hydroxymethyl)tetrahydro-2H-pyran-2-yl)oxy)benzyl)oxy)carbonyl)amino)-1-(2-hydroxypropyl)-1H-imidazo[4,5-c]quinoline-7-carboxylate (2). Compound 2 was prepared using similar procedures as those used to prepare compound 1 by reacting compound S7 with 2,5-dioxopyrrolidin-1-yl 3-(2,5-dioxo-2,5-dihydro-1H-pyrrol-1-yl)propanoate (mp-OSu from TCI). Compound 2 was isolated as TFA salt, white solid. LCMS: m/z [M+H]⁺=1604.76 (theoretical); 1604.88 (observed). HPLC retention time=1.48 min (Method C).

### Example 4

### Synthesis of a Drug-Linker Conjugate

This example covers synthesis of a complex with a toll-like receptor 7 and toll like receptor 8 (TLR7/8) agonist coupled to a linker by a C4 amine of its imidazoquinoline core and methyl ester functionalization at its imidazoquinoline C₇ position, namely 4-amino-2-butyl-1-(4-(((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)benzyl)oxy)carbonyl)amino)methyl)benzyl)-1H-imidazo[4,5-c]quinoline-7-carboxylic acid (3).

Synthesis of (2R,3S,4S,5R,6R)-2-(2-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-((((4-((4-amino-2-butyl-7-(methoxycarbonyl)-1H-imidazo[4,5-c]quinolin-1-yl)methyl)benzyl)carbamoyl)oxy)methyl)phenoxy)-6-(acetoxymethyl)tetrahydro-2H-pyran-3,4,5-triyl triacetate (S8). Synthesis of intermediate S8 with compound S8a is summarized in SCHEME 6. Compound S8a was prepared according to the procedures in Larson, et al. ACS Med. Chem. Lett. Vol. 8, No. 11, 1148-1152 (2017). Compounds S1 (33.0 mg, 0.198 mmol), S8a (5.5 mg, 0.132 mmol) and DIPEA (6.9 μL, 0.04 mmol) were dissolved in anhydrous DMA (0.5 mL) and the reaction mixture was stirred for 5 min at room temperature. After 5 min, the reaction mixture was diluted with DMSO/water and purified by RP-HPLC (Method A) to give intermediate S8 (23.2 mg, 0.011 mmol, 87.2%) as TFA salt, white solid. LCMS: m/z [M+H]⁺=1905.88 (theoretical); 1905.21 (observed). HPLC retention time=1.86 min (Method C).

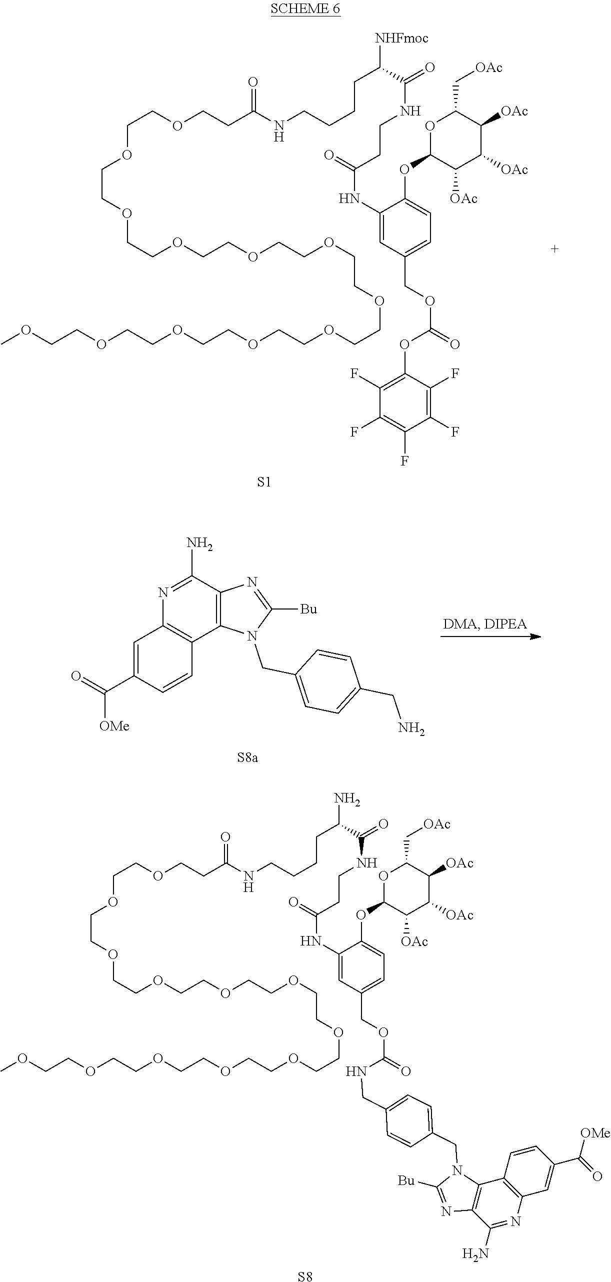

Synthesis of 4-amino-1-(4-(((((3-((S)-44-amino-38,45-dioxo-2,5,8,11,14,17,20,23,26,29,32,35-dodecaoxa-39,46-diazanonatetracontan-49-amido)-4-(((2R,3 S,4 S,5 S,6R)-3,4,5-trihydroxy-6-(hydroxymethyl)tetrahydro-2H-pyran-2-yl)oxy)benzyl)oxy)carbonyl)amino)methyl)benzyl)-2-butyl-1H-imidazo [4,5-c]quinoline-7-carboxylic acid (S9). SCHEME 7 outlines a synthesis of S9 from S8. Intermediate S8 (20.0 mg, 0.105 mmol) was dissolved in tetrahydrofuran (THF, 0.6 mL) and an aqueous lithium hydroxide (LiOH) solution (0.2 M, 0.525 mL, 0.105 mmol) was added. The reaction mixture was stirred at room temperature for 1 h, upon which LCMS analysis indicated full conversion. The reaction mixture was quenched with glacial acetic acid (HOAc, 6 μL) and the solvent was removed in vacuo. The resulted residue was diluted with DMSO/water and purified by RP-HPLC (Method A) to give intermediate S9 (11.4 mg, 0.007 mmol, 67.3%) as TFA salt, white solid. LCMS: m/z [M+H]⁺=1500.75 (theoretical); 1500.56 (observed). HPLC retention time=1.43 min (Method C).

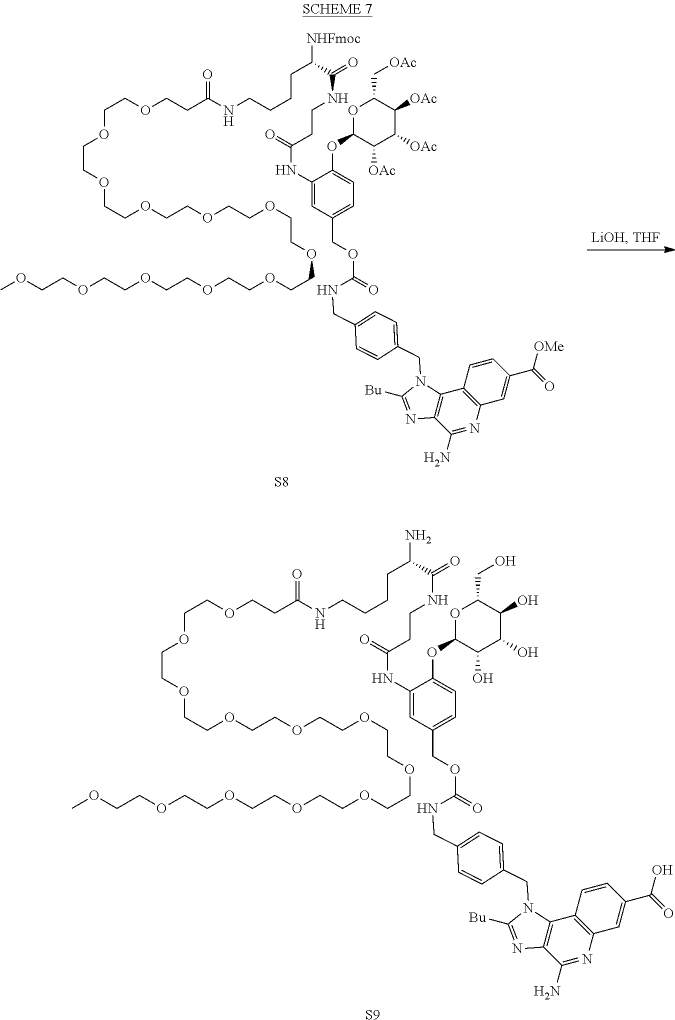

Synthesis of 4-amino-2-butyl-1-(4-(((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)methyl)benzyl)-1H-imidazo[4,5-c]quinoline-7-carboxylic acid (3). Synthesis of compound 3 from S9 is outlined in SCHEME 8. Intermediate S9 (6.6 mg, 4.09 μmol) and mp-OSu (TCI, 1.31 mg, 4.91 μmol) were dissolved in anhydrous DMA (0.3 mL) and DIPEA (2.2 μL, 0.012 mmol) at room temperature. The reaction mixture was stirred at the same temperature for 30 min. After 30 min, the reaction mixture was quenched with HOAc (5 μl), diluted with DMSO/water and purified by RP-HPLC (Method A) to give compound 3 (4.9 mg, 0.003 mmol, 67.9%) as TFA salt, white solid. LCMS: m/z [M+H]⁺=1651.78 (theoretical); 1652.49 (observed). HPLC retention time=1.32 min (Method C).

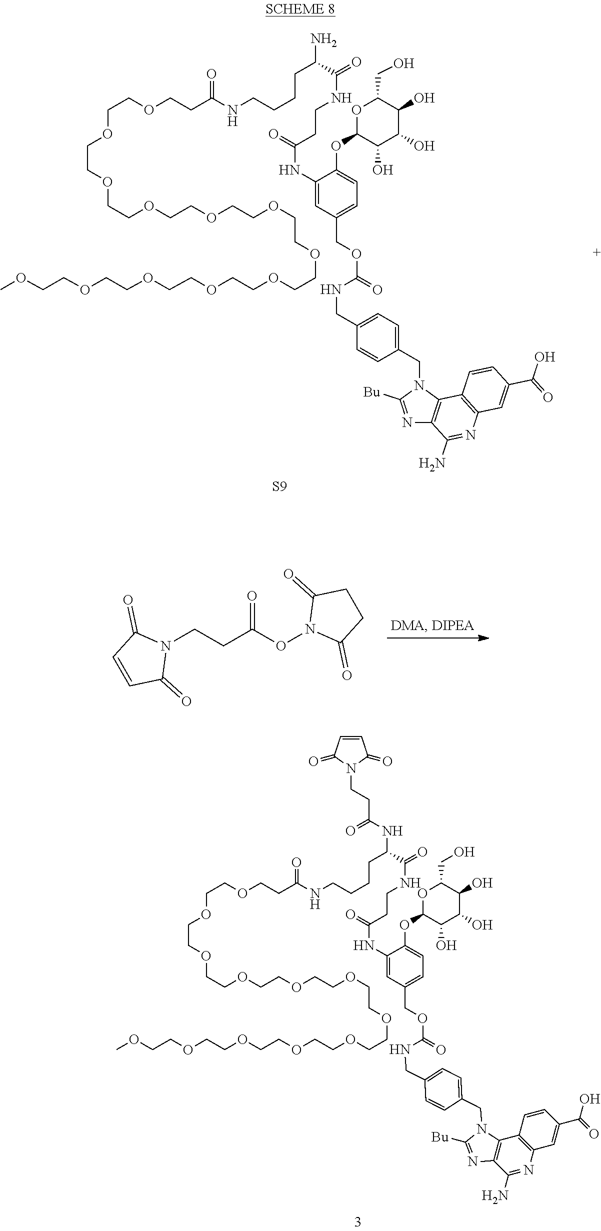

### Example 5

### Synthesis of a drug, Linker Conjugate

This example covers synthesis of a complex with a toll-like receptor 7 and toll like receptor 8 (TLR7/8) agonist coupled to a linker by an N1 of its imidazoquinoline core and capable of coupling to a protein. The TLR7/8 agonist contains methyl ester functionalization at its imidazoquinoline C₇ position.

Synthesis of methyl 4-amino-1-(4-(((((3-((S)-44-amino-38,45-dioxo-2,5,8,11,14,17,20,23,26,29,32,35-dodecaoxa-39,46-diazanonatetracontan-49-amido)-4-(((2R,3 S,4S,5 S,6R)-3,4,5-trihydroxy-6-(hydroxymethyl)tetrahydro-2H-pyran-2-yl)oxy)benzyl)oxy)carbonyl)amino)methyl)benzyl)-2-butyl-1H-imidazo [4,5-c]quinoline-7-carboxylate (S10). Synthesis of intermediate S10 from intermediate S8 is summarized in SCHEME 9. Intermediate S8 (19.3 mg, 11.5 μmol) was dissolved in 0.8 mL of a 4:1 (v/v) mixture of DCM and Et₂NH, and the resulting solution was stirred at room temperature for 15 min. After 15 min, solvents were removed in vacuo and the reaction mixture was re-dissolved in MeOH (0.5 mL), followed by the addition of sodium methoxide (NaOMe, 0.5 M solution in MeOH, 0.138 mL, 0.069 mmol). The reaction mixture was stirred at room temperature for 30 min and then quenched with HOAc (7 μL). Solvents were then removed in vacuo. The resulting residue was diluted with DMSO/water and purified by RP-HPLC (Method A) to give intermediate S10 (14.0 mg, 0.008 mmol, 69.9%) as TFA salt, white solid. LCMS: m/z [M+H]⁺=1514.77 (theoretical); 1514.80 (observed). HPLC retention time=1.28 (Method C).

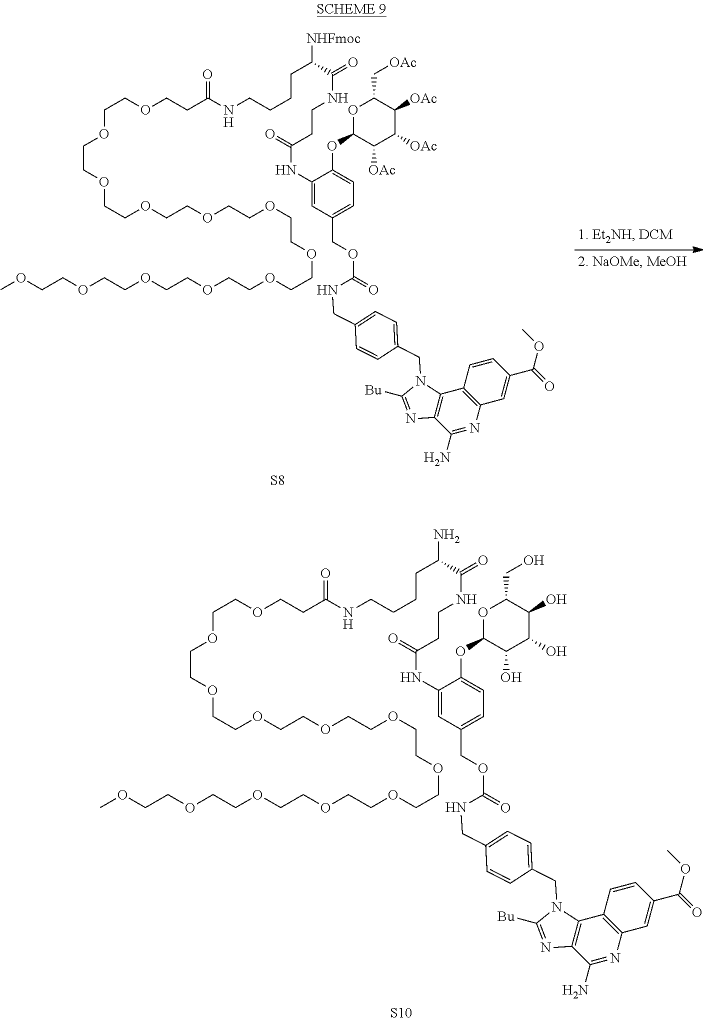

Synthesis of methyl 4-amino-2-butyl-1-(4-(((((3-((S)-44-(3-(2,5-dioxo-2,5-dihydro-1H-pyrrol-1-yl)propanamido)-38,45-dioxo-2, 5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)methyl)benzyl)-1H-imidazo[4,5-c]quinoline-7-carboxylate (4). SCHEME 10 outlines a synthesis of compound 4 from intermediate S10. Intermediate S10 (14.0 mg, 0.008 mmol) and mp-OSu (3.21 mg, 0.012 mmol) were dissolved in anhydrous DMA (0.3 mL) and DIPEA (7.0 μL, 0.04 mmol) at room temperature. The reaction mixture was stirred at the same temperature for 15 min. After 15 min, the reaction mixture was quenched with HOAc (5 μl), diluted with DMSO/water, and purified by RP-HPLC (Method A) to give compound 4 (10.3 mg, 0.006 mmol, 72.0%) as TFA salt, white solid. LCMS: m/z [M+H]⁺=1665.80 (theoretical); 1666.46 (observed). HPLC retention time=1.35 min (Method C).

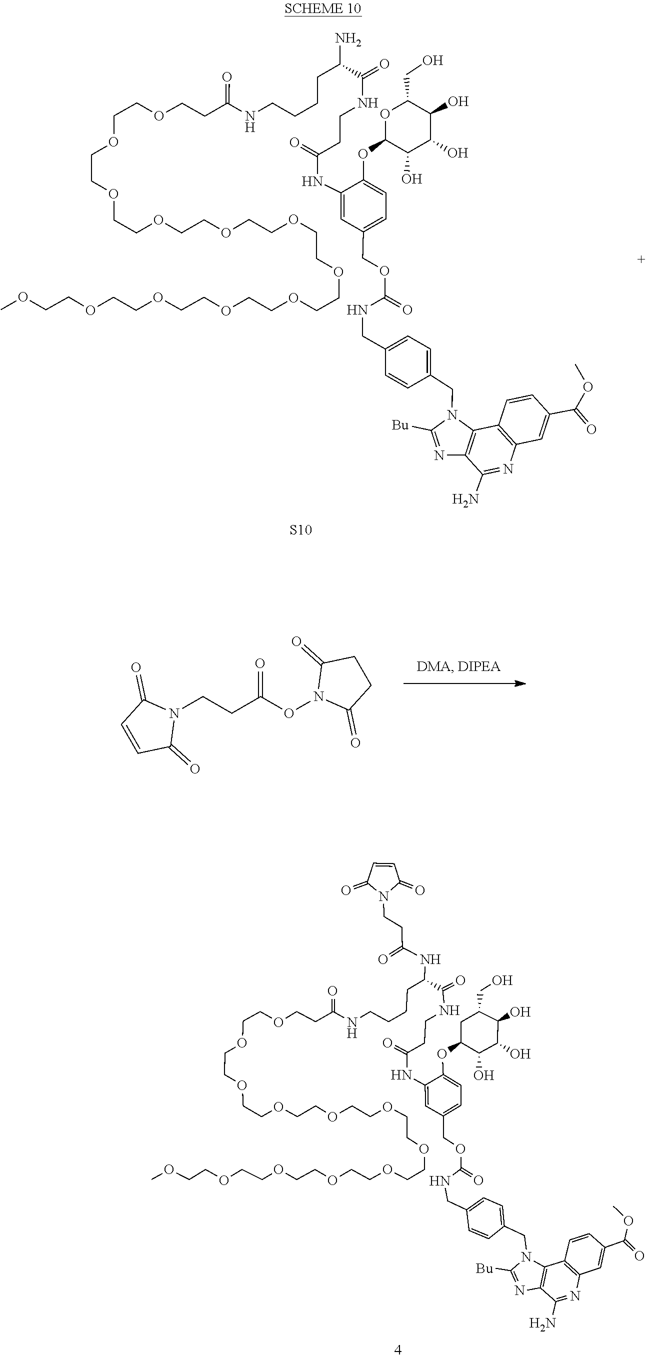

### Example 6

### Syntheses of Two Drug-Linker Conjugates

This example covers syntheses of complexes with TLR7/8 agonists coupled to linkers by an N1 of imidazoquinoline cores and capable of coupling to a protein. The TLR7/8 agonists contain variable functionalization at position C₇ of their imidazoquinoline cores, with compound 5 containing methyl ester functionalization at this position and compound 6 containing carboxylic acid functionalization at this position.

Syntheses of methyl 4-amino-2-butyl-1-(2-((((3-((S)-44-(3-(2,5-dioxo-2,5-dihydro-1H-pyrrol-1-yl)propanamido)-38,45-dioxo-2, 5,8,11,14,17,20,23,26,29,32,35-dodecaoxa-39,46-diazanonatetracontan-49-amido)-4-(((2R,3 S,4 S,5 S,6R)-3,4,5-trihydroxy-6-(hydroxymethyl)tetrahydro-2H-pyran-2-yl)oxy)benzyl)oxy)carbonyl)amino)ethyl)-1H-imidazo[4,5-c]quinoline-7-carboxylate (5) and 4-amino-2-butyl-1-(2-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4S,5 S,6R)-3,4,5-trihydroxy-6-(hydroxymethyl)tetrahydro-2H-pyran-2-yl)oxy)benzyl)oxy)carbonyl)amino)ethyl)-1H-imidazo[4,5-c]quinoline-7-carboxylic acid (6).

These syntheses are summarized in SCHEME 11. The syntheses of compounds 5 and 6 were similar to the syntheses of compound 4 and compound 3, respectively.

The synthesis of compound 5 and compound 6 was similar to the synthesis of compound 4 and compound 3, respectively.

Compound S11a was prepared according to the procedures as described in Larson, et al. ACS Med.

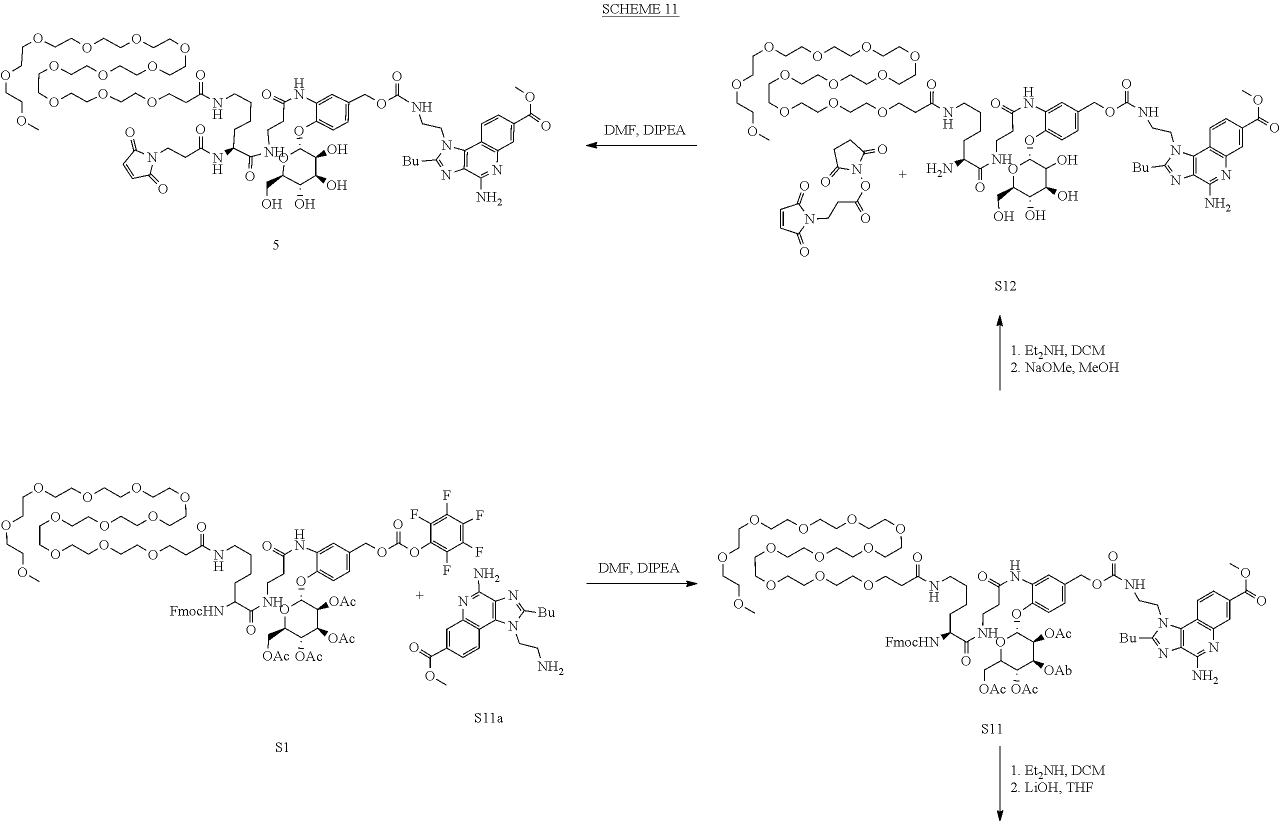

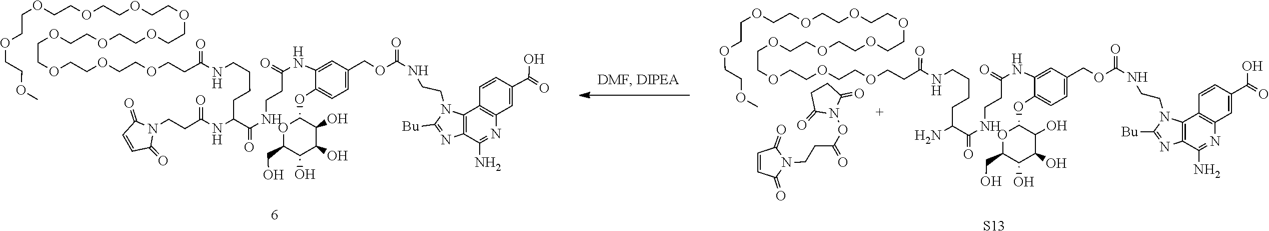

(2R,3S,4S,5R,6R)-2-(2-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-((((2-(4-amino-2-butyl-7-(methoxycarbonyl)-1H-imidazo[4,5-c]quinolin-1-yl)ethyl)carbamoyl)oxy)methyl)phenoxy)-6-(acetoxymethyl)tetrahydro-2H-pyran-3,4,5-triyl triacetate (S11) as TFA salt, white solid. LCMS: m/z [M+H]⁺=1828.85 (theoretical); 1828.95 (observed). HPLC retention time=1.74 min (Method C).

Methyl 4-amino-1-(2-((((3-((S)-44-amino-38,45-dioxo-2,5,8,11,14,17,20,23,26,29,32,35-dodecaoxa-39,46-diazanonatetracontan-49-amido)-4-(((2R,3 S,4S,5 S,6R)-3,4,5-trihydroxy-6-(hydroxymethyl)tetrahydro-2H-pyran-2-yl)oxy)benzyl)oxy)carbonyl)amino)ethyl)-2-butyl-1H-imidazo[4,5-c] quinoline-7-carboxylate (S12) as TFA salt, white solid. LCMS: m/z [M+H]⁺=1438.74 (theoretical); 1438.85 (observed). HPLC retention time=1.33 min (Method C).

Methyl 4-amino-2-butyl-1-(2-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)ethyl)-1H-imidazo[4,5-c]quinoline-7-carboxylate (5) as TFA salt, white solid. LCMS: m/z [M+H]⁺=1589.77 (theoretical); 1589.87 (observed). HPLC retention time=1.39 min (Method C).

Synthesis of 4-amino-1-(2-((((3-((S)-44-amino-38,45-dioxo-2,5,8,11,14,17,20,23,26,29,32,35-dodecaoxa-39,46-diazanonatetracontan-49-amido)-4-(((2R,3 S,4S,5 S,6R)-3,4,5-trihydroxy-6-(hydroxymethyl)tetrahydro-2H-pyran-2-yl)oxy)benzyl)oxy)carbonyl)amino)ethyl)-2-butyl-1H-imidazo[4,5-c] quinoline-7-carboxylic acid (S13). Compound S13 was prepared using similar procedures as those used to prepare compound S10, and was isolated as TFA salt, white solid. LCMS: m/z [M+H]⁺=1424.72 (theoretical); 1424.83 (observed). HPLC retention time=1.28 (Method C). 4-amino-2-butyl-1-(2-((((3-((S)-44-(3-(2,5-dioxo-2,5-dihydro-1H-pyrrol-1-yl)propanamido)-38,45-dioxo-2, 5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)ethyl)-1H-imidazo[4,5-c]quinoline-7-carboxylic acid (6) as TFA salt, white solid. LCMS: m/z [M+H]⁺=1575.75 (theoretical); 1576.58 (observed). HPLC retention time=1.22 min (Method C).

### Example 7

### Syntheses of Two Drug-Linker Conjugates

This example covers syntheses of complexes with TLR7/8 agonists coupled to linkers by an N1 of imidazoquinoline cores and capable of coupling to a protein. The TLR7/8 agonists contain variable functionalization at position C₇ of their imidazoquinoline cores, with compound 7 containing methyl ester functionalization at this position and compound 8 containing carboxylic acid functionalization at this position.

Syntheses of methyl 4-amino-2-butyl-1-(5-((((3-((S)-44-(3-(2,5-dioxo-2,5-dihydro-1H-pyrrol-1-yl)propanamido)-38,45-dioxo-2, 5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)pentyl)-1H-imidazo[4,5-c]quinoline-7-carboxylate (7) and 4-amino-2-butyl-1-(5-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4S,5 S,6R)-3,4,5-trihydroxy-6-(hydroxymethyl)tetrahydro-2H-pyran-2-yl)oxy)benzyl)oxy)carbonyl)amino)pentyl)-1H-imidazo[4,5-c]quinoline-7-carboxylic acid (8). The syntheses of compounds 7 and 8 were similar to the synthesis of compound 4 and compound 3, respectively, and are outlined in SCHEME 12 below.

The synthesis of compound 7 and compound 8 was similar to the synthesis of compound 4 and compound 3, respectively.

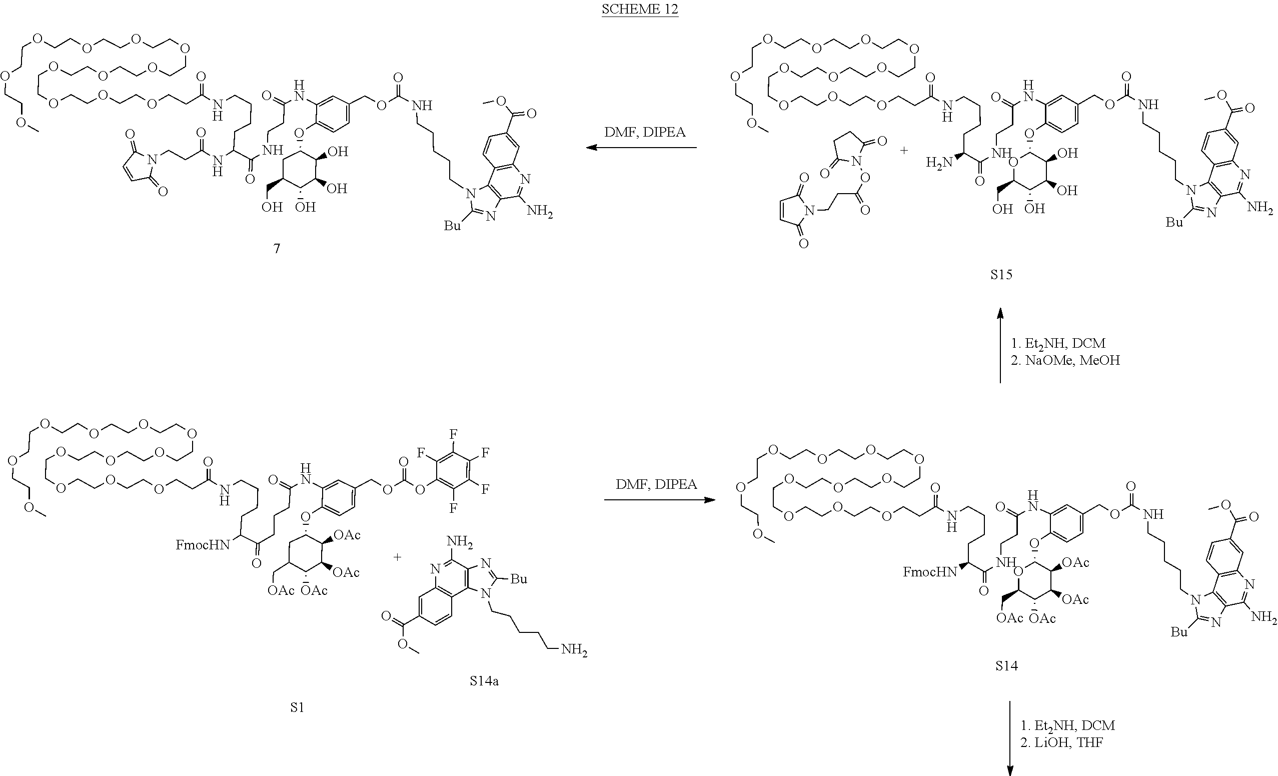

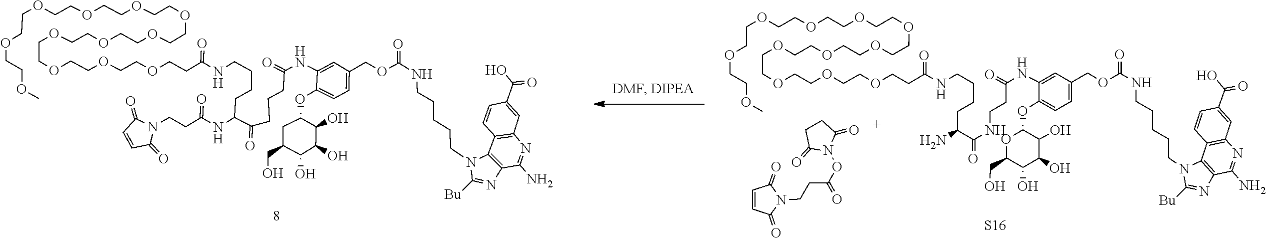

(2R,3S,4S,5R,6R)-2-(2-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-((((5-(4-amino-2-butyl-7-(methoxycarbonyl)-1H-imidazo[4,5-c]quinolin-1-yl)pentyl)carbamoyl)oxy)methyl)phenoxy)-6-(acetoxymethyl)tetrahydro-2H-pyran-3,4,5-triyl triacetate (S14) as TFA salt, white solid. LCMS: m/z [M+H]⁺=1871.90 (theoretical); 1871.74 (observed). HPLC retention time=1.93 min (Method C).

Methyl 4-amino-1-(5-((((3-((S)-44-amino-38,45-dioxo-2,5,8,11,14,17,20,23,26,29,32,35-dodecaoxa-39,46-diazanonatetracontan-49-amido)-4-(((2R,3 S,4S,5 S,6R)-3,4,5-trihydroxy-6-(hydroxymethyl)tetrahydro-2H-pyran-2-yl)oxy)benzyl)oxy)carbonyl)amino)pentyl)-2-butyl-1H-imidazo[4,5-c] quinoline-7-carboxylate (S15) as TFA salt, white solid. LCMS: m/z [M+H]⁺=1480.79 (theoretical); 1481.55 (observed). HPLC retention time=1.45 min (Method C).

Methyl 4-amino-2-butyl-1-(5-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)pentyl)-1H-imidazo[4,5-c]quinoline-7-carboxylate (7) as TFA salt, white solid. LCMS: m/z [M+H]⁺=1631.81 (theoretical); 1632.22 (observed). HPLC retention time=1.47 min (Method C).

4-amino-1-(5-((((3-((S)-44-amino-38,45-dioxo-2,5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)pentyl)-2-butyl-1H-imidazo[4,5-c]quinoline-7-carboxylic acid (S16) as TFA salt, white solid. LCMS: m/z [M+H]⁺=1466.77 (theoretical); 1466.42 (observed). HPLC retention time=1.17 min (Method C).

4-amino-2-butyl-1-(5-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)pentyl)-1H-imidazo[4,5-c]quinoline-7-carboxylic acid (8) as TFA salt, white solid. LCMS: m/z [M+H]+=1617.80 (theoretical); 1617.66 (observed). HPLC retention time=1.28 min (Method C).

### Example 8

### Syntheses of Two Drug-Linker Conjugates

This example covers syntheses of a complex comprising a linker coupled to an N1 position of an imidazoquinoline TLR7/8 agonist with carboxylic acid functionalization at its C₇ position, namely N-(4-((4-amino-2-butyl-7-carboxy-1H-imidazo[4,5-c]quinolin-1-yl)methyl)benzyl)-1-(3-((S)-44-(3-(2,5-dioxo-2,5-dihydro-1H-pyrrol-1-yl)propanamido)-38,45-dioxo-2, 5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)phenyl)-N,N-dimethylmethanaminium (9).

Synthesis of (2R,3S,4S,5R,6R)-2-(2-(3-((((9H-fluoren-9-yl)methoxy)carbonyl)amino)propanamido)-4-(bromomethyl)phenoxy)-6-(acetoxymethyl)tetrahydro-2H-pyran-3,4,5-triyl triacetate (S17). SCHEME 13 outlines a synthetic scheme for intermediate S17. Fmoc-mann-PAB-OH (Wuxi AppTech, 200 mg, 0.262 mmol) and N-bromosuccinimide (NBS, Sigma-Aldrich, 70 mg, 0.393 mmol) were dissolved in DCM (8 mL) at 0° C. followed by the addition of triphenylphosphine (PPh₃, Sigma-Aldrich, 103.2 mg, 0.393 mmol). The reaction mixture was stirred at 0° C. for 10 min then warmed up to room temperature and stirred for an additional 4 h, after which solvents were removed in vacuo. The crude reaction mixture was purified by NP-Biotage (Method B) to provide S17 (154 mg, 0.187 mmol, 71.1%) as white solid. LCMS: m/z [M+H]⁺=825.19 (theoretical); 825.20 (observed). HPLC retention time=1.89 min (Method C).

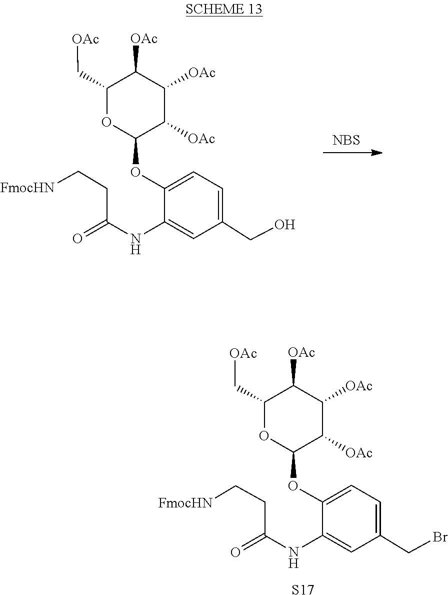

Synthesis of N-(3-(3-((((9H-fluoren-9-yl)methoxy)carbonyl)amino)propanamido)-4-(((2R,3 S,4S,5R,6R)-3,4,5-triacetoxy-6-(acetoxymethyl)tetrahydro-2H-pyran-2-yl)oxy)benzyl)-1-(4-((4-amino-2-butyl-7-(methoxycarbonyl)-1H-imidazo[4,5-c]quinolin-1-yl)methyl)phenyl)-N,N-dimethylmethanaminium (S18). A synthesitic route for intermediate S18 is provided in SCHEME 14 below. Compound 518a was prepared according to the procedures as described in Larson, et al. ACS Med. Chem. Lett. Vol. 8, No. 11, 1148-1152 (2017). Compound S17 (97.8 mg, 0.119 mmol) and S18a (44 mg, 0.099 mmol) were dissolved in DMA (1 mL). The reaction mixture was heated at 45° C. for 3 h and the reaction progress monitored by LCMS. After 3 h, the reaction mixture was diluted with DMSO/water and purified by RP-HPLC (Method A) to provide S18 (128.9 mg, 0.091 mmol, 92%) as TFA salt, white solid. LCMS: m/z [M]⁺=1190.51 (theoretical); 1190.75 (observed). HPLC retention time=1.81 min (Method D).

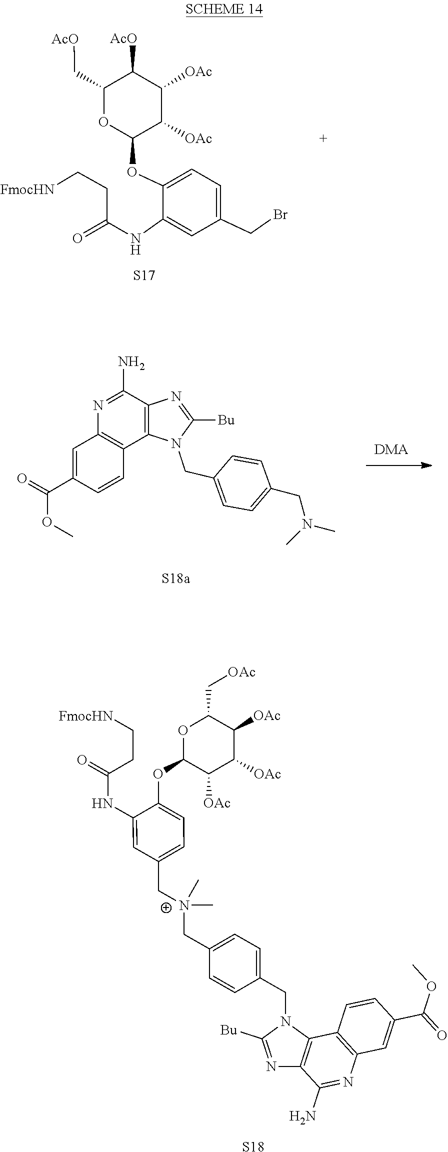

Synthesis of N-(4-((4-amino-2-butyl-7-carboxy-1H-imidazo[4,5-c]quinolin-1-yl)methyl)benzyl)-1-(3-(3-aminopropanamido)-4-(((2R,3 S,4 S, 5 S, 6R)-3,4,5-trihydroxy-6-(hydroxymethyl)tetrahydro-2H-pyran-2-yl)oxy)phenyl)-N,N-dimethylmethanaminium (S19). SCHEME 15 provides a synthetic scheme for generating intermediate S19. Intermediate S18 (87.9 mg, 0.062 mmol) was dissolved in THE (1.0 mL) and an aqueous LiOH solution (0.5 M, 1.24 mL, 0.62 mmol) was added. The reaction mixture was stirred at room temperature for 5 h, upon which LCMS indicated full conversion. The reaction mixture was quenched with HOAc (54 μl) and the THF was removed in vacuo. The remaining reaction mixture was diluted with DMSO/water and purified by RP-HPLC (Method A) to give intermediate S19 (50.4 mg, 0.045 mmol, 72.1%) as TFA salt, white solid. LCMS: m/z [M]⁺=786.38 (theoretical); 786.53 (observed). HPLC retention time=1.09 min (Method D).

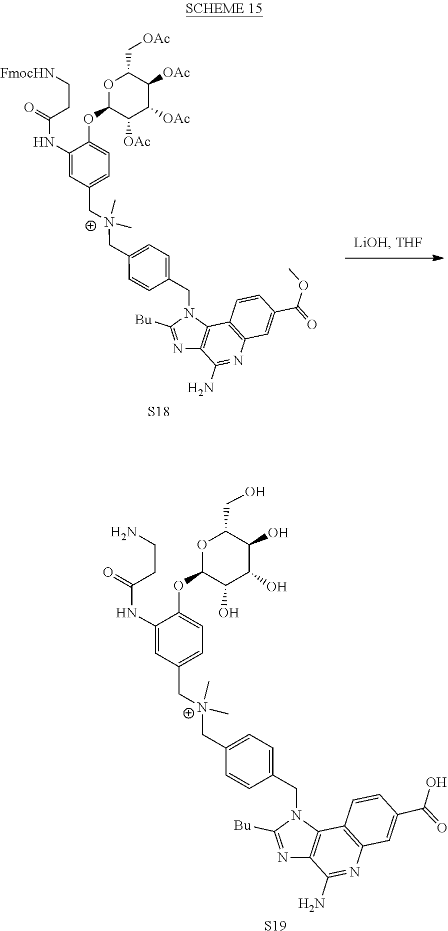

Synthesis of N-(4-((4-amino-2-butyl-7-carboxy-1H-imidazo[4,5-c]254uinoline-1-yl)methyl)benzyl)-1-(3-((S)-44-amino-38,45-di oxo-2, 5,8, 11,14,17,20,23,26,29,32,35-dodecaoxa-39,46-diazanonatetracontan-49-amido)-4-(((2R,3 S,4 S, 5 S, 6R)-3,4,5-trihydroxy-6-(hydroxymethyl)tetrahydro-2H-pyran-2-yl)oxy)phenyl)-N,N-dimethylmethanaminium (S21). SCHEME 16 summarizes a synthetic scheme for generating intermediate S21. Intermediate S19 (23.4 mg, 0.023 mmol) and Sla (35.8 mg, 0.035 mmol) were dissolved in anhydrous DMA (0.5 mL) and DIPEA (0.024 mL, 0.138 mmol) at room temperature. The reaction mixture was stirred at room temperature for 90 min. After 90 min, the crude reaction mixture was diluted with DMSO/water and purified by RP-HPLC (Method A) to give intermediate S21 (32.1 mg, 0.017 mmol, 71.9%) as TFA salt, white solid. LCMS: m/z [M]⁺=1706.87 (theoretical); 1706.91 (observed). HPLC retention time=1.71 min (Method D).

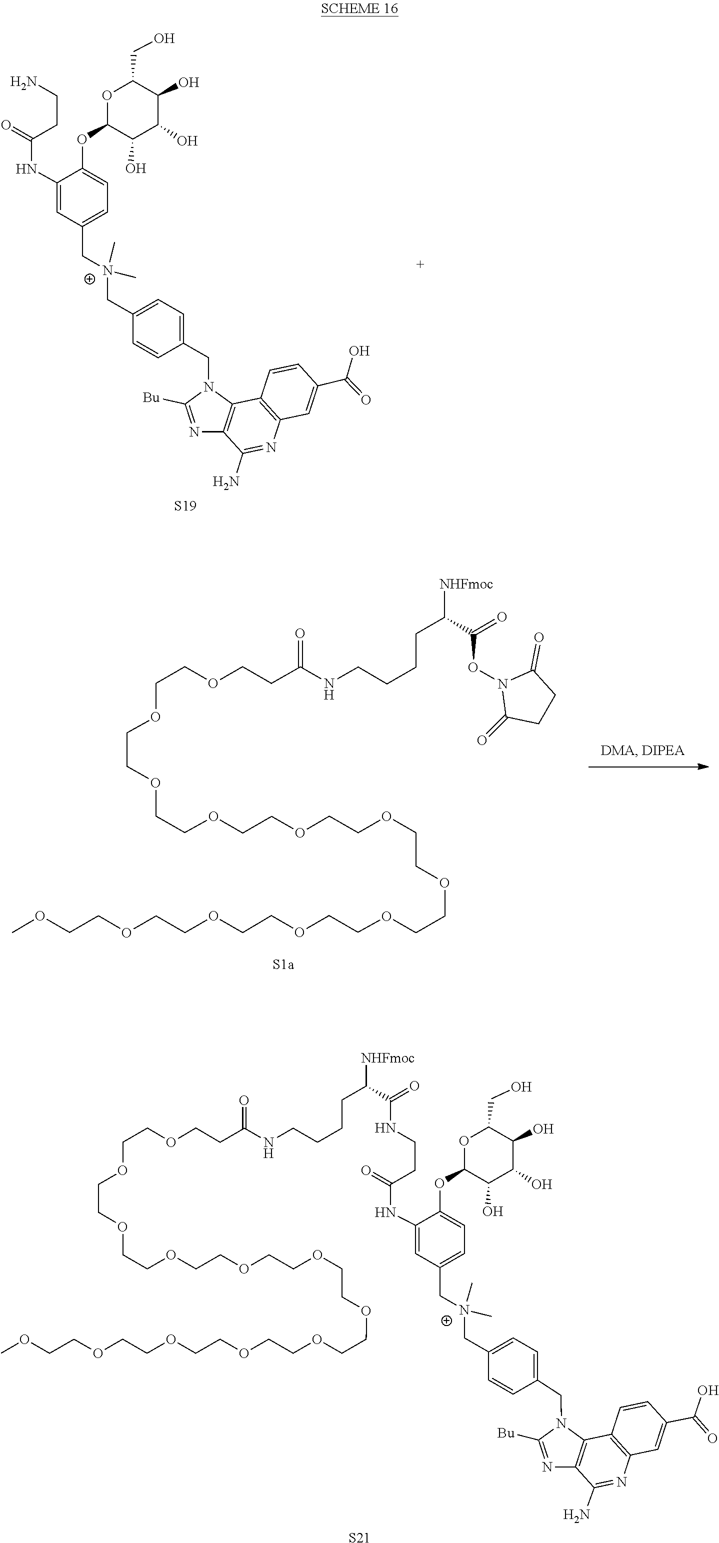

Synthesis of N-(4-((4-amino-2-butyl-7-carboxy-1H-imidazo[4,5-c]quinolin-1-yl)methyl)benzyl)-1-(3-((S)-44-amino-38,45-dioxo-2, 5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)phenyl)-N,N-dimethylmethanaminium (S22). SCHEME 17 summarizes a synthetic scheme for generating intermediate S22. Intermediate S21 (37.1, 0.022 mmol) was dissolved in 1 mL of a 4:1 (v/v) mixture of DCM/Et₂NH. The reaction mixture was stirred at room temperature for 45 min. After 45 min, solvents were removed in vacuo and the crude reaction mixture was diluted with DMSO/water and purified by RP-HPLC. (Method A) to give intermediate S22 (33.9 mg, 0.018 mmol, 82.9%) as TFA salt, white solid. LCMS: m/z [M]⁺=1484.80 (theoretical); 1484.62 (observed). HPLC retention time=1.46 min (Method D).

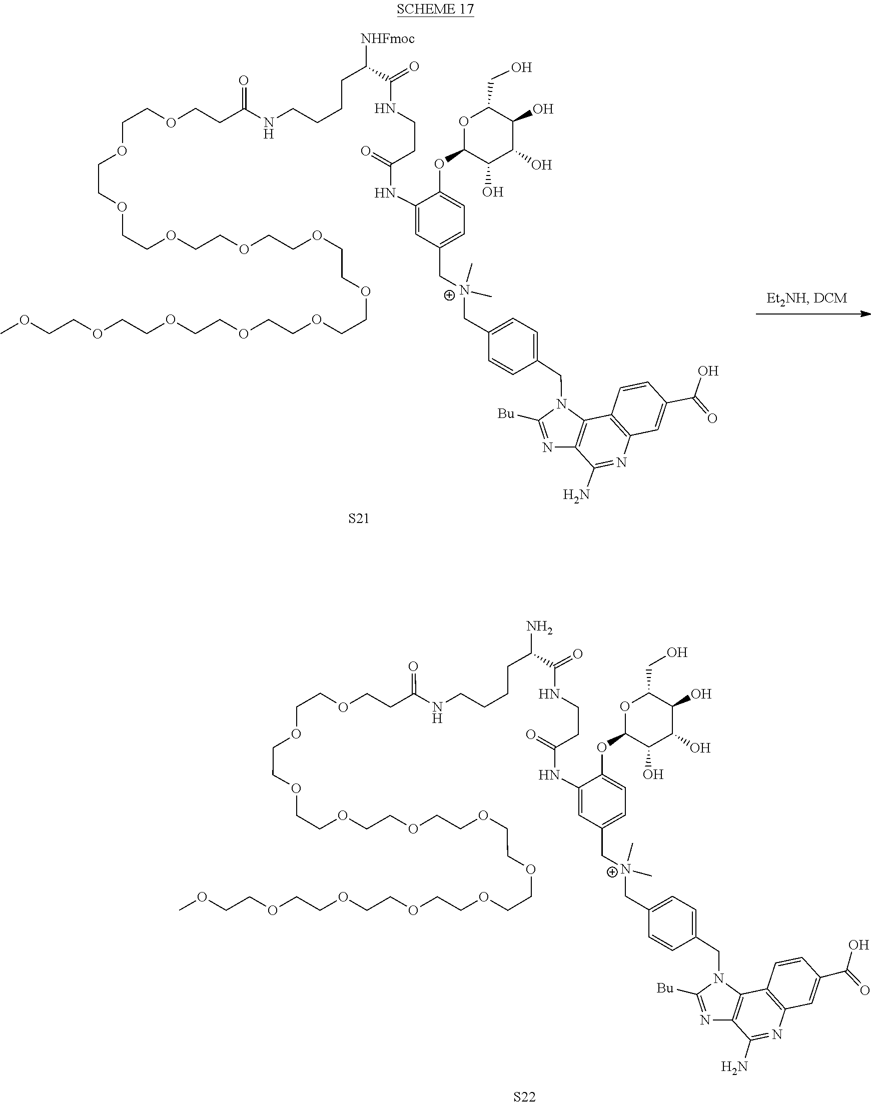

Synthesis of N-(4-((4-amino-2-butyl-7-carboxy-1H-imidazo[4,5-c]quinolin-1-yl)methyl)benzyl)-1-(3-((S)-44-(3-(2,5-di oxo-2,5-dihydro-1H-pyrrol-1-yl)propanamido)-38,45-di oxo-2, 5,8,11,14,17,20,23,26,29,32,35-dodecaoxa-39,46-diazanonatetracontan-49-amido)-4-(((2R,3 S,4 S,5 S,6R)-3,4, 5-trihydroxy-6-(hydroxymethyl)tetrahydro-2H-pyran-2-yl)oxy)phenyl)-N,N-dimethylmethanaminium (9). SCHEME 18 summarizes a synthetic scheme for generating compound 9. Intermediate S22 (33.9 mg, 0.018 mmol) and mp-OSu (6.99 mg, 0.026 mmol) were dissolved in anhydrous DMA (0.5 mL) and DIPEA (18.3 μL, 0.105 mmol) at room temperature. The reaction mixture was stirred at the same temperature for 30 min. After 30 min, the reaction mixture was quenched with HOAc (10 μl), diluted with DMSO/water, and purified by RP-HPLC (Method A) to give compound 9 (29.6 mg, 0.016 mmol, 90.7%) as TFA salt, white solid. LCMS: m/z [M]⁺=1635.83 (theoretical); 1637.38 (observed). HPLC retention time=1.51 min (Method D).

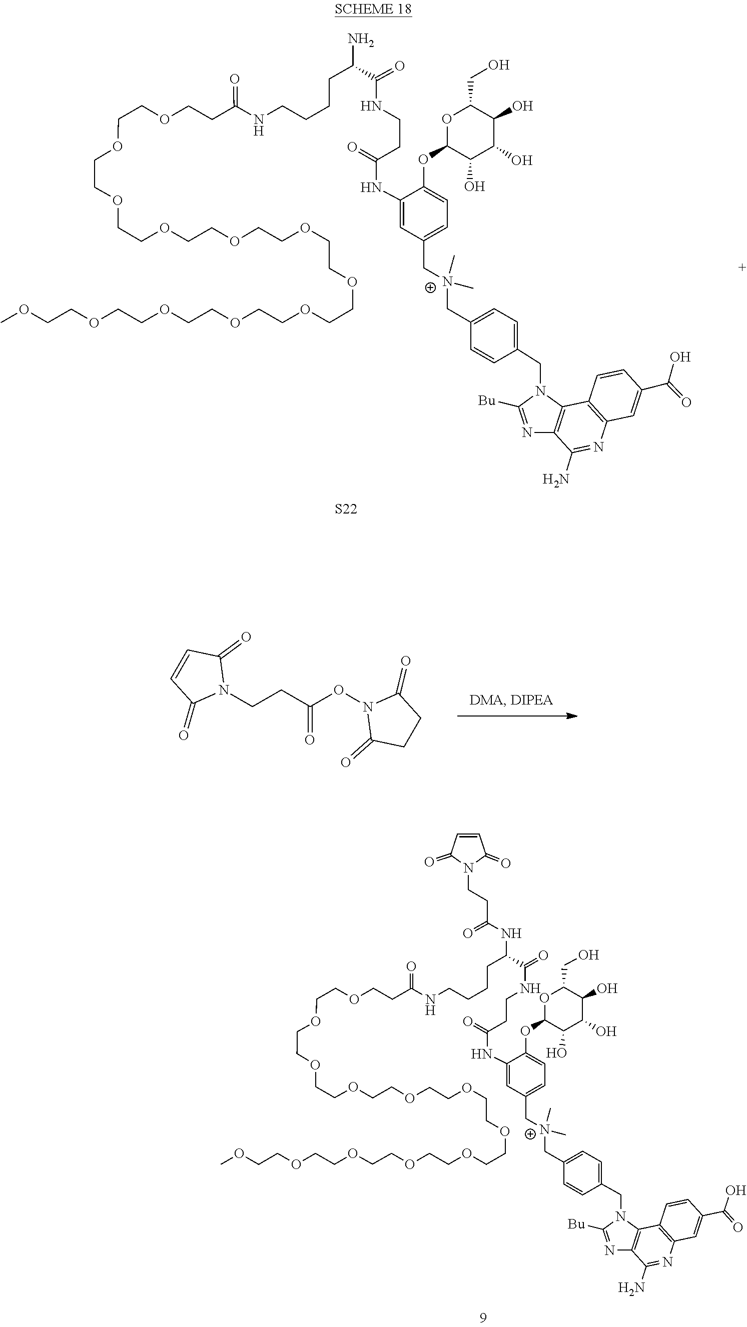

### Example 9

### Syntheses of a Drug-Linker Conjugate

This example covers syntheses of a complex comprising a linker coupled to an N1 position of an imidazoquinoline TLR7/8 agonist with methyl ester functionalization at its C₇ position, namely N-(4-((4-amino-2-butyl-7-(methoxycarbonyl)-1H-imidazo[4,5-c]quinolin-1-yl)methyl)benzyl)-1-(3-((S)-44-(3-(2,5-dioxo-2,5-dihydro-1H-pyrrol-1-yl)propanamido)-38,45-dioxo-2, 5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)phenyl)-N,N-dimethylmethanaminium (10).

Synthesis of N-(4-((4-amino-2-butyl-7-(methoxycarbonyl)-1H-imidazo[4,5-c]quinolin-1-yl)methyl)benzyl)-1-(3-(3-aminopropanamido)-4-(((2R,3 S,4 S, 5 S,6R)-3,4,5-trihydroxy-6-(hydroxymethyl)tetrahydro-2H-pyran-2-yl)oxy)phenyl)-N,N-dimethylmethanaminium (S23). SCHEME 19 outlines a synthetic scheme for generating intermediate S23. The intermediate S18 (14.2 mg, 0.01 mmol) was dissolved in MeOH (0.4 mL) and cooled at 0° C. for 5 min. NaOMe solution (0.5M in MeOH, 0.12 mL, 0.06 mmol) was added. The reaction was mixture stirred at 0° C. for 30 min and then at room temperature for another 2 h. After 2 hours, the solution was neutralized with HOAc (11.6 μL) and purified by RP-HPLC (Method A) to give intermediate S23 (7.8 mg, 0.008 mmol, 76.8%) as TFA salt, white solid. LCMS: m/z [M]⁺=800.40 (theoretical); 800.54 (observed). HPLC retention time=1.51 min (Method D).

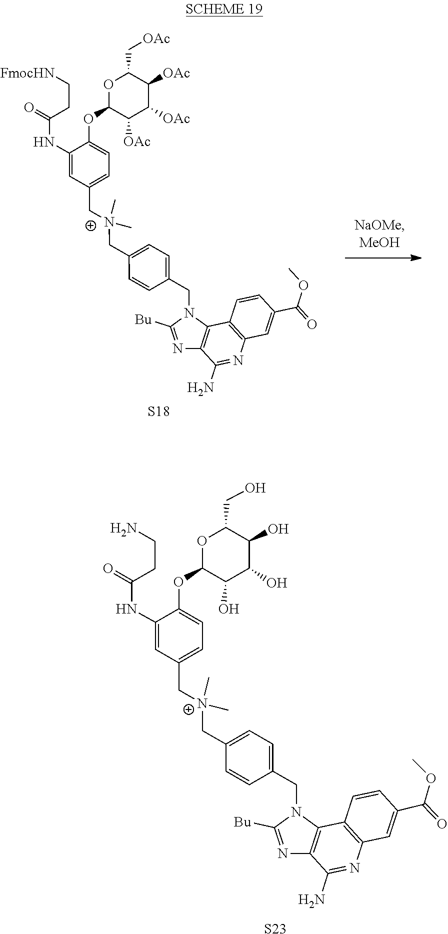

Synthesis of N-(3-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-(((2R,3 S,4S,5 S,6R)-3,4,5-trihydroxy-6-(hydroxymethyl)tetrahydro-2H-pyran-2-yl)oxy)benzyl)-1-(4-((4-amino-2-butyl-7-(methoxycarbonyl)-1H-imidazo[4,5-c]quinolin-1-yl)methyl)phenyl)-N,N-dimethylmethanaminium (S24). SCHEME 20 outlines a synthetic route for intermediate S24. Intermediates S23 (15.6 mg, 15.2 μmol) and Sla (23.6 mg, 22.8 μmol) were dissolved in anhydrous DMA (0.8 mL) and DIPEA (13.2 μL, 0.076 mmol) at room temperature. The reaction mixture was stirred at room temperature for 30 min. After 30 min, the crude reaction mixture was diluted with DMSO/water and purified by RP-HPLC (Method A) to give intermediate S24 (17.2 mg, 0.012 mmol, 70.3%) as TFA salt, white solid. LCMS: m/z [M+H]⁺=1721.89 (theoretical); 1722.34 (observed). HPLC retention time=1.51 min (Method D).

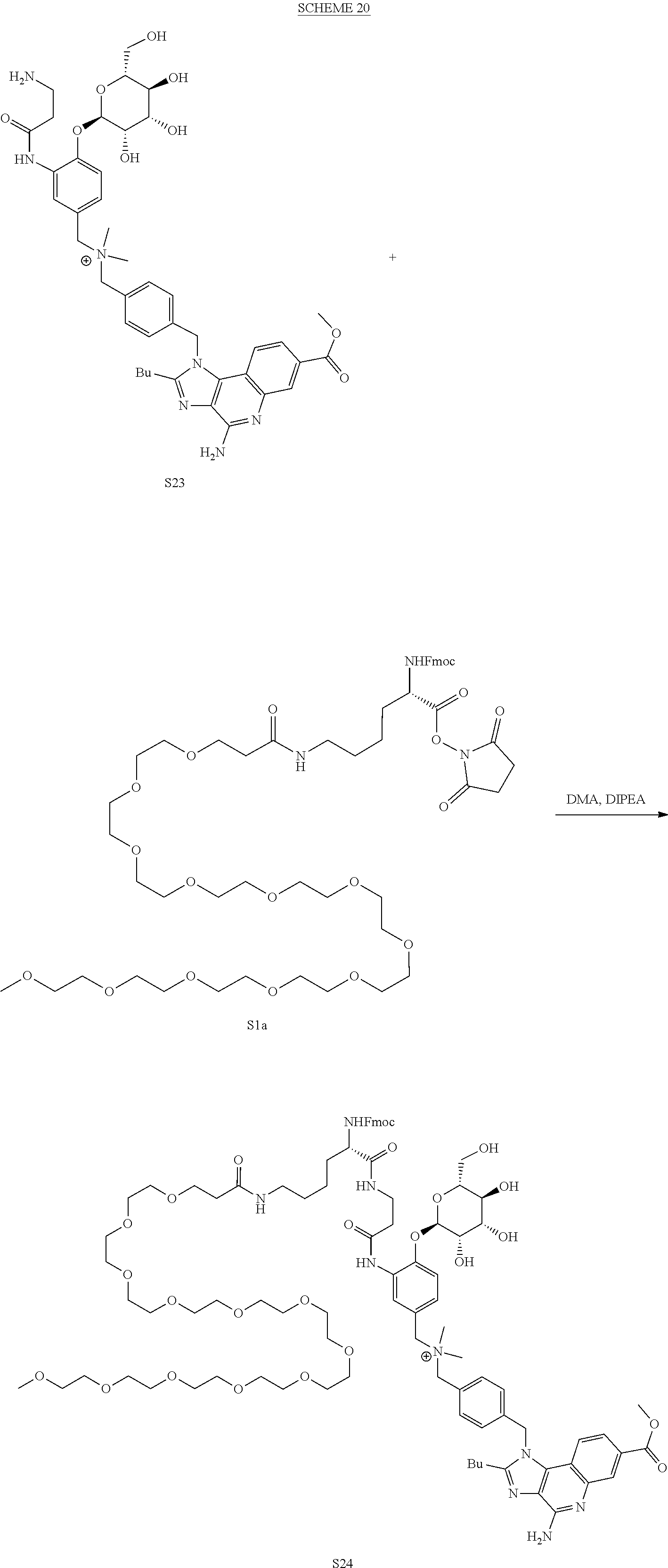

Synthesis of N-(4-((4-amino-2-butyl-7-(methoxycarbonyl)-1H-imidazo[4,5-c]quinolin-1-yl)methyl)benzyl)-1-(3-((S)-44-amino-38,45-dioxo-2,5,8,11,14,17,20,23,26,29,32,35-dodecaoxa-39,46-diazanonatetracontan-49-amido)-4-(((2R,3 S,4S,5 S,6R)-3,4,5-trihydroxy-6-(hydroxymethyl)tetrahydro-2H-pyran-2-yl)oxy)phenyl)-N,N-dimethylmethanaminium (S25). SCHEME 21 outlines a synthetic route for generating intermediate S25. Intermediate S24 (17.2, 0.012 mmol) was dissolved in 1 mL of a 4:1(v/v) mixture of DCM/Et₂NH. The reaction mixture was stirred at room temperature for 1 h. After 1 hour, solvents were removed in vacuo and the crude reaction mixture was diluted with DMSO/water and purified by RP-HPLC (Method A) to give intermediate S25 (14.4 mg, 9.6 μmol, 80.1%) as TFA salt, white solid. LCMS: m/z [M]⁺=1498.82 (theoretical); 1499.12 (observed). HPLC retention time=1.33 min (Method D).

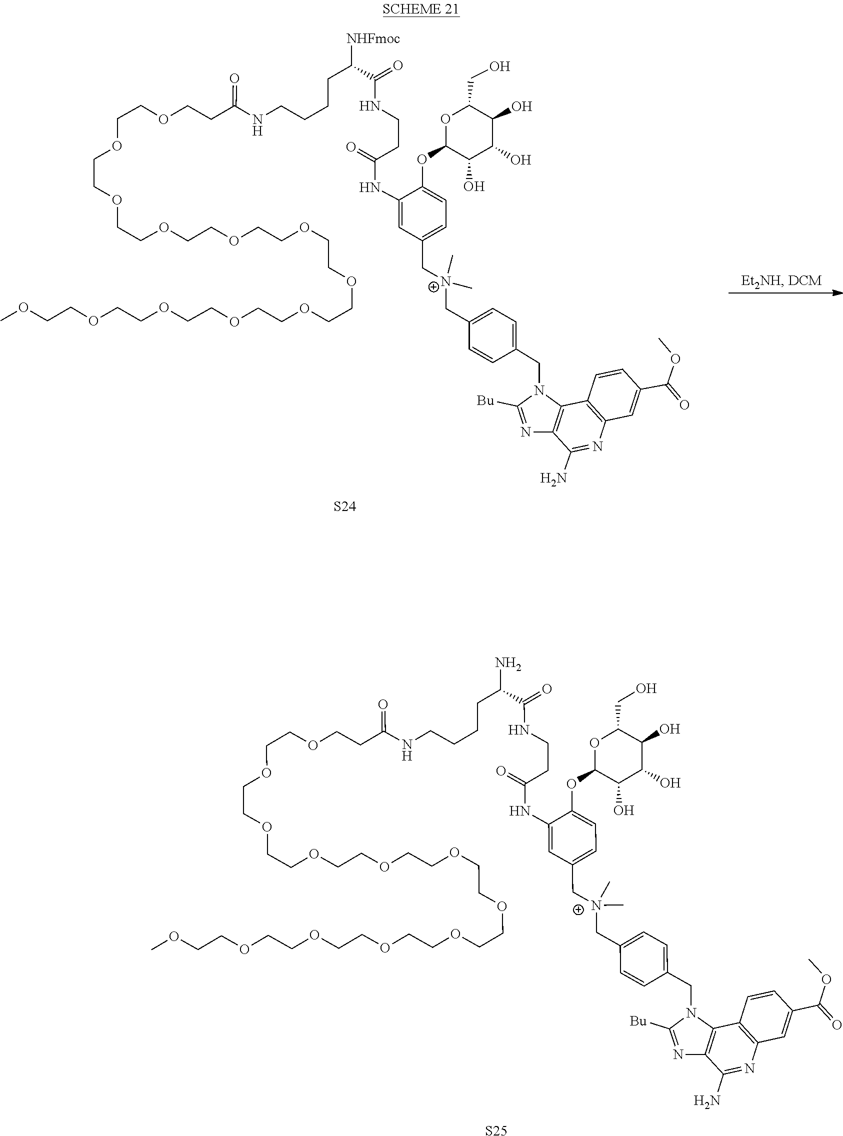

Synthesis of N-(4-((4-amino-2-butyl-7-(methoxycarbonyl)-1H-imidazo[4,5-c]quinolin-1-yl)methyl)benzyl)-1-(3-((S)-44-(3-(2,5-di oxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)phenyl)-N,N-dimethylmethanaminium (10). SCHEME 22 outlines a synthesis of compound 10 from intermediate S25. Intermediate S25 (9.5 mg, 0.006 mmol) and mp-OSu (2.35 mg, 8.83 μmol) were dissolved in anhydrous DMA (0.2 mL) and DIPEA (5.13 μL, 0.029 mmol) at room temperature. The reaction mixture was stirred at the same temperature for 30 min. After 30 min, the reaction mixture was quenched with HOAc (10 μl), diluted with DMSO/water and purified by RP-HPLC (Method A) to give compound 10 (8.6 mg, 0.005 mmol, 82.8%) as TFA salt, white solid. LCMS: m/z [M]⁺=1649.8 (theoretical); 1650.2 (observed). HPLC retention time=1.41 min (Method D).

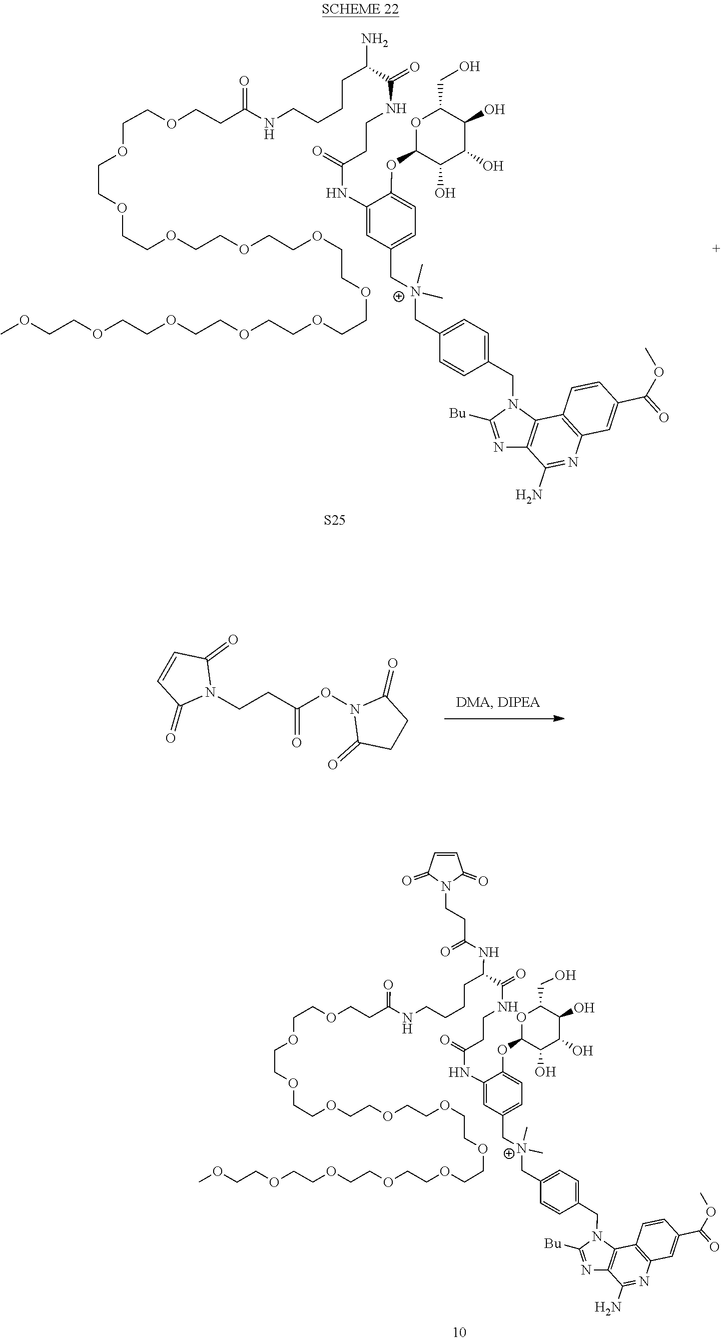

### Example 10

### Syntheses of a Drug-Linker Conjugate

This example covers syntheses of a complex comprising a linker coupled to a C₄ amine of an imidazoquinoline TLR7/8 agonist with methyl ester functionalization at its C₇ position and phenyl substitution at N1, namely 2-butyl-1-(4-((dimethylamino)methyl)benzyl)-4-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)-1H-imidazo[4,5-c]quinoline-7-carboxylic acid (11).

Synthesis of (2R,3S,4S,5R,6R)-2-(2-(3-((((9H-fluoren-9-yl)methoxy)carbonyl)amino)propanamido)-4-((((2-butyl-1-(4-((dimethylamino)methyl)benzyl)-7-(methoxycarbonyl)-1H-imidazo[4,5-c]259uinoline-4-yl)carbamoyl)oxy)methyl)phenoxy)-6-(acetoxymethyl)tetrahydro-2H-pyran-3,4,5-triyl triacetate (S27). Intermediate S27 was synthesized according to SCHEME 23. Compound 518a (75 mg, 0.168 mmol) was dissolved in anhydrous THE (1.5 mL) and cooled to 0° C. 1,1′-carbonyl-do-(1,2,4-triazole) (CDT, 38.7 mg, 0.236 mmol) was added and the reaction mixture was warmed up to room temperature and stirred for 30 min. After 30 min, compound S26 (191 mg, 0.421 mmol) was added to the reaction mixture. The reaction was stirred at room temperature for 2 h. After 2 hours, solvents were removed in vacuo and the crude reaction mixture was purified by normal phase HPLC (Method B, EtOAc/hexane as eluents) to give the desired product S27 (125.7 mg, 0.136 mmol, 80.6%) as white solid. LCMS: m/z [M+H]⁺=1234.49 (theoretical); 1234.91 (observed). HPLC retention time=2.13 min (Method D).

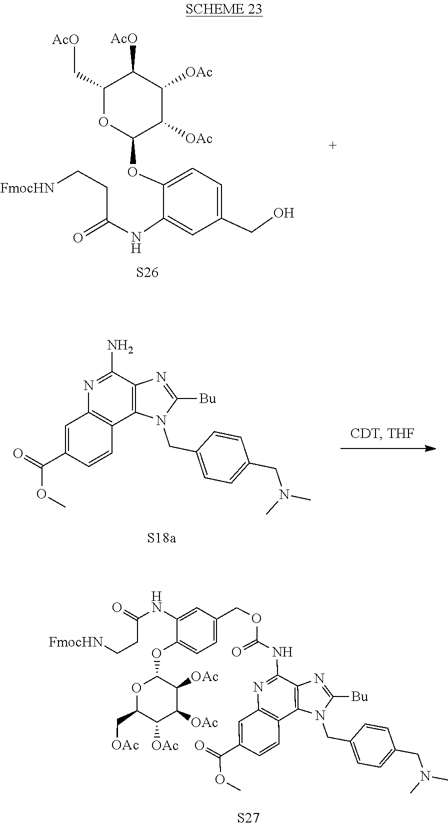

Synthesis of 4-((((3-(3-aminopropanamido)-4-(((2R,3S,4S,5 S,6R)-3,4,5-trihydroxy-6-(hydroxymethyl)tetrahydro-2H-pyran-2-yl)oxy)benzyl)oxy)carbonyl)amino)-2-butyl-1-(4-((dimethylamino)methyl)benzyl)-1H-imidazo[4,5-c]quinoline-7-carboxylic acid (S28). Intermediate S28 was synthesized according to SCHEME 24. Compound S27 (110.0 mg, 0.082 mmol) was dissolved in a 4:1 mixture of Et₂NH and DCM (1.25 mL). The reaction was stirred at room temperature for 30 min. After 30 min, solvents were removed in vacuo and the residue was re-dissolved in THF (1.3 mL), followed by the addition of an aqueous LiOH solution (0.5 M, 1.3 mL, 0.653 mmol). The reaction mixture was stirred at room temperature for 6 h. After 6 h, the reaction mixture was acidified with glacial acetic acid (70 μL), THF was removed in vacuo and the residue was diluted with DMSO/water and purified by RP-HPLC (Method A) to provide S28 (62.5 mg, 0.0591 mmol, 72.4%) as TFA salt, white solid. LCMS: m/z [M+H]⁺=830.37 (theoretical); 830.62 (observed). HPLC retention time=1.34 min (Method D).

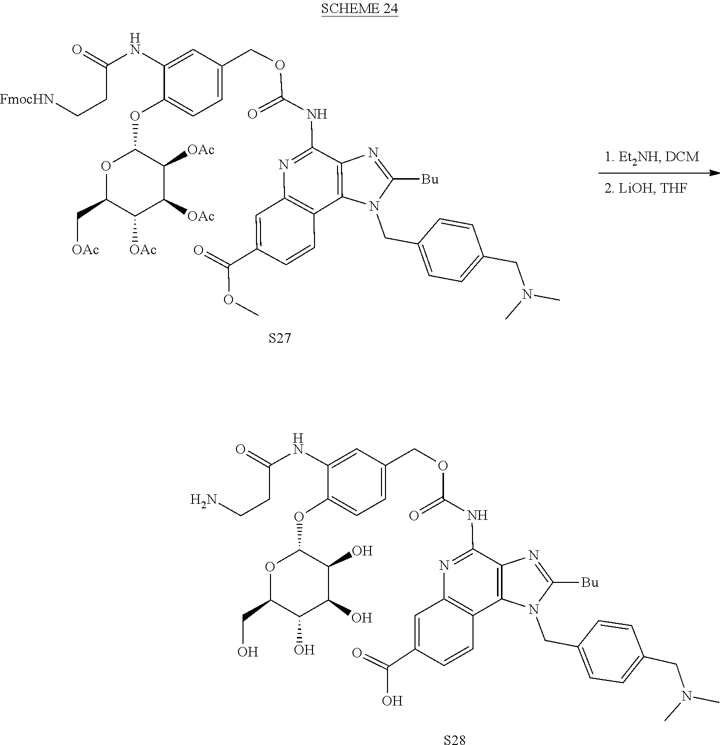

Synthesis of 4-((((3-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-(((2R,3 S,4S,5 S,6R)-3,4,5-trihydroxy-6-(hydroxymethyl)tetrahydro-2H-pyran-2-yl)oxy)benzyl)oxy)carbonyl)amino)-2-butyl-1-(4-((dimethylamino)methyl)benzyl)-1H-imidazo[4,5-c]quinoline-7-carboxylic acid (S29). SCHEME 25 provides a synthetic route for generating intermediate S29. Intermediates S28 (20.0 mg, 0.019 mmol) and Sla (29.4 mg, 0.028 mmol) were dissolved in anhydrous DMA (0.6 mL) and DIPEA (16.5 μL, 0.095 mmol) at room temperature. The reaction mixture was stirred at room temperature for 1 h. After 1 h, the crude reaction mixture was diluted with DMSO/water and purified by RP-HPLC (Method A) to give intermediate S29 (21.4 mg, 0.0122 mmol, 64.7%) as TFA salt, white solid. LCMS: m/z [M+H]⁺=1750.85 (theoretical); 1752.29 (observed). HPLC retention time=1.52 min (Method D).

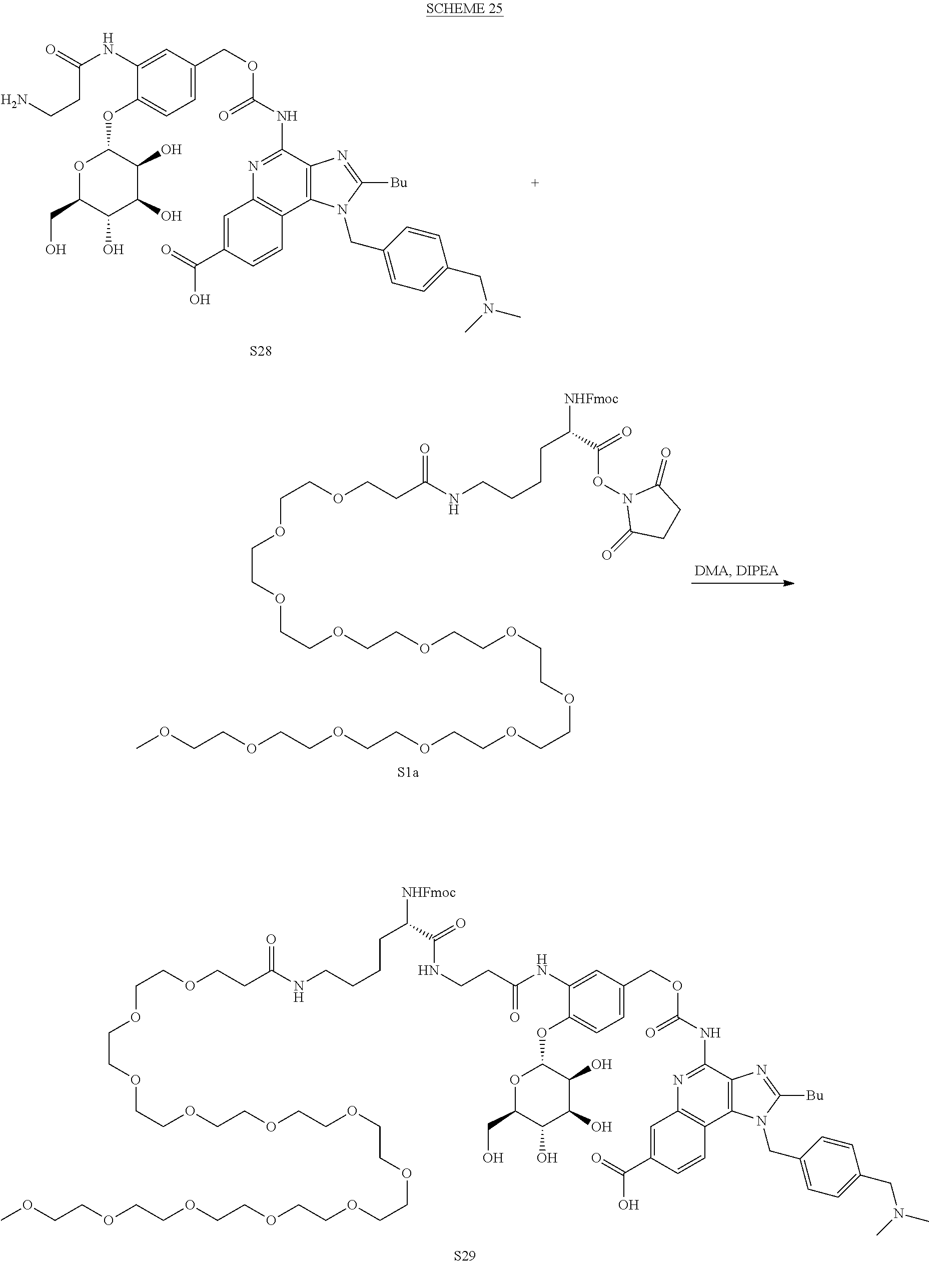

Synthesis of 4-((((3-((S)-44-amino-38,45-dioxo-2,5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)-2-butyl-1-(4-((dimethylamino)methyl)benzyl)-1H-imidazo[4,5-c]quinoline-7-carboxylic acid (S30). Intermediate S30 was synthesized according to SCHEME 26. Intermediate S29 (21.4, 0.0122 mmol) was dissolved in 1 mL of a 4:1 (v/v) mixture of DCM/Et₂NHmL. The reaction mixture was stirred at room temperature for 40 min. After 40 min, solvents were removed in vacuo and the crude reaction mixture was diluted with DMSO/water and purified by RP-HPLC (Method A) to give intermediate S30 (17.4 mg, 9.92 μmol, 81.3%) as TFA salt, white solid. LCMS: m/z [M+H]⁺=1528.79 (theoretical); 1529.16 (observed). HPLC retention time=1.36 min (Method D).

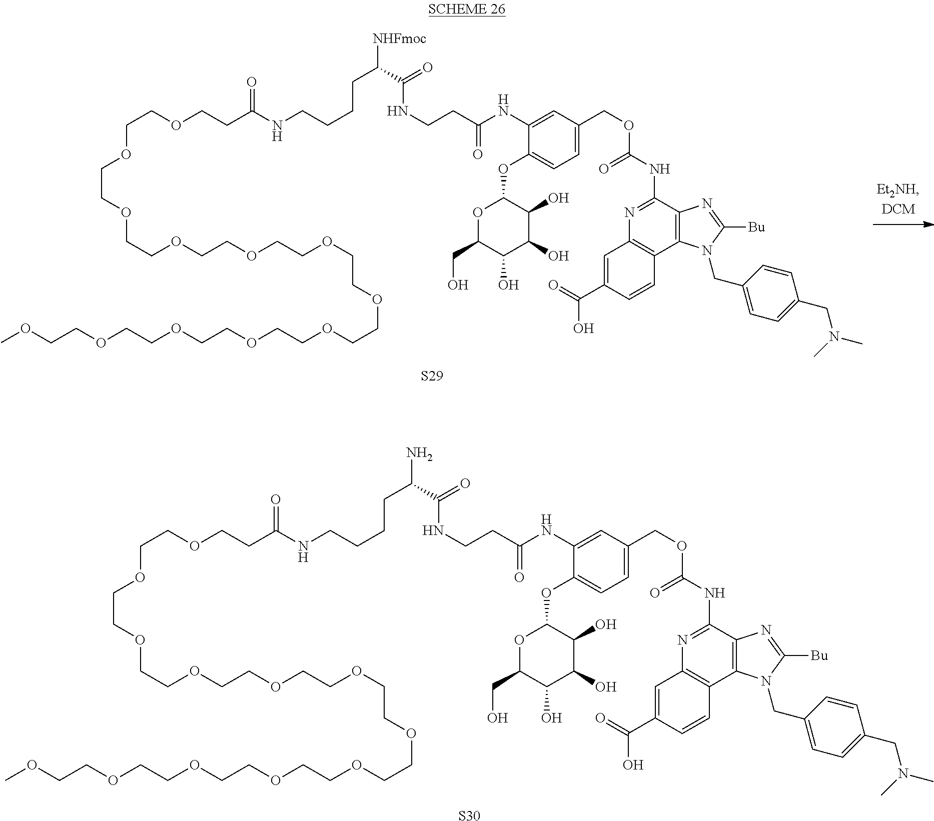

Synthesis of 2-butyl-1-(4-((dimethylamino)methyl)benzyl)-4-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4S,5 S,6R)-3,4,5-trihydroxy-6-(hydroxymethyl)tetrahydro-2H-pyran-2-yl)oxy)benzyl)oxy)carbonyl)amino)-1H-imidazo[4,5-c]quinoline-7-carboxylic acid (11). Compound 11 was synthesized according to SCHEME 27. Intermediate S30 (23.7 mg, 0.0144 mmol) and mp-OSu (5.76 mg, 0.0216 mmol) were dissolved in anhydrous DMA (0.5 mL) and DIPEA (12.6 μL, 0.072 mmol) at room temperature. The reaction mixture was stirred at the same temperature for 30 min. After 30 min, the reaction mixture was quenched with HOAc (15 μl), diluted with DMSO/water and purified by RP-HPLC (Method A) to give compound 11 (21.0 mg, 0.0117 mmol, 81.1%) as TFA salt. LCMS: m/z [M+H]⁺=1679.81 (theoretical); 1680.16 (observed). HPLC retention time=1.46 min (Method D).

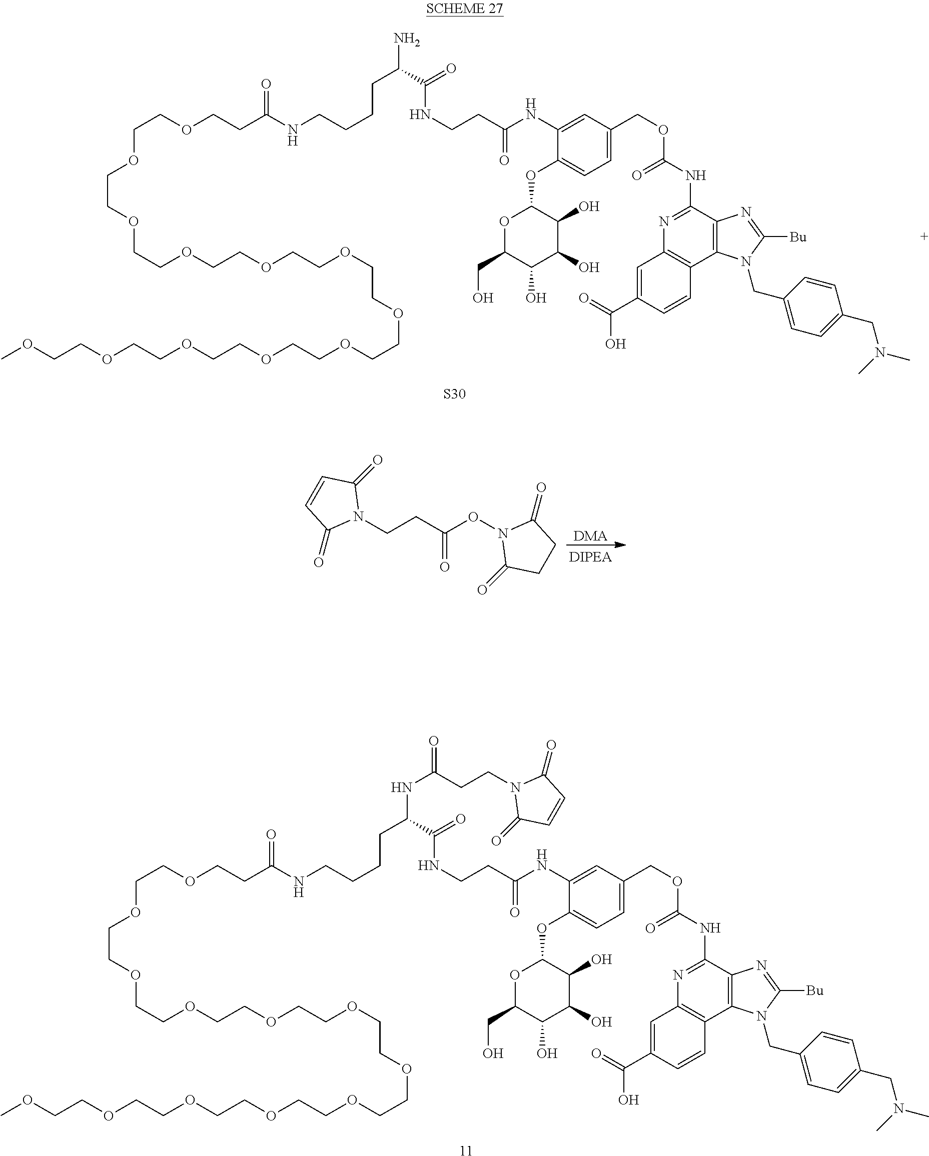

### Example 11

### Syntheses of a Drug-Linker Conjugate

This example covers syntheses of a complex comprising a linker coupled to a C₄ amine of an imidazoquinoline TLR7/8 agonist with methyl ester functionalization at its C_{7 position and phenyl substitution at N}1, namely methyl 2-butyl-1-(4-((dimethylamino)methyl)benzyl)-4-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)-1H-imidazo[4,5-c]quinoline-7-carboxylate (12).

Synthesis of methyl 4-((((3-(3-aminopropanamido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-2-butyl-1-(4-((dimethylamino)methyl)benzyl)-1H-imidazo[4,5-c]quinoline-7-carboxylate (S31). Intermediate S31 was generated according to SCHEME 28. Intermediate S28 (39.3 mg, 0.029 mmol) was dissolved in a 4:1 (v/v) mixture of Et₂NH and DCM (1.25 mL). The reaction mixture was stirred at room temperature for 30 min, After 30 min, solvents were removed in vacuo and the residue was re-dissolved in MeOH (0.4 mL) followed by the addition of a NaOMe solution (0.5 M in MeOH, 0.35 mL, 0.175 mmol). The reaction mixture was stirred at room temperature for 1 h. After 1 hour, the reaction mixture was acidified with glacial acetic acid (15 μL), THE was removed in vacuo and the residue was diluted with DMSO/water and purified by RP-HPLC (Method A) to provide S31 (14.3 mg, 0.013 mmol, 45.4%) as TFA salt, white solid. LCMS: m/z [M+H]⁺=844.38 (theoretical); 844.62 (observed). HPLC retention time=1.47 min (Method D).

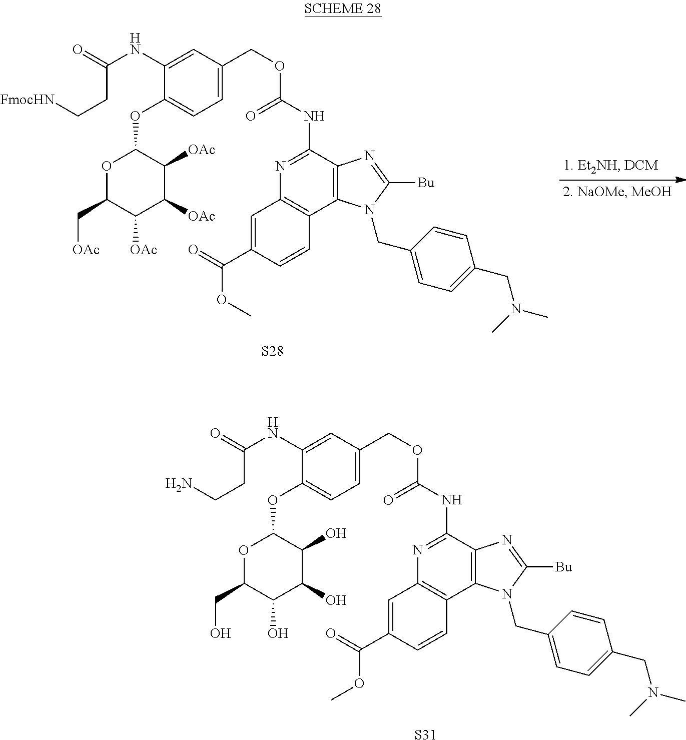

Synthesis of methyl 4-((((3-((S)-44-((((9H-fluoren-9-yl)methoxy)carbonyl)amino)-38,45-dioxo-2, 5,8,11,14,17,20,23,26,29,32,35-dodecaoxa-39,46-diazanonatetracontan-49-amido)-4-(((2R,3 S,4 S, 5 S,6R)-3,4,5-trihydroxy-6-(hydroxymethyl)tetrahydro-2H-pyran-2-yl)oxy)benzyl)oxy)carbonyl)amino)-2-butyl-1-(4-((dimethylamino)methyl)benzyl)-1H-imidazo[4,5-c] quinoline-7-carboxylate (S32). Intermediate S32 was generated according to SCHEME 29. Intermediates S31 (14.3 mg, 0.013 mmol) and Sla (20.7 mg, 0.02 mmol) were dissolved in anhydrous DMA (0.6 mL) and DIPEA (13.9 μL, 0.08 mmol) at room temperature. The reaction mixture was stirred at room temperature for 1 h. After 1 h, the crude reaction mixture was diluted with DMSO/water and purified by RP-HPLC (Method A) to give intermediate S32 (16.1 mg, 9.1 μmol, 68.4%) as TFA salt, white solid. LCMS: m/z [M+H]⁺=1764.87 (theoretical); 1766.33 (observed). HPLC retention time=1.84 min (Method D).

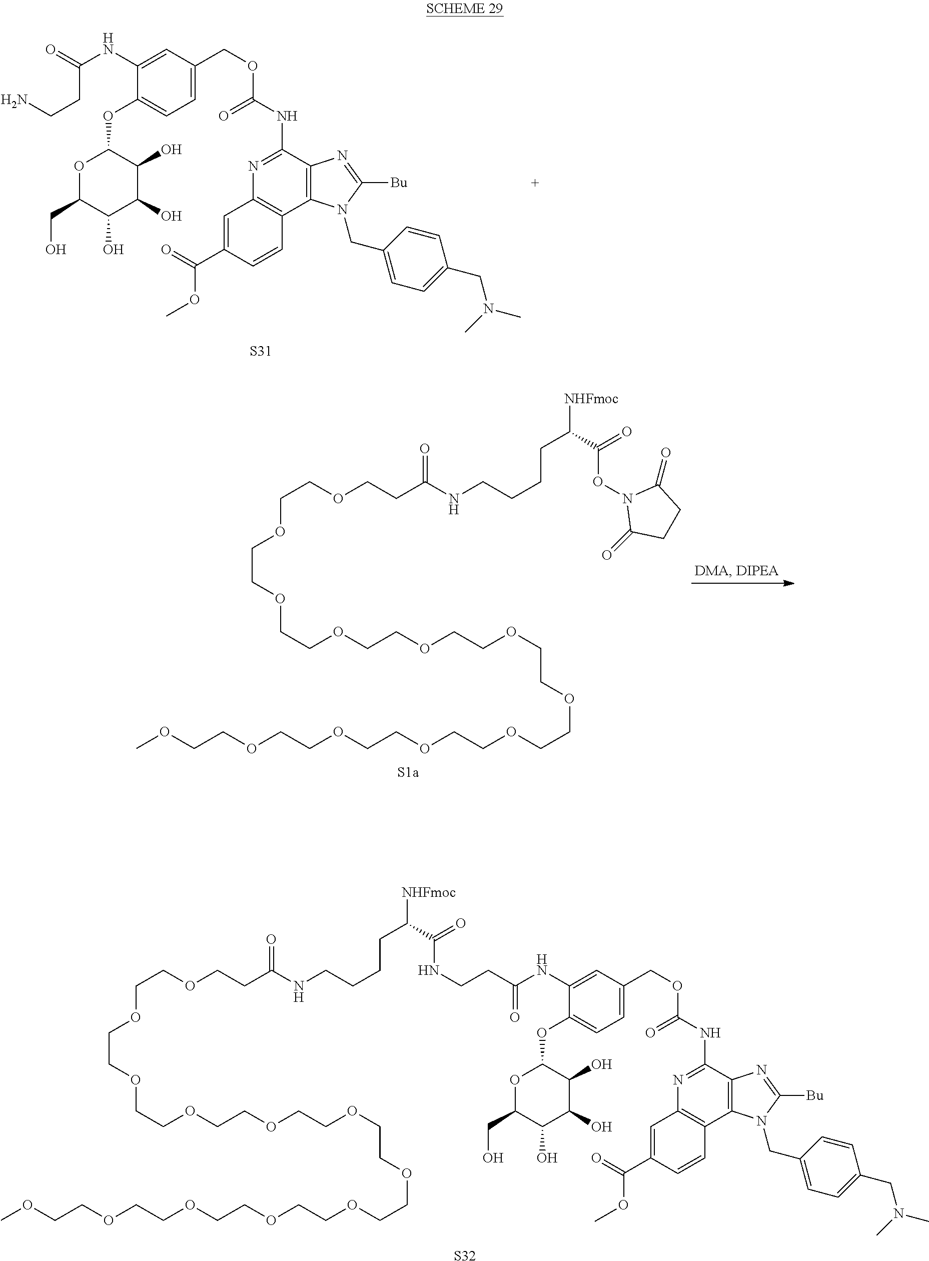

Synthesis of methyl 4-((((3-((S)-44-amino-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-2-butyl-1-(4-((dimethylamino)methyl)benzyl)-1H-imidazo[4,5-c]quinoline-7-carboxylate (S33). Intermediate S33 was synthesized according to SCHEME 30. Intermediate S32 (16.1 mg, 9.1 μmol) was dissolved in 1 mL of a 4:1 (v/v) mixture of DCM/Et₂NHmL. The reaction mixture was stirred at room temperature for 1 h. After 1 hour, solvents were removed in vacuo and the crude reaction mixture was diluted with DMSO/water, and purified by RP-HPLC (Method A) to give intermediate S33 (10.2 mg, 6.6 μmol, 72.2%) as TFA salt, white solid. LCMS: m/z [M+H]⁺=1542.80 (theoretical); 1543.27 (observed). HPLC retention time=1.56 min (Method D).

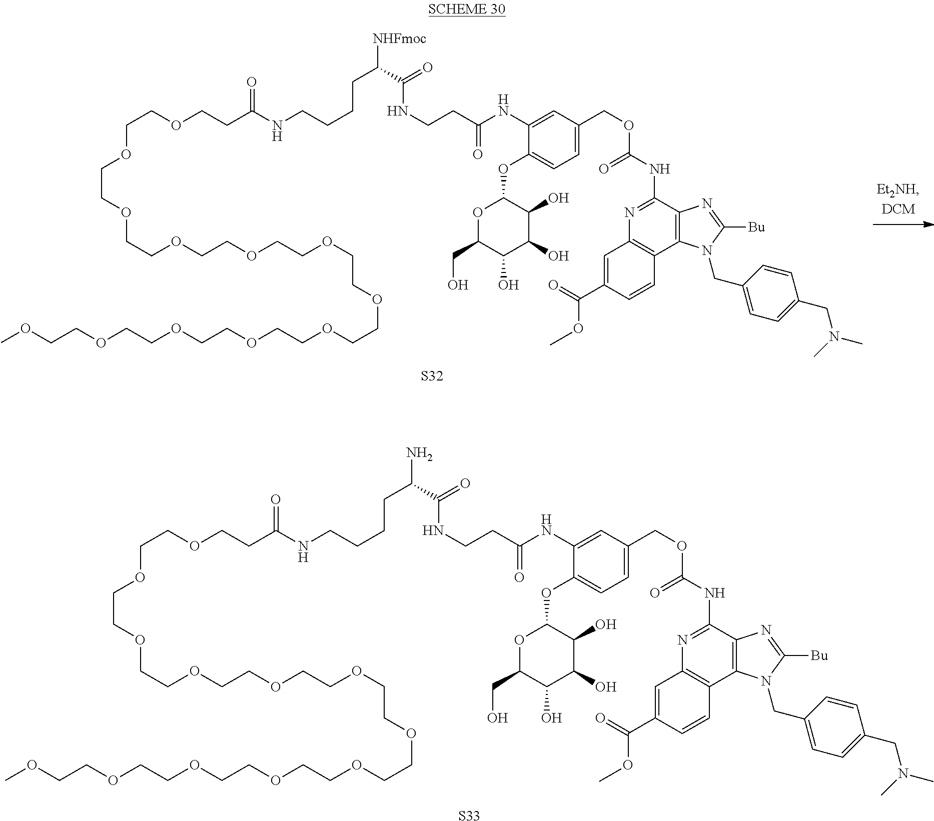

Synthesis of methyl 2-butyl-1-(4-((dimethylamino)methyl)benzyl)-4-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4S,5S,6R)-3,4,5-trihydroxy-6-(hydroxymethyl)tetrahydro-2H-pyran-2-yl)oxy)benzyl)oxy)carbonyl)amino)-1H-imidazo[4,5-c]quinoline-7-carboxylate (12). Compound 12 was generated according to SCHEME 31. Intermediate S33 (17.7 mg, 0.01 mmol) and mp-OSu (3.99 mg, 0.015 mmol) were dissolved in anhydrous DMA (0.5 mL) and DIPEA (10.4 μL, 0.06 mmol) at room temperature. The reaction mixture was stirred at the same temperature for 45 min. After 45 min, the reaction mixture was quenched with HOAc (10 μL), diluted with DMSO/water, and purified by RP-HPLC (Method A) to give compound 12 (14.9 mg, 0.008 mmol, 82.5%) as TFA salt, white solid. LCMS: m/z [M+H]⁺=1693.8 (theoretical); 1694.4 (observed). HPLC retention time=1.66 min (Method D).

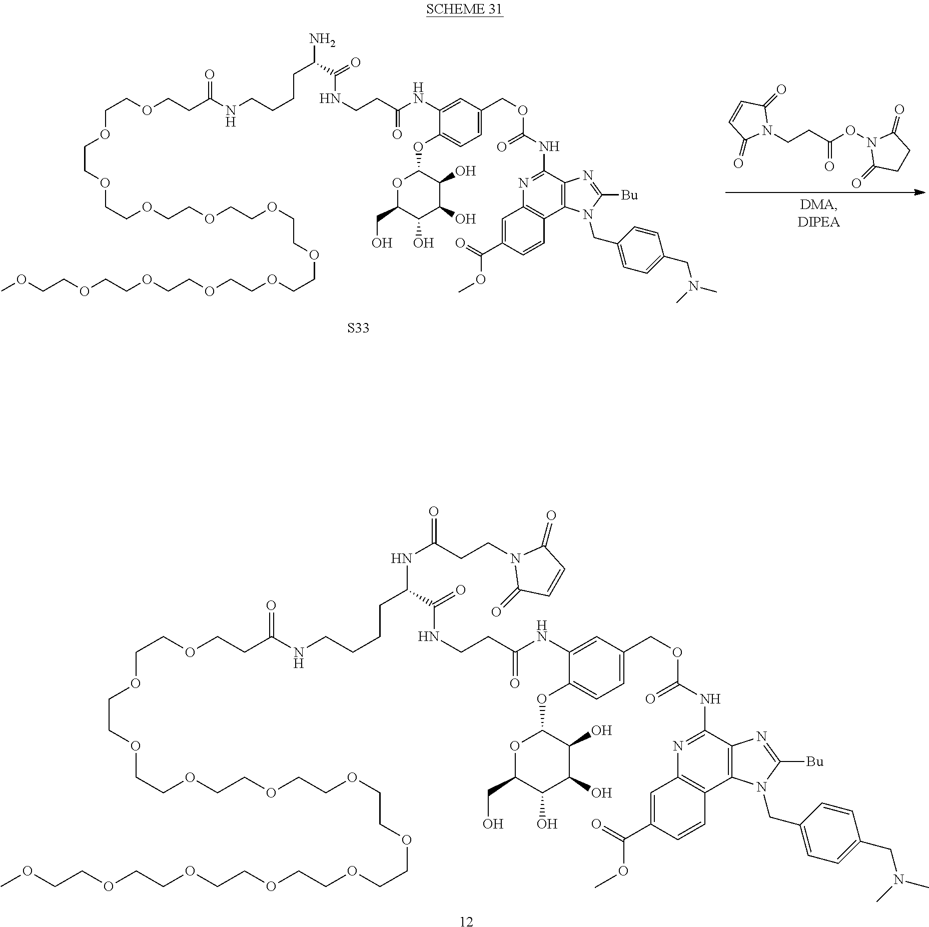

### Example 12

### Syntheses of Two Drug-Linker Conjugates

This example covers syntheses of two complexes comprising a linker coupled to a N1 positions of imidazoquinoline TLR7/8 agonists with either carboxylic acid or methyl ester functionalization at their C₇ positions, namely N-(4-((4-amino-2-butyl-7-(methoxycarbonyl)-1H-imidazo[4,5-c]quinolin-1-yl)methyl)benzyl)-1-(3-(3-(3-(2,5-dioxo-2,5-dihydro-1H-pyrrol-1-yl)propanamido)propanamido)-4-(((2R,3 S,4S,5S,6R)-3,4,5-trihydroxy-6-(hydroxymethyl)tetrahydro-2H-pyran-2-yl)oxy)phenyl)-N,N-dimethylmethanaminium (13) and N-(4-((4-amino-2-butyl-7-carboxy-1H-imidazo[4,5-c]quinolin-1-yl)methyl)benzyl)-1-(3-(3-(3-(2,5-dioxo-2,5-dihydro-1H-pyrrol-1-yl)propanamido)propanamido)-4-(((2R,3 S,4S,5S,6R)-3,4,5-trihydroxy-6-(hydroxymethyl)tetrahydro-2H-pyran-2-yl)oxy)phenyl)-N,N-dimethylmethanaminium (14).

Synthesis of N-(4-((4-amino-2-butyl-7-(methoxycarbonyl)-1H-imidazo[4,5-c]quinolin-1-yl)methyl)benzyl)-1-(3-(3-(3-(2,5-dioxo-2,5-dihydro-1H-pyrrol-1-yl)propanamido)propanamido)-4-(((2R,3 S,4S,5S,6R)-3,4,5-trihydroxy-6-(hydroxymethyl)tetrahydro-2H-pyran-2-yl)oxy)phenyl)-N,N-dimethylmethanaminium (13). Compound 13 was synthesized according to SCHEME 32. Intermediate S23 (1.9 mg, 1.85 μmol) and mp-OSu (0.64 mg, 0.002 mmol) were dissolved in anhydrous DMA (0.1 mL) and DIPEA (1.61 μLμL, 0.009 mmol) at room temperature. The reaction mixture was stirred at the same temperature for 90 min. After 90 min, the reaction mixture was quenched with HOAc (10 μL), diluted with DMSO/water, and purified by RP-HPLC (Method A) to give compound 13 (1.8 mg, 0.002 mmol, 91.4%) as TFA salt, white solid. LCMS: m/z [M]⁺=951.43 (theoretical); 951.62 (observed). HPLC retention time=1.27 min (Method D).

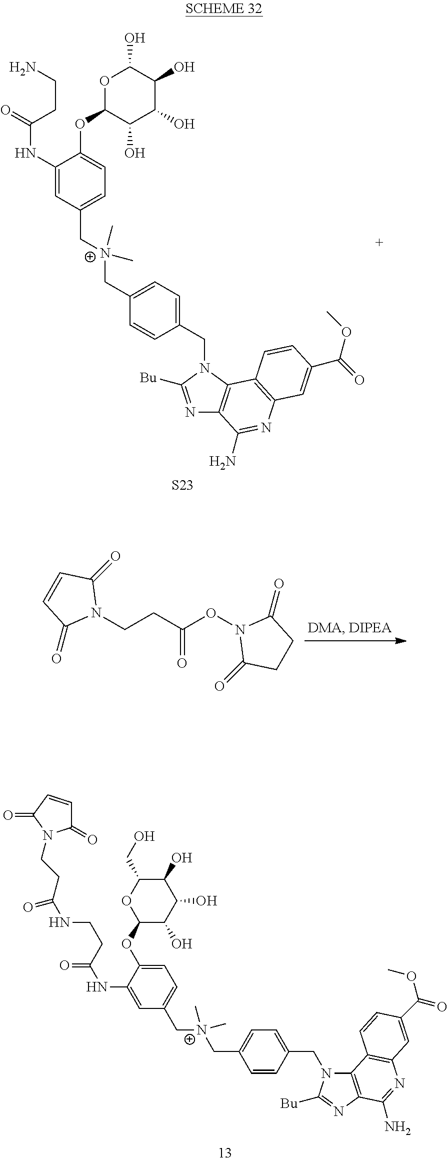

N-(4-((4-amino-2-butyl-7-carboxy-1H-imidazo[4,5-c]quinolin-1-yl)methyl)benzyl)-1-(3-(3-(3-(2,5-dioxo-2,5-dihydro-1H-pyrrol-1-yl)propanamido)propanamido)-4-(((2R,3S,4S,5S,6R)-3,4,5-trihydroxy-6-(hydroxymethyl)tetrahydro-2H-pyran-2-yl)oxy)phenyl)-N,N-dimethylmethanaminium (14). Synthesis of compound 14 was performed according to SCHEME 33. Intermediate S19 (3.5 mg, 3.45 μmol) and mp-OSu (1.38 mg, 0.005 mmol) were dissolved in anhydrous DMA (0.2 mL) and DIPEA (3.0 μL, 0.017 mmol) at room temperature. The reaction mixture was stirred at the same temperature for 90 min. After 90 min, the reaction mixture was quenched with HOAc (10 μL), diluted with DMSO/water, and purified by RP-HPLC (Method A) to give compound 14 (1.7 mg, 0.002 mmol, 52.6%) as TFA salt, white solid. LCMS: m/z [M]⁺=937.41 (theoretical); 937.58 (observed). HPLC retention time=1.21 min (Method D).

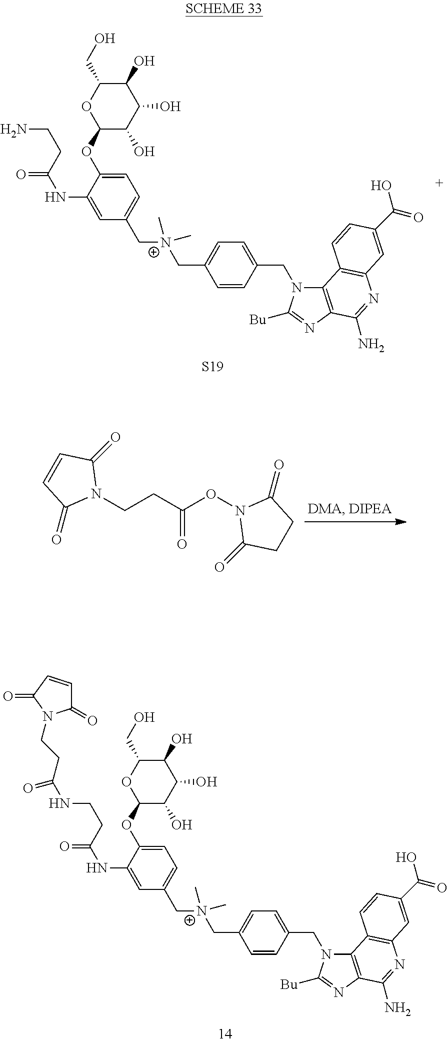

### Example 13

### Syntheses of a Drug-Linker Conjugate

This example covers synthesis of a complex comprising a linker coupled to a C4 amine of an imidazoquinoline TLR7/8 agonist with carboxylic acid functionalization at its C₇ position and N1 phenyl functionalization, namely 2-butyl-1-(4-((dimethylamino)methyl)benzyl)-4-((((3-(3-(3-(2,5-dioxo-2,5-dihydro-1H-pyrrol-1-yl)propanamido)propanamido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-1H-imidazo[4,5-c]quinoline-7-carboxylic acid (15). This synthesis was performed according to SCHEME 34. Intermediate S28 (25.5 mg, 0.024 mmol) and mp-OSu (9.62 mg, 0.036 mmol) were dissolved in anhydrous DMA (1.5 mL) and DIPEA (21 μL, 120.5 mmol) at room temperature. The reaction mixture was stirred at the same temperature for 30 min. After 30 min, the reaction mixture was quenched with HOAc (25 μL), diluted with DMSO/water, and purified by RP-HPLC (Method A) to give compound 15 (20.6 mg, 21.0 mmol, 87.1%) as TFA salt, white solid. LCMS: m/z [M+H]⁺=981.39 (theoretical); 981.61 (observed). HPLC retention time=1.32 min (Method D).

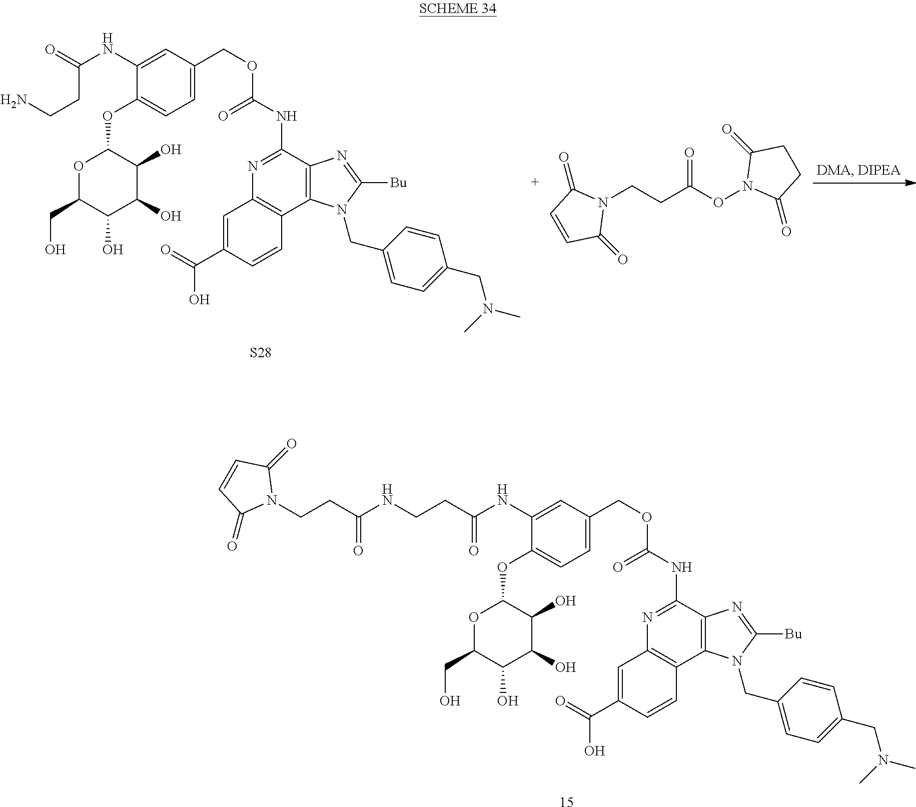

### Example 14

### Syntheses of a Drug-Linker Conjugate

This example covers synthesis of a complex comprising a linker coupled to a C₄ amine of an imidazoquinoline TLR7/8 agonist with carboxylic acid functionalization at its C₇ position and N1 phenyl functionalization, namely 2-butyl-1-(4-((dimethylamino)methyl)benzyl)-4-((((3-(1-(2,5-dioxo-2,5-dihydro-1H-pyrrol-1-yl)-3,31-dioxo-7,10,13,16,19,22,25,28-octaoxa-4,32-diazapentatriacontan-35-amido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-1H-imidazo[4,5-c]quinoline-7-carboxylic acid (16). This synthesis is summarized in SCHEME 35. Intermediate S28 (7.1 mg, 67 μmmol) and S28a (mp-PEG8-OSu, Broadpharm, 6.94 mg, 0.01 mmol) were dissolved in anhydrous DMA (1.3 mL) and DIPEA (7.0 μL, 0.04 mmol) at room temperature. The reaction mixture was stirred at the same temperature for 1 h. After 1 h, the reaction mixture was quenched with HOAc (10 μL), diluted with DMSO/water, and purified by RP-HPLC (Method A) to give compound 16 (5.8 mg, 38 μmol, 56.9%) as TFA salt, white solid. LCMS: m/z [M+H]⁺=1404.64 (theoretical); 1405.09 (observed). HPLC retention time=1.56 min (Method D).

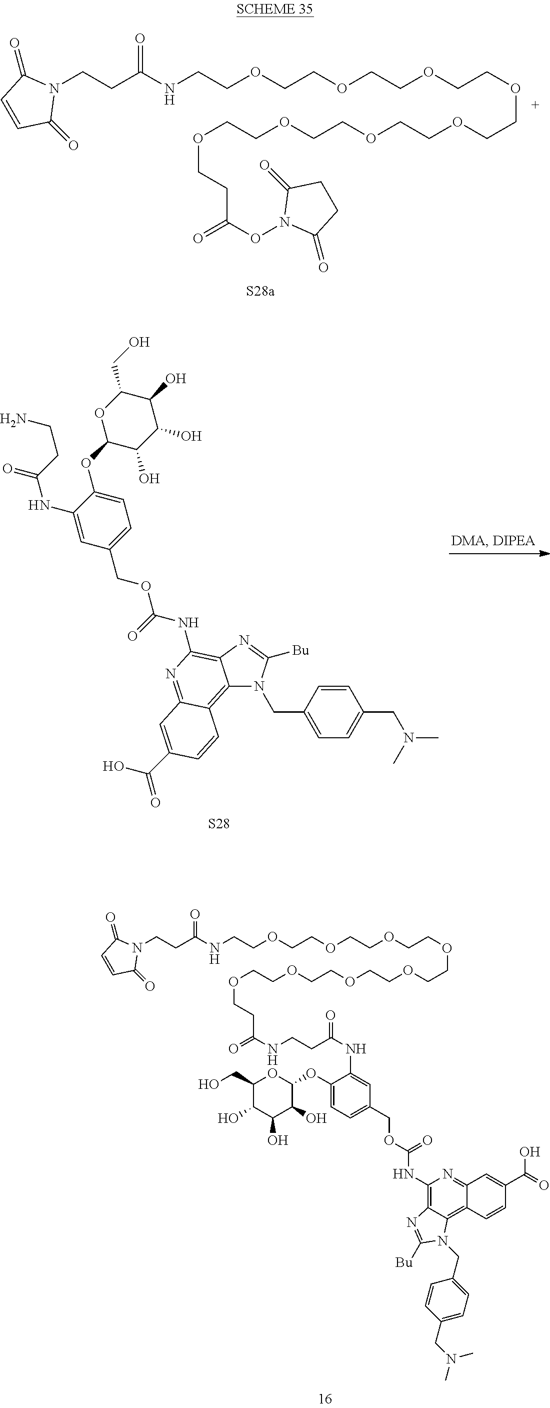

### Example 15

### Syntheses of Two Drug-Linker Conjugates

This example covers syntheses of complexes comprising linkers coupled to a N1 positions of imidazoquinoline TLR7/8 agonists with carboxylic acid or methyl ester functionalization at their C₇ positions, namely N-(4-((4-amino-2-butyl-7-(methoxycarbonyl)-1H-imidazo[4,5-c]quinolin-1-yl)methyl)benzyl)-1-(4-(((2S,3R,4S,5S,6S)-6-carboxy-3,4,5-trihydroxytetrahydro-2H-pyran-2-yl)oxy)-3-(3-(3-(2,5-dioxo-2,5-dihydro-1H-pyrrol-1-yl)propanamido)propanamido)phenyl)-N,N-dimethylmethanaminium (17) and N-(4-((4-amino-2-butyl-7-carboxy-1H-imidazo[4,5-c]quinolin-1-yl)methyl)benzyl)-1-(4-(((2S,3R,4S,5S,6S)-6-carboxy-3,4,5-trihydroxytetrahydro-2H-pyran-2-yl)oxy)-3-(3-(3-(2,5-dioxo-2,5-dihydro-1H-pyrrol-1-yl)propanamido)propanamido)phenyl)-N,N-dimethylmethanaminium (18). As outlined in SCHEME 36, the syntheses of compounds 18 and 17 was similar to the syntheses of compounds 13 and 14, respectively.

The synthesis of compound 18 and compound 17 was similar to the synthesis of compound 13 and compound 14, respectively.

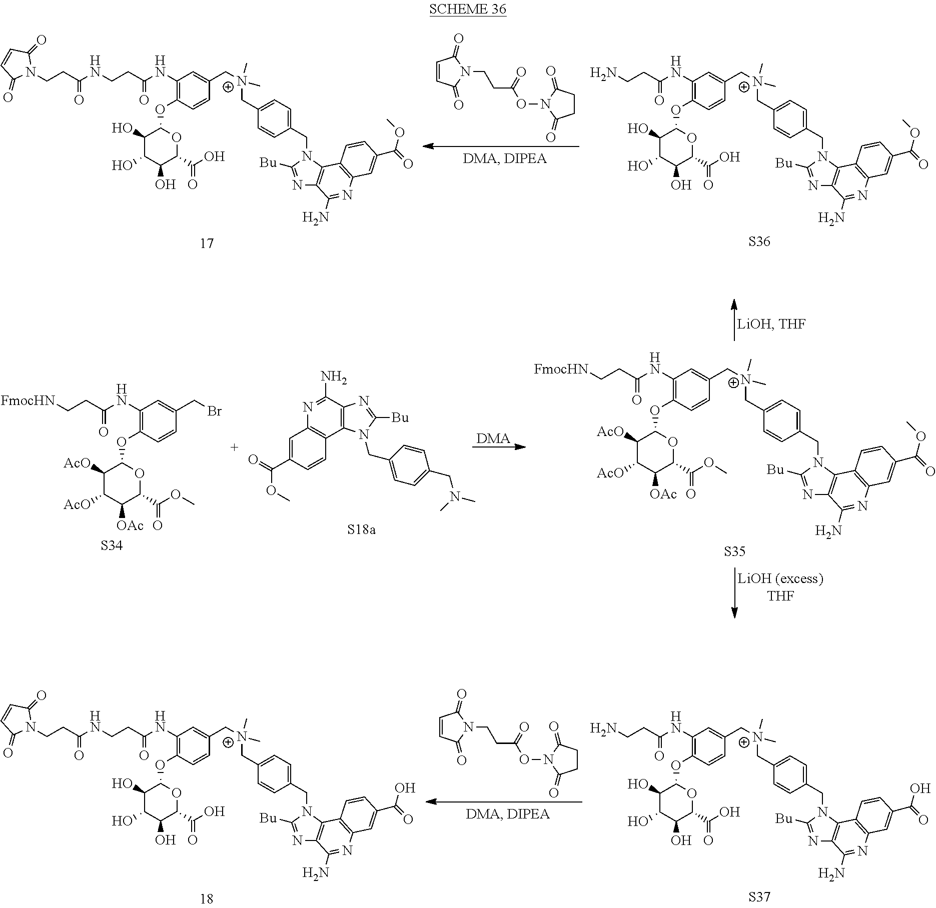

Compound S34 was prepared according to the procedures as described in Burke, et al. Mol. Cancer Ther. Vol. 17, 1752-1760 (2018).

N-(3-(3-((((9H-fluoren-9-yl)methoxy)carbonyl)amino)propanamido)-4-(((2S,3R,4S,5S,6S)-3,4,5-triacetoxy-6-(methoxycarbonyl)tetrahydro-2H-pyran-2-yl)oxy)benzyl)-1-(4-((4-amino-2-butyl-7-(methoxycarbonyl)-1H-imidazo[4,5-c]quinolin-1-yl)methyl)phenyl)-N,N-dimethylmethanaminium (S35) as TFA salt, white solid. LCMS: m/z [M]⁺=1176.49 (theoretical); 1176.77 (observed). HPLC retention time=1.83 min (Method D).

N-(4-((4-amino-2-butyl-7-(methoxycarbonyl)-1H-imidazo[4,5-c]quinolin-1-yl)methyl)benzyl)-1-(3-(3-aminopropanamido)-4-(((2 S,3R,4 S, 5S, 6S)-6-carboxy-3,4, 5-trihydroxytetrahydro-2H-pyran-2-yl)oxy)phenyl)-N,N-dimethylmethanaminium (S36) as TFA salt, white solid. LCMS: m/z [M]⁺=814.38 (theoretical); 814.56 (observed). HPLC retention time=1.23 min (Method D).

N-(4-((4-amino-2-butyl-7-carboxy-1H-imidazo[4,5-c]quinolin-1-yl)methyl)benzyl)-1-(3-(3-aminopropanamido)-4-(((2S,3R,4S,5S,6S)-6-carboxy-3,4,5-trihydroxytetrahydro-2H-pyran-2-yl)oxy)phenyl)-N,N-dimethylmethanaminium (S37) as TFA salt, white solid. LCMS: m/z [M]⁺=800.36 (theoretical); 800.53 (observed). HPLC retention time=1.12 min (Method D).

N-(4-((4-amino-2-butyl-7-(methoxycarbonyl)-1H-imidazo[4,5-c]quinolin-1-yl)methyl)benzyl)-1-(4-(((2S,3R,4S,5S,6S)-6-carboxy-3,4,5-trihydroxytetrahydro-2H-pyran-2-yl)oxy)-3-(3-(3-(2,5-dioxo-2,5-dihydro-1H-pyrrol-1-yl)propanamido)propanamido)phenyl)-N,N-dimethylmethanaminium (17) as TFA salt, white solid. LCMS: m/z [M]⁺=965.40 (theoretical); 965.42 (observed). HPLC retention time=1.43 min (Method D).

N-(4-((4-amino-2-butyl-7-carboxy-1H-imidazo[4,5-c]quinolin-1-yl)methyl)benzyl)-1-(4-(((2S,3R,4S,5S,6S)-6-carboxy-3,4,5-trihydroxytetrahydro-2H-pyran-2-yl)oxy)-3-(3-(3-(2,5-dioxo-2,5-dihydro-1H-pyrrol-1-yl)propanamido)propanamido)phenyl)-N,N-dimethylmethanaminium (18) as TFA salt, white solid. LCMS: m/z [M]⁺=951.39 (theoretical); 951.60 (observed). HPLC retention time=1.31 min (Method D).

### Example 16

### Syntheses of Two Drug-Linker Conjugates

This example covers syntheses of complexes comprising linkers coupled to a N1 positions of imidazoquinoline TLR7/8 agonists with carboxylic acid or methyl ester functionalization at their C₇ positions, namely N-(4-((4-amino-2-butyl-7-(methoxycarbonyl)-1H-imidazo[4,5-c]quinolin-1-yl)methyl)benzyl)-1-(4-(((2S,3R,4S,5S,6S)-6-carboxy-3,4,5-trihydroxytetrahydro-2H-pyran-2-yl)oxy)-3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)phenyl)-N,N-dimethylmethanaminium (19) and N-(4-((4-amino-2-butyl-7-carboxy-1H-imidazo[4,5-c]quinolin-1-yl)methyl)benzyl)-1-(4-(((2S,3R,4S,5S,6S)-6-carboxy-3,4,5-trihydroxytetrahydro-2H-pyran-2-yl)oxy)-3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)phenyl)-N,N-dimethylmethanaminium (20). The syntheses of compound 20 and 19 are similar to the syntheses of compounds 9 and 10, respectively. Syntheses of intermediates S38 and S39 and compound 19 are outlined in SCHEME 37. Synthesis of intermediates S40 and S41 and compound 20 are outlined in SCHEME 38.

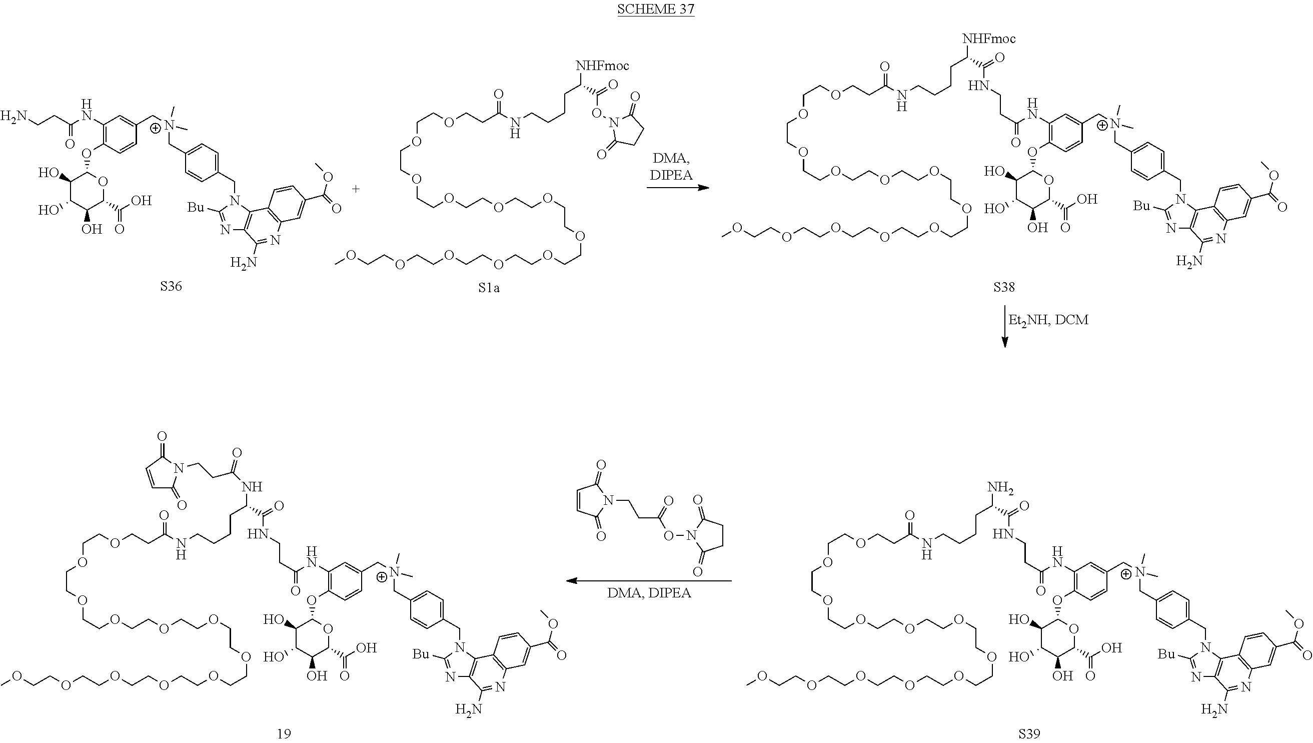

N-(3-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-(((2S,3R,4S,5S,6S)-6-carboxy-3,4,5-trihydroxytetrahydro-2H-pyran-2-yl)oxy)benzyl)-1-(4-((4-amino-2-butyl-7-(methoxycarbonyl)-1H-imidazo[4,5-c]quinolin-1-yl)methyl)phenyl)-N,N-dimethylmethanaminium (S38) as TFA salt, white solid. LCMS: m/z [M]⁺=1734.87 (theoretical); 1735.30 (observed). HPLC retention time=1.73 min (Method D).

N-(4-((4-amino-2-butyl-7-(methoxycarbonyl)-1H-imidazo[4,5-c]quinolin-1-yl)methyl)benzyl)-1-(3-((S)-44-amino-38,45-dioxo-2,5,8,11,14,17,20,23,26,29,32,35-dodecaoxa-39,46-diazanonatetracontan-49-amido)-4-(((2S,3R,4S,5S,6S)-6-carboxy-3,4,5-trihydroxytetrahydro-2H-pyran-2-yl)oxy)phenyl)-N,N-dimethylmethanaminium (S39) as TFA salt, white solid. LCMS: m/z [M]⁺=1511.79 (theoretical); 1513.12 (observed). HPLC retention time=1.40 min (Method D).

N-(4-((4-amino-2-butyl-7-(methoxycarbonyl)-1H-imidazo[4,5-c]quinolin-1-yl)methyl)benzyl)-1-(4-(((2S,3R,4S,5S,6S)-6-carboxy-3,4,5-trihydroxytetrahydro-2H-pyran-2-yl)oxy)-3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)phenyl)-N,N-dimethylmethanaminium (19) as TFA salt, white solid. LCMS: m/z [M]⁺=1663.82 (theoretical); 1664.24 (observed). HPLC retention time=1.53 min (Method D).

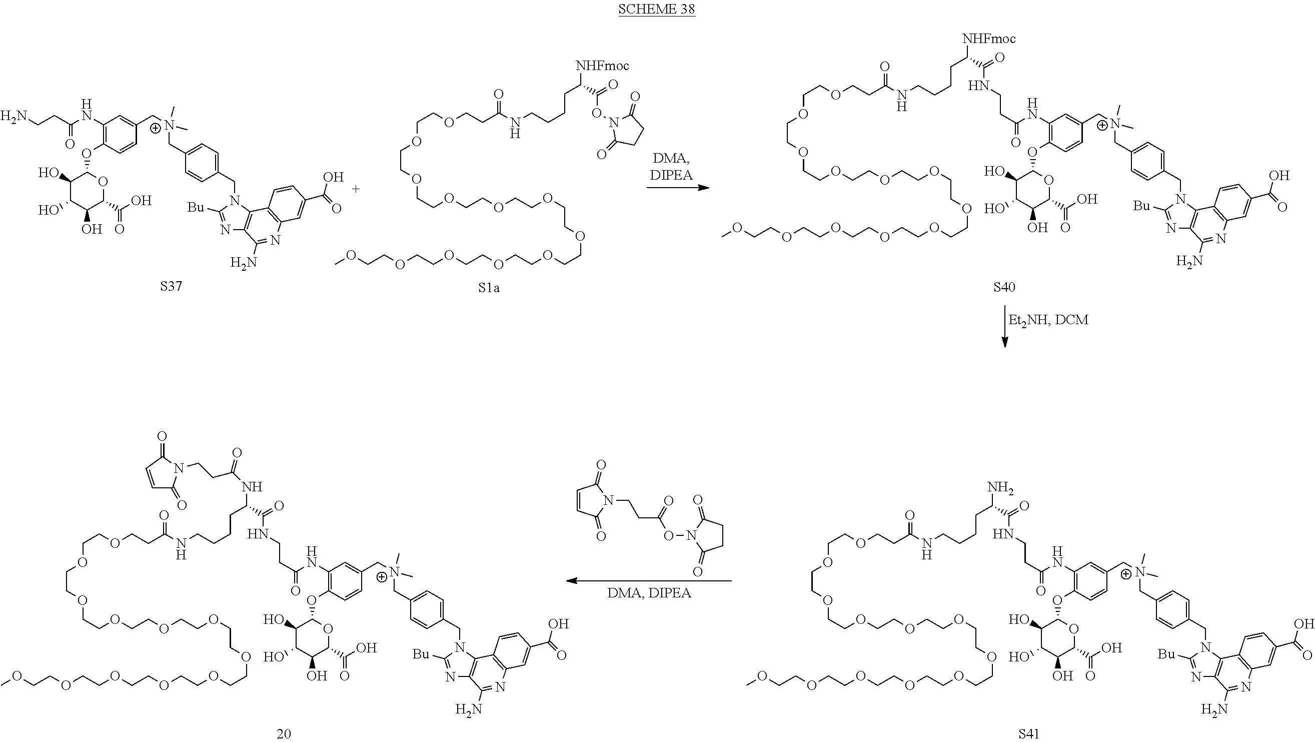

N-(3-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-(((2S,3R,4S,5S,6S)-6-carboxy-3,4,5-trihydroxytetrahydro-2H-pyran-2-yl)oxy)benzyl)-1-(4-((4-amino-2-butyl-7-carboxy-1H-imidazo[4,5-c]quinolin-1-yl)methyl)phenyl)-N,N-dimethylmethanaminium (S40) as TFA salt, white solid. LCMS: m/z [M]⁺=1720.85 (theoretical); 1721.22 (observed). HPLC retention time=1.58 min (Method D).

N-(4-((4-amino-2-butyl-7-carboxy-1H-imidazo[4,5-c]quinolin-1-yl)methyl)benzyl)-1-(3-((S)-44-amino-38,45-dioxo-2,5,8,11,14,17,20,23,26,29,32,35-dodecaoxa-39,46-diazanonatetracontan-49-amido)-4-(((2S,3R,4S,5S,6S)-6-carboxy-3,4,5-trihydroxytetrahydro-2H-pyran-2-yl)oxy)phenyl)-N,N-dimethylmethanaminium (S41) as TFA salt, white solid. LCMS: m/z [M]⁺=1498.78 (theoretical); 1499.19 (observed). HPLC retention time=1.37 min (Method D).

N-(4-((4-amino-2-butyl-7-carboxy-1H-imidazo[4,5-c]quinolin-1-yl)methyl)benzyl)-1-(4-(((2S,3R,4S,5S,6S)-6-carboxy-3,4,5-trihydroxytetrahydro-2H-pyran-2-yl)oxy)-3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)phenyl)-N,N-dimethylmethanaminium (20) as TFA salt, white solid. LCMS: m/z [M]⁺=1649.81 (theoretical); 1650.22 (observed). HPLC retention time=1.48 min (Method D).

### Example 17

### Syntheses of Two Drug-Linker Conjugates

This example covers synthesis of complexes comprising linkers coupled to C4 amines of imidazoquinoline TLR7/8 agonists with C₇ carboxylic acid functionalization, namely 2-butyl-4-((((4-(((2S,3R,4S,5S,6S)-6-carboxy-3,4,5-trihydroxytetrahydro-2H-pyran-2-yl)oxy)-3-(3-(3-(2,5-dioxo-2,5-dihydro-1H-pyrrol-1-yl)propanamido)propanamido)benzyl)oxy)carbonyl)amino)-1-(4-((dimethylamino)methyl)benzyl)-1H-imidazo[4,5-c]quinoline-7-carboxylic acid (21) and 2-butyl-4-((((4-(((2S,3R,4S,5S,6S)-6-carboxy-3,4,5-trihydroxytetrahydro-2H-pyran-2-yl)oxy)-3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)benzyl)oxy)carbonyl)amino)-1-(4-((dimethylamino)methyl)benzyl)-1H-imidazo[4,5-c]quinoline-7-carboxylic acid (22). The syntheses of compounds 21 and 22 are outlined in SCHEMES 39 and 40, respectively. The syntheses of compounds 21 and 22 were similar to the syntheses of compounds 11 and 15, respectively.

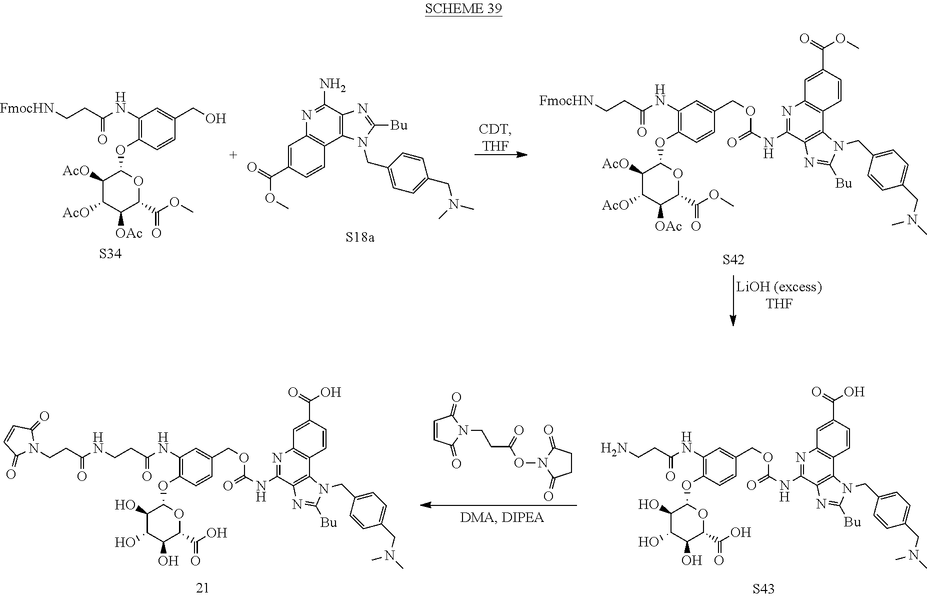

(2S,3R,4S,5S,6S)-2-(2-(3-((((9H-fluoren-9-yl)methoxy)carbonyl)amino)propanamido)-4-((((2-butyl-1-(4-((dimethylamino)methyl)benzyl)-7-(methoxycarbonyl)-1H-imidazo[4,5-c]quinolin-4-yl)carbamoyl)oxy)methyl)phenoxy)-6-(methoxycarbonyl)tetrahydro-2H-pyran-3,4,5-triyl triacetate (S42) as TFA salt, white solid. LCMS: m/z [M+H]⁺=1220.48 (theoretical); 1220.92 (observed). HPLC retention time=1.70 min (Method E).

4-((((3-(3-aminopropanamido)-4-(((2S,3R,4S,5S,6S)-6-carboxy-3,4,5-trihydroxytetrahydro-2H-pyran-2-yl)oxy)benzyl)oxy)carbonyl)amino)-2-butyl-1-(4-((dimethylamino)methyl)benzyl)-1H-imidazo[4,5-c]quinoline-7-carboxylic acid (S43) as TFA salt, white solid. LCMS: m/z [M+H]⁺=844.34 (theoretical); 844.66 (observed). HPLC retention time=1.30 min (Method D).

2-butyl-4-((((4-(((2S,3R,4S,5S,6S)-6-carboxy-3,4,5-trihydroxytetrahydro-2H-pyran-2-yl)oxy)-3-(3-(3-(2,5-dioxo-2,5-dihydro-1H-pyrrol-1-yl)propanamido)propanamido)benzyl)oxy)carbonyl)amino)-1-(4-((dimethylamino)methyl)benzyl)-1H-imidazo[4,5-c]quinoline-7-carboxylic acid (21) as TFA salt, white solid. LCMS: m/z [M+H]⁺=995.37 (theoretical); 995.73 (observed). HPLC retention time=1.44 min (Method D).

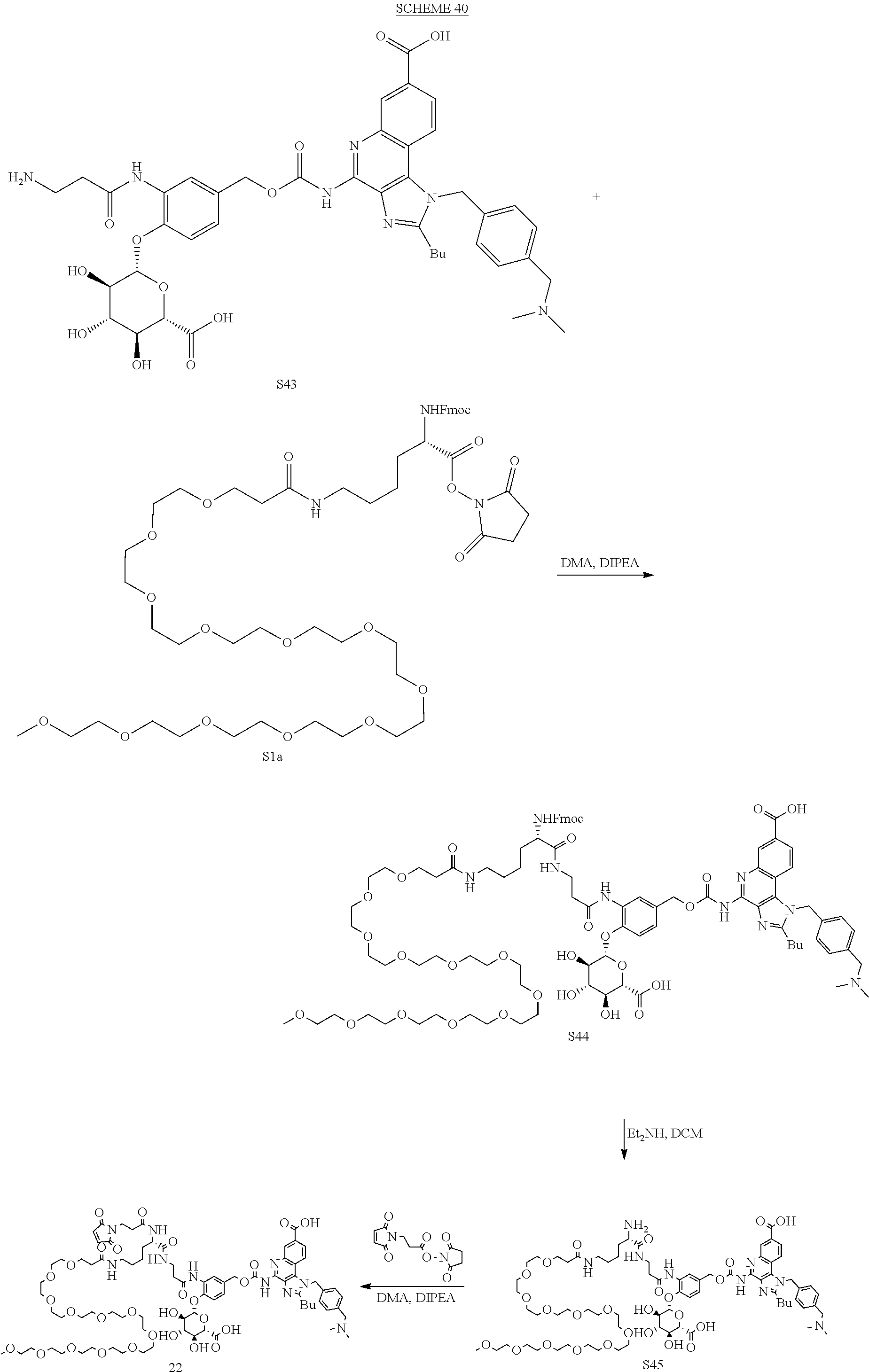

(2S,3S,4S,5R,6S)-6-(2-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-((((2-butyl-1-(4-((dimethylamino)methyl)benzyl)-7-(methoxycarbonyl)-1H-imidazo[4,5-c]quinolin-4-yl)carbamoyl)oxy)methyl)phenoxy)-3,4,5-trihydroxytetrahydro-2H-pyran-2-carboxylic acid (S44) as TFA salt, white solid. LCMS: m/z [M+H]⁺=1764.83 (theoretical); 1766.24 (observed). HPLC retention time=1.76 min (Method D).

4-((((3-((S)-44-amino-38,45-dioxo-2,5,8,11,14,17,20,23,26,29,32,35-dodecaoxa-39,46-diazanonatetracontan-49-amido)-4-(((2S,3R,4S,5S,6S)-6-carboxy-3,4,5-trihydroxytetrahydro-2H-pyran-2-yl)oxy)benzyl)oxy)carbonyl)amino)-2-butyl-1-(4-((dimethylamino)methyl)benzyl)-1H-imidazo[4,5-c]quinoline-7-carboxylic acid (S45) as TFA salt, white solid. LCMS: m/z [M+H]⁺=1542.76 (theoretical); 1543.22 (observed). HPLC retention time=1.51 min (Method D).

2-butyl-4-((((4-(((2S,3R,4S,5S,6S)-6-carboxy-3,4,5-trihydroxytetrahydro-2H-pyran-2-yl)oxy)-3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)benzyl)oxy)carbonyl)amino)-1-(4-((dimethylamino)methyl)benzyl)-1H-imidazo[4,5-c]quinoline-7-carboxylic acid (22) as TFA salt, white solid. LCMS: m/z [M+H]⁺=1693.79 (theoretical); 1694.36 (observed). HPLC retention time=1.61 min (Method D).

### Example 18

### Synthesis of a Drug-Linker Conjugate

This example covers synthesis of a complex comprising a linker coupled to an N1 position of an imidazoquinoline TLR7/8 agonist with C₇ methyl ester functionalization, namely N-(4-((4-amino-2-butyl-7-(methoxycarbonyl)-1H-imidazo[4,5-c]quinolin-1-yl)methyl)benzyl)-1-(4-((S)-2-((S)-2-(3-(2,5-dioxo-2,5-dihydro-1H-pyrrol-1-yl)propanamido)-3-methylbutanamido)propanamido)phenyl)-N,N-dimethylmethanaminium (23). This synthesis is outlined in SCHEME 41.

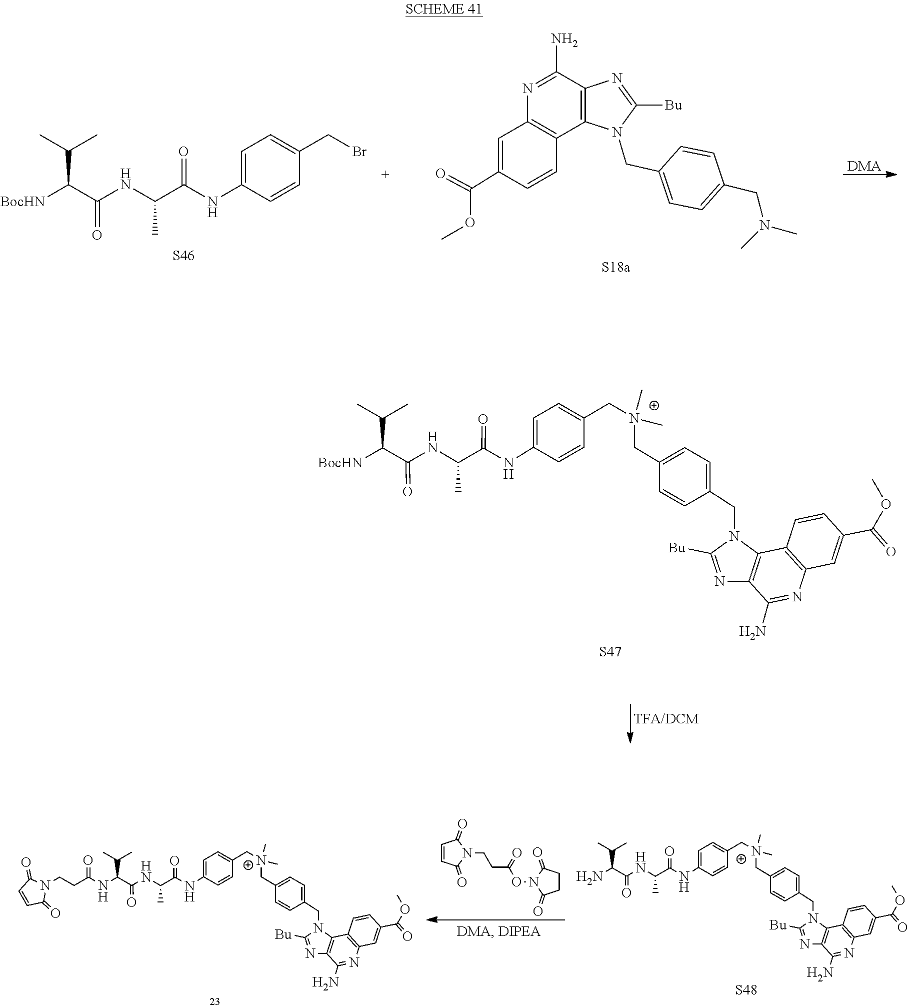

Compound S46 was prepared according to the procedures as described in WO2016040684.

N-(4-((4-amino-2-butyl-7-(methoxycarbonyl)-1H-imidazo[4,5-c]quinolin-1-yl)methyl)benzyl)-1-(4-((S)-2-((S)-2-((tert-butoxycarbonyl)amino)-3-methylbutanamido)propanamido)phenyl)-N,N-dimethylmethanaminium (S47). Compound S46 (16.6 mg, 0.036 mmol) and S18a (13.0 mg, 0.030 mmol) were dissolved in DMA (0.6 mL). The reaction was heated at 50° C. for 2 h and the reaction progress was monitored by LCMS. After 2 h, the reaction mixture was diluted with DMSO/water, and purified by RP-HPLC (Method A) to provide S47 (18.6 mg, 0.022 mmol, 77.6%) as TFA salt, white solid. LCMS: m/z [M]⁺=821.47 (theoretical); 821.66 (observed). HPLC retention time=1.62 min (Method D).

N-(4-((4-amino-2-butyl-7-(methoxycarbonyl)-1H-imidazo[4,5-c]quinolin-1-yl)methyl)benzyl)-1-(4-((S)-2-((S)-2-amino-3-methylbutanamido)propanamido)phenyl)-N,N-dimethylmethanaminium (S48). Intermediate S47 (18.6 mg, 0.022 mmol) was dissolved in 0.6 mL of a 5:1 (v/v) mixture of DCM/TFA and stirred at room temperature for 30 min, after which the solvents were removed in vacuo. The crude reaction mixture was diluted with DMSO/water and purified by RP-HPLC (Method A) to provide S48 (13.5 mg, 0.019 mmol, 82.5%) as TFA salt, white solid. LCMS: m/z [M]⁺=721.42 (theoretical); 721.56 (observed). HPLC retention time=1.26 min (Method D).

N-(4-((4-amino-2-butyl-7-(methoxycarbonyl)-1H-imidazo[4,5-c]quinolin-1-yl)methyl)benzyl)-1-(4-((S)-2-((S)-2-(3-(2,5-dioxo-2,5-dihydro-1H-pyrrol-1-yl)propanamido)-3-methylbutanamido)propanamido)phenyl)-N,N-dimethylmethanaminium (23). Intermediate S48 (3.20 mg, 0.004 mmol) and mp-OSu (1.53 mg, 0.006 mmol) were dissolved in anhydrous DMA (0.2 mL) and DIPEA (3.33 μL, 0.019 mmol) at room temperature. The reaction mixture was stirred at the same temperature for 30 min. After 30 min, the reaction mixture was quenched with HOAc (5 μl), diluted with DMSO/water, and purified by RP-HPLC (Method A) to give compound 23 (1.6 mg, 0.02 mmol, 42.3%) as TFA salt, white solid. LCMS: m/z [M]⁺=872.45 (theoretical); 872.64 (observed). HPLC retention time=1.51 min (Method D).

### Example 19

### Synthesis of a Drug-Linker Conjugate

This example covers synthesis of a complex comprising a linker coupled to an N1 position of an imidazoquinoline TLR7/8 agonist with C₇ carboxylic acid functionalization, namely N-(4-((4-amino-2-butyl-7-carboxy-1H-imidazo[4,5-c]quinolin-1-yl)methyl)benzyl)-1-(4-((S)-2-((S)-2-(3-(2,5-dioxo-2,5-dihydro-1H-pyrrol-1-yl)propanamido)-3-methylbutanamido)propanamido)phenyl)-N,N-dimethylmethanaminium (24). This synthesis is outlined in SCHEME 42.

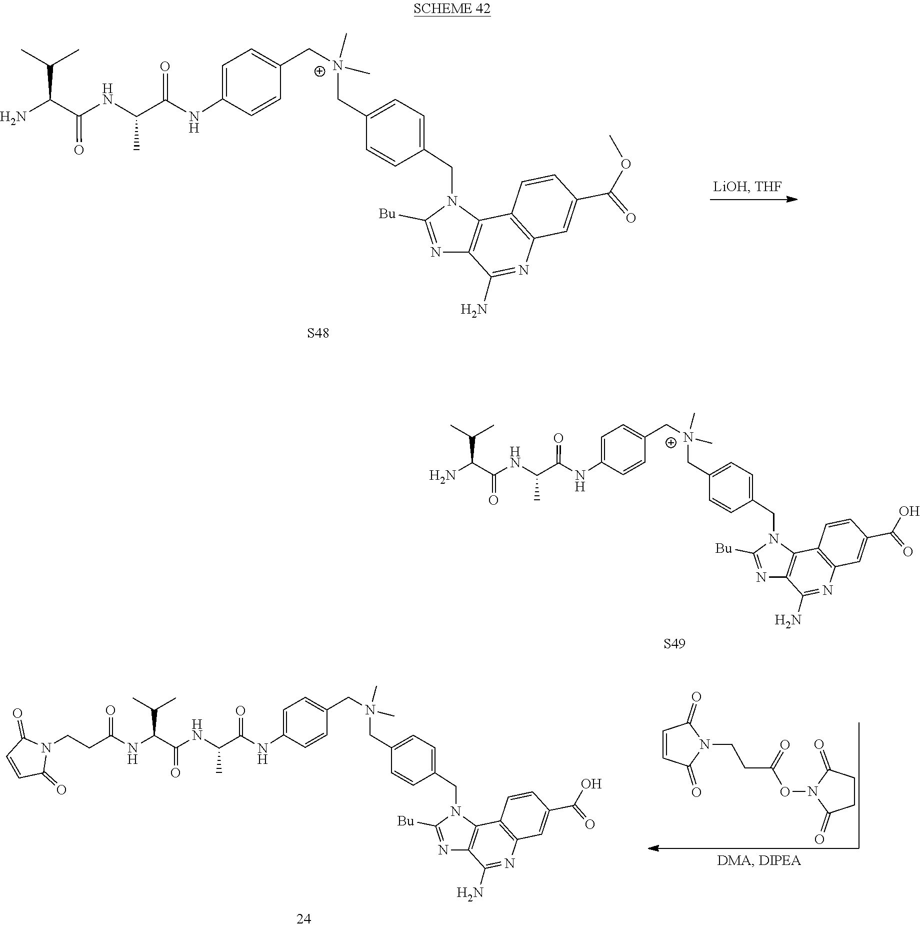

Synthesis of N-(4-((4-amino-2-butyl-7-carboxy-1H-imidazo[4,5-c]quinolin-1-yl)methyl)benzyl)-1-(4-((S)-2-((S)-2-amino-3-methylbutanamido)propanamido)phenyl)-N,N-dimethylmethanaminium (S49). Intermediate S48 (9.1 mg, 0.011 mmol) was dissolved in THF/MeOH (0.3 mL/0.05 mL) followed by the addition of an aqueous LiOH solution (0.5M, 0.109 mL, 0.054 mmol). The reaction mixture was stirred at room temperature for 90 min. After 90 min, the reaction mixture was quenched with HOAc (6.5 μL). THF was removed in vacuo and the crude product was diluted with DMSO/water and purified by RP-HPLC (Method A) to give intermediate S49 (4.6 mg, 0.006 mmol, 59.5%) as TFA salt, white solid. LCMS: m/z [M]⁺=707.40 (theoretical); 707.55 (observed). HPLC retention time=1.25 min (Method D).

Synthesis of N-(4-((4-amino-2-butyl-7-carboxy-1H-imidazo[4,5-c]quinolin-1-yl)methyl)benzyl)-1-(4-((S)-2-((S)-2-(3-(2,5-dioxo-2,5-dihydro-1H-pyrrol-1-yl)propanamido)-3-methylbutanamido)propanamido)phenyl)-N,N-dimethylmethanaminium (24). Intermediate S49 (3.90 mg, 0.005 mmol) and mp-OSu (1.90 mg, 0.007 mmol) were dissolved in anhydrous DMA (0.2 mL) and DIPEA (4.13 μL, 0.024 mmol) at room temperature. The reaction mixture was stirred at the same temperature for 30 min. After 30 min, the reaction mixture was quenched with HOAc (5 μL), diluted with DMSO/water, and purified by RP-HPLC (Method A) to give compound 24 (2.7 mg, 0.03 mmol, 66.2%) as TFA salt, white solid. LCMS: m/z [M]⁺=858.43 (theoretical); 858.62 (observed). HPLC retention time=1.48 min (Method D).

### Example 20

### Synthesis of a Drug-Linker Conjugate

This example covers synthesis of a complex comprising a linker coupled to an N1 position of an imidazoquinoline TLR7/8 agonist with C₇ carboxylic acid functionalization, namely N-(4-((4-amino-2-butyl-7-carboxy-1H-imidazo[4,5-c]quinolin-1-yl)methyl)benzyl)-1-(4-((44S,47S,50S)-44-(3-(2,5-dioxo-2,5-dihydro-1H-pyrrol-1-yl)propanamido)-47-isopropyl-50-methyl-38,45,48-trioxo-2,5,8,11,14,17,20,23,26,29,32,35-dodecaoxa-39,46,49-triazahenpentacontan-51-amido)phenyl)-N,N-dimethylmethanaminium (25). As outlined in SCHEME 43, synthesis of compound 25 was similar to the synthesis of compound 9.

N-(4-((44S,47S,50S)-44-((((9H-fluoren-9-yl)methoxy)carbonyl)amino)-47-isopropyl-50-methyl-38,45,48-trioxo-2,5,8,11,14,17,20,23,26,29,32,35-dodecaoxa-39,46,49-triazahenpentacontan-51-amido)benzyl)-1-(4-((4-amino-2-butyl-7-carboxy-1H-imidazo[4, 5-c]quinolin-1-yl)methyl)phenyl)-N,N-dimethylmethanaminium (S50) as TFA salt, white solid. L CMS: m/z [M+H]⁺=1628.89 (theoretical); 1629.27 (observed). HPLC retention time=1.70 min (Method D).

N-(4-((4-amino-2-butyl-7-carboxy-1H-imidazo[4,5-c]quinolin-1-yl)methyl)benzyl)-1-(4-((44S,47S,50S)-44-amino-47-isopropyl-50-methyl-38,45,48-trioxo-2,5,8,11,14,17,20,23,26,29,32,35-dodecaoxa-39,46,49-triazahenpentacontan-51-amido)phenyl)-N,N-dimethylmethanaminium (S51) as TFA salt, white solid. LCMS: m/z [M]⁺=1405.82 (theoretical); 1406.17 (observed). HPLC retention time=1.28 min (Method D).

N-(4-((4-amino-2-butyl-7-carboxy-1H-imidazo[4,5-c]quinolin-1-yl)methyl)benzyl)-1-(4-((44S,47S,50S)-44-(3-(2,5-dioxo-2,5-dihydro-1H-pyrrol-1-yl)propanamido)-47-isopropyl-50-methyl-38,45,48-trioxo-2,5,8,11,14,17,20,23,26,29,32,35-dodecaoxa-39,46,49-triazahenpentacontan-51-amido)phenyl)-N,N-dimethylmethanaminium (25) as TFA salt, white solid. LCMS: m/z [M]⁺=1556.85 (theoretical); 1556.83 (observed). HPLC retention time=1.50 min (Method D).

### Example 21

### Syntheses of Two Drug-Linker Conjugates

This example covers syntheses of two complexes comprising linkers coupled to C₄ amines of imidazoquinoline TLR7/8 agonists with C₇ carboxylic acid functionalization, namely 2-butyl-1-(4-((dimethylamino)methyl)benzyl)-4-((((4-((44S,47S,50S)-44-(3-(2,5-dioxo-2,5-dihydro-1H-pyrrol-1-yl)propanamido)-47-isopropyl-50-methyl-38,45,48-trioxo-2,5,8,11,14,17,20,23,26,29,32,35-dodecaoxa-39,46,49-triazahenpentacontan-51-amido)benzyl)oxy)carbonyl)amino)-1H-imidazo[4,5-c]quinoline-7-carboxylic acid (26) and 2-butyl-1-(4-((dimethylamino)methyl)benzyl)-4-((((4-((S)-2-((S)-2-(3-(2,5-dioxo-2,5-dihydro-1H-pyrrol-1-yl)propanamido)-3-methylbutanamido)propanamido)benzyl)oxy)carbonyl)amino)-1H-imidazo[4,5-c]quinoline-7-carboxylic acid (27). The syntheses of compounds 26 and 27 were similar to the syntheses of compounds 11 and 15, respectively, and are outlined in SCHEME 44.

Compound S52 was prepared according to the procedures as described in Wang et al. Int. J. Mol. Sci. Vol. 18, No. 9, 1860 (2017).

Methyl 4-((((4-((S)-2-((S)-2-((((9H-fluoren-9-yl)methoxy)carbonyl)amino)-3-methylbutanamido)propanamido)benzyl)oxy)carbonyl)amino)-2-butyl-1-(4-((dimethylamino)methyl)benzyl)-1H-imidazo[4,5-c]quinoline-7-carboxylate (S53) as TFA salt, white solid. LCMS: m/z [M+H]⁺=987.41 (theoretical); 987.51 (observed). HPLC retention time=1.68 min (Method D).

4-((((4-((S)-2-((S)-2-amino-3-methylbutanamido)propanamido)benzyl)oxy)carbonyl)amino)-2-butyl-1-(4-((dimethylamino)methyl)benzyl)-1H-imidazo[4,5-c]quinoline-7-carboxylic acid (S54) as TFA salt, white solid. LCMS: m/z [M+H]⁺=751.39 (theoretical); 751.42 (observed). HPLC retention time=1.31 min (Method D).

4-((((4-((44S,47S,50S)-44-((((9H-fluoren-9-yl)methoxy)carbonyl)amino)-47-isopropyl-50-methyl-38,45,48-trioxo-2,5,8,11,14,17,20,23,26,29,32,35-dodecaoxa-39,46,49-triazahenpentacontan-51-amido)benzyl)oxy)carbonyl)amino)-2-butyl-1-(4-((dimethylamino)methyl)benzyl)-1H-imidazo[4,5-c]quinoline-7-carboxylic acid (S55) as TFA salt, white solid. LCMS: m/z [M+H]⁺=1671.87 (theoretical); 1671.68 (observed). HPLC retention time=1.92 min (Method D).

2-butyl-1-(4-((dimethylamino)methyl)benzyl)-4-((((4-((44S,47S,50S)-44-(3-(2,5-dioxo-2,5-dihydro-1H-pyrrol-1-yl)propanamido)-47-isopropyl-50-methyl-38,45,48-trioxo-2,5,8,11,14,17,20,23,26,29,32,35-dodecaoxa-39,46,49-triazahenpentacontan-51-amido)benzyl)oxy)carbonyl)amino)-1H-imidazo[4,5-c]quinoline-7-carboxylic acid (26) as TFA salt, white solid. LCMS: m/z [M+H]⁺=1600.83 (theoretical); 1600.57 (observed). HPLC retention time=1.59 min (Method D).

2-butyl-1-(4-((dimethylamino)methyl)benzyl)-4-((((4-((S)-2-((S)-2-(3-(2,5-dioxo-2,5-dihydro-1H-pyrrol-1-yl)propanamido)-3-methylbutanamido)propanamido)benzyl)oxy)carbonyl)amino)-1H-imidazo[4,5-c]quinoline-7-carboxylic acid (27) as TFA salt, white solid. LCMS: m/z [M+L¹]⁺=924.46 (theoretical); 924.28 (observed). HPLC retention time=1.58 min (Method D).

### Example 22

### Synthesis of a Drug-Linker Conjugate

This example covers synthesis of a complex comprising a PEGylated linker coupled to a C4 amine of an imidazoquinoline TLR7/8 agonist lacking C₇ functionalization, namely (2S,3S,4S,5R,6S)-6-(2-((S)-44-((S)-3-amino-2-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-((((2-(ethoxymethyl)-1-(2-hydroxy-2-methylpropyl)-1H-imidazo[4,5-c]quinolin-4-yl)carbamoyl)oxy)methyl)phenoxy)-3,4,5-trihydroxytetrahydro-2H-pyran-2-carboxylic acid (28).

Synthesis of (2S,3R,4S,5S,6S)-2-(2-(3-((((9H-fluoren-9-yl)methoxy)carbonyl)amino)propanamido)-4-((((perfluorophenoxy)carbonyl)oxy)methyl)phenoxy)-6-(methoxycarbonyl)tetrahydro-2H-pyran-3,4,5-triyl triacetate (S56). Synthesis of intermediate S56 is outlined in SCHEME 45. Intermediate S34 (2.6 g, 3.47 mmol) and bis-(pentafluorophenyl) carbonate (Combi-Blocks Inc. 1.64 g, 4.16 mmol) were dissolved in DCM (30 mL), followed by the addition of DIPEA (1.21 ml, 7.94 mmol) at room temperature. The reaction mixture was stirred for 16 h at room temperature and the reaction progress was monitored by LCMS. Solvent was removed and the crude product was purified by Biotage (Method B) to give S56 as white solid (2.78 g, 2.90 mmol, 83.5%). LCMS: m/z [M+H]⁺=959.2298 (theoretical); 959.0625 (observed). HPLC retention time=1.81 min (Method C).

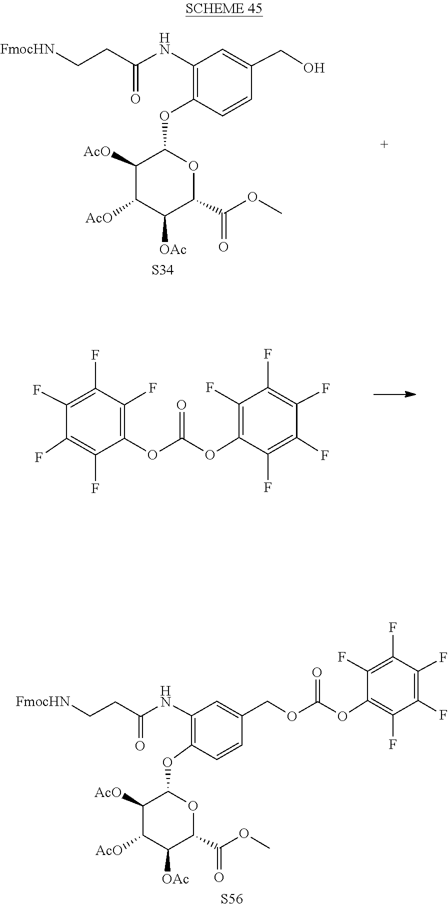

Synthesis of (2S,3R,4S,5S,6S)-2-(2-(3-((((9H-fluoren-9-yl)methoxy)carbonyl)amino)propanamido)-4-((((2-(ethoxymethyl)-1-(2-hydroxy-2-methylpropyl)-1H-imidazo[4,5-c]quinolin-4-yl)carbamoyl)oxy)methyl)phenoxy)-6-(methoxycarbonyl)tetrahydro-2H-pyran-3,4,5-triyl triacetate (S57). Intermediate S57 was synthesized as outlined in SCHEME 46. Resiquimod (Asta Tech, 100 mg, 0.318 mmol) and S56 (457.5 mg, 0.477 mmol) were dissolved in anhydrous DMF (3 mL) followed by the addition of DIPEA (0.166 mL, 0.954 mmol) and 1-hydroxy-7-azabenzotriazole (HOAt, 22 mg, 0.159 mmol). The reaction mixture was warmed up to 30° C. and stirred for 16 h, after which, LCMS indicated ˜90% conversion of the starting material. Solvent was removed in vacuo and the crude reaction mixture was purified by RP-HPLC (Method A) to provide intermediate S57 (236.5 mg, 0.217 mmol, 68.3%) as TFA salt. LCMS: m/z [M+H]⁺=1089.40 (theoretical); 1089.47 (observed). HPLC retention time=1.44 min (Method C.

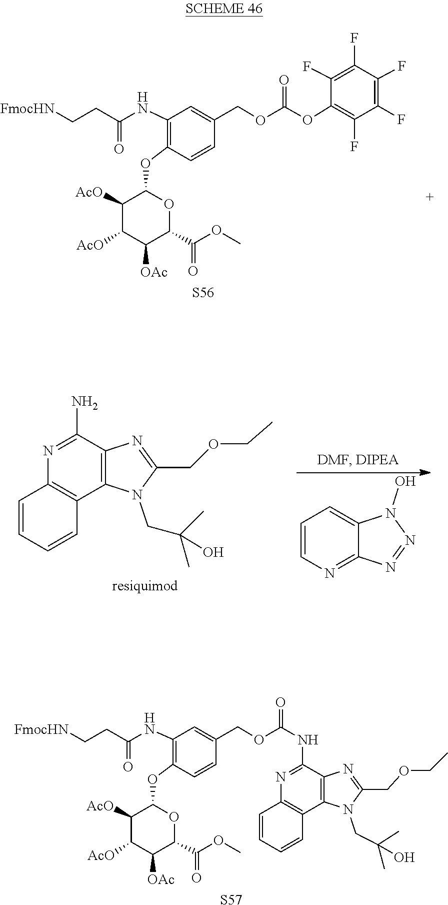

Synthesis of (2S,3S,4S,5R,6S)-6-(2-(3-aminopropanamido)-4-((((2-(ethoxymethyl)-1-(2-hydroxy-2-methylpropyl)-1H-imidazo[4,5-c]quinolin-4-yl)carbamoyl)oxy)methyl)phenoxy)-3,4,5-trihydroxytetrahydro-2H-pyran-2-carboxylic acid (S58). Intermediate S58 was synthesized as outlined in SCHEME 47. Intermediate S57 (473 mg, 0.474 mmol) was dissolved in DCM (5 mL) at room temperature followed by the addition of Et₂NH (1 mL). The reaction mixture was stirred at room temperature for 4 h. After 4 h, solvents were removed in vacuo and the crude reaction mixture was re-dissolved in THF (9 mL) at 0° C. and an aqueous LiOH solution (0.2 M, 10.9 mL, 2.172 mmol) was added. The resulting reaction mixture was stirred at 0° C. for 90 min. After 30 min, HOAc (0.124 mL, 2.172 mmol) was added to neutralize the reaction mixture. Solvents were removed in vacuo and the crude product was diluted with water/DMSO and purified by RP-HPLC (method A) to give intermediate S58 (292 mg, 0.308 mmol, 70.9%) as TFA salt. LCMS: m/z [M+H]⁺=727.29 (theoretical); 727.24 (observed). HPLC retention time=1.31 min (Method C).

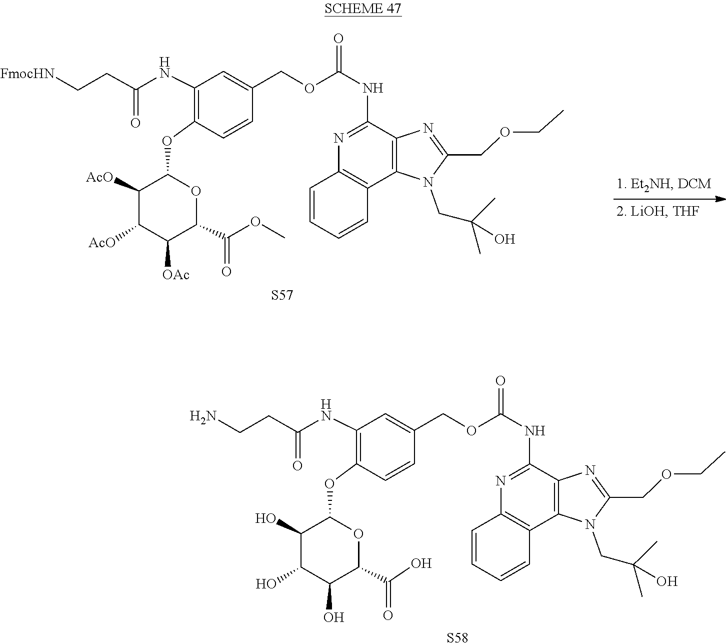

Synthesis of (2S,3S,4S,5R,6S)-6-(2-((S)-44-amino-38,45-dioxo-2,5,8,11,14,17,20,23,26,29,32,35-dodecaoxa-39,46-diazanonatetracontan-49-amido)-4-((((2-(ethoxymethyl)-1-(2-hydroxy-2-methylpropyl)-1H-imidazo[4,5-c]quinolin-4-yl)carbamoyl)oxy)methyl)phenoxy)-3,4,5-trihydroxytetrahydro-2H-pyran-2-carboxylic acid (S60). Intermediate S60 was synthesized according to SCHEME 48. Intermediates S58 (180 mg, 0.184 mmol) and S20 (247.2 mg, 0.239 mmol) were dissolved in anhydrous DMA (0.5 mL) and DIPEA (0.127 mL, 0.734 mmol) at room temperature. The reaction mixture was stirred at room temperature for 20 min. After 20 min, the crude reaction mixture was diluted with DMSO/water and purified by RP-HPLC (Method A) to give intermediate S59 (209 mg, 0.127 mmol, 69.1%) as white solid. LCMS: m/z [M+H]⁺=1647.77 (theoretical); 1648.32 (observed). HPLC retention time=1.49 min (Method C). Intermediate S59 was dissolved in DCM/Et₂NH mixture and the reaction mixture was stirred at room temperature for 30 min, upon which LCMS analysis indicated full conversion of the starting material. Solvents were removed in varuo and the crude product was dissolved with DMSO/water, and purified by RP-HPLC (Method A) to give intermediate S60 (139.4 mg, 0.097 mmol, 77.1%) as TFA salt. LCMS: m/z [M+H]⁺=1425.71 (theoretical); 1425.77 (observed). HPLC retention time=1.13 min (Method C).

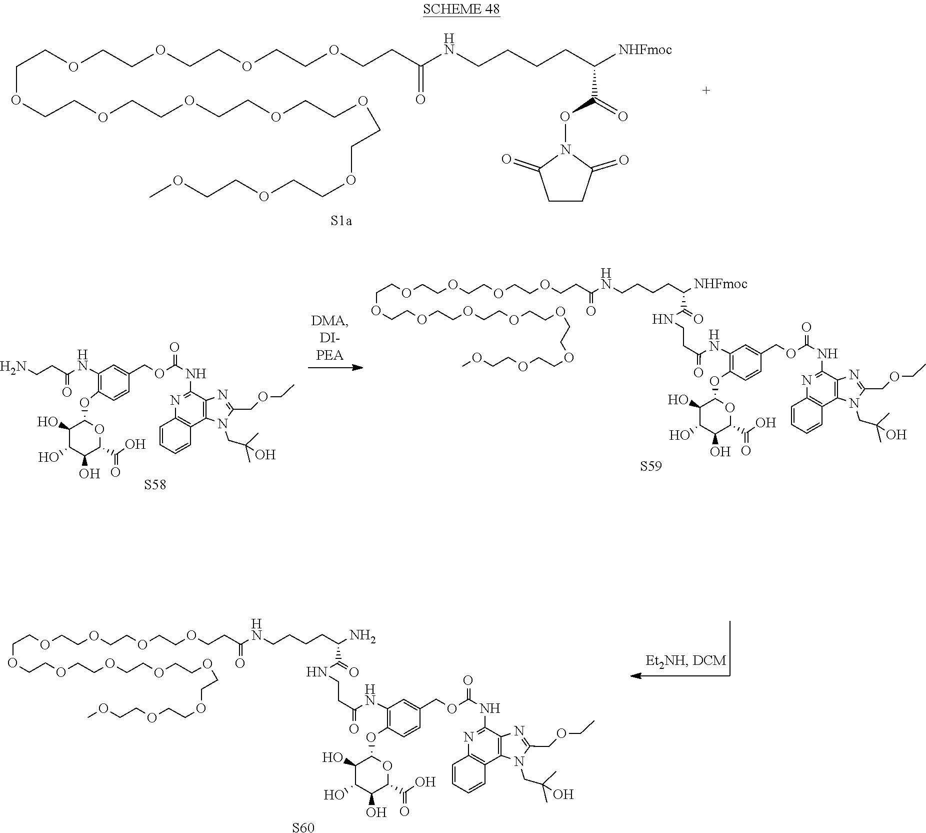

Synthesis of (2S,3S,4S,5R,6S)-6-(2-((S)-44-((S)-3-amino-2-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-((((2-(ethoxymethyl)-1-(2-hydroxy-2-methylpropyl)-1H-imidazo[4,5-c]quinolin-4-yl)carbamoyl)oxy)methyl)phenoxy)-3,4,5-trihydroxytetrahydro-2H-pyran-2-carboxylic acid (28). Compound 28 was synthesized as outlined in SCHEME 49. Intermediates S60 (47.2 mg, 0.031 mmol) and S4 (15.2 mg, 0.04 mmol) were dissolved in anhydrous DMA (0.3 mL) and DIPEA (0.016 mL, 0.092 mmol) at room temperature. The reaction was stirred at room temperature for 1 h. After 1 hour, solvent was removed in vacuo and the crude reaction mixture was re-dissolved in DCM/TFA (10:1 (v/v), 0.5 mL), and the reaction mixture was stirred at room temperature for 30 min. Solvent was then removed and the crude reaction mixture was diluted with DMSO/water, and purified by RP-HPLC (Method A) to give compound 28 (26.2 mg, 0.015 mmol, 50.1%) as TFA salt. LCMS: m/z [M+H]⁺=1591.74 (theoretical); 1592.05 (observed). HPLC retention time=1.16 min (Method C).

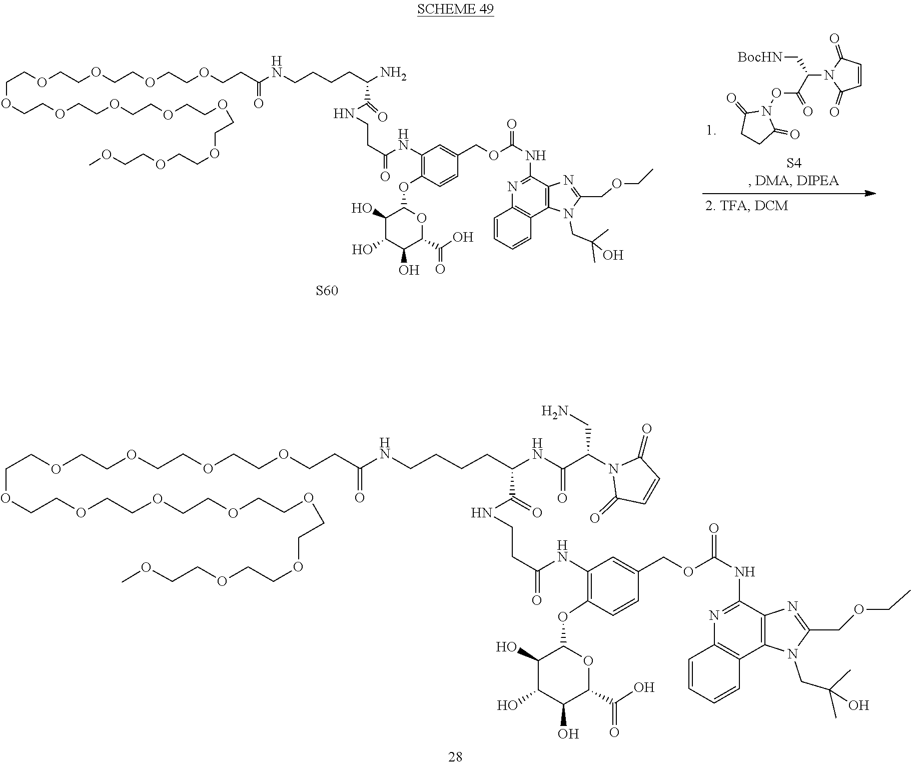

### Example 23

### Synthesis of a Drug-Linker Conjugate

This example covers synthesis of a complex comprising a linker coupled to a C₂ position of an imidazoquinoline TLR7/8 agonist lacking C₇ functionalization, namely (2S,3S,4S,5R,6S)-6-(4-(((((4-amino-1-(2-hydroxy-2-methylpropyl)-1H-imidazo[4,5-c]quinolin-2-yl)methyl)(ethyl)carbamoyl)oxy)methyl)-2-(3-((S)-3-amino-2-(2,5-dioxo-2,5-dihydro-1H-pyrrol-1-yl)propanamido)propanamido)phenoxy)-3,4,5-trihydroxytetrahydro-2H-pyran-2-carboxylic acid (29). Synthesis of (2S,3R,4S,5S,6S)-2-(2-(3-((((9H-fluoren-9-yl)methoxy)carbonyl)amino)propanamido)-4-(((((4-amino-1-(2-hydroxy-2-methylpropyl)-1H-imidazo[4,5-c]quinolin-2-yl)methyl)(ethyl)carbamoyl)oxy)methyl)phenoxy)-6-(methoxycarbonyl)tetrahydro-2H-pyran-3,4,5-triyl triacetate (S61). SCHEME 50 outlines a synthetic scheme for intermediate S61. Gardiquimod (Sigma-Aldrich, 10.0 mg, 0.032 mmol) and S56 (34.99 mg, 0.038 mmol) were dissolved in anhydrous DMF (0.3 mL), followed by the addition of pyridine (0.06 mL) and HOAt (2.0 mg, 0.013 mmol). The reaction mixture was stirred for 3 h, upon which LCMS indicated full conversion of the starting material. The crude reaction mixture was diluted, and purified by RP-HPLC (Method A) to provide intermediate S61 (20.2 mg, 0.019 mmol, 58.2%) as TFA salt. LCMS: m/z [M+H]⁺=1088.42 (theoretical); 1088.67 (observed). HPLC retention time=1.56 min (Method D).

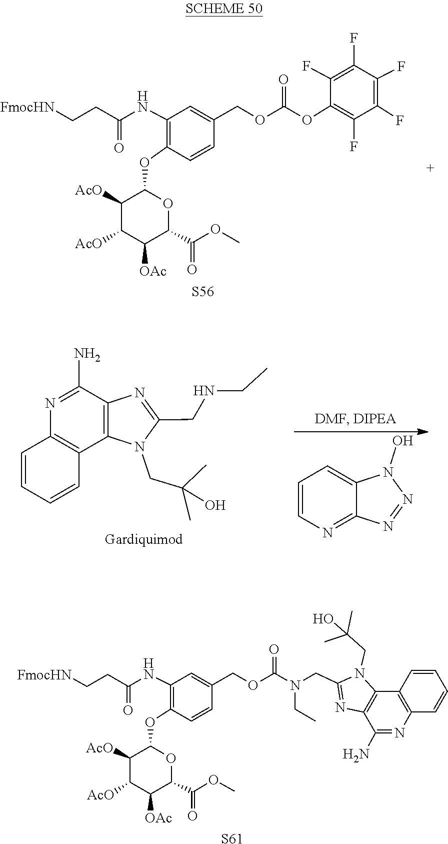

Synthesis of (2S,3S,4S,5R,6S)-6-(4-(((((4-amino-1-(2-hydroxy-2-methylpropyl)-1H-imidazo[4,5-c]quinolin-2-yl)methyl)(ethyl)carbamoyl)oxy)methyl)-2-(3-aminopropanamido)phenoxy)-3,4,5-trihydroxytetrahydro-2H-pyran-2-carboxylic acid (S62). SCHEME 51 outlines a synthetic scheme for generating intermediate S62. Intermediate S61 (20.2 mg, 0.019 mmol) was dissolved in 1.0 mL of a 1:1 (v/v) THF/MeOH mixture and the resulting reaction mixture was stirred at 0° C. for 5 min, followed by the addition of aqueous LiOH (0.2M, 0.928 mL). The reaction mixture was stirred at 0° C. for 30 min, and was then warmed up to room temperature and stirred for another 4 h. After 4 h, the reaction mixture was neutralized with HOAc (5 μL), diluted with water/DMSO, and purified by RP-HPLC (Method A) to give intermediate S62 (16 mg, 0.019 mmol, 100%) as TFA salt. LCMS: m/z [M+H]⁺=726.30 (theoretical); 726.49 (observed). HPLC retention time=0.62 min (Method D).

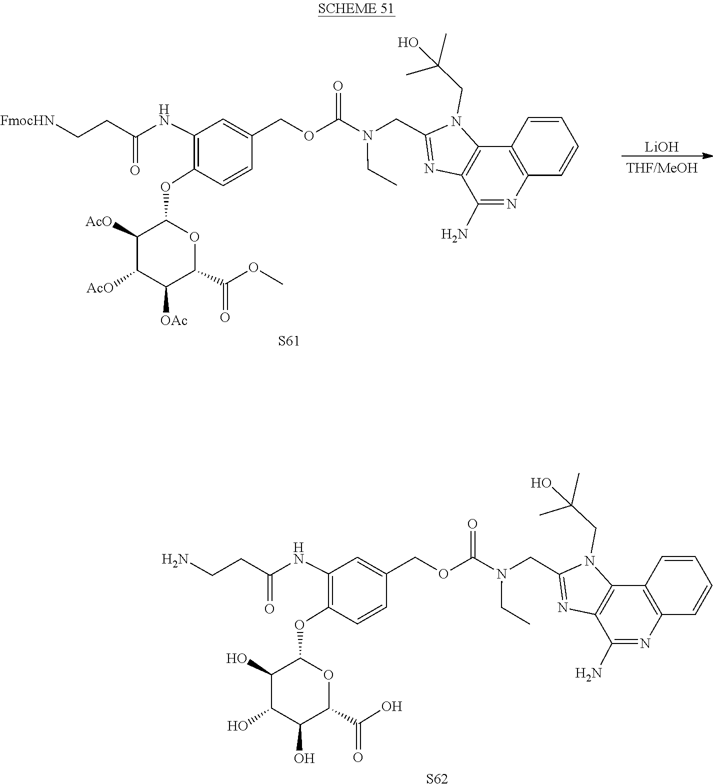

Synthesis of (2S,3S,4S,5R,6S)-6-(4-(((((4-amino-1-(2-hydroxy-2-methylpropyl)-1H-imidazo[4,5-c]quinolin-2-yl)methyl)(ethyl)carbamoyl)oxy)methyl)-2-(3-((S)-3-amino-2-(2,5-dioxo-2,5-dihydro-1H-pyrrol-1-yl)propanamido)propanamido)phenoxy)-3,4,5-trihydroxytetrahydro-2H-pyran-2-carboxylic acid (29). Compound 29 was synthesized as outlined in SCHEME 52. Intermediates S62 (16.0 mg, 0.019 mmol) and S4 (7.99 mg, 0.021 mmol) were dissolved in anhydrous DMF (0.2 mL) and DIPEA (0.01 mL, 0.057 mmol) at room temperature. The reaction mixture was stirred at room temperature for 15 min. After 15 min, the crude reaction mixture was neutralized with acetic acid (2 μL), diluted with DMSO/water and purified by RP-HPLC (Method A) to give the Boc-protected compound 29 (9.0 mg, 0.009 mmol, 47.6%) as TFA salt, which was used in subsequent step without further purification. LCMS: m/z [M+H]⁺=992.39 (theoretical); 992.62 (observed). HPLC retention time=1.01 min (Method D). The crude Boc-protected compound 29 (9.0 mg, 0.009 mmol) was dissolved in DCM (1 mL) and TFA (0.25 mL) and the reaction mixture was stirred at room temperature for 30 min. After 30 min, solvents were removed in vacuo and the crude reaction mixture was re-dissolved in DMSO and purified by RP-HPLC (Method A) to give compound 29 (4.6 mg, 0.005 mmol, 50.4%) as TFA salt. LCMS: m/z [M+H]⁺=892.34 (theoretical); 892.55 (observed). HPLC retention time=0.68 min (Method D).

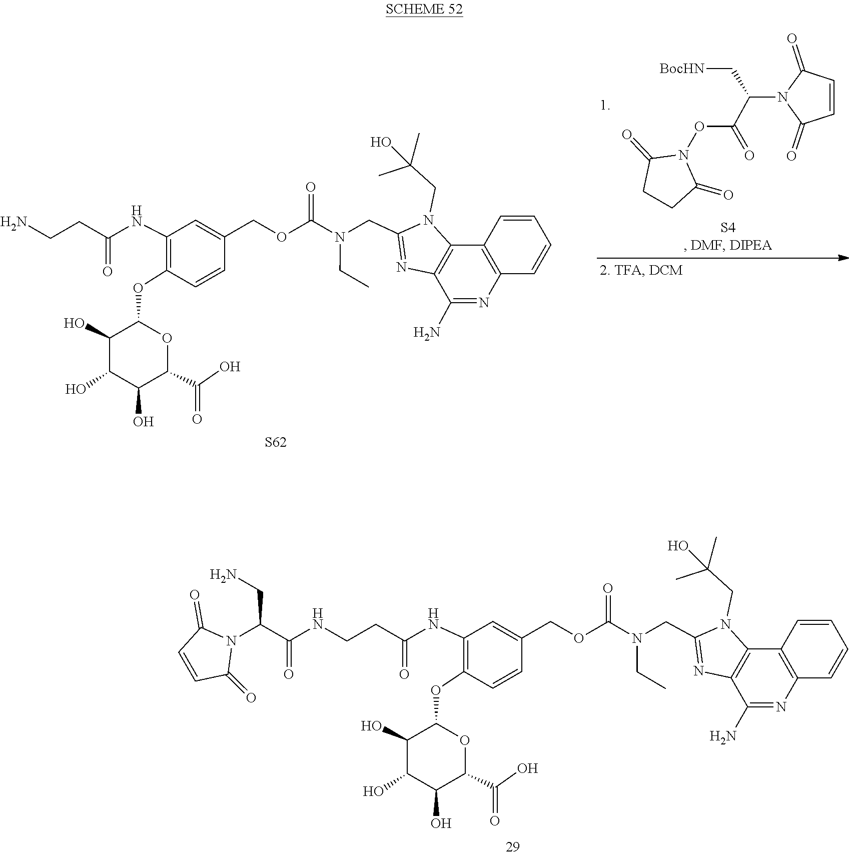 [SMILES: CCN(Cc1nc2c(N)nc3ccccc3c2n1CC(C)(C)O)C(=O)OCc1ccc(O[C@@H]2O[C@H](C(=O)O)[C@@H](O)[C@H](O)[C@H]2O)c(NC(=O)CCN)c1 ; CC(C)(C)OC(=O)NC[C@@H](C(=O)ON1C(=O)CCC1=O)N1C(=O)C=CC1=O ; "S62" ; CCN(Cc1nc2c(N)nc3ccccc3c2n1CC(C)(C)O)C(=O)OCc1ccc(O[C@@H]2O[C@H](C(=O)O)[C@@H](O)[C@H](O)[C@H]2O)c(NC(=O)CCNC(=O)[C@H](CN)N2C(=O)C=CC2=O)c1]

### Example 24

### Synthesis of a Drug-Linker Conjugate

This example covers synthesis of a complex comprising a linker coupled to an N1 position of an imidazoquinoline TLR7/8 agonist with C₇ carboxylic acid functionalization, namely 4-amino-2-butyl-1-(4-(21-(2,5-dioxo-2,5-dihydro-1H-pyrrol-1-yl)-3,19-dioxo-6,9,12,15-tetraoxa-2,18-diazahenicosyl)benzyl)-1H-imidazo[4,5-c]quinoline-7-carboxylic acid (30).

Synthesis of 4-amino-1-(4-(aminomethyl)benzyl)-2-butyl-1H-imidazo[4,5-c]quinoline-7-carboxylic acid (S8b). The synthesis of intermediate S8b is outlined in SCHEME 53. Intermediate S8a (8.3 mg, 0.02 mmol) was dissolved in THE (0.4 mL), followed by the addition of 0.5M LiOH (0.2 ml, 0.1 mmol) and the resulting mixture was stirred at room temperature for 2 h, after which the reaction was quenched with AcOH (6 μL) and solvents were removed in vacuo. The crude reaction mixture was diluted with DMSO/water and purified by RP-HPLC (Method A) to provide S8b (7.5 mg, 0.012 mmol, 59.7%) as 2× TFA salt, white solid. LCMS: m/z [M+H]⁺=404.21 (theoretical); 404.26 (observed). HPLC retention time=1.12 min (Method D).

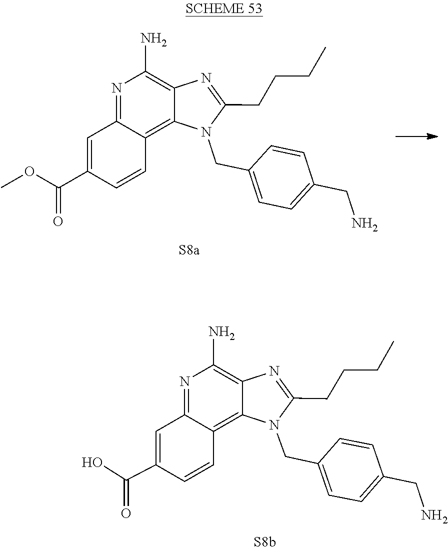 [SMILES: CCCCc1nc2c(N)nc3cc(C(=O)OC)ccc3c2n1Cc1ccc(CN)cc1 ; CCCCc1nc2c(N)nc3cc(C(=O)O)ccc3c2n1Cc1ccc(CN)cc1]

Synthesis of 4-amino-2-butyl-1-(4-(21-(2,5-dioxo-2,5-dihydro-1H-pyrrol-1-yl)-3,19-dioxo-6,9,12,15-tetraoxa-2,18-diazahenicosyl)benzyl)-1H-imidazo[4,5-c]quinoline-7-carboxylic acid (30). Compound 30 was synthesized as outlined in SCHEME 54. 2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate (mp-PEG4-OSu, Broadpharm, 1.31 mg, 0.003 mmol) and S8b were dissolved in DMA (0.3 mL), followed by the addition of DIPEA (1.85 μL, 0.011 mmol). The reaction mixture was stirred at room temperature for 30 min. After 30 min, the crude reaction mixture was neutralized with acetic acid (2 μL), diluted with DMSO/water, and purified by RP-HPLC (Method A) to give compound 30 (1.0 mg, 0.001 mmol, 58.7%) as TFA salt. LCMS: m/z [M+H]⁺=802.37 (theoretical); 802.41 (observed). HPLC retention time=1.56 min (Method C).

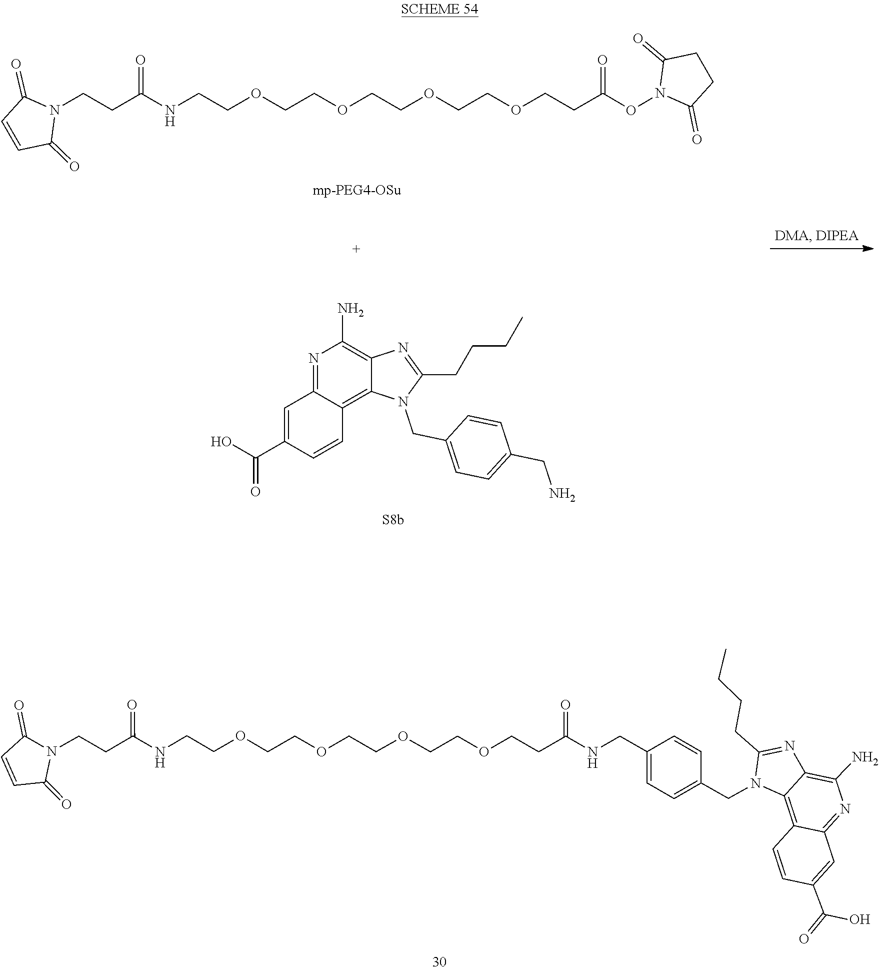 [SMILES: O=C(CCN1C(=O)C=CC1=O)NCCOCCOCCOCCOCCC(=O)ON1C(=O)CCC1=O ; CCCCc1nc2c(N)nc3cc(C(=O)O)ccc3c2n1Cc1ccc(CN)cc1 ; CCCCc1nc2c(N)nc3cc(C(=O)O)ccc3c2n1Cc1ccc(CNC(=O)CCOCCOCCOCCOCCNC(=O)CCN2C(=O)C=CC2=O)cc1]

### Example 25

### Synthesis of a Drug-Linker Conjugate

This example covers synthesis of a complex comprising a linker coupled to a C₄ amine of an imidazoquinoline TLR7/8 agonist with C₇ methyl ester functionalization, namely methyl 2-butyl-4-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-1-(4-hydroxybutyl)-1H-imidazo[4,5-c]quinoline-7-carboxylate (31). As outlined in SCHEME 55, the synthesis of compound 31 was similar to the synthesis of compound 1.

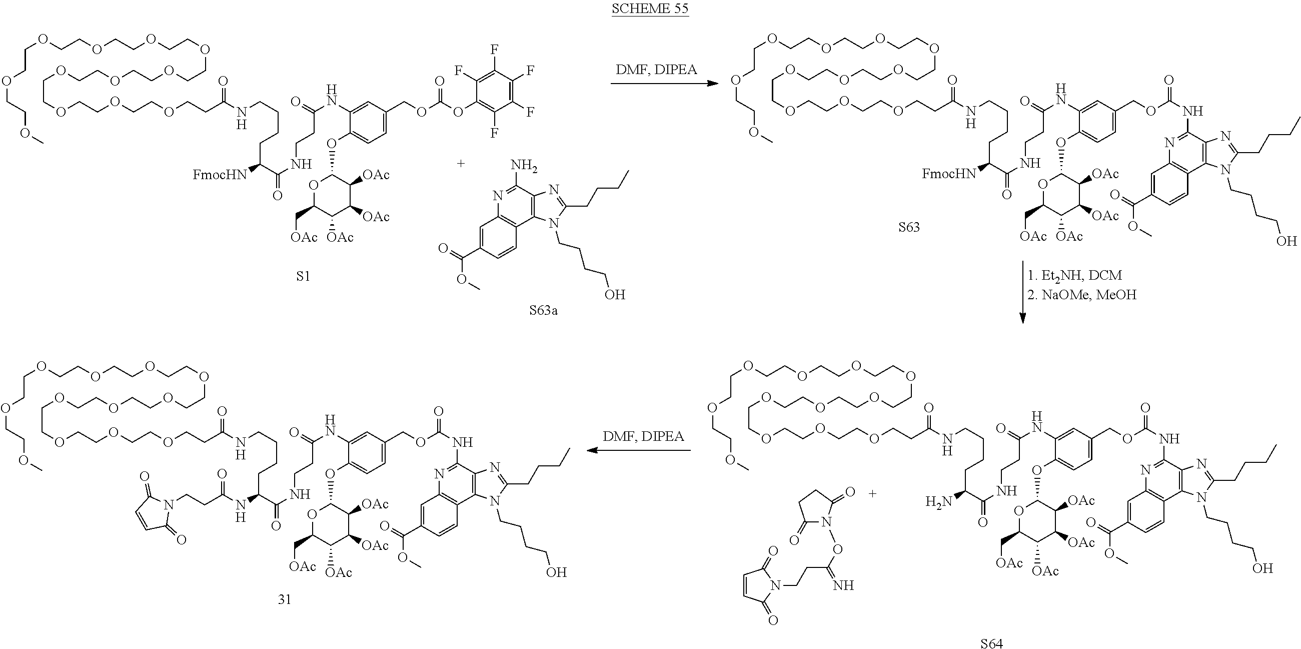

Compound S63a was prepared according to the procedures as described in Larson, et al. ACS Med. Chem. Lett. Vol. 8, No. 11, 1148-1152 (2017).

(2R,3S,4S,5R,6R)-2-(2-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-((((2-butyl-1-(4-hydroxybutyl)-7-(methoxycarbonyl)-1H-imidazo[4,5-c]quinolin-4-yl)carbamoyl)oxy)methyl)phenoxy)-6-(acetoxymethyl)tetrahydro-2H-pyran-3,4,5-triyl triacetate (S63) as TFA salt, white solid. LCMS: m/z [M+H]⁺=1857.86 (theoretical); 1857.97 (observed). HPLC retention time=2.02 min (Method C).

Methyl 4-((((3-((S)-44-amino-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-2-butyl-1-(4-hydroxybutyl)-1H-imidazo[4,5-c]quinoline-7-carboxylate (S64) as TFA salt, white solid. LCMS: m/z [M+H]⁺=1467.75 (theoretical); 1468.94 (observed). HPLC retention time=1.27 min (Method C).

Methyl 2-butyl-4-((((3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-1-(4-hydroxybutyl)-1H-imidazo[4,5-c]quinoline-7-carboxylate (31) as TFA salt, white solid. LCMS: m/z [M+H]⁺=1618.78 (theoretical); 1619.60 (observed). HPLC retention time=1.33 min (Method C).

### Example 26

### Synthesis of a Drug-Linker Conjugate

This example covers synthesis of a complex comprising a linker coupled to an N1 position of an imidazoquinoline TLR7/8 agonist with C₇ carboxylic acid functionalization, namely 1-(4-((4-amino-2-butyl-7-carboxy-1H-imidazo[4,5-c]quinolin-1-yl)methyl)benzyl)-1-(3-(3-(3-(2,5-dioxo-2,5-dihydro-1H-pyrrol-1-yl)propanamido)propanamido)-4-(((2R,3 S,4S,5S,6R)-3,4,5-trihydroxy-6-(hydroxymethyl)tetrahydro-2H-pyran-2-yl)oxy)benzyl)pyrrolidin-1-ium (32). As outlined in SCHEME 56, the synthesis of compound 32 was similar to the synthesis of compound 14.

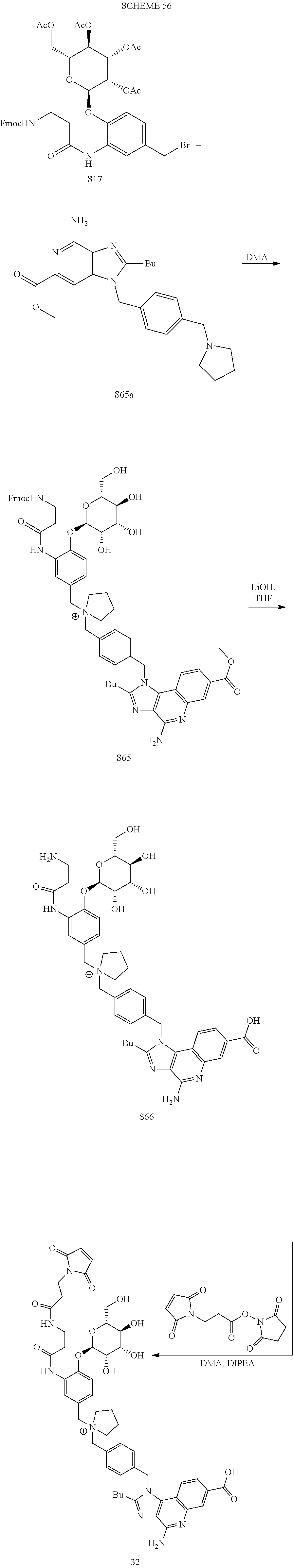

Compound S65a was prepared according to the procedures as described in Larson, et al. ACS Med. Chem. Lett. Vol. 8, No. 11, 1148-1152 (2017).

1-(3-(3-((((9H-fluoren-9-yl)methoxy)carbonyl)amino)propanamido)-4-(((2R,3S,4S,5R,6R)-3,4,5-triacetoxy-6-(acetoxymethyl)tetrahydro-2H-pyran-2-yl)oxy)benzyl)-1-(4-((4-amino-2-butyl-7-(methoxycarbonyl)-1H-imidazo[4,5-c]quinolin-1-yl)methyl)benzyl)pyrrolidin-1-ium (S65) as TFA salt, white solid. LCMS: m/z [M]⁺=1216.52 (theoretical); 1216.38 (observed). HPLC retention time=1.51 min (Method D).

1-(4-((4-amino-2-butyl-7-carboxy-1H-imidazo[4,5-c]quinolin-1-yl)methyl)benzyl)-1-(3-(3-aminopropanamido)-4-(((2R,3 S,4S,5S,6R)-3,4,5-trihydroxy-6-(hydroxymethyl)tetrahydro-2H-pyran-2-yl)oxy)benzyl)pyrrolidin-1-ium (S66) as TFA salt, white solid. LCMS: m/z [M]⁺=812.40 (theoretical); 812.40 (observed). HPLC retention time=1.16 min (Method D). 1-(4-((4-amino-2-butyl-7-carboxy-1H-imidazo[4,5-c]quinolin-1-yl)methyl)benzyl)-1-(3-(3-(3-(2,5-dioxo-2,5-dihydro-1H-pyrrol-1-yl)propanamido)propanamido)-4-(((2R,3 S,4S,5S,6R)-3,4,5-trihydroxy-6-(hydroxymethyl)tetrahydro-2H-pyran-2-yl)oxy)benzyl)pyrrolidin-1-ium (32) as TFA salt, white solid. LCMS: m/z [M]⁺=963.43 (theoretical); 963.47 (observed). HPLC retention time=1.29 min (Method D).

### Example 27

### Synthesis of a Drug-Linker Conjugate

This example covers synthesis of a complex comprising a linker coupled to a C₄ amine of an imidazoquinoline TLR7/8 agonist with C₇ carboxylic acid functionalization, namely 2-butyl-4-((((3-(3-(3-(2,5-dioxo-2,5-dihydro-1H-pyrrol-1-yl)propanamido)propanamido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-1-(4-(pyrrolidin-1-ylmethyl)benzyl)-1H-imidazo[4,5-c]quinoline-7-carboxylic acid (33). As outlined in SCHEME 57, the synthesis of compound 33 was similar to the synthesis of compound 15.

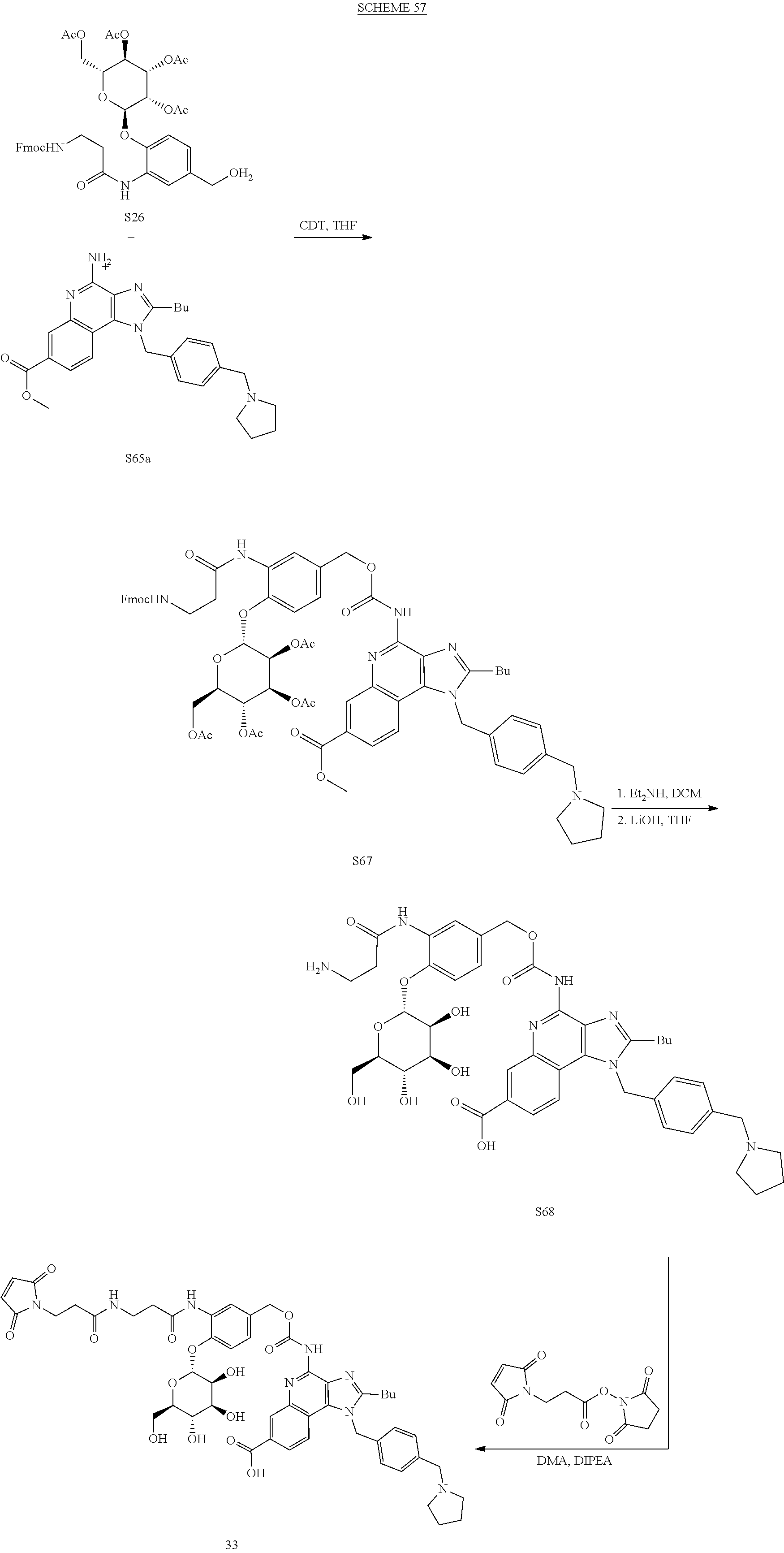

(2R,3 S,4S,5R,6R)-2-(2-(3-((((9H-fluoren-9-yl)methoxy)carbonyl)amino)propanamido)-4-((((2-butyl-7-(methoxycarbonyl)-1-(4-(pyrrolidin-1-ylmethyl)benzyl)-1H-imidazo[4,5-c]quinolin-4-yl)carbamoyl)oxy)methyl)phenoxy)-6-(acetoxymethyl)tetrahydro-2H-pyran-3,4,5-triyl triacetate (S67) as TFA salt, white solid. LCMS: m/z [M+H]⁺=1260.51 (theoretical); 1260.59 (observed). HPLC retention time=2.09 min (Method D).

4-((((3-(3-aminopropanamido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-2-butyl-1-(4-(pyrrolidin-1-ylmethyl)benzyl)-1H-imidazo[4,5-c]quinoline-7-carboxylic acid (S68) as TFA salt, white solid. LCMS: m/z [M+Na]⁺=878.38 (theoretical); 878.38 (observed). HPLC retention time=1.25 min (Method D).

2-butyl-4-((((3-(3-(3-(2,5-dioxo-2,5-dihydro-1H-pyrrol-1-yl)propanamido)propanamido)-4-(((2R,3S,4S,5S,6R)-3,4,5-trihydroxy-6-(hydroxymethyl)tetrahydro-2H-pyran-2-yl)oxy)benzyl)oxy)carbonyl)amino)-1-(4-(pyrrolidin-1-ylmethyl)benzyl)-1H-imidazo[4,5-c]quinoline-7-carboxylic acid (33) as TFA salt, white solid. LCMS: m/z [M+H]⁺=1007.41 (theoretical); 1007.42 (observed). HPLC retention time=1.38 min (Method D).

### Example 28

### Synthesis of a Drug-Linker Conjugate

This example covers synthesis of a complex comprising a linker coupled to an N1 position of an imidazoquinoline TLR7/8 agonist with C₇ carboxylic acid functionalization, namely 4-amino-2-butyl-1-(4-(((((4-(((2S,3R,4S,5S,6S)-6-carboxy-3,4,5-trihydroxytetrahydro-2H-pyran-2-yl)oxy)-3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)benzyl)oxy)carbonyl)amino)methyl)benzyl)-1H-imidazo[4,5-c]quinoline-7-carboxylic acid (34). As outlined in SCHEME 58, the synthesis of compound 34 was similar to the synthesis of compound 3.

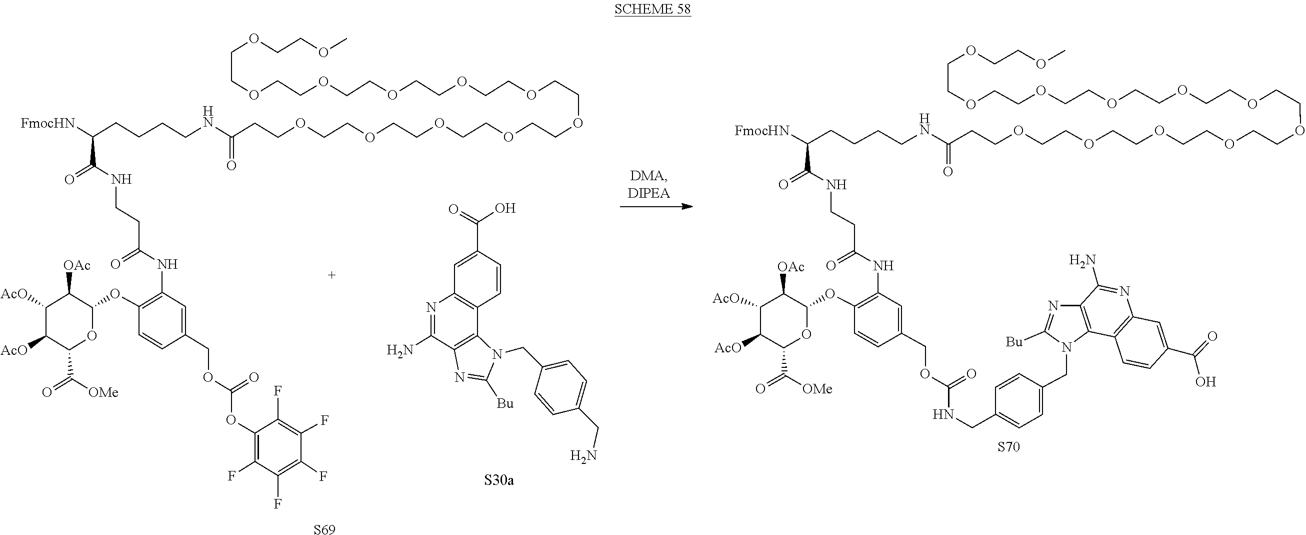

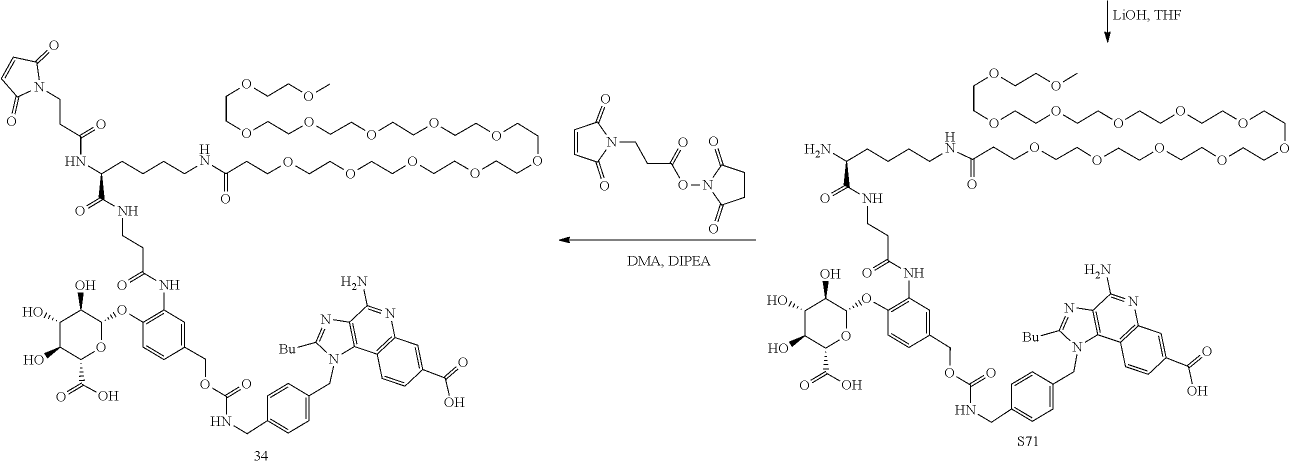

### Example 29

### Synthesis of a PEGylated Linker

This example covers synthesis of a PEGylated linker capable of dual binding to imidazoquinoline compounds and proteins, namely (2S,3R,4S,5S,6S)-2-(2-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-((((perfluorophenoxy)carbonyl)oxy)methyl)phenoxy)-6-(methoxycarbonyl)tetrahydro-2H-pyran-3,4,5-triyl triacetate (S69). This synthesis is summarized in SCHEME 59.

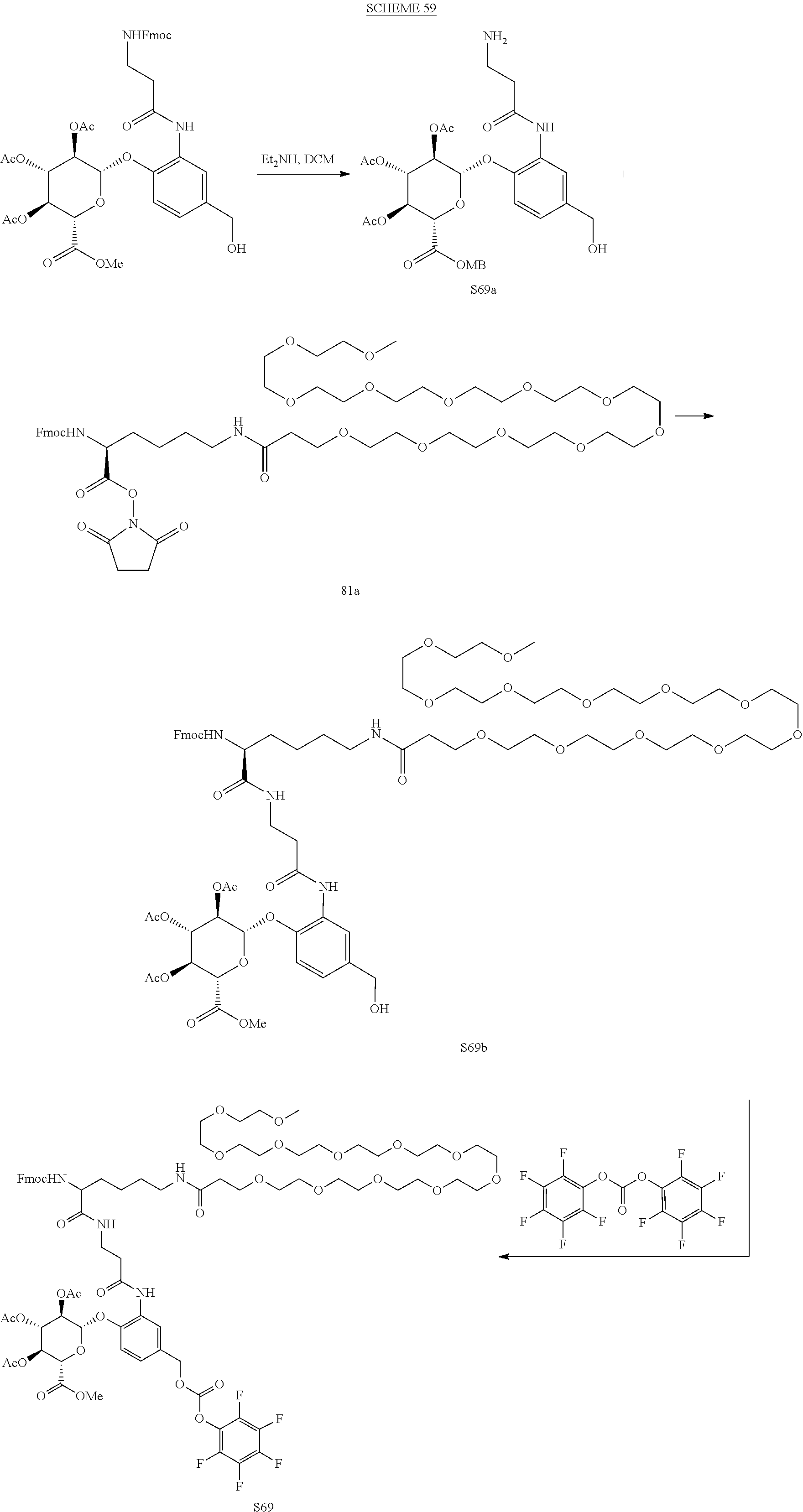

Compound S69 was prepared using similar procedures as those used in the synthesis of S1.

(2S,3R,4S,5S,6S)-2-(2-(3-aminopropanamido)-4-(hydroxymethyl)phenoxy)-6-(methoxycarbonyl)tetrahydro-2H-pyran-3,4,5-triyl triacetate (NH₂-Gluc-PAB-OH) (S69a): as light yellow solid LCMS: m/z [M+H]⁺=527.19 (theoretical); 527.32 (observed). HPLC retention time=1.58 min (Method C).

(2S,3R,4S,5S,6S)-2-(2-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-(hydroxymethyl)phenoxy)-6-(methoxycarbonyl)tetrahydro-2H-pyran-3,4,5-triyl triacetate (NH₂—Fmoc-LysPEG12-Gluc-OH) (S69b): as colorless liquid. LCMS: m/z [M+H]⁺=1447.68 (theoretical); 1447.44 (observed). HPLC retention time=1.85 min (Method C).

(2S,3R,4S,5S,6S)-2-(2-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-((((perfluorophenoxy)carbonyl)oxy)methyl)phenoxy)-6-(methoxycarbonyl)tetrahydro-2H-pyran-3,4,5-triyl triacetate (S69) as colorless sticky liquid. LCMS: m/z [M+H]⁺=1657.65 (theoretical); 1657.91 (observed). HPLC retention time=2.21 min (Method C).

1-(4-(((((3-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-(((2S,3R,4S,5S,6S)-3,4,5-triacetoxy-6-(methoxycarbonyl)tetrahydro-2H-pyran-2-yl)oxy)benzyl)oxy)carbonyl)amino)methyl)benzyl)-4-amino-2-butyl-1H-imidazo[4,5-c]quinoline-7-carboxylic acid (S70) as TFA salt, white solid. LCMS: m/z [M+H]⁺=1877.85 (theoretical); 1877.27 (observed). HPLC retention time=1.80 min (Method C).

4-amino-1-(4-(((((3-((S)-44-amino-38,45-dioxo-2,5,8,11,14,17,20,23,26,29,32,35-dodecaoxa-39,46-diazanonatetracontan-49-amido)-4-(((2S,3R,4S,5S,6S)-6-carboxy-3,4,5-trihydroxytetrahydro-2H-pyran-2-yl)oxy)benzyl)oxy)carbonyl)amino)methyl)benzyl)-2-butyl-1H-imidazo[4,5-c]quinoline-7-carboxylic acid (S71) as TFA salt, white solid. LCMS: m/z [M+H]⁺=1514.73 (theoretical); 1515.51 (observed). HPLC retention time=1.21 min (Method C).

4-amino-2-butyl-1-(4-(((((4-(((2S,3R,4S,5S,6S)-6-carboxy-3,4,5-trihydroxytetrahydro-2H-pyran-2-yl)oxy)-3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)benzyl)oxy)carbonyl)amino)methyl)benzyl)-1H-imidazo[4,5-c]quinoline-7-carboxylic acid (34) as TFA salt, white solid. LCMS: m/z [M+H]⁺=1665.76 (theoretical); 1666.27 (observed). HPLC retention time=1.34 min (Method C).

### Example 30

### Synthesis of a Drug-Linker Conjugate

This example covers synthesis of a complex comprising a linker coupled to a C₄ amine of an imidazoquinoline TLR7/8 agonist with C₇ carboxylic acid functionalization, namely (2S,3S,4S,5R,6S)-6-(2-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-((((2-(ethoxymethyl)-1-(2-hydroxy-2-methylpropyl)-1H-imidazo[4,5-c]quinolin-4-yl)carbamoyl)oxy)methyl)phenoxy)-3,4,5-trihydroxytetrahydro-2H-pyran-2-carboxylic acid (35). As outlined in SCHEME 60, compound 35 was generated from intermediate S60. Brieflt, intermediate S60 (12.5 mg, 0.009 mmol) and mp-OSu (2.80 mg, 0.011 mmol) were dissolved in anhydrous DMA (0.5 mL) and DIPEA (1.83 μL, 0.011 mmol) at room temperature. The reaction mixture was stirred at the same temperature for 90 min. After 90 min, the reaction mixture was quenched with HOAc (5 μL), diluted with DMSO/water, and purified by RP-HPLC (Method A) to give compound 33 (9.1 mg, 0.006 mmol, 65.8%) as TFA salt, white solid. LCMS: m/z [M+H]⁺=1576.73 (theoretical); 1577.11 (observed). HPLC retention time=1.25 min (Method C).

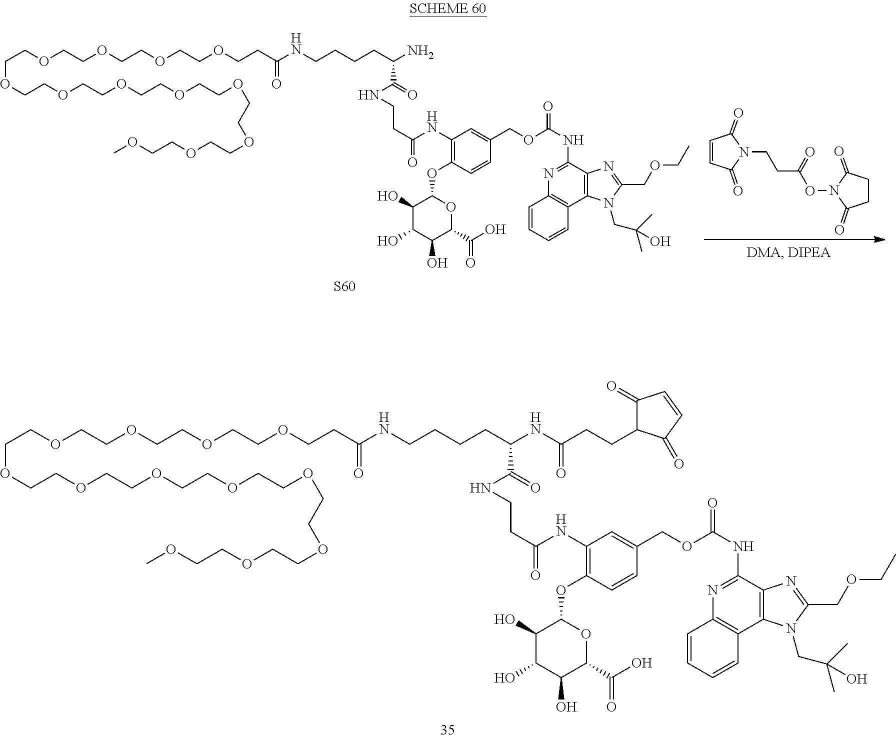

### Example 31

### Synthesis of a Drug-Linker Conjugate

This example covers synthesis of a complex comprising a linker coupled to a C₄ amine of an imidazoquinoline TLR7/8 agonist lacking C₇ functionalization, namely 3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)benzyl (2-(ethoxymethyl)-1-(2-hydroxy-2-methylpropyl)-1H-imidazo[4,5-c]quinolin-4-yl)carbamate (36). As outlined in SCHEME 61, the synthesis of compound 36 was similar to the synthesis of compound 35.

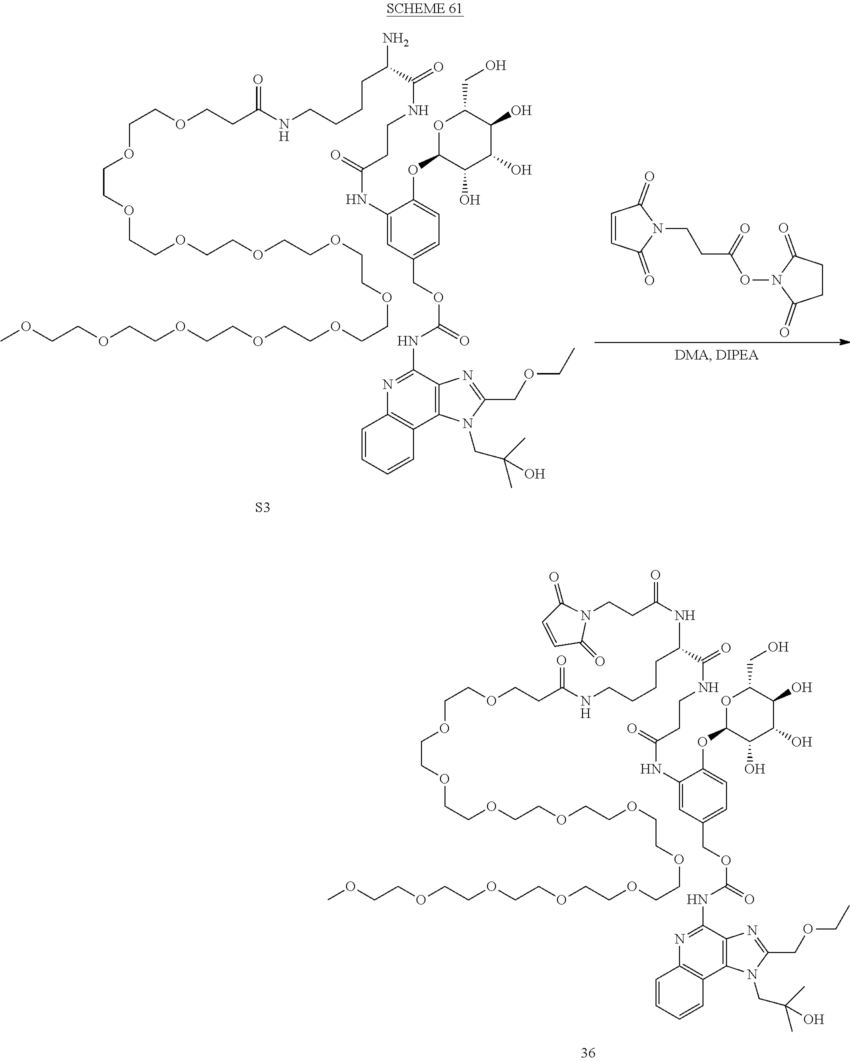

Synthesis of 3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3 S,4S,5S,6R)-3,4,5-trihydroxy-6-(hydroxymethyl)tetrahydro-2H-pyran-2-yl)oxy)benzyl (2-(ethoxymethyl)-1-(2-hydroxy-2-methylpropyl)-1H-imidazo[4,5-c]quinolin-4-yl)carbamate (36) as TFA salt, white solid. LCMS: m/z [M+H]⁺=1562.75 (theoretical); 1562.75 (observed). HPLC retention time=1.23 min (Method C).

### Example 32

### Synthesis of a Drug-Linker Conjugate

This example covers synthesis of a complex comprising a linker coupled to an N1 position of an imidazoquinoline TLR7/8 agonist with carboxylic acid C₇ functionalization, namely N-(4-((4-amino-2-butyl-7-carboxy-1H-imidazo[4,5-c]quinolin-1-yl)methyl)benzyl)-1-(3-((S)-44-(6-(2,5-dioxo-2,5-dihydro-1H-pyrrol-1-yl)hexanamido)-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)phenyl)-N,N-dimethylmethanaminium (37).

Compound 37 was synthesized according to SCHEME 62. Intermediate 22 (10.3 mg, 0.0064 mmol) and S86 (mc-OSu, AmBeed, 2.97 mg, 0.0097 mmol) were dissolved in anhydrous DMA (0.4 ml) and DIPEA (4.50 μl, 0.026 mmol) at room temperature. The reaction was stirred at the same temperature for 3 h. After 3 h n, the reaction mixture was quenched with HOAc (5 ul), diluted with DMSO/water and purified by RP-HPLC (Method A) to give 37 (6.7 mg, 0.004 mmol, 62.0%) as TFA salt, white solid. LCMS: m/z [M+H]⁺=1678.89 (theoretical); 1679.13 (observed). HPLC retention time=1.69 min (Method D).

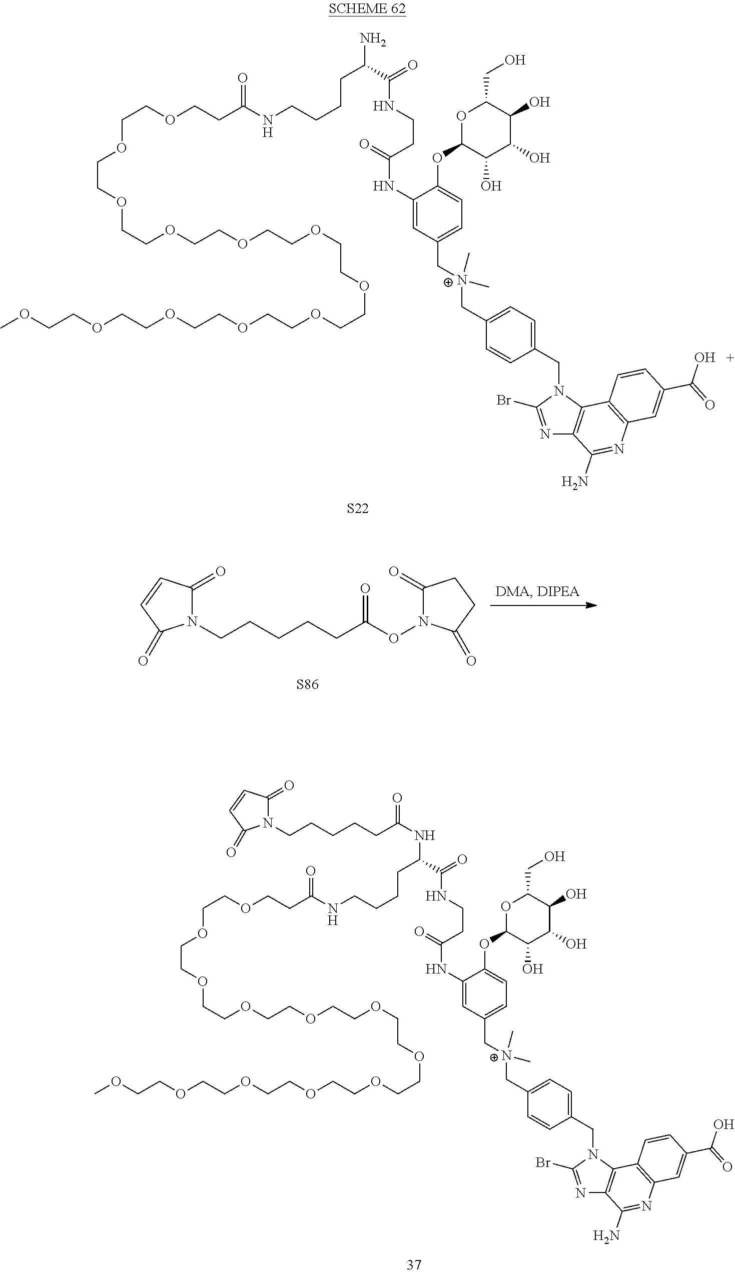 [SMILES: COCCOCCOCCOCCOCCOCCOCCOCCOCCOCCOCCOCCC(=O)NCCCC[C@H](N)C(=O)NCCC(=O)Nc1cc(C[N+](C)(C)Cc2ccc(Cn3c(Br)nc4c(N)nc5cc(C(=O)[OH2+])ccc5c43)cc2)ccc1O[C@H]1O[C@H](CO)[C@@H](O)[C@H](O)[C@@H]1O ; "S22" ; O=C(CCCCCN1C(=O)C=CC1=O)ON1C(=O)CCC1=O ; "S86" ; COCCOCCOCCOCCOCCOCCOCCOCCOCCOCCOCCOCCC(=O)NCCCC[C@H](NC(=O)CCCCCN1C(=O)C=CC1=O)C(=O)NCCC(=O)Nc1cc(C[N+](C)(C)Cc2ccc(Cn3c(Br)nc4c(N)nc5cc(C(=O)O)ccc5c43)cc2)ccc1O[C@H]1O[C@H](CO)[C@@H](O)[C@H](O)[C@@H]1O]

### Example 33

### Synthesis of a Drug-Linker Conjugate

This example covers synthesis of a complex comprising a linker coupled to an N1 position of an imidazoquinoline TLR7/8 agonist with carboxylic acid C₇ functionalization, namely N-(3-((S)-44-((S)-3-amino-2-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)benzyl)-1-(4-((4-amino-2-butyl-7-carboxy-1H-imidazo[4,5-c]quinolin-1-yl)methyl)phenyl)-N,N-dimethylmethanaminium (38). Compound 38 synthesis was performed according to SCHEME 63. Intermediates S22 (16.0 mg, 0.01 mmol) and S4 (5.72 mg, 0.015 mmol) were dissolved in anhydrous DMA (0.4 mL) and DIPEA (0.009 mL, 0.05 mmol) at room temperature. The reaction was stirred at room temperature for 2 h. After 2 h, solvent was removed in vacuo and the crude reaction mixture was re-dissolved in DCM/TFA (5:1 (v/v), 0.5 mL), and the reaction mixture was stirred at room temperature for 20 min. Solvent was then removed and the crude reaction mixture was diluted with DMSO/water and purified by RP-HPLC (Method A) to give compound 38 (16.5 mg, 0.006 mmol, 61.1%) as TFA salt. LCMS: m/z [M+H]⁺=1651.85 (theoretical); 1652.10 (observed). HPLC retention time=1.40 min (Method D).

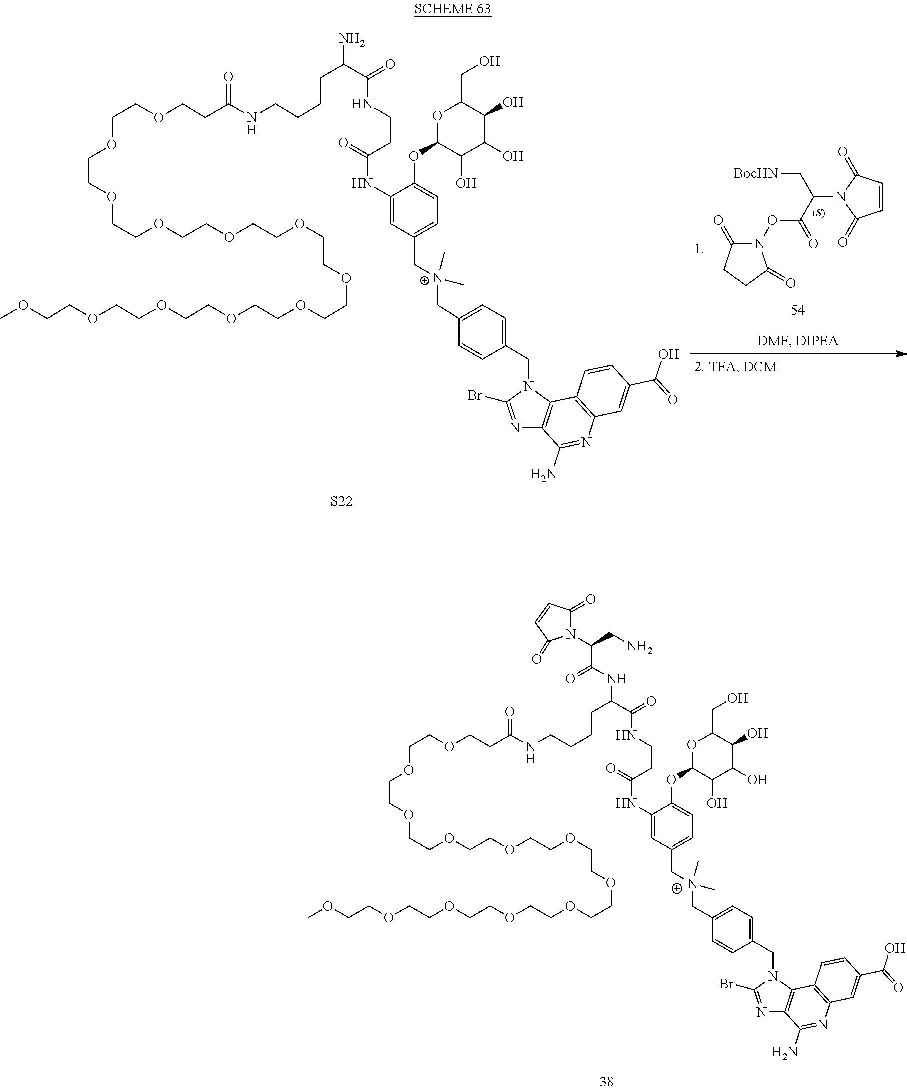

### Example 34

### Synthesis of a Drug-Linker Conjugate

This example covers synthesis of a complex comprising a linker coupled to an N1 position of an imidazoquinoline TLR7/8 agonist with carboxylic acid C₇ functionalization and a hydrolysable group coupled to a C4 amine, namely N-(4-((2-butyl-7-carboxy-4-((((4-(((2R,3S,4S,5S,6R)-3,4,5-trihydroxy-6-(hydroxymethyl)tetrahydro-2H-pyran-2-yl)oxy)benzyl)oxy)carbonyl)amino)-1H-imidazo[4,5-c]quinolin-1-yl)methyl)benzyl)-1-(3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)phenyl)-N,N-dimethylmethanaminium (40).

Compound S87 was prepared according to the procedures as described in Angew. Chem. Int. Ed. 2006, 45, 5345-5348.

Synthesis of (2R,3R,4S,5S,6R)-2-(acetoxymethyl)-6-(4-((((2-butyl-1-(4-((dimethylamino)methyl)benzyl)-7-(methoxycarbonyl)-1H-imidazo[4,5-c]quinolin-4-yl)carbamoyl)oxy)methyl)phenoxy)tetrahydro-2H-pyran-3,4,5-triyl triacetate (S80a) is outlined in SCHEME 64. Compound S18a (105 mg, 0.236 mmol) was dissolved in anhydrous THF (2.5 mL) and cooled to 0° C. 1,1′-carbonyl-do-(1,2,4-triazole) (CDT, 54.1 mg, 0.330 mmol) was added and the reaction mixture was warmed up to room temperature and stirred for 30 min. After 30 min, compound S87 (267.7 mg, 0.589 mmol) was added to the reaction mixture. The reaction was stirred at room temperature for 45 min. After 45 min, solvents were removed in vacuo and the crude reaction mixture was purified by normal phase HPLC (Method B, EtOAc/hexane as eluents) to give the desired product S80a (192.1 mg, 0.208 mmol, 88.0%) as white solid. LCMS: m/z [M+H]⁺=926.38 (theoretical); 926.37 (observed). HPLC retention time=1.88 min (Method D).

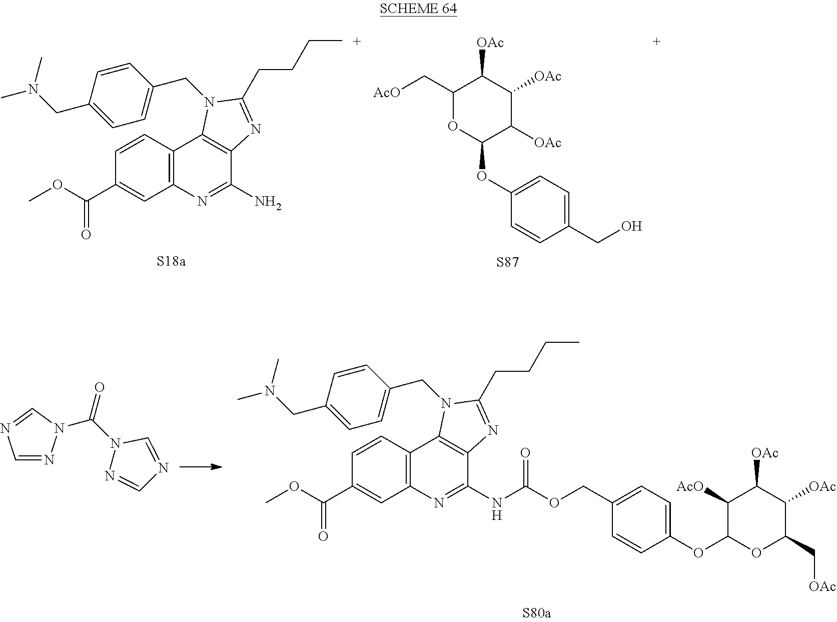

Synthesis of N-(3-(3-((((9H-fluoren-9-yl)methoxy)carbonyl)amino)propanamido)-4-(((2R,3S,4S,5R,6R)-3,4,5-triacetoxy-6-(acetoxymethyl)tetrahydro-.2H-pyran-2-yl)oxy)benzyl)-1-(4-((2-butyl-7-(methoxycarbonyl)-4-((((4-(((2R,3S,4S,5R,6R)-3,4,5-triacetoxy-6-(acetoxymethyl)tetrahydro-2H-pyran-2-yl)oxy)benzyl)oxy)carbonyl)amino)-1H-imidazo[4,5-c]quinolin-1-yl)methyl)phenyl)-N,N-dimethylmethanaminium (S88a) is outlined in SCHEME 65. Compound S17 (252.8 mg, 0.306 mmol) and S80a (189.0 mg, 0.204 mmol) were dissolved in DMA (7.5 mL). The reaction mixture was heated at 50° C. for 90 min and the reaction progress monitored by LCMS. After 90 min, the reaction mixture was diluted with DMSO/water and purified by RP-HPLC (Method A) to provide S83a (285.1 mg, 0.171 mmol, 83.6%) as TFA salt, white solid. LCMS: m/z [M]⁺=1670.63 (theoretical); 1670.63 (observed). HPLC retention time=2.34 min (Method D).

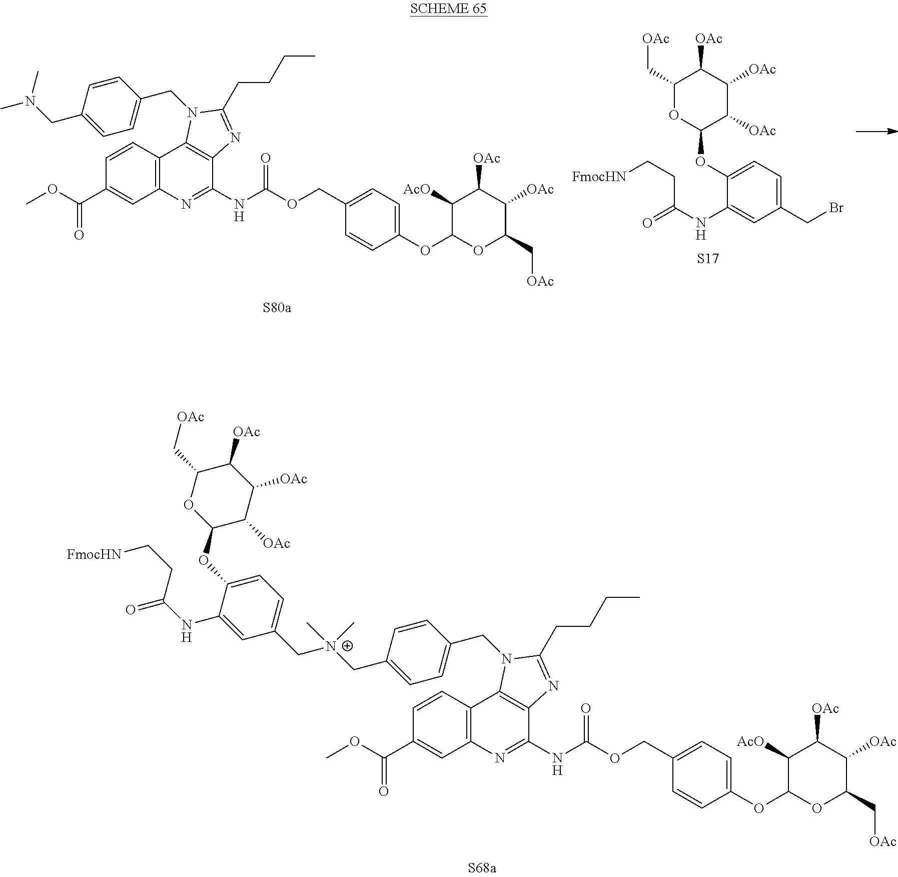

Synthesis of N-(3-(3-aminopropanamido)-4-(((2R,3S,4S,5S,6R)-3,4,5-trihydroxy-6-(hydroxymethyl)tetrahydro-2H-pyran-2-yl)oxy)benzyl)-1-(4-((2-butyl-7-carboxy-4-((((4-(((2R,3S,4S,5S,6R)-3,4,5-trihydroxy-6-(hydroxymethyl)tetrahydro-2H-pyran-2-yl)oxy)benzyl)oxy)carbonyl)amino)-1H-imidazo[4,5-c]quinolin-1-yl)methyl)phenyl)-N,N-dimethylmethanaminium (S89b) is outlined in SCHEME 66. Intermediate S88a (85.0 mg, 0.051 mmol) was dissolved in dichloromethane (DCM, 3.0 mL) at rom temperature (RT) followed by the addition of dimethylamine (Et₂NH, 1.0 mL). The reaction mixture was stirred at RT for 45 min. After 45 min, solvents were removed in vacuo and the crude reaction mixture was re-dissolved in THF (1.0 mL) at 0° C. and a lithium hydroxide (LiOH) solution (0.5 M in MeOH, 1.0 mL, 0.509 mmol) was added. The resulting reaction mixture was stirred at 0° C. for 90 min, upon which, glacial acetic acid (AcOH, 60.0 uL, 1.02 mmol) was added to neutralize the reaction mixture. The reaction mixture was diluted with water/DMSO and purified by preparative RP-HPLC (Method A) to give intermediate S89b (39.7 mg, 0.036 mmol, 71.0%) as TFA salt, white solid. LCMS: m/z [M]⁺=1098.47 (theoretical); 1098.41 (observed). HPLC retention time=1.28 min (Method D).

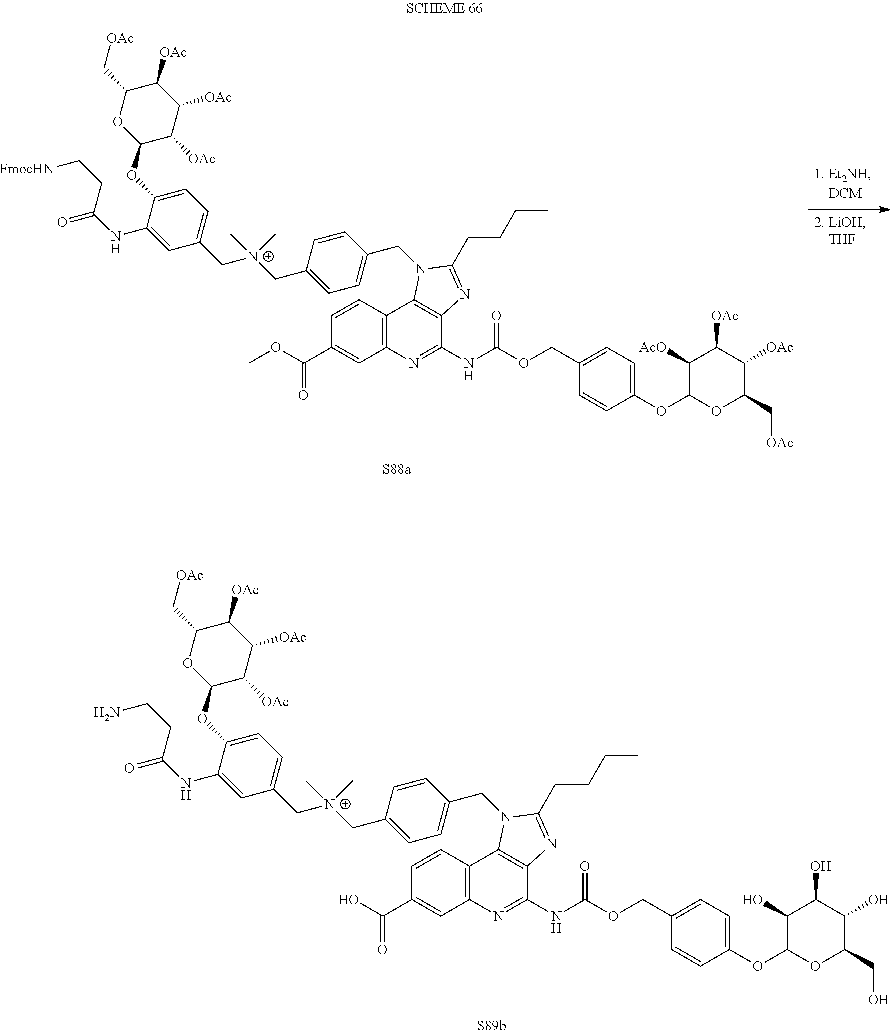

Synthesis of N-(4-((2-butyl-7-carboxy-4-((((4-(((2R,3S,4S,5S,6R)-3,4,5-trihydroxy-6-(hydroxymethyl)tetrahydro-2H-pyran-2-yl)oxy)benzyl)oxy)carbonyl)amino)-1H-imidazo[4,5-c]quinolin-1-yl)methyl)benzyl)-1-(3-(3-(3-(2,5-dioxo-2,5-dihydro-1H-pyrrol-1-yl)propanamido)propanamido)-4-(((2R,3S,4S,5S,6R)-3,4,5-trihydroxy-6-(hydroxymethyl)tetrahydro-2H-pyran-2-yl)oxy)phenyl)-N,N-dimethylmethanaminium (39) is outlined in SCHEME 67. Intermediate S89b (40.1 mg, 0.033 mmol) and mp-OSu (13.2 mg, 0.050 mmol) were dissolved in anhydrous DMA (1.0 mL) and DIPEA (21.3 μL, 0.165 mmol) at room temperature. The reaction mixture was stirred at the same temperature for 30 min. After 30 min, the reaction mixture was quenched with HOAc (10 μl), diluted with DMSO/water, and purified by RP-HPLC (Method A) to give compound 39 (20.6 mg, 0.017 mmol, 49.8%) as TFA salt, white solid. LCMS: m/z [M]⁺=1249.49 (theoretical); 1249.51 (observed). HPLC retention time=1.41 min (Method D).

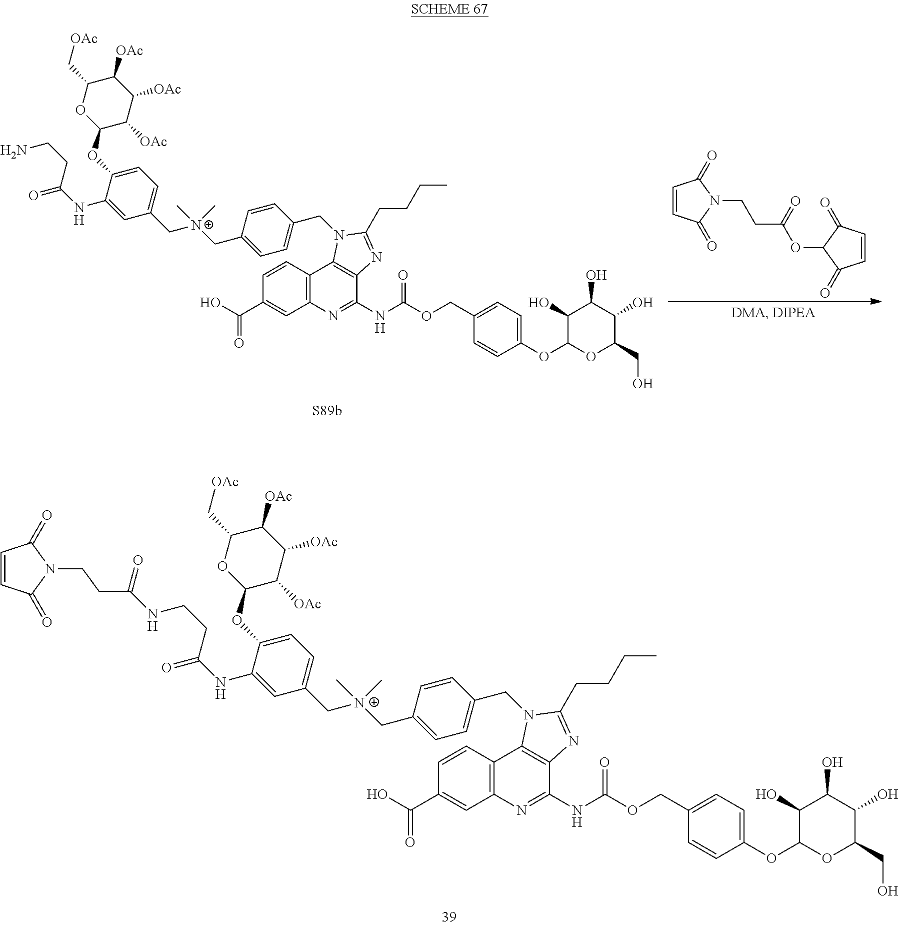

### Example 35

### Synthesis of a Drug-Linker Conjugate

This example covers synthesis of a complex comprising a linker coupled to an N1 position of an imidazoquinoline TLR7/8 agonist with carboxylic acid C₇ functionalization and a hydrolysable group coupled to a C4 amine, namely N-(4-((2-butyl-7-carboxy-4-((((4-(((2R,3S,4S,5S,6R)-3,4,5-trihydroxy-6-(hydroxymethyl)tetrahydro-2H-pyran-2-yl)oxy)benzyl)oxy)carbonyl)amino)-1H-imidazo[4,5-c]quinolin-1-yl)methyl)benzyl)-1-(3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)phenyl)-N,N-dimethylmethanaminium (40).

Synthesis of N-(3-((S)-44-((((9H-fluoren-9-yl)methoxy)carbonyl)amino)-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)benzyl)-1-(4-((2-butyl-7-carboxy-4-((((4-(((2R,3S,4S,5S,6R)-3,4,5-trihydroxy-6-(hydroxymethyl)tetrahydro-2H-pyran-2-yl)oxy)benzyl)oxy)carbonyl)amino)-1H-imidazo[4,5-c]quinolin-1-yl)methyl)phenyl)-N,N-dimethylmethanaminium (S90b). Compound S86b was prepared using similar procedures as those used in the synthesis of S60. (S90b) as TFA salt, white solid. LCMS: m/z [M+H]⁺=2019.96 (theoretical); 2019.90. (observed). HPLC retention time=1.70 min (Method D).

Synthesis of N-(3-((S)-44-amino-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)benzyl)-1-(4-((2-butyl-7-carboxy-4-((((4-(((2R,3S,4S,5S,6R)-3,4,5-trihydroxy-6-(hydroxymethyl)tetrahydro-2H-pyran-2-yl)oxy)benzyl)oxy)carbonyl)amino)-1H-imidazo[4,5-c]quinolin-1-yl)methyl)phenyl)-N,N-dimethylmethanaminium (S91b) is outlined in SCHEME 68. (S91b) as TFA salt, white solid. LCMS: m/z [M+H]⁺=1797.89 (theoretical); 1797.85. (observed). HPLC retention time=1.47 min (Method D).

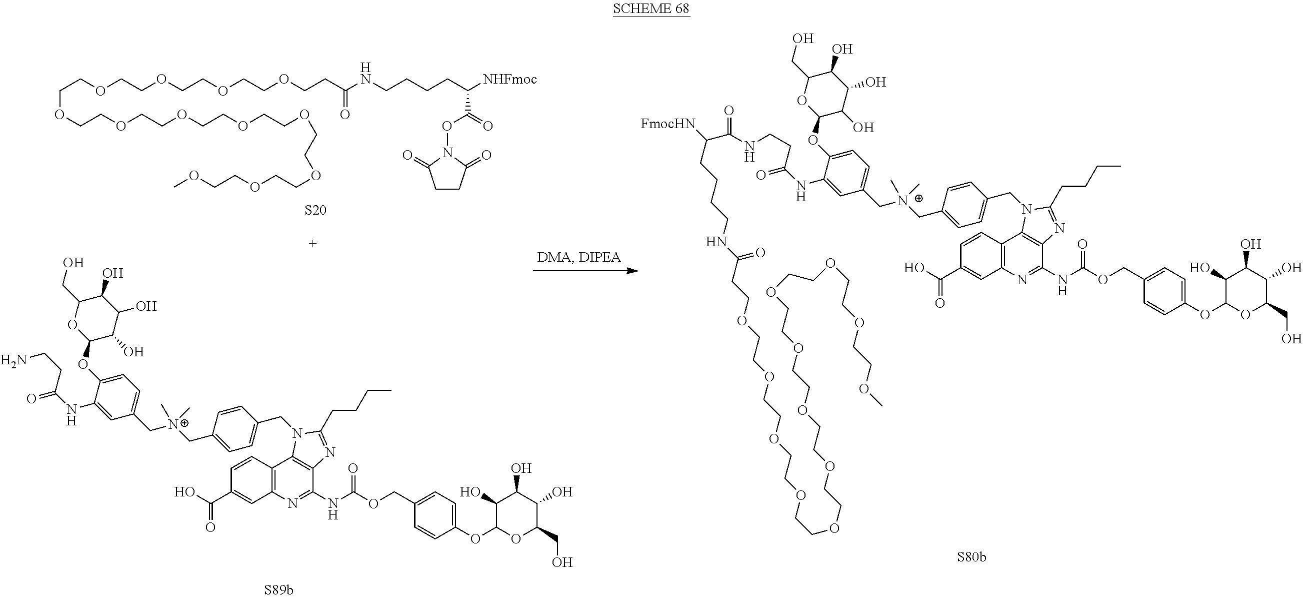

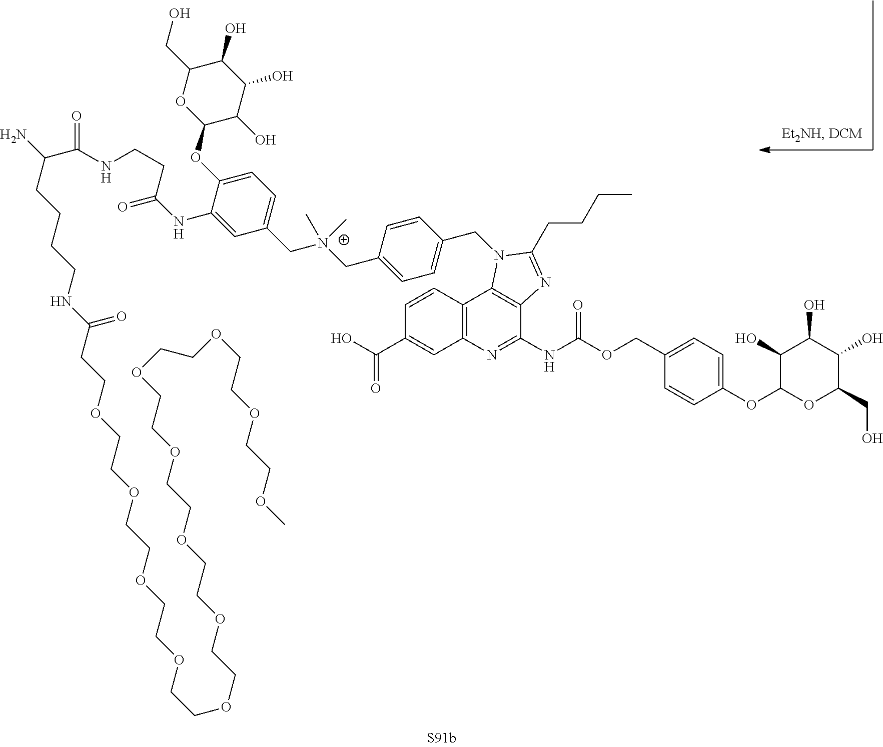 [SMILES: CCCCc1nc2c(NC(=O)OCc3ccc(OC4O[C@H](CO)[C@@H](O)[C@H](O)[C@@H]4O)cc3)nc3cc(C(=O)O)ccc3c2n1Cc1ccc(C[N+](C)(C)Cc2ccc(O[C@H]3OC(CO)[C@@H](O)[C@H](O)C3O)c(NC(=O)CCNC(=O)C(N)CCCCNC(=O)CCOCCOCCOCCOCCOCCOCCOCCOCCOCCOCCOCCOC)c2)cc1]

Synthesis of N-(4-((2-butyl-7-carboxy-4-((((4-(((2R,3S,4S,5S,6R)-3,4,5-trihydroxy-6-(hydroxymethyl)tetrahydro-2H-pyran-2-yl)oxy)benzyl)oxy)carbonyl)amino)-1H-imidazo[4,5-c]quinolin-1-yl)methyl)benzyl)-1-(3-((S)-44-(3-(2,5-dioxo-2,5-dihydro-1H-pyrrol-1-yl)propanamido)-38,45-dioxo-2,5,8,11,14,17,20,23,26,29,32,35-dodecaoxa-39,46-diazanonatetracontan-49-amido)-4-(((2R,3S,4S,5S,6R)-3,4,5-trihydroxy-6-(hydroxymethyl)tetrahydro-2H-pyran-2-yl)oxy)phenyl)-N,N-dimethylmethanaminium (40) is outlined in SCHEME 69. Intermediate S91b (28.2 mg, 0.016 mmol) and mp-OSu (6.26 mg, 0.024 mmol) were dissolved in anhydrous DMA (0.5 mL) and DIPEA (21.9 μL, 0.126 mmol) at room temperature. The reaction mixture was stirred at the same temperature for 30 min. After 30 min, the reaction mixture was quenched with HOAc (10 pil), diluted with DMSO/water, and purified by RP-HPLC (Method A) to give compound 40 (21.4 mg, 0.017 mmol, 70.0%) as TFA salt, white solid. LCMS: m/z [M+H]⁺=1948.92 (theoretical); 1948.83 (observed). HPLC retention time=1.51 min (Method D).

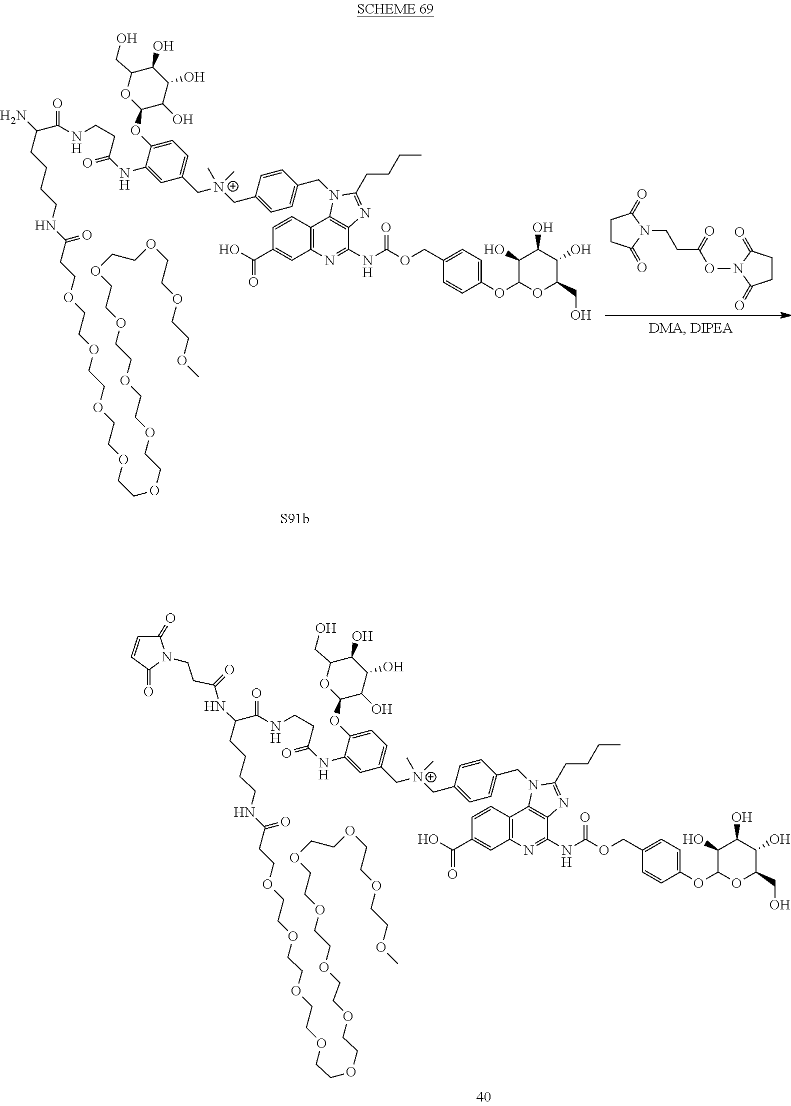

### Example 36

### Synthesis of Multiple Imidazoquinoline Complexes

Compounds S72a, S73a, S74a, S75a, S76a, S77a, and S78a were prepared analogously to procedures described in U.S. Publ. No. 2017/0217960; Larson, et al. ACS Med. Chem. Lett. Vol. 8, No. 11, 1148-1152 (2017); and Schiaffo, et al., J. Med. Chem. Vol. 57, No. 2, 339-347 (2014), each of which is incorporated by reference in its entirety.

### Example 37

### Biological Activity of TLR Agonists and Antibody Drug Conjugates

This example covers dose dependent human immune cell activation by small molecule TLR7/8 agonists. Primary human PBMCs were isolated from peripheral whole blood from human donors and were subjected to treatment with increasing concentrations of the agonists diluted in PBS. Cell culture supernatants were harvested 24 h after treatment and analyzed for induction of cytokines via multiplex ELISAs (Luminex).

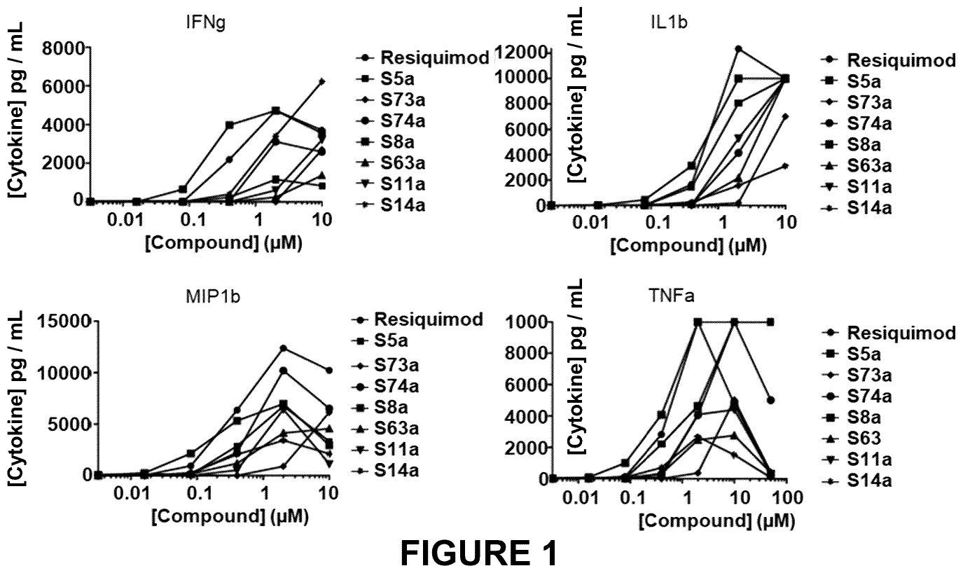

FIG. 1 summarizes dose dependent interferon gamma (IFNg, top left panel), interleukin 1 beta (IL1IP, top right panel), macrophage inflammatory protein 1 beta (MIP1IP, bottom left panel), and tumour necrosis factor alpha (TNFα, bottom right panel) induction in primary human PBMCs by Resiquimod (a 4-imidazoquinoline compound lacking C₇ functionalization) and a series of 4-imidazoquinoline compounds with C₇ functionalization, namely S5a, S73a, S74a, S8a, S63a, S11a, and S14a. When compared to the commercially available compound resiquimod, several of the compounds displayed similar potency. Notably, the compound S8a exhibited the highest potency for cytokine induction (FIG. 1), eliciting cytokine responses at sub 0.1 μM concentrations.

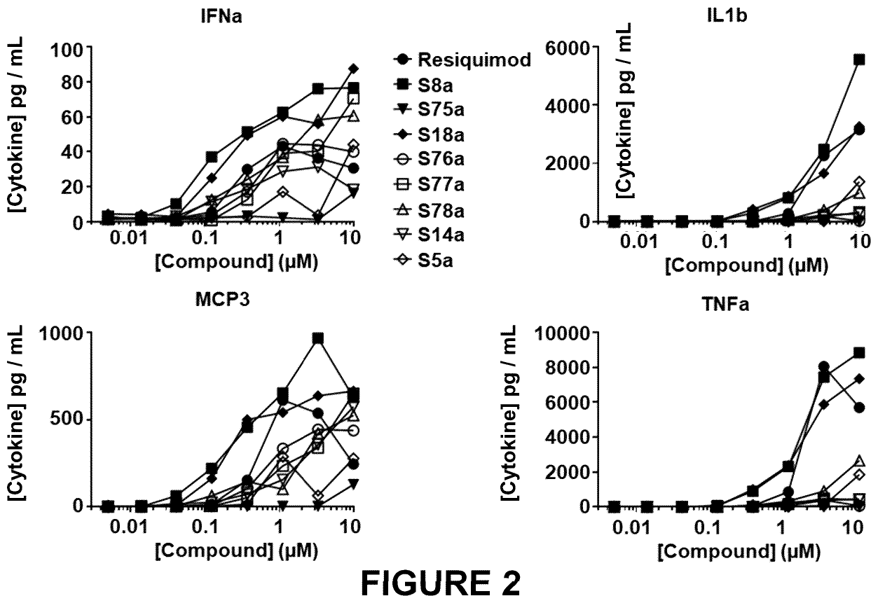

FIG. 2 summarizes dose dependent cytokine induction with additional 4-imidazoquinoline methyl ester TLR agonists. Induction of interferon alpha (IFNA, top left panel), IL1β (top right panel), monocyte chemotactic protein-3 (MCP3, bottom left panel), and TNFα (bottom right panel) responses in primary human PBMCs were measured as a function of agonist concentration to assess potency and efficacy at activating these immune cells. This series of assays utilized Reisuimod and compounds S8a, S75a, S14a, S76a, S77a, S78a, S14a, and S5a. The agonists S18a and S8a displayed some of the most potent ability to activate these cells, exhibiting activities at sub 0.1 μM concentrations, and indicating that phenylmethanamine substitution may be important for TLR activation by imidazoquinoline agonists.

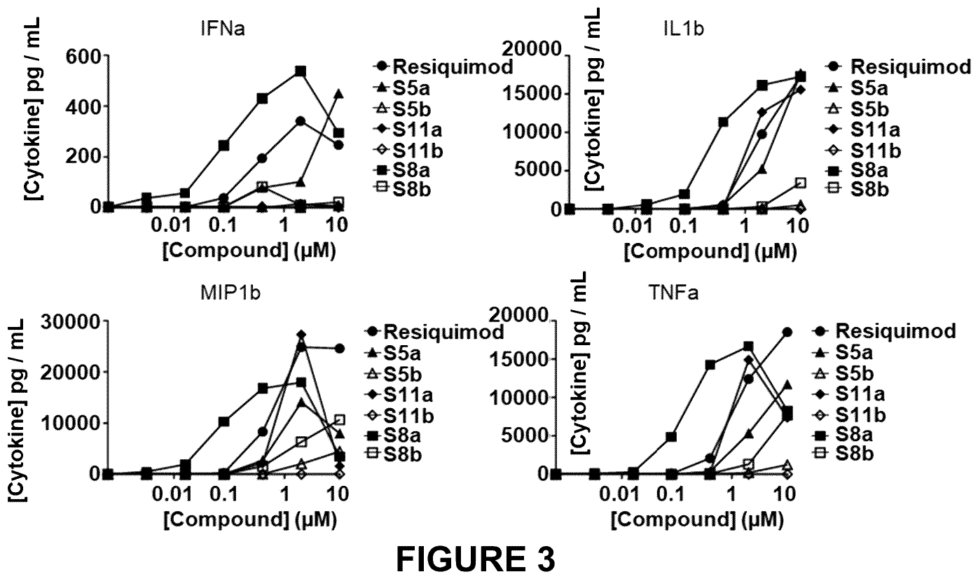

To compare the effects of C₇ carboxylation and carboxymethylation in imidazoquinoline TLR antagonists, PBMC cytokine responses were compared for multiple molecules of each type. FIG. 3 summarizes IFNα (top left panel), IL1β (top right panel), MIP1β (bottom left panel), and TNFα (bottom right panel) induction in PBMCs with multiple methyl ester imidazoquinoline TLR agonists (S5a, S8a, S11a) and their carboxyl counterparts (S5b, S8b, S11b), which were modified to include a carboxylic acid instead of a methyl ester on the C₇ position. When these carboxylic acid derivatives (S5b, S11b and S8b) were tested for their ability to stimulate cytokine production from human peripheral blood cells, a striking phenotype was seen. Each carboxylic acid compounds exhibited markedly lower potency and activity than its corresponding parent methyl ester compound for all cytokines assessed. These results suggest that, in some cases, C₇ carboxylic acids confer lower activity than C₇ methyl esters, rendering them less active as small molecule innate immune agonists.

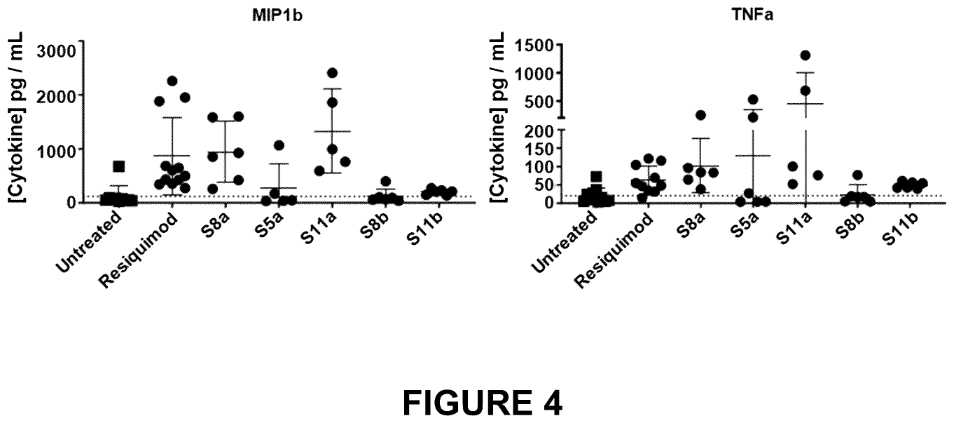

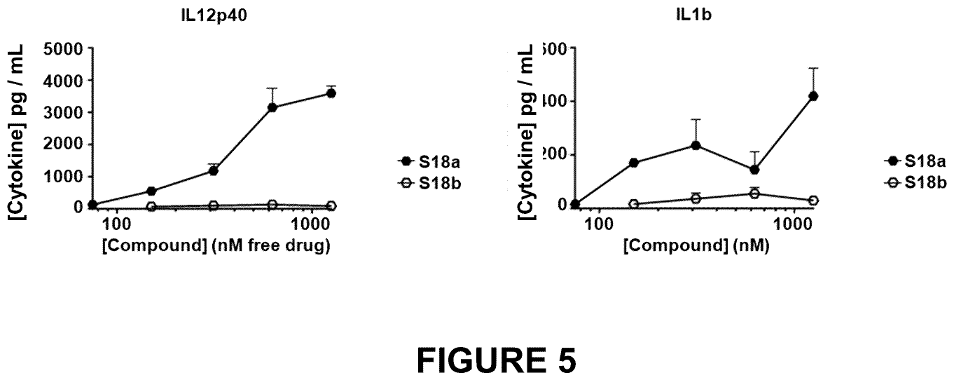

Differences in cytokine induction by C₇ carboxylated and carboxymethylated antagonists are further highlighted in FIGS. 4-5. FIG. 4 provides MIP1β (left panel) and TNFα (right panel) responses generated with Resiquimod and compounds S8a, S5a, S11a S8b, and S11b. S8a and S11a elicited similar MIP1β responses as Resiquimod, while S8b and S11b exhibit lower activities than their methyl ester counterparts. FIG. 5 provides a comparison of interleukin 12 subunit beta (IL12p40, left panel) and IL1β (right panel) responses generated with the C₇ carboxymethyl compound 518a and its C₇ carboxyl analogue S18b. The carboxylic acid derivative S18a affected lower responses of both cytokines as compared to its methyl ester analogue S18b.

As one possibility for the reduced activity of the carboxylic acid derivative TLR7/8 agonists (as compared to the corresponding methyl ester compounds) may be due to lower permeability, it was hypothesized that conjugating these molecules to antibodies to improve cellular uptake could render these compounds more active. Cells were stimulated with the various TLR7/8 agonist-antibody conjugates at different concentrations, and resulting supernatants were harvested and analyzed using a multiplex analyte kit (Luminex).

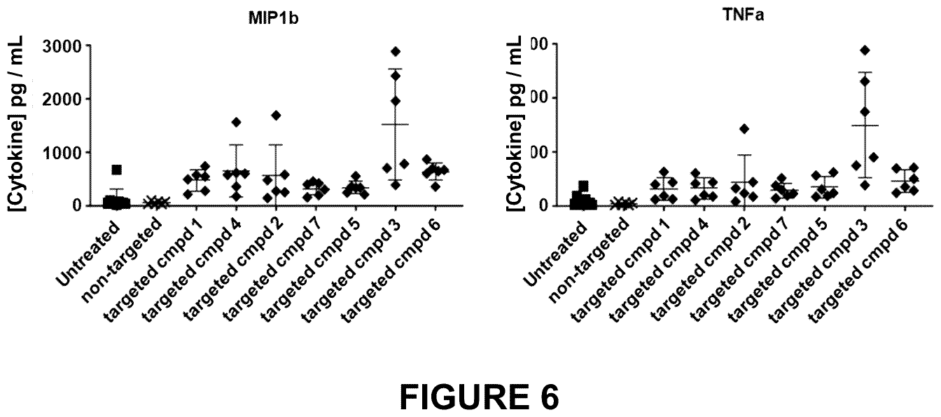

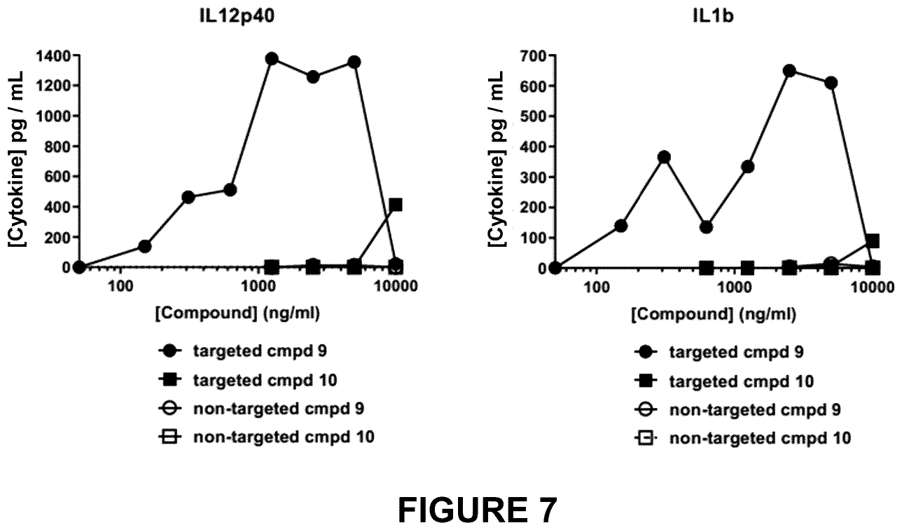

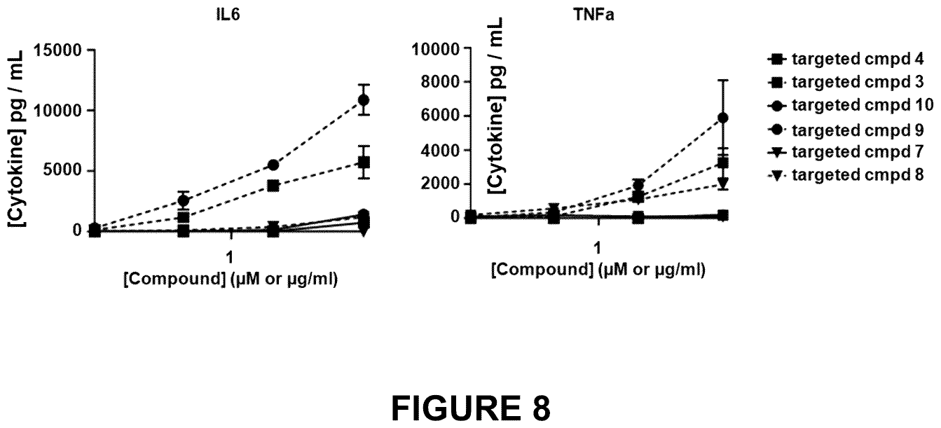

Immune cells were treated with 5 μg/mL of an immune-targeting antibody, TLR agonist compounds, and antibody conjugated with different TLR agonist compounds (to thereby form ADCs with average drug:antibody ratios of about 8). FIG. 6 summarizes MIP1β (left panel) and TNFα (right panel) responses generated by untreated immune cells, a TLR agonist coupled to a non-targeting antibody (‘Non-targeted’), and TLR agonist compounds conjugated with immune-targeted antibodies (‘targeted cmpd’ 1-7). Targeted compound 1 corresponds to Resiquimod coupled to an immunostimulatory antibody by a cleavable linker. Targeted compounds 2, 4, 5, and 7 correspond to C₇ carboxymethylated imidazoquinoline compounds coupled to immunostimulatory antibodies by cleavable linkers. Targeted compounds 3 and 6 correspond to C₇ carboxylated imidazoquinoline compounds coupled to immunostimulatory antibodies by cleavable linkers. FIG. 7 provides dose-dependent IL12p40 (left panel) and IL1β (right panel) responses generated with non-targeted (i.e., coupled to an isotype antibody) and immune targeting antibody conjugated compounds, with ‘cmpd 9’ corresponding to a C₇ carboxylated imidazoquinoline compound and ‘cmpo10’ corresponding to its C₇ carboxymethyl analogue. FIG. 8 provides dose-dependent interleukin 6 (IL6, left panel) and TNFα (right panel) responses with ADCs comprising immune cell targeting antibodies coupled to either C₇ carboxylated (cmpd 3, 8, 9) or C₇ carboxymethylated (cmpd 4, 7, 10) imidazoquinoline compounds.

Unlike unconjugated C₇ carboxylated imidazoquinoline compounds, which were unable to activate immune cells (FIGS. 4 and 5), ADCs comprising these compounds (S8b, S11b and 518b) were just as, if not more potent at activating immune cells than their methyl ester analogues (FIGS. 6, 7, and 8). For example, in FIG. 8, the C₇ carboxylated compounds (indicated with dashed lines) generated much stronger responses than their C₇ carboxymethyl counterparts (solid lines). Importantly the non targeting conjugated molecules did not show ability to drive activation suggesting the conjugates had immunospecificity. These results suggest that the C₇ carboxylated compounds may have strong TLR7 agonistic activity but low uptake, such that conjugation to a targeting antibody could be a way to deliver these impermeable agonists to affect immune stimulating activity.

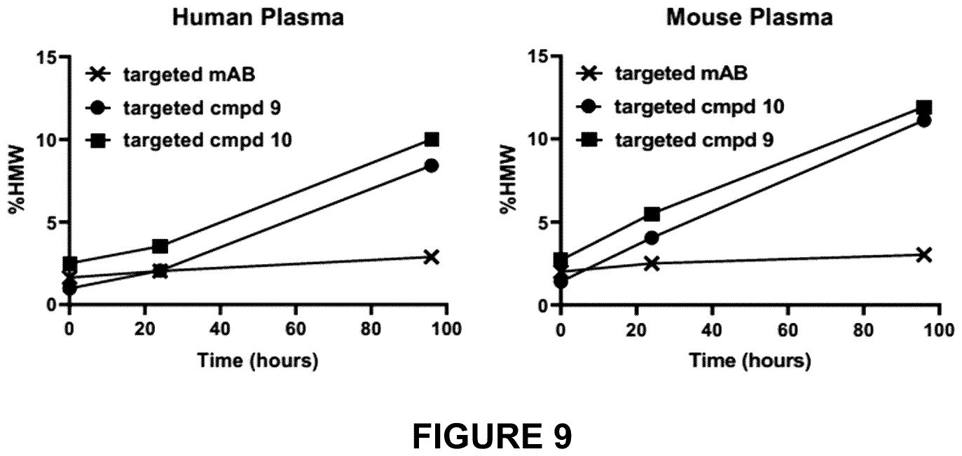

To determine if any of these differences in activity potentially resulted from increases in aggregation as a subsequent result of changes in charge, SEC analysis was performed. ADCs containing either S18a (methyl ester) or S18b (carboxylic acid) were incubated with both human or mouse plasma and assessed for the formation of high molecular weight species (HMW) over time. While the conjugates did form more aggregates over time than naked monoclonal antibody (mAB) (FIG. 9), there was no significant difference in the amounts of aggregation between the methyl ester and carboxylic acid containing ADCs. Therefore, it does not appear that the differences their activity is due to physical changes in the antibody drug conjugate species.

### Example 38

### In Vivo Activity of Antibody Drug Conjugates with TLR7/8 Agonists

This example covers in vivo assessments of anti-tumor activity of ADCs with TLR7/8 agonists. An in vivo syngeneic system was used to assess the ability of the ADCs having TLR ⅞ agonists as the payload to induce immune responses and drive an anti-tumor immune response using a subcutaneous, heterotopic mouse colon carcinoma model (a CT26 system). Female Balb/c mice were implanted with 1×10⁵ CT26 cells sub-cutaneously in the flank on day 0. When mean tumor size of 100 mm³ (measured using the formula: Volume (mm³)=0.5*Length*Width² where length is the longer dimension) was reached, the mice were randomized into treatment groups of 6 mice per group. Animals were then treated intraperitoneally with the indicated treatment every 7 days, for 3 doses total. Stock concentrations of the ADCs were diluted to the appropriate concentration and injected into animals in 100 μL volumes. Tumor length and width and mouse weights were measured throughout the study and tumor volume was calculated using the formula above. Animals were followed until tumor volume reached 1000 mm³, when animals were then euthanized.

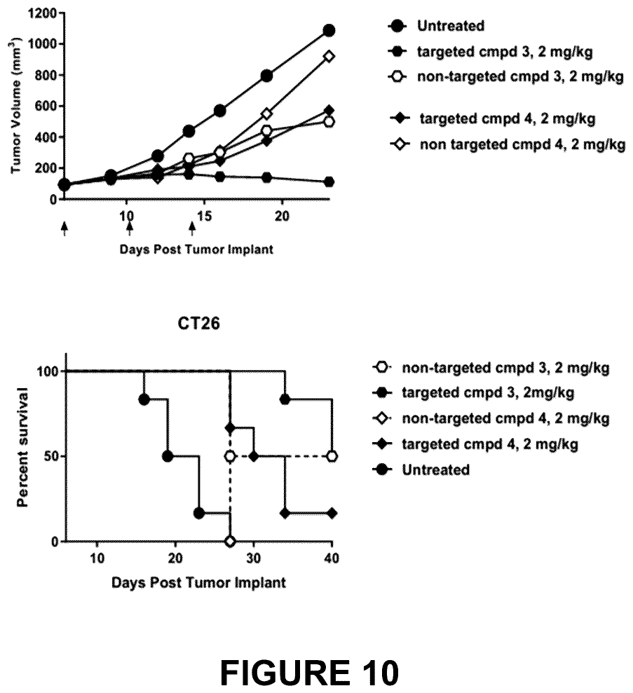

FIG. 10 summarizes tumor volume (top panel) and percent survival in 5 treatment groups. As shown, animals were treated with the ADC containing the TLR7/8 agonist S5a (cmpd 4), some tumor growth delay and survival benefit was seen with ⅙ of the mice surviving at the end of the study. This effect was enhanced with the targeted ADC over the non-targeted control conjugate. When the corresponding acid derivative (S5b, cmpd 3) was used as a payload on the same antibodies, greatly enhanced anti-tumor efficacy was seen with profound tumor growth delay and survival at study end of up to 50% of the animals (FIG. 10).

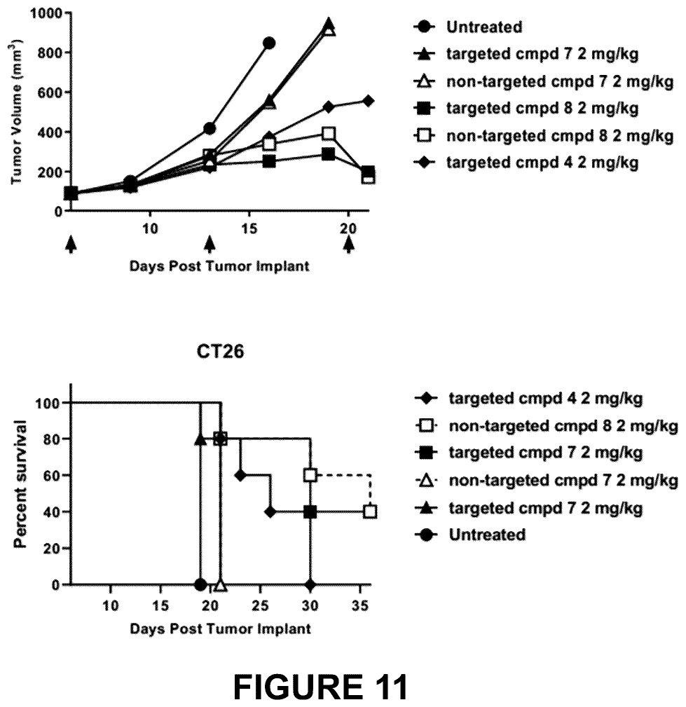

To confirm these results and assess if ADCs containing acid derivatives of different TLR7/8 agonists induce similar increase in anti-tumor activity and potency, another CT26 syngeneic experiment was performed similar to the one described in FIG. 10, with results summarized in FIG. 11. In this figure, the top panel summarizes tumor volume while the bottom panel provides percent survival across 6 treatment groups. Again, it was clear that the ADCs containing the methyl ester derivative of the TLR ⅞ agonist (compound S14a, cmpd 7) afforded some tumor growth delay over the untreated group, though no animals ultimately survived. However, conjugation of the acid derivative S14b (cmpd 8) appended to the targeting antibody, greatly increased tumor growth delay and survival of the mice bearing these tumors was seen (FIG. 11). Again, these data support the conclusion that modification of the C₇ position to carboxylic acid increases the potency and anti-tumor efficacy of a TLR7/8 ADC.

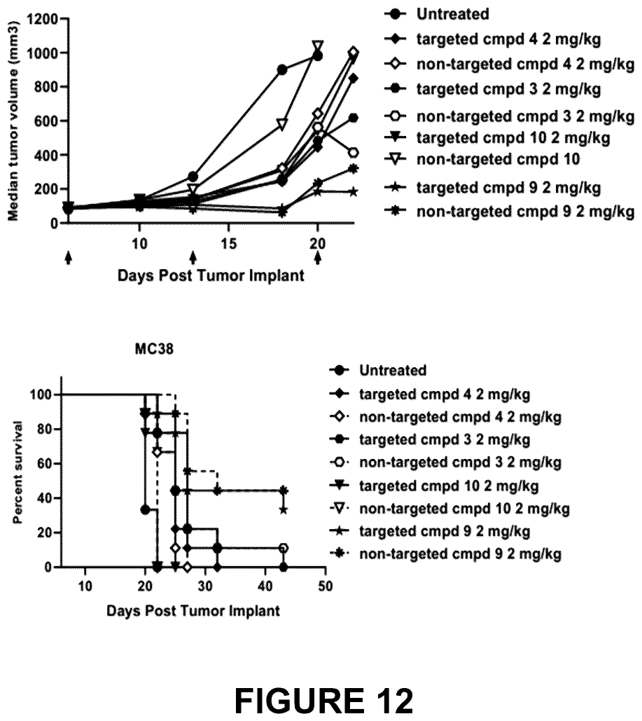

The activity of several conjugated TLR7/8 molecules in both methyl ester and carboxylic acid forms were also assessed in another colon tumor model, MC38. Results of this analysis are summarized in FIG. 12, which the top panel summarizing tumor volume and the bottom panel providing percent survival across 9 treatment groups. For ADCs containing S8a (cmpd 4, methyl ester) or S8b (cmpd 3, carboxylic acid), in this tumor model, there was a slight increased survival advantage with the acid derivative (FIG. 12, ♦ vs. hexagon). However, the increase in anti-tumor activity of the carboxylic acid version of the ADC was much more pronounced in this model for the S18a (methyl ester) or S18b (carboxylic acid) TLR7/8 agonist. Here we see a distinct tumor growth delay and overall survival benefit with ADC containing S18b over the ADC containing S18a (FIG. 12, ▾ vs. * or star).

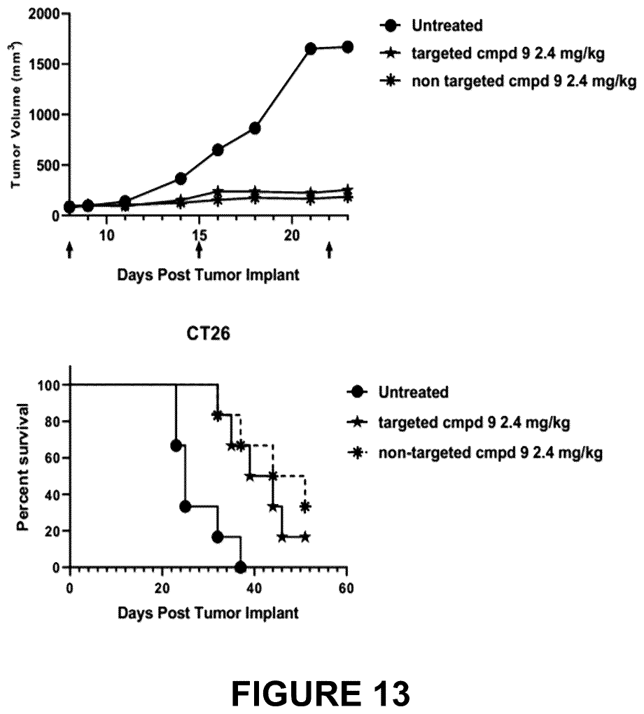

The activity of this most active conjugate (ADC containing S18b) was further assessed in the CT26 colon tumor model; where potent tumor growth delay and a long-term overall survival benefit were also observed. FIG. 13 summarizes the results of this analysis, with the top panel summarizing tumor volume and the bottom panel providing percent survival for untreated, targeted ADC with cmpd 9-treated, and non targeted ADC with cmpd 9-treated mice.

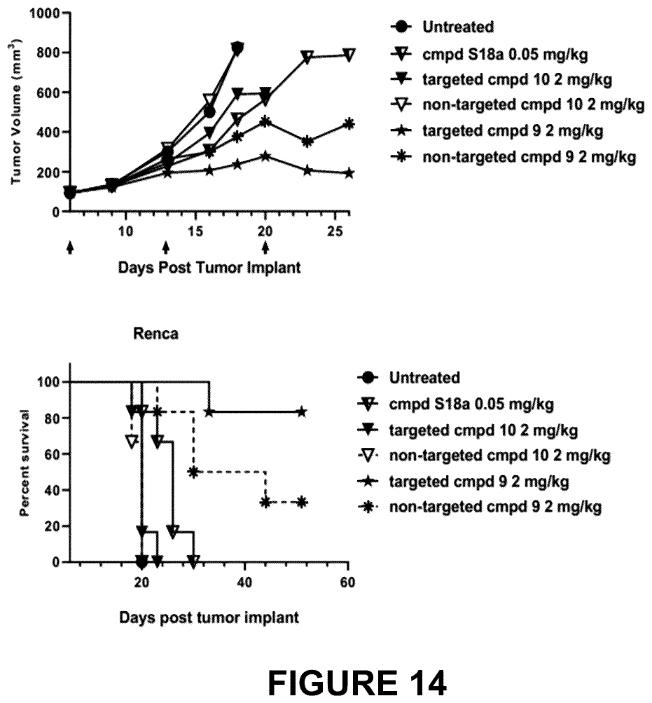

The difference in anti-tumor activity of the S18a and S18b conjugates was also assessed in the Renca kidney model. In this model, mice bearing Renca tumors were treated q7dx3 when tumors reached ˜100 mm³ as indicated, and followed for mean group tumor growth (FIG. 14, top) and survival (FIG. 14, bottom) over time. Again, very clearly it was demonstrated that the conjugated S18a (a C₇ carboxymethyl imidazoquinoline compound) provided some tumor growth benefit though no real survival benefit. However, the ADC containing S18b (a C₇ carboxyl imidazoquinoline compound) had very good tumor growth delay and superior overall survival advantage (FIG. 14).

### Example 39

### Effects of C4 and N1 Conjugation on Imidazoquinoline Compound Activity

While certain studies disclosed herein utilized conjugates where the linkage of the TLR7/8 agonist drug to a linker was at the N1 position of the imidazoquinoline core, the payloads can also be conjugated to a linker at the C4 amino group as an alternative linkage site. To compare the activity of imidazoquinoline compounds made from the same drug and linker, but linked at these two different positions, imidazoquinoline compounds with N1- or C4-coupled linkers were conjugated to an immune-targeted antibody. These ADCs were administered to non-tumor bearing mice and systemic immune activation was assessed through induction of plasma cytokines. ADCs with S18b linked via the N1 or C4 position were administered to non-tumor bearing Balb/c mice intraperitoneally at 2 mg/kg. Blood was isolated 3/6/24 h post dose and plasma was analyzed for cytokine induction via multiple cytokine analysis.

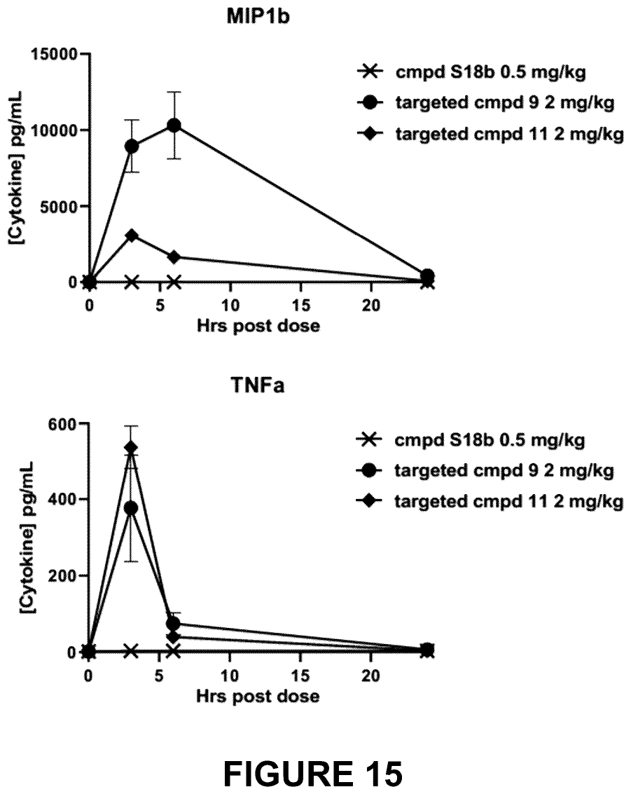

FIG. 15 provides MIP1b (top panel) and TNFα (bottom panel) responses generated with N1-(cmpd 9) and C4-(cmpd 11) linked compounds conjugated to targeting antibodies, as well as for the non-conjugated TLR agonist. For the majority of cytokines assessed (MCP1, IP10, MIP1a, MIP1b, RANTES), changing the linkage site of the drug to the linker from N1 to C4 greatly reduced the systemic cytokine induction by an average of 4.8-fold (FIG. 15). Interestingly no changes in IL6 or TNFα levels were seen between these conjugates (FIG. 15, bottom). The results from this study suggested that changing the linkage site of the linker to the TLR7/8 carboxylic acid drugs can greatly affect their potency and systemic cytokine induction.

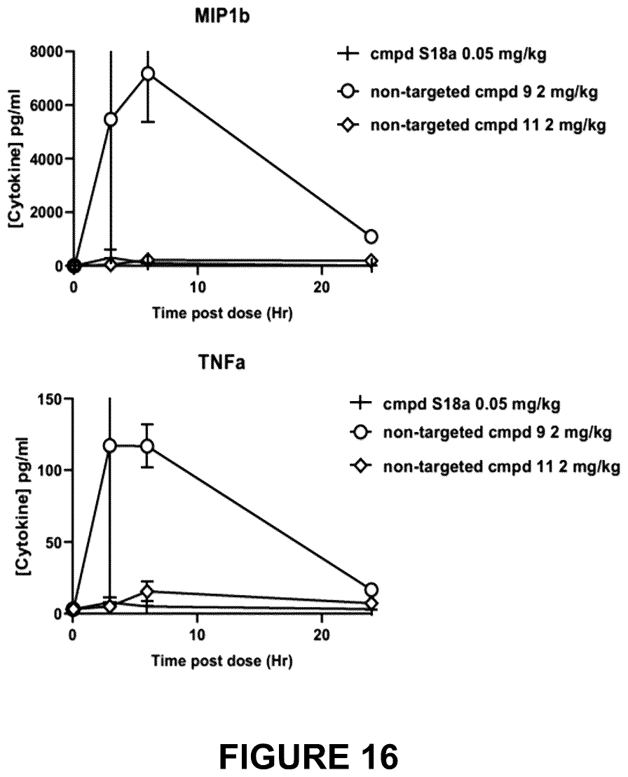

This study was repeated, again in non-tumor bearing animals, with non-targeting ADCs containing TLR7/8 agonists with linkers attached at either the N1 or C4 positions (compounds 9 and 11, respectively). FIG. 16 provides MIP1b (top panel) and TNFα (bottom panel) responses generated with N1-(cmpd 9) and C4-(cmpd 11) linked compounds conjugated to non-targeting antibodies, as well as for the non-conjugated TLR agonist. As outlined in FIG. 16, in this follow up study a very similar decrease in systemic cytokines seen by linking the linker at the C4 position vs. the N1 position of the imidazoquinoline core was seen, such that there was greatly diminished systemic cytokines and non-specific uptake of a non-targeted mAB with the TLR7/8 payload when conjugate at the C4 position.

Interestingly, in this experiment, a clear benefit of the C4-linked conjugates was observed for all cytokines monitored (shown here by MIP1β and TNFα) as opposed to the previous experiment where the majority of cytokines showed benefit but IL6 and TNFα did not. This may be due to different antibodies being used in the two studies, where the study described in FIG. 15 used a targeted mAB and the study described in FIG. 16 used a non-binding control.

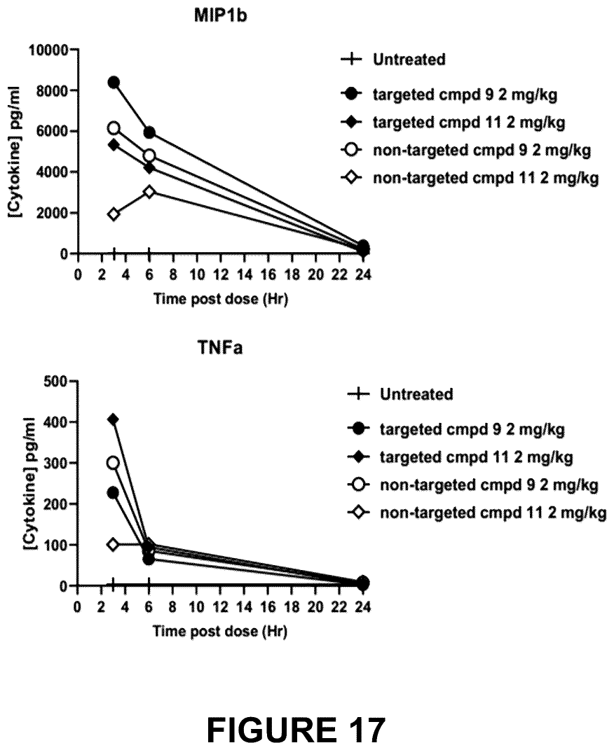

In addition to analyzing this response in non-tumor bearing animals simply on systemic immune activation, the C₄/N1-linked TLR7/8 drug antibody conjugates were also administered to CT26-tumor bearing animals to determine how the linkage site affects cytokine response and anti-tumor response. The results are shown in FIG. 17, which provides MIP1b (top panel) and TNFα (bottom panel) responses generated with N1-(cmpd 9) and C4-(cmpd 11) linked compounds conjugated to targeting and non-targeting antibodies, as well as for untreated cells. It was again demonstrated that for the majority of cytokines evaluated there was a reduced systemic activation seen when the TLR7/8 carboxylic acid drug payload was linked at the C4 position. This was true for targeted and non-targeted control conjugates. As noted above this benefit of the C4 linkage was greater for all the cytokines for the control conjugate but less universal for the targeted antibody conjugate, where N1 and C4 were equivalent for TNFα but showed benefit for MIP1β.

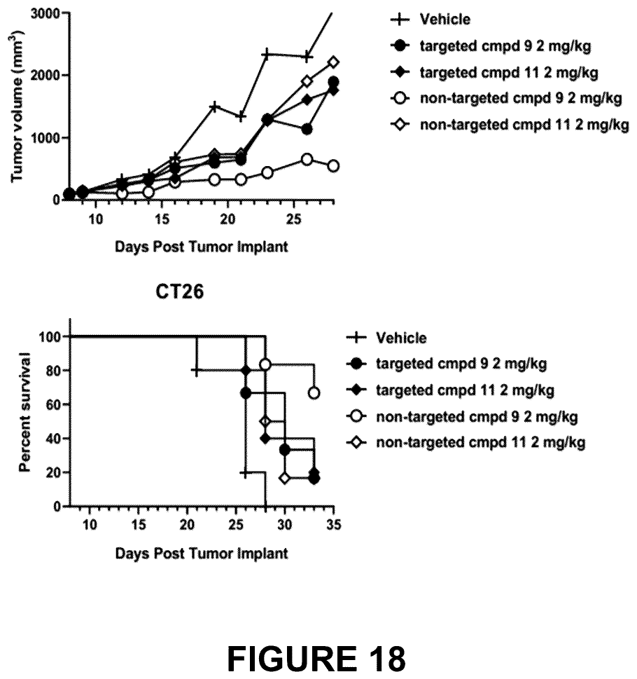

In addition to assessing cytokine induction, the test subjects were also followed for tumor growth and survival over time. The data from these analyses are summarized in FIG. 18, which provides tumor volume (top panel) and survival (bottom panel) as functions of days post tumor implant. While the N1 linked conjugates of the MAb control provided substantial benefits in tumor growth reduction and survival over time, (even better than conjugates of the targeted MAb), the C4 linked control MAb conjugate did not provide the same benefits. These data provide support for the hypothesis that a TLR7/8 payload linked at the N1 position to a non-targeted mAB drives non-specific uptake and activity, which can be significantly diminished by changing the linkage site to C₄. In contrast, both the C4-linked and N1-linked antibody conjugates of a targeted MAb demonstrated similar anti-tumor activity, suggesting that the linkage position does not have significant impact on the anti-tumor activity of targeted conjugates. These data suggest that changing the linkage site of these TLR7/8 antibody conjugates from N1 to C4 can greatly reduce non-specific/off target activity and systemic cytokine induction, while not affecting anti-tumor responses for targeted conjugates.

The comparison of the anti-tumor activity and non-specific cytokine induction between conjugates with N1 or C4 linkage was repeated in a disparate tumor model. Renca is a syngeneic kidney carcinoma model with a distinct microenvironment than CT26 (Mosely, S. et al. Cancer Immunol. Res., 2017, 5:29-14), and is often resistant to treatments effective in CT26; and thus could have a different response to treatment.

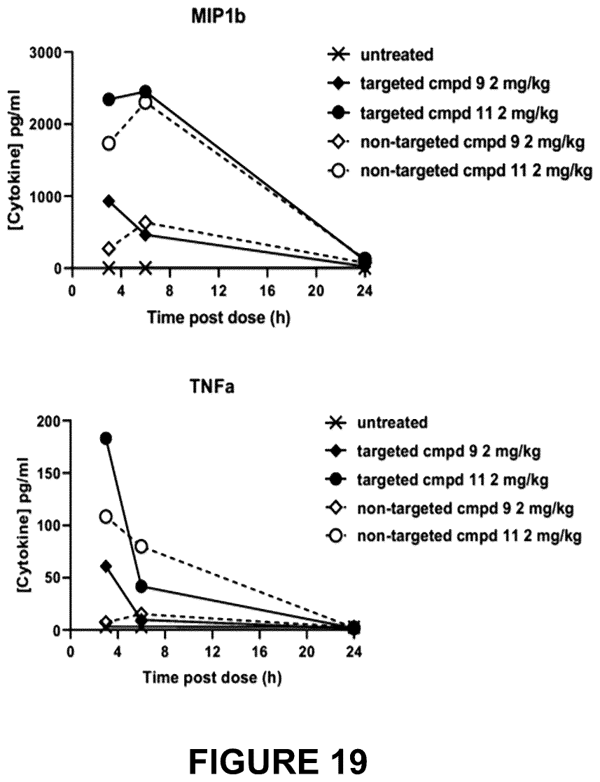

FIG. 19 summarizes cytokine responses induced with C4- and N1-conjugates of targeted and non-targeted antibodies, with the top panel providing MIP1β and the bottom panel providing TNFα levels at various times following dosing. In the Renca-bearing animals, similar systemic cytokine induction was observed in response to treatment with both of the C4- or N1-linked conjugates of targeted MAb and non-targeted MAb treatments, as was seen in the CT26 model (FIG. 17). Similar to the CT26 model, conjugates (of both targeted and non-targeted monoclonal antibodies (MAb)) with the C4 linkage afforded lower systemic cytokine induction than those with N1 linkage.

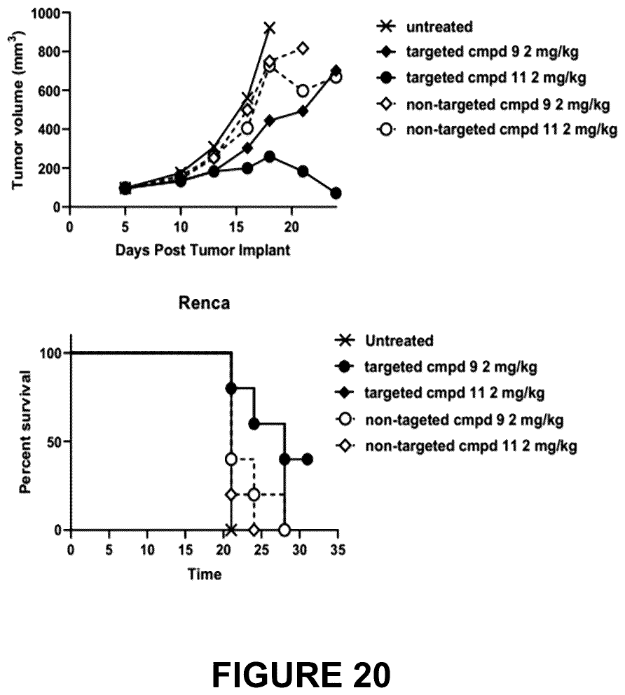

However, unlike in the CT26 model, where there was no significant difference in efficacy between the C4 and N1 linked conjugates of compound S18b with a targeted MAb (FIG. 18), in the Renca model there was a clear increase in anti-tumor efficacy when the S18b was linked at the N1 position. As summarized in FIG. 20, which shows tumor volume (top panel) and survival rates (bottom panel) following tumor implantation (x-axes in units of days), the N1-linked isotype showed superior, non-specific anti-tumor activity in the CT26 model, in the Renca model there was good immunological specificity of both the N1- and C4-linked conjugates. In this model it appeared that the N1-linked conjugate demonstrated superior anti-tumor activity accompanied by higher systemic cytokines.

In order to ascertain whether the difference in activity of the N1 vs. C4 linked conjugates seen in the Renca model was due to variable potency, a dose range experiment was performed. The N1 and C4 linked targeted (ADC) and non-targeted conjugates were administered in a wide dose range (0.001 to 10 μg/mL) to exogenously derived human immune cells. Cells were stimulated with various TLR/⅞ agonists at different concentrations. Supernatants were harvested and analyzed using a multiplex analyte kit (Luminex).

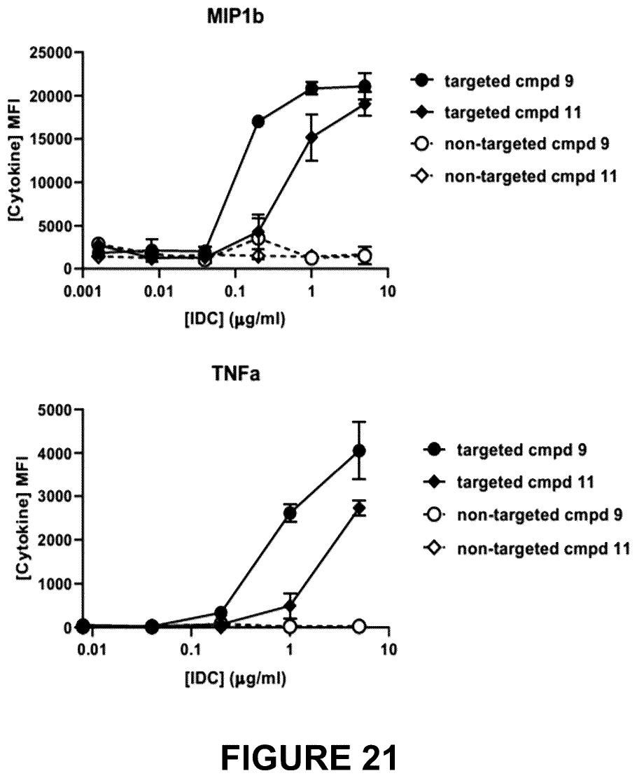

FIG. 21 shows MIP1b (top panel) and TNFα (bottom panel) responses affected by targeted and non-targeted imidazoquinoline-conjugated ADCs with either C4- or N1-linkages. Activation of the immune cells was observed as evidenced by cytokine induction over the entire dose range. In this experiment, it was noted that there was no activity of either of the non-targeted conjugates, but that both of the targeted conjugates were active at inducing immune cell activation. However, it was noted that the C4 linked conjugate with a targeted antibody appeared to lose some potency with a ˜4× loss in EC50.

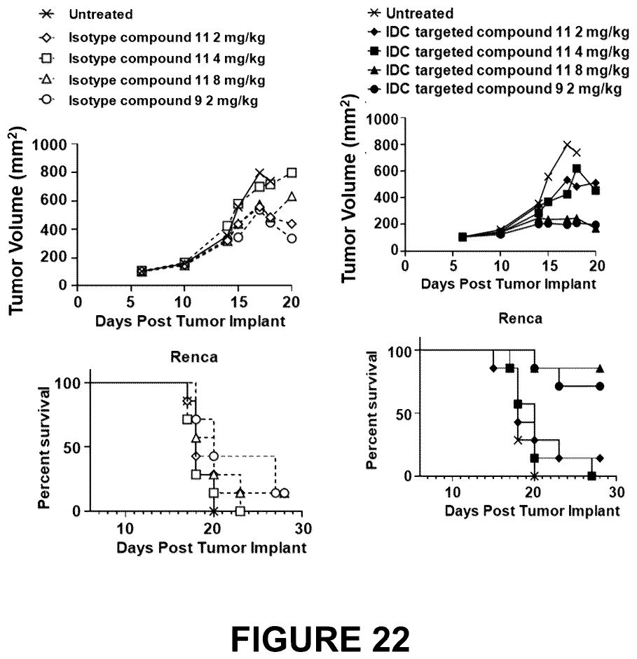

To further understand if this difference observed in cell culture translates to in vivo models, Renca-bearing animals were treated with a 2 mg/kg dose of conjugates made with compound 9 or increasing doses of the conjugate with a C4-linked payload (Compound 11) and a targeted MAb. The results of these analyses are shown in FIG. 22, which provides tumor volume (top) and survival rates (bottom) for non-targeted (left) and targeted (right) ADCs. Increasing the concentration of the C4-linked ADC enabled the recapitulation of the anti-tumor activity seen with the N1-linked conjugate, and the increase in the dose of the C₄-linked conjugate required to restore similar activity suggests an approximately 4× loss of potency of the C4-linked conjugate. Again, in this tumor model, there was very little activity of the conjugates made with the non-targeted control, suggesting good immunological specificity here that was not seen in the CT26 model (FIG. 22, left).

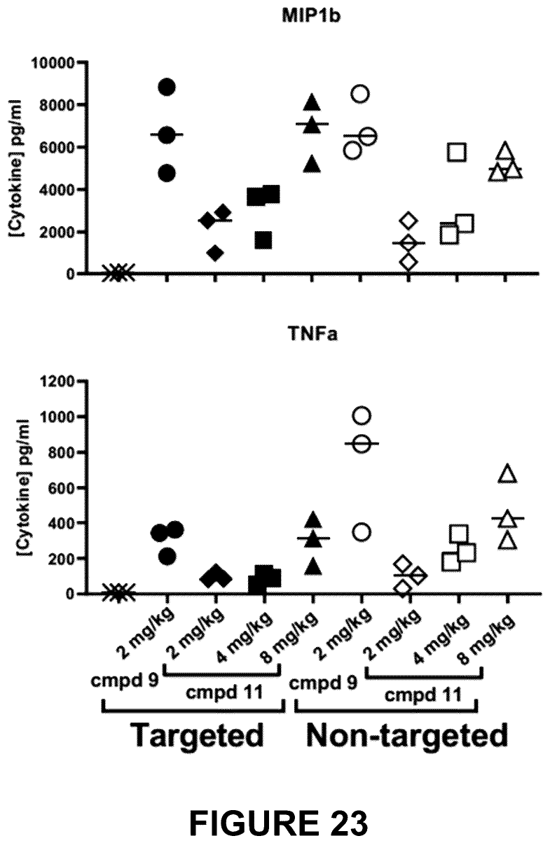

In addition to evaluating anti-tumor activity, the ability to induce systemic cytokine production as effected by imidazoquinoline-antibody conjugates was evaluated at different doses. FIG. 23 shows MIP1b (top panel) and TNFα (bottom panel) responses induced with various doses of targeted and non-targeted antibodies conjugated to N1- and C4-linked imidazoquinoline compounds. Again, at the same dose of 2 mg/kg, the C4-linked conjugate demonstrated markedly lower cytokine induction when conjugated to a targeted MAb, compared to the corresponding N1-linked conjugate. However, when the dose of the C₄-linked conjugate was increased four-fold to 8 mg/kg, systemic cytokine levels in response to this immunostimulatory drug conjugate were similar to those seen with 2 mg/kg of the N1-linked conjugate (FIG. 22, circles vs. triangles). Interestingly though, when on the non-targeted control antibody even at 8 mg/kg of the C4 lower systemic cytokine levels were seen than with the N1 conjugate. These data suggest that while changing the linkage from N1 to C4 leads to a 4× decrease in potency, as observed in both in vitro model (FIG. 21) and in vivo model (FIG. 22) when these conjugates are targeted to immune cells, the non specific activity observed with the N1-linked conjugate is still greater than that observed with the C4-linked conjugate. This further suggests a potential better safety margin for a C4-linked conjugate.

### Example 40

### Assessment of TLR7 and TLR8 Selectivity for Small Molecule TLR7/8 Agonists

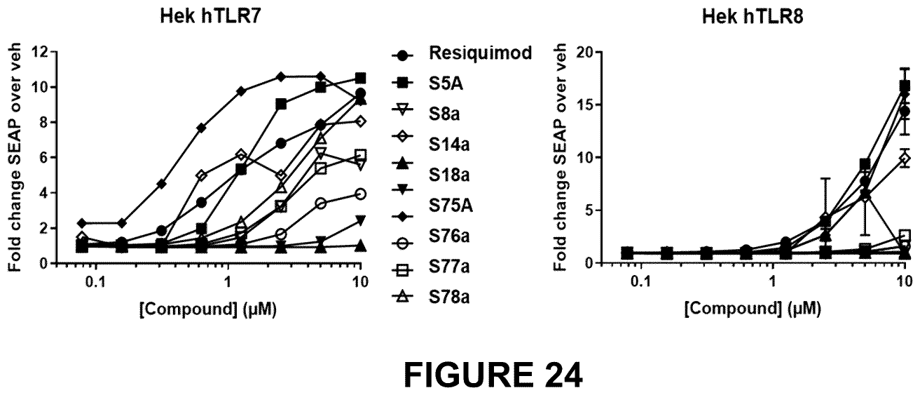

This example covers toll-like receptor 7 (TLR7) and toll-like receptor 8 (TLR8) selectivity of imidazoquinoline agonists. Assessment of TLR7 and TLR8 selectivity for the free TLR7/8 agonists was performed using HEK Blue hTLR7 and hTLR8 cells over a large dose range of compounds. FIG. 24 provides human toll-like receptor 7 (hTLR7, panel A) and human toll-like receptor 8 (hTLR8, panel B) activities for resiquimod (‘R848’), S5a, S8a, S18a, S72a, S75a, S76a, S77a, and S78a. Activities were determined based on calculated EC50 (in μM) in each cell line, and are summarized in TABLE 4 below, with columns providing targets and rows providing compounds. Compounds ranged in both potency for each cell line and also in their TLR7 vs TLR8 skewing.

TABLE 4

| | hTLR7 | hTLR8 | TLR7/TLR8 |
|---|---|---|---|
| Resiquimod | 1.59 | 11.38 | 0.14 |
| S5a | 0.59 | 5.08 | 0.12 |
| S8a | 1.32 | 6.19 | 0.21 |
| S14a | 2.53 | 8.84 | 0.28 |
| S18a | 0.44 | 10.27 | 0.04 |
| S76a | 3.44 | N/A | N/A |
| S77a | 2.82 | N/A | N/A |
| S78a | 3.78 | N/A | N/A |

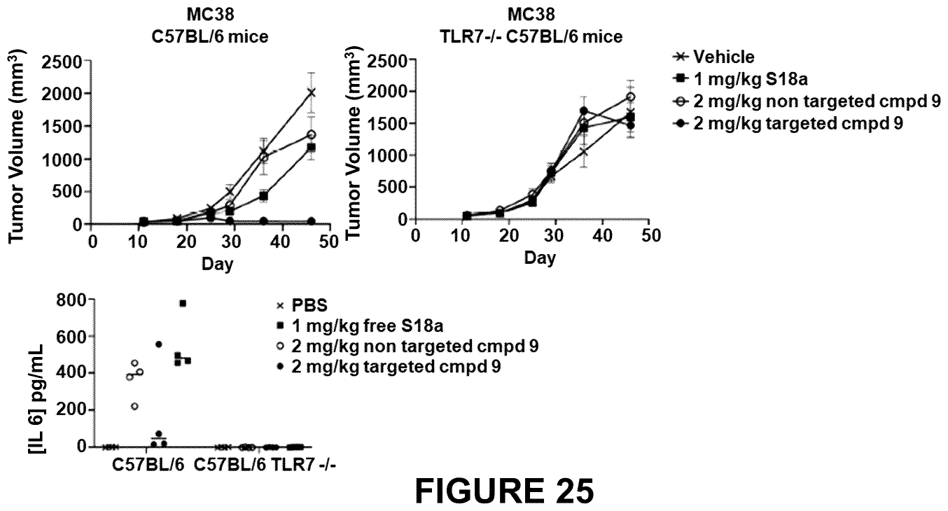

To assess the selectivity of the agonists in vivo, free molecule and conjugated drug were administered to C₅₇BL/6 mice bearing subcutaneous MC38 tumors, which have non functional TLR8 and only rely on TLR7 to drive activity against agonists, and TLR7 knockout animals that do not have either receptor. FIG. 25 summarizes tumor volume as a function of time post tumor implantation for C₅₇BL/6 (top left) and TLR7 knockout C₅₇BL/6 (top right) mice, as well as IL6 responses in both groups (bottom). Activity measured via systemic cytokine induction (FIG. 25, bottom) demonstrated that the large IL6 induction seen with free agonist was abrogated in TLR7 knockout animals. Similar loss of systemic cytokine induction was also seen in response to the ADC, though to a lesser extent, demonstrating that the targeted drug conjugate responses are TLR7 and TLR8 specific as well. This loss of response to the TLR7/8 free agonist and conjugated drug translated into loss of anti-tumor activity in the TLR7−/− mice compared to wild type mice (FIG. 25, top left and top right).

### Example 41

### Effects of Drug Linker Site and Type on Tumor Treatment Efficacy

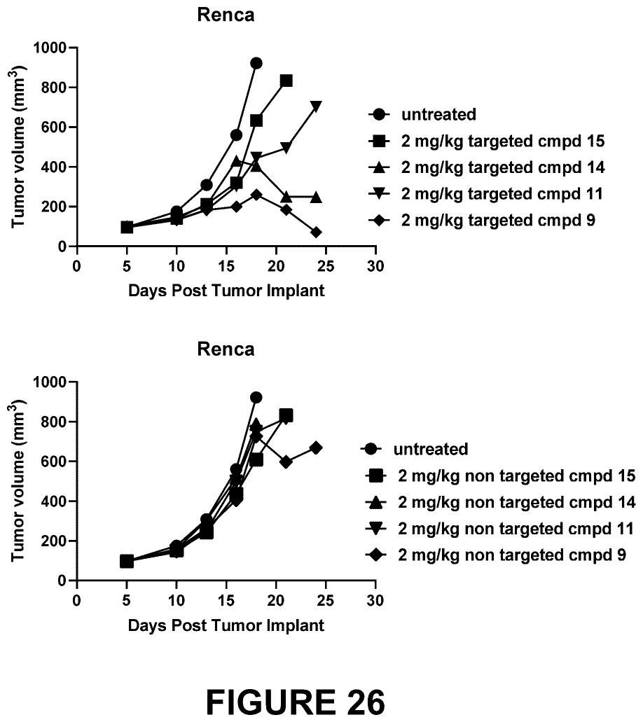

Balb/c mice bearing Renca tumors were treated with non-targeted or targeted S18a payload linked via different linker sites and with or without a PEG group in the linker (Compounds 9, 11, 14, and 15). FIG. 26 provides tumor volumes as a function of days post tumor implantation for mice treated with various targeted (left) and untargeted (right) ADCs. As previously demonstrated, the non-targeted conjugates demonstrated little to no activity. The targeted payload demonstrated the greatest potency when linked via the N1 position (Compounds 9 and 14) and when linked via a PEG group, as described herein (Compound 9).

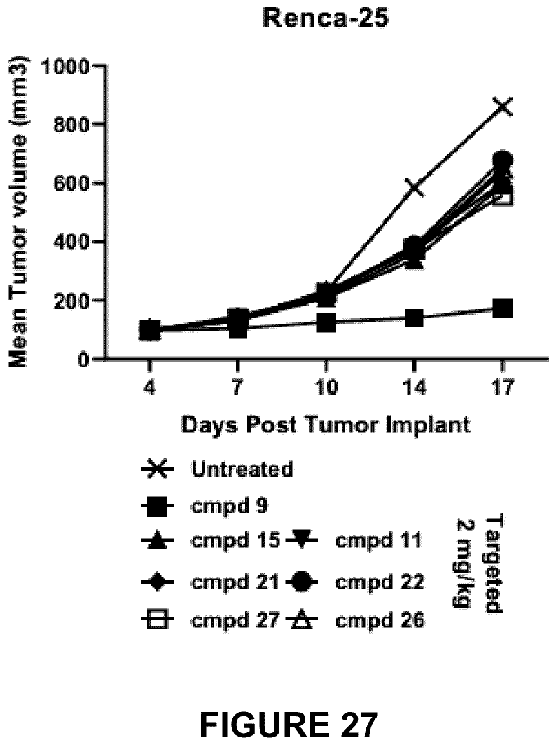

Balb/c mice bearing Renca tumors were treated with targeted S18a payload linked on the N1 site (Compound 9) or the C4 site via different linkers. FIG. 27 summarizes tumor volume as a function of days post tumor implantation for the various treatment groups. While the N1 linkage site demonstrated superior potency, all C4 linked molecules demonstrated similar anti-tumor activity.

### Example 42

### Anti-Tumor Activity of Immune and Tumor Targeted TLR7/8 Agonists

This example covers TLR7 and TLR8 (TLR7/8) agonists targeted to both the tumor and immune cells. To determine the difference in targeting intra-tumoral immune cells only as compared to immune and tumor cells a TLR7/8 agonist was conjugated to an immune targeting or a tumor and immune targeting antibody. 4T1 syngeneic breast tumors were left untreated or treated, starting at 100 mm³, with a naked tumor and immune targeted antibody, a tumor and immune targeted TLR7/8 agonist IDC, the cognate isotype non-targeted IDC or a TLR7/8 agonist IDC against intra-tumoral immune cells. The tumors were followed over time for growth.

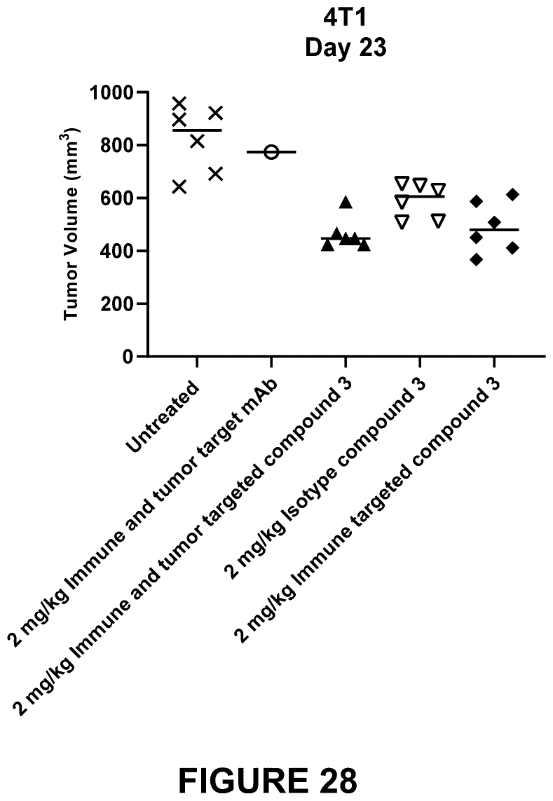

Tumor sizes at day 23 post tumor implant in each group are shown in FIG. 28. Most antibody treated animals had succumbed to tumor burden and/or treatment (likely as a result of the human IgG1 backbone and anti-drug antibodies formed; Oncoimmunology. 2016 Feb.; 5(2): e1075114.) by day 23. The animals treated with the ADC version of this mAb fared much better showing greatly decreased tumor size and tumor growth delay. This was greater than what was seen with the non-targeted isotype control and similar to the immune targeted IDC. The data demonstrate that conjugated TLR7/8 agonists can have activity when delivered on an immune target or a tumor/immune target.

### Example 43

### Human Immune Cell Activation by TLR7/8 Agonists

Several small molecule TLR7/8 agonists were assessed for their ability to activate human immune cells in a dose dependent manner. Primary human PBMCs were isolated from peripheral whole blood from human donors and were subjected to treatment with increasing concentrations of the agonists diluted in PBS. 24 h after treatment cell culture supernatants were harvested and analyzed for induction of cytokines via multiplex ELISAs (Luminex).

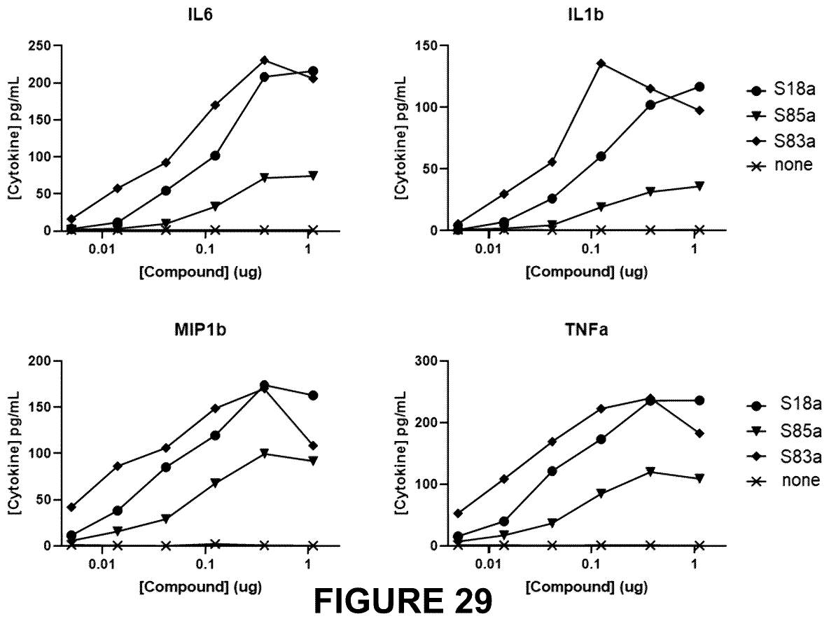

FIG. 29 provides IL6 (top left), IL1b (top right), MIP1b (bottom left), and TNFα (bottom right) responses in PBMCs generated with compounds 8a, S85a, and S83a. Compound S83a, which resembles compound S18a but with an ethyl linkage to the dimethyl nitrogen off the benzyl position at the N1 position, displays slightly enhanced potency to activate human PBMS when several different cytokines were evaluated. This suggests it has enhanced TLR7 and TLR8 potency. Compound S85a which is also similar to compound S18a but with a cyclohexyl group however displayed slightly decreased potency, in terms of half maximal effective concentration as well as decreased maximal cytokine level induced, creating an overall decreased ability to activate human PBMCs.

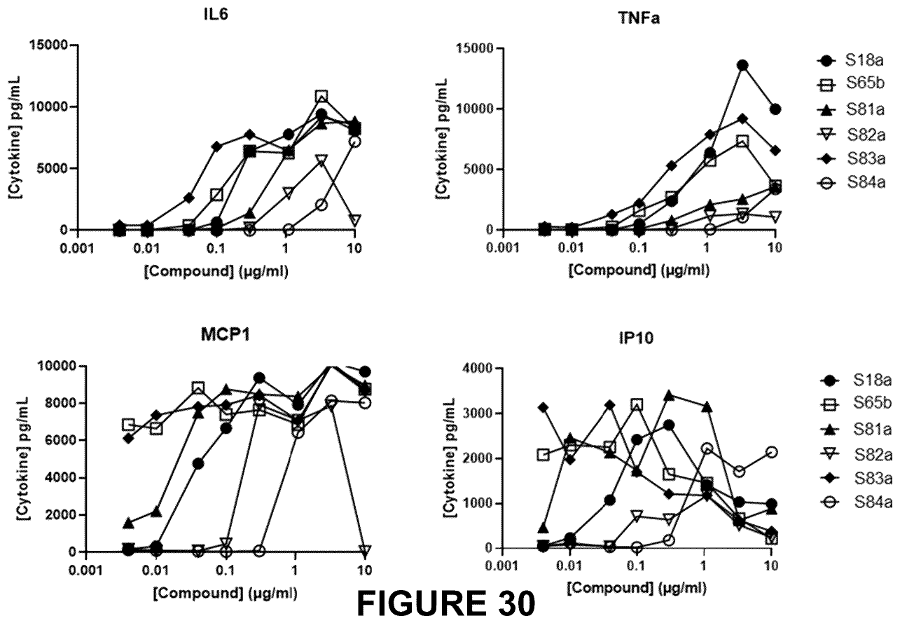

FIG. 30 summarizes human immune cell activation by additional small molecules (compounds S18a, S65a, S81a, S82a, S83a, and S84a) screened in the human PBMC assay, with the top left panel summarizing IL6 responses, the top right panel summarizing TNFα responses, the bottom left panel summarizing MCP1 responses, and the bottom right panel summarizing IP10 responses. The compounds demonstrated a wide range of potencies and activities. Compound S83a, which carries N,N-diethyl group, routinely demonstrated the highest maximal cytokine and potency to induce cytokine of all compounds tested. The other two most potent compounds across the range of cytokines evaluated were S18a and S65a. Compound S65a did demonstrate enhanced potency to induce MCP1 and IP10 compared to compounds S18a, suggesting the cycloalkyl group off the terminal nitrogen increases the TLR7 potency. The remaining compounds all demonstrated decreasing levels of activity with compound S81a having greater activity than compound S82a, and compound S82b having greater activity than compound S84a. Compound S81a induced the same maximal level of IL6, MCP1 and IP10 as the other compounds, but was not able to reach the same maximum level of IL10 (not shown), IL1b (not shown) or TNFα. Coupled with the strong, very potent ability to drive induction of MCP1 and IP10 suggests that compound S81a has a TLR7 vs TLR8 skewed binding profile.

### Example 44

### Imidazoquinoline Compound Specificity for TLR7/8

To assess if the small molecule and conjugated agonist are specific for the toll like 7 and/or 8 receptors, various small molecules and ADCs were tested on TLR7 receptor knockout animals. As TLR8 is normally nonfunctional in mice, in TLR7−/− mice there is a total loss of ability to respond to compounds that work through these receptors. To assess whether compounds are TLR7/8 specific, MC38 tumors were implanted into either wild type C₅₇B1/6 mice or cognate TLR7−/− mice. When tumors reached ˜50 mm3 mice animals were treated q7dx3 with vehicle (PBS), 1 mg/kg of the free small molecule S18a, the isotype/non-targeted ADC with cmpd 9 or with an immune cell targeted version of the cmpd 9 IDC.

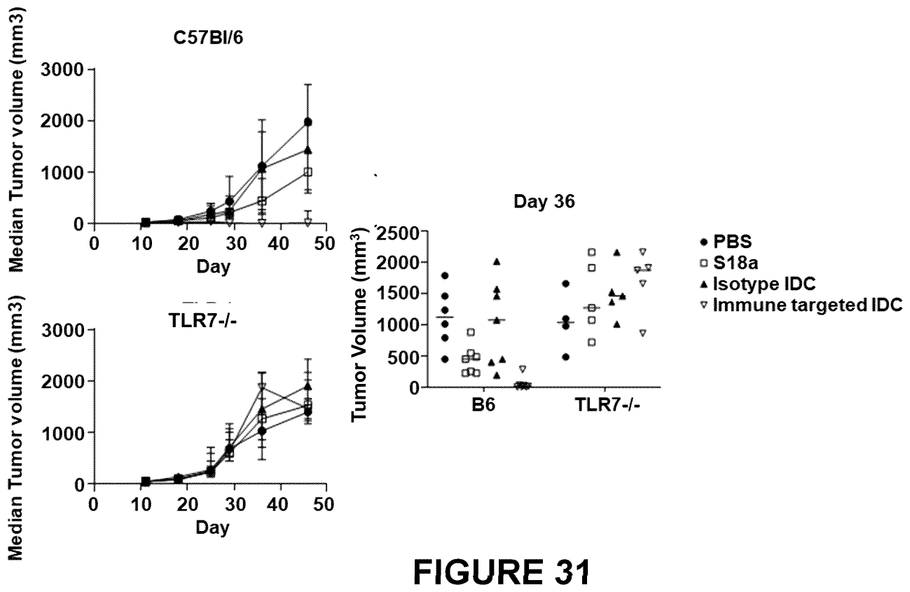

Tumor growth over time is summarizes in FIG. 31, in which the top left panel summarizes tumor growth in C₅₇B1/6 mice, the bottom left panel summarizes tumor growth in TLR7−/− mice, and the right panel summarizes tumor volumes on Day 36 for all mice. In the wild type B6 mice there was good MC38 tumor growth delay with the TLR7/8 agonist small molecule and delay and cures in the targeted ADC treated animals, with only minimal responses seen with a non targeted ADC control. However, in the animals that lack TLR7 and have nonfunctional TLR8 there was no anti-tumor response seen in response to any treatment. Cytokines from the plasma of these animals were also evaluated post dose and showed no evidence of induction of any cytokine response. These results clearly demonstrate that these agonists and linked molecules work exclusively through engagement of TLR7 and TLR8 receptors to drive anti-tumor immunity.

To determine the anti-tumor activity of a TLR7/8 agonist conjugate on additional targeting antibodies the anti-tumor activity in a Renca renal syngeneic model that exogenously expresses a tumor antigen was also evaluated. For this experiment the cmpd 9 linker was conjugated to an antibody targeting the exogenously expressed tumor antigen on either a mIgG2a Fc null backbone or wildtype backbone. Animals were treated Q7dx3 with these therapies when their tumors were 100 mm³ and followed for tumor growth over time.

To determine how changing the loading effects the efficacy of the TLR7/8 agonist IDCs, immune targeted IDCs containing either cmpd 9 or cmpd 14 linked agonist were conjugated with a drug antibody ratio (DAR) of 2, 4 or 10. These were administered at varying doses Q7dx3 to mice bearing Renca tumors that were ˜100 mm3 in size and tumor size was followed over time.

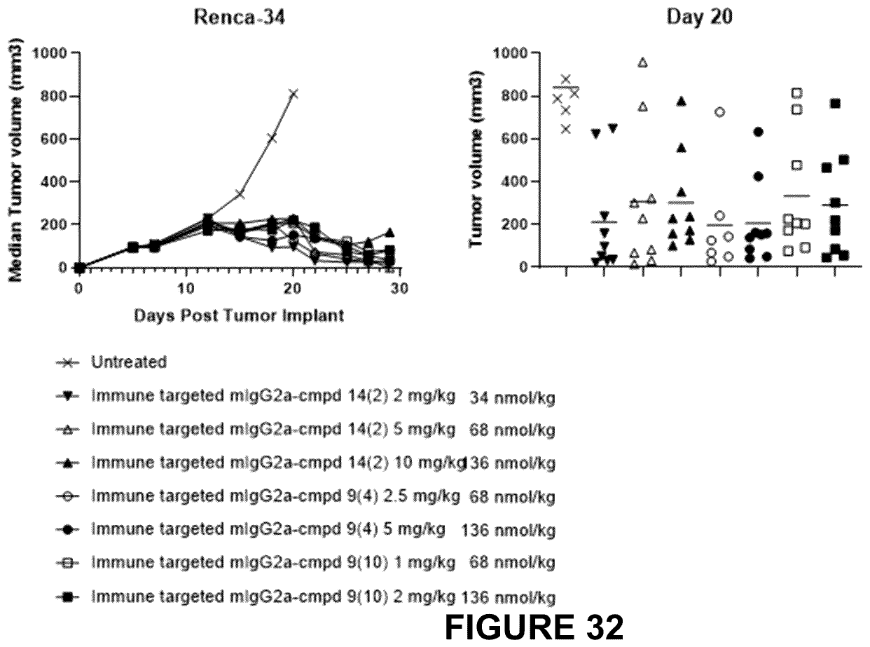

FIG. 32 summarizes the results of these analyses. Notably, all treatments seemed to drive very similar anti-tumor activity that were not statistically different from one another. This is interesting since no IDCs seem to have diminished activity despite delivering lower quantities of drug (denoted in nmol/kg doses) demonstrating the strong potency of these treatments. Furthermore, the DAR2 IDC at the lowest dose tested, which was 4× lower dose than that given with the higher doses of the higher DAR species, showed the same activity; these data perhaps suggests that the lower loaded IDC may show enhanced potency.

### Example 45

### EphA2-Targeted TLR Agonist Cancer Treatment Efficacies

This example covers cancer treatment with EphA2 targeted TLR agonists. EphA2 is a murine tumor antigen that has been found to be overexpressed in several mouse carcinoma cell lines, including CT26, MCA205, 4T1, and Renca cells (Rios-Doria, J. et al. Cancer research 2017; 77:2686-2698.) and can be targeted by murine cross-reactive antibodies. Balb/c mice were implanted with Renca tumor cells and when cells reached 100 mm³ they were treated Q7dx3 treatments with 2.4 mg/kg of the indicated IDC, either non targeting isotype or an EphA2 targeting mAb.

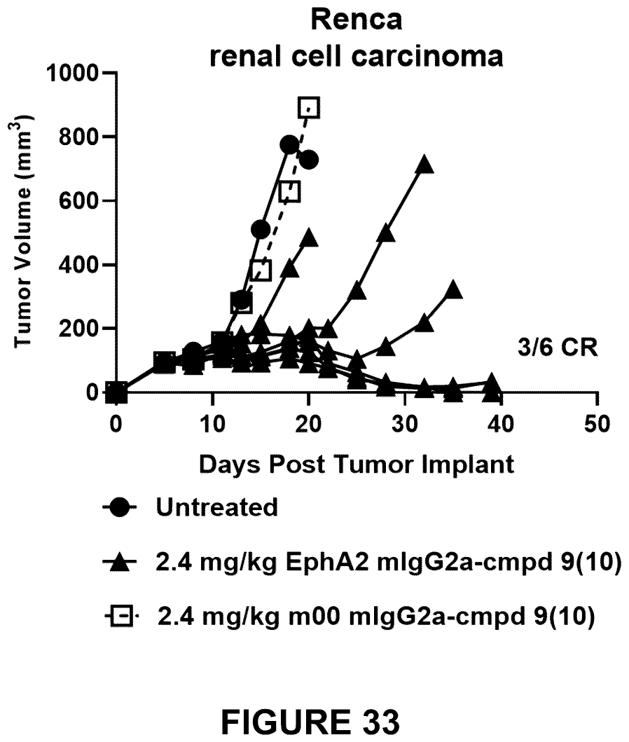

FIG. 33 tracks Renca tumor volumes in untreated, tumor (EphA2) targeted TLR7/8 agonist treated, and non-targeted TLR7/8 agonist treated mice. When Renca tumor growth was followed over time post treatment with EphA2 targeted TLR7/8 IDC, a marked tumor growth delay was noted. Additionally, the tumor-targeted TLR treatment resulted in 50% durable complete tumor cures in the treated animals. Notably, this anti-tumor activity was not seen in animals treated with a non-targeted isotype control TLR7/8 IDC demonstrating specificity of the treatment in this tumor model.

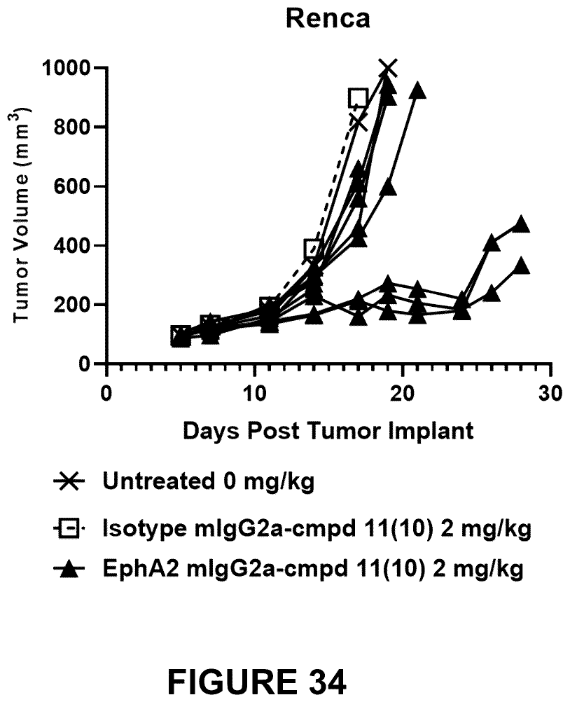

To determine whether EphA2 delivered TLR7/8 agonist to drive anti-tumor activity in the Renca model, Renca tumor-bearing mice were treated with the alternate TLR7/8 drug linker cmpd 11, which is similar to the TLR7/8 agonist used in FIG. 33, but is antibody linked through its C4 position. The results of this analysis are summarized in FIG. 34, which provides tumor volumes in untreated, non-targeted TLR7/8 agonist treated (“Isotype”), and tumor (EphA2) targeted TLR7/8 agonist treated mice. This drug linker generally shows ˜4× decrease in potency. Nonetheless, when dosed at a similar dose of 2 mg/kg, it was still able to drive anti-tumor activity with substantial tumor growth delayed noted for several of the animals when delivered via EphA2.

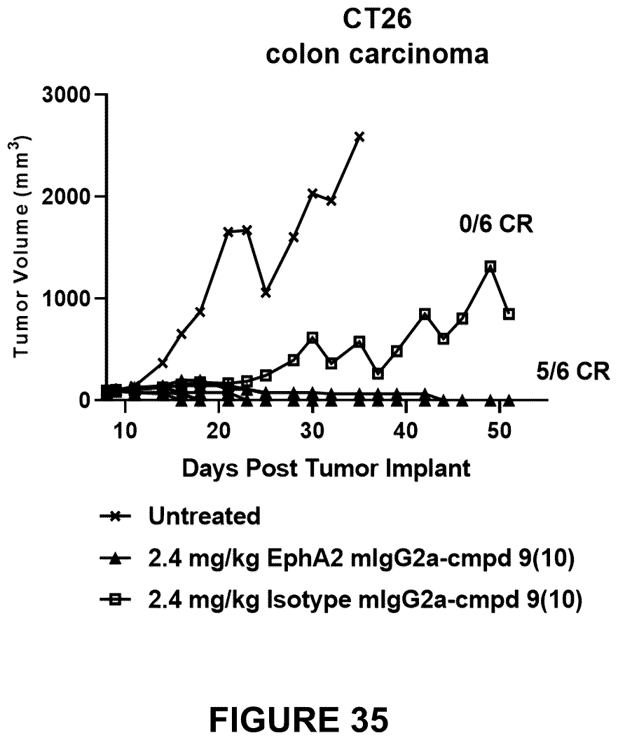

To determine if this tumor-targeted TLR7/8 IDC would have activity in other tumor models, Balb/c mice containing CT26 murine colon carcinoma tumors were treated with tumor (EphA2) targeted TLR7/8 agonist and non-targeted TLR7/8 agonist complexes. The results of this analysis are provided in FIG. 35. EphA2 targeted TLR7/8 IDC exhibited substantial anti-tumor activity and growth delay with 83% of the animals reaching durable complete tumor cures. In this model, the non-targeted isotype control TLR7/8 IDC also resulted in some tumor growth delaying, though it was decreased vs the targeted molecule and did not drive any curative activity.

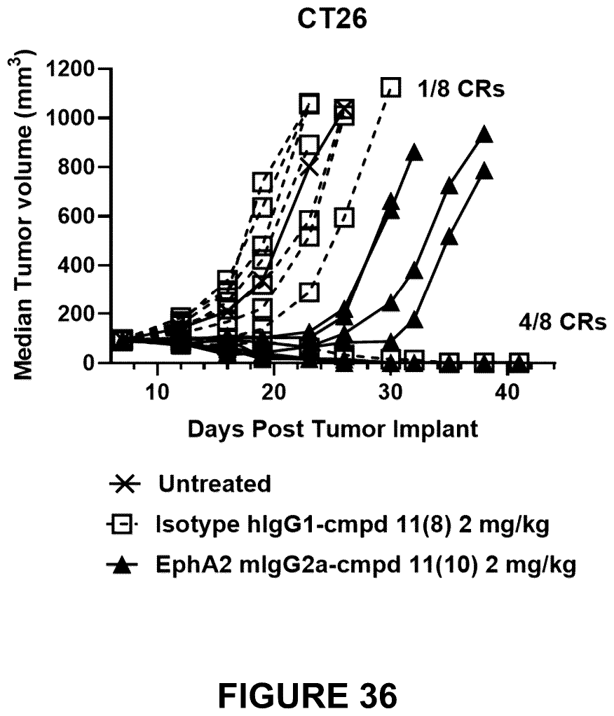

As summarized in FIG. 36, this mouse model was also subjected to treatment with a similar dose of the less potent TLR7/8 drug linker cmpd 11. As seen in the Renca tumor model, the EphA2 targeted cmpd 11 TLR7/8 agonist IDC was also able to drive substantial anti-tumor activity in a subset of animals with 50% of the animals achieving full cures and all animals benefiting from tumor growth delay. Conversely, non-targeted, isotype conjugated TLR7/8 agonist demonstrated greatly decreased anti-tumor activity with only one out of the eight animals showing any response to treatment. These data support that tumor targeting of TLR7/8 agonists, with varying drug linkers, can drive specific, potent anti-tumor activity across a range of tumor models.

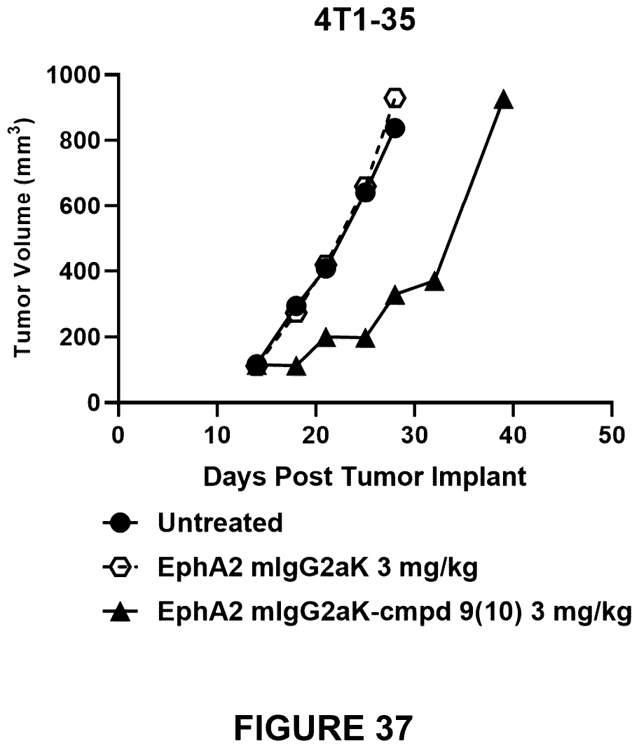

Activity in an additional EphA2-expressing model, the breast carcinoma model 4T1, was also assessed. The results of this analysis are provided in FIG. 37, with tumor volumes summarized for untreated, bare EphA2-targeted antibody treated, and TLR7/8 agonist EphA2-targeted antibody conjugate treated mice. For this model, animals were treated with a 3 mg/kg dose of the EphA2 antibody alone (to determine if the mAb alone can drive activity in this model) or the targeted TLR7/8 IDC. No tumor growth delay or activity was seen with the antibody alone suggesting that treatment with this antibody does not drive any of the anti-tumor activity seen. However, use of the EphA2 targeted TLR7/8 IDC did result in some tumor growth delay. The response to the tumor targeted TLR7/8 IDC in this model was muted compared to that seen in CT26 or Renca, likely concomitant with the differing baseline TMEs between the models.

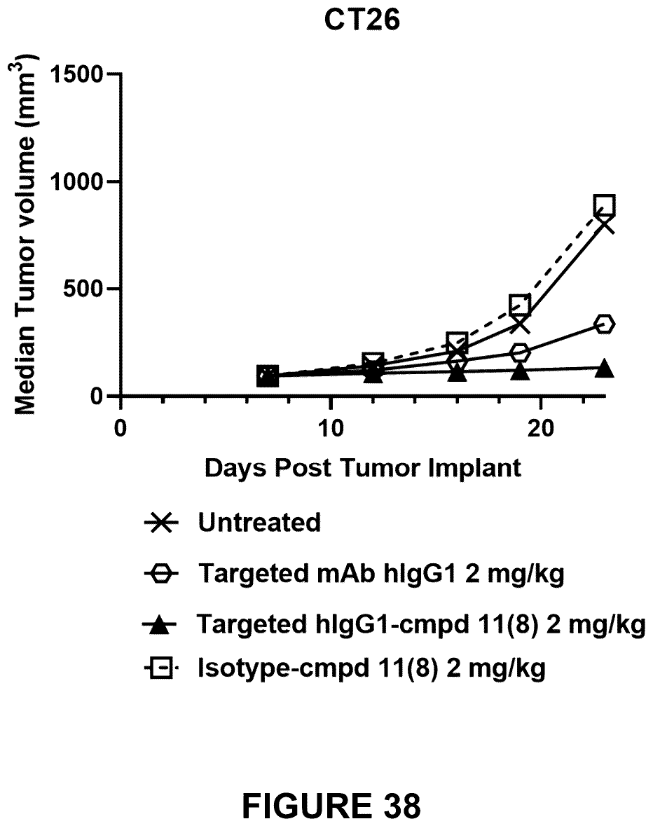

In addition to assessing how tumors respond to either immune targeted or tumor targeted TLR7/8 agonist delivered via IDC, tumor volume responses with agonist directly delivered to both tumor and immune cells were assessed. This analysis leveraged a target that is known to be expressed on both cell types in the intratumoral microenvironment. Specifically, Balb/c mice were implanted with CT26 syngeneic colon carcinoma cells and when tumors reached 100 mm³ tumor were either left untreated or were treated with the antibody alone, a non-targeted, isotype antibody conjugated to the TLR7/8 agonist drug linker cmpd 11 or drug linker conjugated to the tumor/immune targeted mAb. Animals were dosed with 2 mg/kg every 7 days for 3 doses total and followed over time for tumor growth and response. The results of these analyses are summarized in FIG. 38. Although no cures were observed with the IDC, 2/8 of the naked antibody treated animals were fully cured. Median tumor volumes per group demonstrate an advantage with the TLR7/8 containing IDC vs the naked tumor/immune targeted mAb in terms of tumor growth delay.

The contents of each of the references cited in the present disclosure are hereby incorporated by reference in their entirety.

A number of embodiments of the present disclosure have been described. Nevertheless, it will be understood that various modifications may be made without departing from the spirit and scope of the disclosure. Accordingly, other embodiments are within the scope of the following claims.

1. (canceled)

2. An antibody drug conjugate (ADC) having the structure:
Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
each D has the structure of Formula (I): 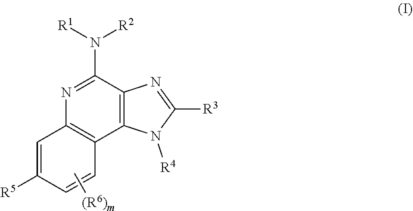
or a pharmaceutically acceptable salt thereof;
wherein:
R¹ is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached, form a 3-6 membered heterocyclyl optionally substituted with 1-3 independently selected C₁-C₆ alkyl;
R³ is (a) the point of covalent attachment to L; or (b) selected from the group consisting of hydrogen, —NR^{A}R^{B}, —C(═0) NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) the point of covalent attachment to L; (b) —OR^{C}; (c) —S(═O)₂R^{C}; (d) —C(═O)NR^{D}R^{E};
(e) —C(═O)OR^{C}; (f) —C(═O)SR^{C}; (g) —C(═S)R^{C}; (h) —PO₃R^{C}; or (j) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H;
wherein when R⁴ is (j), the C₁-C₆ alkyl, or a substituent thereof, may be further substituted with the point of covalent attachment to L;
R⁵ is selected from the group consisting of —C(═O)OR^{E}, —NO₂, —CN, —CF₃, —C(═O)NR^{G}R^{H}, —S(O₂)NR^{G}R^{H}, —N(R′)—C(═O)R^{J}, —N(R¹)—S(O₂)R^{K}, and SO₃R^{K};
each R⁶ is (a) the point of covalent attachment to L; or (b) independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R⁶ is the point of covalent attachment to L;
subscript m is 0, 1, 2, or 3;
each R^{A} and R^{B} is (a) the point of covalent attachment to L, (b) independently selected from the group consisting of hydrogen and C₁-C₆ alkyl; or R^{A} and R^{B} taken together with the nitrogen atom to which they are attached, form a 3-6 membered heterocyclyl optionally substituted with 1-3 independently selected C₁-C₆ alkyl; wherein only one of R^{A} and R^{B} is the point of covalent attachment to L;
R^{C} is (a) the point of covalent attachment to L; or (b) selected from the group consisting of hydrogen, phenyl, and C₁-C₁₀ alkyl optionally substituted with phenyl or 1-3 independently selected halogen;
each R^{D}, R^{E}, R^{G}, and R^{H} are (a) the point of covalent attachment to L; or (b) independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached, form a 3-6 membered heterocyclyl optionally substituted with 1-3 independently selected C₁-C₆ alkyl; wherein only one of R^{D}, R^{E}, R^{G}, and R^{H} is the point of covalent attachment to L;
R^{F} is (a) the point of covalent attachment to L; or (b) selected from the group consisting of hydrogen, trifluoromethyl, C₂-C₆ alkenyl, C₂-C₆ alkynyl, C₃-C₈ cycloalkyl, aryl, aryl(C₁-C₆ alkyl), and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is (a) the point of covalent attachment to L; or (b) independently selected from the group consisting of hydrogen and C₁-C₆ alkyl; wherein only one of R^{I}, R^{J}, and R^{K} is the point of covalent attachment to L;
wherein only one of R¹, R², R³, R⁴, R⁵, R⁶, R^{A}, R^{B}, R^{C}, R^{D}, R^{E}, R^{F}, R^{G}, R^{H}, R^{I}, R^{J} and R^{K} is the point of covalent attachment to L;
R¹ and R⁴ are each optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide; and
wherein each D has only one point of covalent attachment to L.

3.-23. (canceled)

24. The ADC of claim 2, wherein subscript p is an integer from 1 to 8.

25. The ADC of claim 2, wherein subscript p is an integer from 4 to 12.

26. The ADC of claim 2, wherein subscript p is an integer from 8 to 16.

27.-28. (canceled)

29. The ADC of claim 2, wherein each L is covalently attached to Ab via a sulfur atom of a cysteine residue.

30. The ADC of claim 2, wherein R¹ is the point of covalent attachment to the linker.

31.-33. (canceled)

34. The ADC of claim 2, wherein the C₁-C₆ alkyl of R⁴, or a substituent thereof, is the point of covalent attachment to the linker.

35.-37. (canceled)

38. The ADC of claim 2, wherein one of R^{A}, R^{B}, R^{C}, R^{D}, R^{E}, R^{F}, R^{G}, R^{H}, R^{J}, and R^{K} is the point of covalent attachment to the linker.

39.-42. (canceled)

43. The ADC of claim 2, wherein R^{D} is the point of covalent attachment to the linker.

44. The ADC of claim 2, wherein R^{E} is the point of covalent attachment to the linker.

45.-53. (canceled)

54. The ADC of claim 2, wherein R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein the C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is unsubstituted.

55. The ADC of claim 2, wherein R¹ is selected from the group consisting of hydrogen and C₁-C₆ alkyl.

56.-61. (canceled)

62. The ADC of claim 2, wherein R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein the C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl are unsubstituted.

63. The ADC of claim 2, wherein R² is selected from the group consisting of hydrogen and unsubstituted C₁-C₆ alkyl.

64.-65. (canceled)

66. The ADC of claim 2, wherein R¹ and R² are both hydrogen.

67.-71. (canceled)

72. The ADC of claim 2, wherein R³ is selected from the group consisting of hydrogen —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, and C₁-C₆ alkanoyloxy; wherein the C₁-C₆ alkyl, C₂-C₆ alkenyl, C₂-C₆ alkynyl, C₁-C₆ alkanoyl, C₁-C₆ alkoxycarbonyl, and C₁-C₆ alkanoyloxy are unsubstituted.

73. (canceled)

74. The ADC of claim 2, wherein R³ is an unsubstituted C₁-C₆ alkyl.

75. (canceled)

76. The ADC of claim 2, wherein R³ is C₁-C₆ alkyl substituted with C₁-C₆ alkoxy.

77. The ADC of claim 2, wherein R⁴ is C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H.

78.-79. (canceled)

80. The ADC of claim 2, wherein R⁴ is C₁-C₆ alkyl substituted with —OR^{C}.

81.-86. (canceled)

87. The ADC of claim 2, wherein R⁴ is —CH₂-(phenyl)-(C₁-C₂ alkyl), wherein the C₁-C₂ alkyl is substituted with —NR^{D}R^{E} or —[N(C₁-C₆ alkyl)R^{D}R^{E}]⁺.

88.-110. (canceled)

111. The ADC of claim 2, wherein R¹ is substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide.

112.-141. (canceled)

142. The ADC of claim 2, wherein R⁵ is selected from the group consisting of —C(═O)OR^{E}, —C(═O)NR^{G}R^{H}, —S(O₂)NR^{G}R^{H}, —N(R′)—C(═O)R^{J}, and —N(R^{T})—S(O₂)R^{K}.

143. The ADC of claim 2, wherein R⁵ is —C(═O)OR^{E}.

144.-146. (canceled)

147. The ADC of claim 143, wherein R^{E} is C₁-C₆ alkyl.

148. (canceled)

149. The ADC of claim 143, R^{F} is hydrogen.

150.-152. (canceled)

153. The ADC of claim 2, wherein each R^{G} and R^{H} are independently selected from the group consisting of hydrogen and C₁-C₆ alkyl.

154.-160. (canceled)

161. The ADC of claim 2, wherein R⁵ is selected from the group consisting of —C(═O)OH, —NO₂, —CN, —CF₃, and —S(O₃)H.

162. (canceled)

163. The ADC of any one of claims 2-7, 9-11, 23-35, 37-46, 48-141, or 157, wherein R¹ and R^{K} are independently selected from the group consisting of hydrogen and C₁-C₆ alkyl.

164. (canceled)

165. The ADC of claim 2, wherein each R⁶ is independently selected from the group consisting of halogen, hydroxyl, nitro, and cyano, and wherein subscript m is 1.

166. The ADC of claim 2, wherein subscript m is 0.

167.-174. (canceled)

175. The ADC of any one of claims 1-37 or 41-174, wherein each R^{A} and R^{B} is independently selected from the group consisting of hydrogen and C₁-C₆ alkyl.

176.-179. (canceled)

180. The ADC of claim 2, wherein R^{C} is selected from the group consisting of hydrogen, phenyl, and C₁-C₁₀ alkyl.

181.-184. (canceled)

185. The ADC of claim 2, wherein each R^{D} and R^{E} are independently selected from the group consisting of hydrogen and C₁-C₆ alkyl.

186.-232. (canceled)

233. The ADC of claim 2, wherein the antibody is a humanized antibody.

234. The ADC of claim 2, wherein the antibody is a monoclonal antibody.

235. The ADC of claim 2, wherein the antibody is fucosylated.

236. The ADC of claim 2, wherein the antibody is afucosylated.

237. A composition comprising a distribution of the ADCs of claim 2, or a pharmaceutically acceptable salt thereof.

238. (canceled)

239. A compound of Formula (IX): 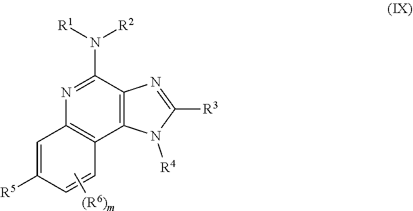
or a pharmaceutically acceptable salt thereof;
wherein:
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁—C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) —OR^{C}; (b) —S(═O)₂R^{C}; (c) —C(═O)NR^{D}R^{E}; (d) —C(═O)OR^{C}; (e) —C(═O)SR^{C}; (f) —C(═S)R^{C}; (g) —PO₃R^{C}; or (h) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
R⁵ is —C(═O)OR^{F};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} is independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl;
R^{F} is hydrogen;
each R^{I}, R^{J}, and R^{K} is independently selected from the group consisting of hydrogen and C₁-C₆ alkyl;
and
each instance of R¹ and R⁴ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide.

240. (canceled)

241. The compound of claim 239, wherein the compound has the structure of Formula (IX-A): 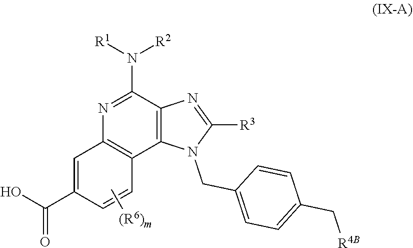
or a pharmaceutically acceptable salt thereof,
wherein:
R¹ is selected from the group consisting of hydrogen and C₁-C₆ alkyl;
R² is selected from the group consisting of hydrogen and C₁-C₆ alkyl; or
R¹ and R², taken together with the nitrogen atom to which they are attached, form a 3-6 membered heterocyclyl optionally substituted with 1-3 independently selected C₁-C₆ alkyl;
R³ is selected from the group consisting of hydrogen and C₁-C₆ alkyl optionally substituted with 1-3 substituents independently selected from the group consisting of hydroxyl, halogen, sulfhydryl, cyano, oxo, oxiranyl, C₁-C₆ alkoxy, and C₁-C₆ alkylthio;
R^{4B} is selected from the group consisting of 5-10 membered heteroaryl, —NR^{D}R^{E}, and —[N(C₁-C₆ alkyl)R^{D}R^{E}]⁺;
R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, and C₁-C₆ haloalkoxy;
subscript m is 0 or 1; and
R^{D} and R^{E} are independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, together with the nitrogen atom to which they are attached, form a 3-6 membered heterocyclyl optionally substituted with 1-3 independently selected C₁-C₆ alkyl.

242. A compound of Formula (IX-B): 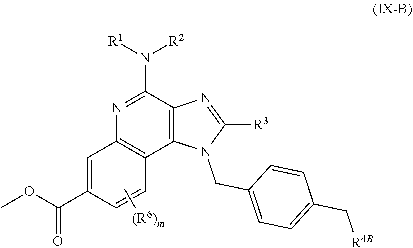
or a pharmaceutically acceptable salt thereof;
wherein:
R¹ is selected from the group consisting of hydrogen and C₁-C₆ alkyl;
R² is selected from the group consisting of hydrogen and C₁-C₆ alkyl; or
R¹ and R², taken together with the nitrogen atom to which they are attached, form a 3-6 membered heterocyclyl optionally substituted with 1-3 independently selected C₁-C₆ alkyl;
R³ is selected from the group consisting of hydrogen and C₁-C₆ alkyl optionally substituted with 1-3 substituents independently selected from the group consisting of hydroxyl, halogen, sulfhydryl, cyano, oxo, oxiranyl, C₁-C₆ alkoxy, and C₁-C₆ alkylthio;
R^{4B} is selected from the group consisting of NR^{D}R^{E} and —[N(C₁-C₆ alkyl)R^{D}R^{E}]⁺;
R⁶ selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, and C₁-C₆ haloalkoxy;
subscript m is 0 or 1; and
R^{D} and R^{E} are independently selected from the group consisting of C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl).

243.-246. (canceled)

247. The compound of claim 241, wherein:
(i) R¹ and R² are both hydrogen;
(ii) R³ is C₁-C₆ alkyl optionally substituted with 1-3 substituents independently selected from the group consisting of hydroxyl, halogen, sulfhydryl, cyano, oxo, oxiranyl, C₁-C₆ alkoxy, and C₁-C₆ alkylthio;
(iii) R^{4B} is —NR^{D}R^{E};
(iv) R^{D} and R^{E} are each independently selected C₁-C₆ alkyl;
(v) subscript m is 0; or
(vi) a combination thereof.

248. (canceled)

249. The compound of claim 242, wherein:
(i) R¹ and R² are both hydrogen;
(ii) R³ is C₁-C₆ alkyl optionally substituted with 1-3 substituents independently selected from the group consisting of hydroxyl, halogen, sulfhydryl, cyano, oxo, oxiranyl, C₁-C₆ alkoxy, and C₁-C₆ alkylthio;
(iii) R^{4B} is —NR^{D}R^{E};
(iv) R^{D} and R^{E} are each independently selected C₁-C₆ alkyl;
(v) subscript m is 0; or
(vi) a combination thereof.

250.-270. (canceled)

271. A method of treating cancer in a subject in need thereof, comprising administering to the subject a therapeutically effective amount of an antibody drug conjugate (ADC) having the structure:
Ab-(L-D)ₚ
or a pharmaceutically acceptable salt thereof;
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
each D has the structure of Formula (I): 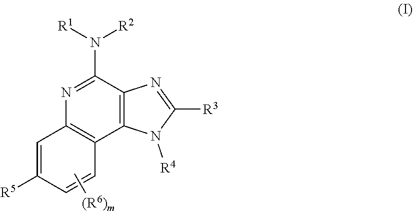
or a pharmaceutically acceptable salt thereof;
wherein:
R¹ is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is (a) the point of covalent attachment to L; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached, form a 3-6 membered heterocyclyl optionally substituted with 1-3 independently selected C₁-C₆ alkyl;
R³ is (a) the point of covalent attachment to L; or (b) selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B},
R⁴ is (a) the point of covalent attachment to L; (b) —OR^{C}; (c) —S(═O)₂R^{C}; (d) —C(═O)NR^{D}R^{E}; (e) —C(═O)OR^{C}; (f) —C(═O)SR^{C}; (g) —C(═S)R^{C}; (h) —PO₃R^{C}; or (j) C₁-C₆ alkyl optionally substituted with:
(i) 1-3 independently selected halogen;
(ii) —OR^{C},
(iii) —SR^{C},
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H;
wherein when R⁴ is (j), the C₁-C₆ alkyl, or a substituent thereof, may be further substituted with the point of covalent attachment to L;
R⁵ is selected from the group consisting of —C(═O)OR^{F}, —NO₂, —CN, —CF₃, —C(═O)NR^{G}R^{H}, —S(O₂)NR^{G}R^{H}, —N(R′)—C(═O)R^{J}, S(O₂)R^{K}, and SO₃R^{K};
each R⁶ is (a) the point of covalent attachment to L; or (b) independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R⁶ is the point of covalent attachment to L;
subscript m is 0, 1, 2, or 3;
each R^{A} and R^{B} is (a) the point of covalent attachment to L, (b) independently selected from the group consisting of hydrogen and C₁-C₆ alkyl; or R^{A} and R^{B} taken together with the nitrogen atom to which they are attached, form a 3-6 membered heterocyclyl optionally substituted with 1-3 independently selected C₁-C₆ alkyl; wherein only one of R^{A} and R^{B} is the point of covalent attachment to L;
R^{C} is (a) the point of covalent attachment to L; or (b) selected from the group consisting of hydrogen, phenyl, and C₁-C₁₀ alkyl optionally substituted with phenyl or 1-3 independently selected halogen;
each R^{D}, R^{E}, R^{G}, and R^{H} are (a) the point of covalent attachment to L; or (b) independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached, form a 3-6 membered heterocyclyl optionally substituted with 1-3 independently selected C₁-C₆ alkyl; wherein only one of R^{D}, R^{E}, R^{G}, and R^{H} is the point of covalent attachment to L;
R^{F} is (a) the point of covalent attachment to L; or (b) selected from the group consisting of hydrogen, trifluoromethyl, C₂-C₆ alkenyl, C₂-C₆ alkynyl, C₃-C₈ cycloalkyl, aryl, aryl(C₁-C₆ alkyl), and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is (a) the point of covalent attachment to L; or (b) independently selected from the group consisting of hydrogen and C₁-C₆ alkyl; wherein only one of R^{I}, R^{J}, and R^{K} is the point of covalent attachment to L;
wherein only one of R¹, R², R³, R⁴, R⁵, R⁶, R^{A}, R^{B}, R^{C}, R^{D}, R^{E}, R^{F}, R^{G}, R^{H}, R^{I}, R^{J}, and R^{K} is the point of covalent attachment to L;
R¹ and R⁴ are each optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide; and
wherein each D has only one point of covalent attachment to L.

272.-274. (canceled)

275. The method of claim 271, wherein the antibody of the ADC targets, and wherein the ADC is configured to internalize within the cell upon binding of the antibody to the surface antigen.

276. (canceled)

277. The method of claim 275, wherein the surface antigen is EphA2.

278.-280. (canceled)

281. The method of claim 271, wherein the linker (L) of the ADC is configured to undergo cleavage subsequent to internalizing within the cell.

282. A compound having the formula L¹-D, or a pharmaceutically acceptable salt thereof, wherein:
L¹ is a linker intermediate; and
D has the structure of Formula (X): 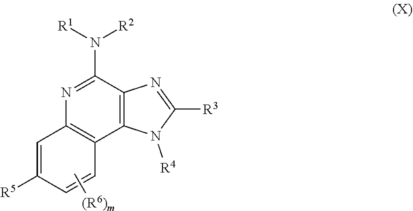
or a pharmaceutically acceptable salt thereof;
wherein:
R¹ is (a) the point of covalent attachment to L¹; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is (a) the point of covalent attachment to L¹; or (b) selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached form a 3-6 membered heterocyclyl optionally substituted with 1-3 independently selected C₁-C₆ alkyl;
R³ is (a) the point of covalent attachment to L¹; or (b) selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R⁴ is (a) the point of covalent attachment to L¹; (b) —OR^{C}; (c) —S(═O)₂R^{C}; (d) —C(═O)NR^{D}R^{E}; (e) —C(═O)OR^{C}; (f) —C(═O)SR^{C}; (g) —C(═S)R^{C}; (h) —PO₃R^{C}; or (j) C₁-C₆ alkyl optionally substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H;
R⁵ is selected from the group consisting of —C(═O)OR^{F}, —NO₂, —CN, —CF₃—C(═O)NR^{G}R^{H}, —S(O₂)NR^{G}R^{H}, —N(R′)—C(═O)R^{J}, —N(R^{I})—S(O₂)R^{K}, and SO₃R^{K};
each R⁶ is (a) the point of covalent attachment to L¹; or (b) independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B}; wherein no more than one R⁶ is the point of covalent attachment to L¹;
wherein when R⁴ is (c), the C₁-C₆ alkyl, or a substituent thereof, may be further substituted with the point of covalent attachment to L¹;
subscript m is 0, 1, 2, or 3;
each R^{A} and R^{B} is (a) the point of covalent attachment to L¹, (b) independently selected from the group consisting of hydrogen and C₁-C₆ alkyl; or R^{A} and R^{B} taken together with the nitrogen atom to which they are attached form a 3-6 membered heterocyclyl optionally substituted with 1-3 independently selected C₁-C₆ alkyl; wherein only one of R^{A} and R^{B} is the point of covalent attachment to L¹;
R^{C} is (a) the point of covalent attachment to L¹; or (b) selected from the group consisting of hydrogen, phenyl, and C₁-C₁₀ alkyl optionally substituted with phenyl or 1-3 independently selected halogen;
each R^{D}, R^{E}, R^{G}, and R^{H} are (a) the point of covalent attachment to L¹; or (b) independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached form a 3-6 membered heterocyclyl optionally substituted with 1-3 independently selected C₁-C₆ alkyl; wherein only one of R^{D}, R^{E}, R^{G}, and R^{H} is the point of covalent attachment to L¹;
R^{F} is (a) the point of covalent attachment to L¹; or (b) selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
each R^{I}, R^{J}, and R^{K} is (a) the point of covalent attachment to L¹; or (b) independently selected from the group consisting of hydrogen and C₁-C₆ alkyl; wherein only one of R^{I}, R^{J}, and R^{K} is the point of covalent attachment to L¹;
each instance of R¹ and R⁴ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide;
wherein only one of R¹, R², R³, R⁴, R⁵, R^{A}, R⁶, R^{B}, R^{C}, R^{D}, R^{E}, R^{F}, R^{G}, R^{H}, R^{I}, R^{J}, and R^{K} is the point of covalent attachment to L¹; and
wherein Formula (X) has only one point of covalent attachment to L¹.

283.-308. (canceled)

309. The compound of claim 282, wherein L¹ has the formula M¹-(A)ₐ-(W)_{w}—(Y)_{y}—(X)ₓ—;
subscript a is 0 or 1;
subscript y is 0 or 1;
subscript w is 0 or 1;
subscript x is 0 or 1;
wherein the sum of subscript a, subscript y, subscript w, and subscript x is greater than or equal to 1;
M¹ is a maleimido, azido, C₁-C₆ alkynyl, cycloalkynyl optionally substituted with 1 or 2 fluoro (e.g., cyclooctynyl or DIFO), sulfhydryl, succinimidyl esters (e.g., N-hydroxysuccinimidyl (NHS) or sulfo-NHS esters), 4-nitrophenyl esters, pentafluorophenyl esters, tetrafluorophenyl esters, anhydrides, acid chlorides, sulfonyl chlorides, isocyanates, isothiocyanates, alpha-haloketones, alpha-O-sulfonate (e.g., mesyl or tosyl) ketones, alkyl hydrazines, hydrazides, and hydroxylamines;
A is a C₂₋₂₀ alkylene optionally substituted with 1-3 R^{a1}; or a 2 to 40 membered heteroalkylene optionally substituted with 1-3 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{c1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{c1}, —C(═O)NR^{d1}R^{c1}, —C(═O)(C₁₋₆ alkyl), and —C(═O)O(C₁₋₆ alkyl);
each R^{d1} and R^{c1} are independently hydrogen or C1₃ alkyl;
W is from 1-12 amino acids or has the structure: 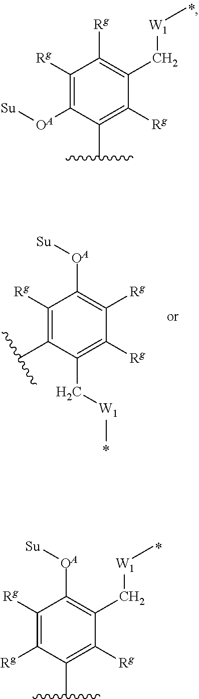
wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—; [symbol]
represents covalent attachment to A or M;
* represents covalent attachment to X, Y, or D;
Y is self-immolative moiety, a non-self-immolative releasable moiety, or a non-cleavable moiety;
X is a C₁-C₆ alkylene or a 3-6 membered heteroalkylene;
L is optionally substituted with a PEG Unit from PEG1 to PEG72.

310.-323. (canceled)

324. An antibody drug conjugate (ADC) having the structure:
Ab-(L-D)p
or a pharmaceutically acceptable salt thereof,
wherein:
Ab is an antibody;
each L is a linker;
wherein each D is conjugated to a linker;
wherein each L is covalently attached to Ab via a sulfur atom of a cysteine residue or an ϵ-amino group of a lysine residue;
subscript p is an integer from 1 to 16;
each D has the structure of Formula (III): 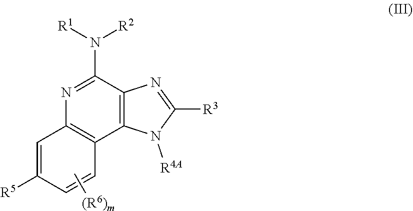
or a pharmaceutically acceptable salt thereof,
wherein:
R¹ is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl;
wherein each C₁-C₆ alkyl, C₂-C₆ alkenyl, C₂-C₆ alkynyl, C₁-C₆ alkanoyl, C₁-C₆ alkoxycarbonyl, C₁-C₆ alkoxythiocarbonyl, C₁-C₆ carbamoyl, C₁-C₆ amidine, C₁-C₆ sulfone, C₁-C₆ thione, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B};
R² is selected from the group consisting of hydrogen, C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl; wherein each C₁-C₆ alkyl, C₂-C₆ alkenyl, C₂-C₆ alkynyl, C₁-C₆ alkanoyl, C₁-C₆ alkoxycarbonyl, C₃-C₆ cycloalkyl, phenyl, and 5-10 membered heteroaryl is optionally substituted with 1-3 substituents independently selected from the group consisting of hydroxyl, halogen, sulfhydryl, cyano, oxiranyl, C₃-C₈ cycloalkyl, phenyl, 5-10 membered heteroaryl, C₁-C₆ alkoxy, C₁-C₆ alkylthio, and —NR^{A}R^{B}; or
R¹ and R², taken together with the nitrogen atom to which they are attached, form a 3-6 membered heterocyclyl optionally substituted with 1-3 independently selected C₁-C₆ alkyl;
R³ is selected from the group consisting of hydrogen, —NR^{A}R^{B}, —C(═O)NR^{A}R^{B}, C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle; wherein each C₁-C₆ alkyl, C₂-C₆ alkenyl, C₂-C₆ alkynyl, C₁-C₆ alkanoyl, C₁-C₆ alkoxycarbonyl, C₁-C₆ alkanoyloxy, C₃-C₆ cycloalkyl, phenyl, 5-10 membered heteroaryl, and 3-12 membered heterocycle is optionally substituted with 1-3 substituents independently selected from the group consisting of hydroxyl, halogen, sulfhydryl, cyano, oxo, oxiranyl, C₃-C₈ cycloalkyl, phenyl, 5-10 membered heteroaryl, C₁-C₆ alkoxy, C₁-C₆ alkylthio, and —NR^{A}R^{B};
R^{4A} is (a) the point of covalent attachment to L or (b) C₁-C₆ alkyl substituted with:
(i) 1-3 independently selected halogen;
(ii) —OR^{C};
(iii) —SR^{C};
(iv) —NH—S(O₂)R^{C};
(v) —OC(═O)R^{C};
(vi) —CO₂H;
(vii) C₁-C₆ alkoxycarbonyl;
(viii) —C(═O)NR^{D}R^{E};
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, or —CO₂H;
wherein when R^{4A} is (b), the C₁-C₆ alkyl, or a substituent thereof, is further substituted with the point of covalent attachment to L;
R⁵ is selected from the group consisting of —C(═O)OR^{F}, —NO₂, —CN, —CF₃—C(═O)NR^{G}R^{H}, —S(O₂)NR^{G}R^{H}, —N(R′)—C(═O)R, —N(R^{I})—S(O₂)R^{K}, and —SO₃R^{K};
each R⁶ is independently selected from the group consisting of halogen, hydroxyl, nitro, cyano, C₁-C₆ alkyl, C₂-C₆ alkenyl, C₂-C₆ alkynyl, C₁-C₆ alkoxy, C₁-C₆ alkanoyl, C₁-C₆ alkanoyloxy, C₁-C₆ alkoxycarbonyl, C₁-C₆ haloalkyl, C₁-C₆ haloalkoxy, and —NR^{A}R^{B};
subscript m is 0, 1, 2, or 3;
each R^{A} and R^{B} independently selected from the group consisting of hydrogen and C₁-C₆ alkyl; or R^{A} and R^{B} taken together with the nitrogen atom to which they are attached, form a 3-6 membered heterocyclyl optionally substituted with 1-3 independently selected C₁-C₆ alkyl;
R^{C} is selected from the group consisting of hydrogen, phenyl, and C₁-C₁₀ alkyl optionally substituted with phenyl or 1-3 independently selected halogen;
each R^{D}, R^{E}, R^{G}, and R^{H} is independently selected from the group consisting of hydrogen, C₁-C₆ alkyl, C₂-C₆ alkenyl, C₃-C₈ cycloalkyl, C₃-C₈ cycloalkyl(C₁-C₆ alkyl)-, aryl, and aryl(C₁-C₆ alkyl)-; or R^{D} and R^{E}, or R^{G} and R^{H}, together with the nitrogen atom to which they are attached, form a 3-6 membered heterocyclyl optionally substituted with 1-3 independently selected C₁-C₆ alkyl;
R^{F} is selected from the group consisting of hydrogen, trifluoromethyl, C₂-C₆ alkenyl, C₂-C₆ alkynyl, C₃-C₈ cycloalkyl, aryl, aryl(C₁-C₆ alkyl)-, and C₁-C₆ alkyl optionally substituted with 1-3 substituents independently selected from the group consisting of halogen, C₁-C₆ alkanoyloxy, C₁-C₆ alkoxy, and C₃-C₈ cycloalkyl;
R¹ is optionally substituted with a solubilizing group (S_{b}) selected from the group consisting of phosphoryl, sulfuryl, nitro, C₅-C₉ monosaccharide, C₁₀-C₁₈ disaccharide, and C₁₅-C₂₇ trisaccharide; and
each R^{I}, R^{J}, and R^{K} is independently selected from the group consisting of hydrogen and C₁-C₆ alkyl.

325. The ADC of claim 324, wherein R^{4A} is C₁-C₆ alkyl substituted with:
(ix) —NR^{D}R^{E};
(x) —[N(C₁-C₆ alkyl)R^{D}R^{E}]⁺;
(xi) -(phenyl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen;
(xii) phenyl substituted with halogen, hydroxyl, C₁-C₆ alkoxy, —C(═O)NR^{D}R^{E} or —CO₂H;
(xiii) -(5-10 membered heteroaryl)C₁-C₆ alkyl, wherein its C₁-C₆ alkyl is substituted with 5-10 membered heteroaryl, —NR^{D}R^{E}, —[N(C₁-C₆ alkyl)R^{D}R^{E}]⁺, or 1-3 independently selected halogen; or
(xiv) 5-10 membered heteroaryl optionally substituted with halogen, —NR^{D}R^{E}, C₁-C₆ alkoxy, —C(═O)NR^{D}R^{E}, —SR^{C}, (C₁-C₆)alkoxycarbonyl, or —CO₂H.

326. The ADC of claim 324, wherein R^{4A} is —CH₂-(phenyl)-(C₁-C₂ alkyl), wherein the C₁-C₂ alkyl is substituted with —NR^{D}R^{E} or —[N(C₁-C₆ alkyl)R^{D}R^{E}]⁺.

327. The ADC of claim 324, wherein R^{D} is the point of covalent attachment to the linker.
